SEQUENCE LISTING (18 sequences)

SEQ ID NO: 1 - DNA, 3978 bp, Artificial Sequence
  Synthetic Construct
atgctttggtgggaggaagtggaggactgctacgagagagaggacgtgcagaagaaaaccttcaccaagtgggtgaacgcccagttcagcaagttcggcaagcagcacatcgagaacctgttcagcgacctgcaggatggcaggagactgctggacctgctggagggcctgaccggccagaagctgcccaaggagaagggcagcaccagagtgcacgccctgaacaacgtgaacaaggccctgagagtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgaggcagagcaccagaaactacccccaggtgaacgtgatcaacttcaccacctcctggagcgacggcctggccctgaacgccctgatccacagccacagacccgacctgttcgactggaacagcgtggtgtgtcagcagagcgccacccagagactggagcacgccttcaacatcgccagataccagctgggcatcgagaagctgctggaccccgaggacgtggacaccacctaccccgacaagaaaagcatcctcatgtacattaccagcctgttccaggtgctgccccagcaggtgtccatcgaggccatccaggaagtggaaatgctgcccaggccccccaaagtgaccaaggaggagcacttccagctgcaccaccagatgcactacagccagcagatcacagtgagcctggcccagggctatgagagaaccagcagccccaagcccagattcaagagctacgcctacacccaggccgcctacgtgaccacctccgaccccaccagaagccccttccccagccagcacctggaggcccccgaggacaagagcttcggcagcagcctgatggagagcgaagtgaacctggacagataccagaccgccctggaggaagtgctgtcctggctgctgagcgccgaggacaccctgcaggcccagggcgagatcagcaacgacgtggaagtggtgaaggaccagttccacacccacgagggctacatgatggatctgaccgcccaccagggcagagtgggcaatatcctgcagctgggcagcaagctgatcggcaccggcaagctgagcgaggacgaggagaccgaagtgcaggagcagatgaacctgctgaacagcagatgggagtgcctgagagtggccagcatggagaagcagagcaacctgcacagagtgctgatggacctgcagaaccagaagctgaaggagctgaacgactggctgaccaagaccgaggagcggaccagaaagatggaggaggagcccctgggccccgacctggaggacctgaagagacaggtgcagcagcacaaagtgctgcaggaggacctggagcaggagcaggtgcgcgtgaacagcctgacccacatggtggtggtcgtggacgagagcagcggcgaccacgccacagccgccctggaagagcagctgaaagtgctgggcgacagatgggccaatatttgtaggtggaccgaggacagatgggtgctgctgcaggaccagcccgacctggcccctggcctgaccaccatcggcgccagccccacccagaccgtgaccctggtgacccagcccgtggtgacaaaggagaccgccatcagcaagctggagatgcccagctccctgatgctggaagtgcccacccaccgcctgctccagcagttccccctggacctggagaagttcctggcctggctgaccgaggccgaaaccaccgccaatgtgctccaggacgccactagaaaggagaggctgctggaggacagcaagggcgtgaaagagctgatgaagcagtggcaggatctgcagggcgaaatcgaggcccacaccgacgtgtaccacaacctggacgagaacagccagaagattctgaggagcctggagggcagcgacgacgccgtcctgctccagaggaggctggacaacatgaacttcaagtggagcgagctgcggaagaagagcctgaacatccggagccacctggaagccagcagcgaccagtggaagagactgcacctgagcctgcaggagctgctggtgtggctgcagctgaaggacgacgagctgagcagacaggcccccatcggcggcgacttccccgccgtgcagaagcagaacgacgtgcaccgggccttcaagagggagctgaaaaccaaggaacccgtgatcatgagcaccctggagacagtgcggatcttcctgaccgagcagcccctggagggactggagaagctgtaccaggagcccagagagctgccccccgaggagagagcccagaacgtgaccaggctgctgagaaagcaggccgaggaagtgaataccgagtgggagaagctgaatctgcacagcgccgactggcagagaaagatcgacgagaccctggagagactccaggaactgcaggaagccaccgacgagctggacctgaagctgagacaggccgaagtgatcaagggcagctggcagcctgtgggcgatctgctgatcgactccctgcaggatcacctggagaaagtgaaggccctgcggggcgagatcgcccccctgaaggagaatgtgagccacgtgaacgacctggccagacagctgaccaccctgggcatccagctgagcccctacaacctgagcacactggaggatctgaacacccggtggaaactgctgcaggtggccgtggaggatagagtgaggcagctgcacgaagcccacagagacttcggccctgcctcccagcacttcctgagcaccagcgtgcagggcccctgggagagagccatctcccccaacaaagtgccctactacatcaaccacgagacccagaccacctgctgggaccaccctaagatgaccgagctgtatcagagcctggccgacctgaacaatgtgcggttcagcgcctacagaaccgccatgaagctgcggagactgcagaaggccctgtgcctggatctgctgagcctgagcgccgcctgcgacgccctggaccagcacaacctgaagcagaatgaccagcccatggacatcctgcagatcatcaactgcctgaccacaatctacgaccggctggaacaggagcacaacaacctggtgaatgtgcccctgtgcgtggacatgtgcctgaattggctgctgaacgtgtacgacaccggcaggaccggcagaatccgcgtgctgagcttcaagaccggcatcatcagcctgtgcaaggcccacctggaggataagtaccgctacctgttcaagcaggtggccagcagcaccggcttctgcgatcagaggagactgggcctgctgctgcacgatagcatccagatccctaggcagctgggcgaagtggccagctttggcggcagcaacatcgagccctctgtgaggagctgcttccagttcgccaacaacaagcccgagatcgaggccgccctgttcctggactggatgaggctggagcctcagagcatggtgtggctgcctgtgctgcacagagtggccgccgccgagaccgccaagcaccaggccaagtgcaatatctgcaaggagtgccccatcatcggcttccggtacaggagcctgaagcacttcaactacgacatctgccagagctgctttttcagcggcagagtggccaagggccacaaaatgcactaccccatggtggagtactgcacccccaccacctccggcgaggatgtgagagacttcgccaaagtgctgaagaataagttccggaccaagcggtactttgccaagcaccccaggatgggctacctgcccgtgcagaccgtgctggaaggcgacaacatggagacctga

SEQ ID NO: 2 - protein, 1325 aa, Artificial Sequence
  Synthetic Construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDQPDLAPGLTTIGASPTQTVTLVTQPVVTKETAISKLEMPSSLMLEVPTHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMET

SEQ ID NO: 3 - DNA, 606 bp, Artificial Sequence
  Synthetic Construct
gaattcggtaccccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagcggccgatccgccacc

SEQ ID NO: 4 - DNA, 759 bp, Artificial Sequence
  Synthetic Construct
gaattcggtaccccactacgggtctaggctgcccatgtaaggaggcaaggcctggggacacccgagatgcctggttataattaaccccaacacctgctgcccccccccccccaacacctgctgcctctaaaaataaccctgtccctggtggatcccctgcatgccccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagcggccgatccgccacc

SEQ ID NO: 5 - DNA, 577 bp, Artificial Sequence
  Synthetic Construct
ccactacgggtctaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcga

SEQ ID NO: 6 - DNA, 67 bp, Artificial Sequence
  Synthetic Construct
tgaggagctcgagaggcctaataaagagctcagatgcatcgatcagagtgtgttggttttttgtgtg

SEQ ID NO: 7 - DNA, 145 bp, Artificial Sequence
  Synthetic Construct
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 8 - DNA, 145 bp, Artificial Sequence
  Synthetic Construct
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 9 - DNA, 54 bp, Artificial Sequence
  Synthetic Construct
aggcctaataaagagctcagatgcatcgatcagagtgtgttggttttttgtgtg

SEQ ID NO: 10 - DNA, 4955 bp, Artificial Sequence
  Synthetic Construct
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtaccccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagcggccgccaccatgctttggtgggaggaagtggaggactgctacgagcgggaggacgtgcagaagaaaaccttcaccaagtgggtgaacgcccagttcagcaagttcggcaagcagcacatcgagaacctgttcagcgacctgcaggacggcaggcggctgctggacctcctggaaggcctgaccggccagaagctgcccaaagagaagggcagcaccagggtgcacgccctgaacaacgtgaacaaggccctgagggtgctgcagaacaacaacgtggacctggtgaacatcggcagcaccgacatcgtggacggcaaccacaagctgaccctgggcctgatctggaacatcatcctgcactggcaggtcaagaacgtgatgaagaacatcatggccggcctgcagcagaccaacagcgagaagatcctgctgtcctgggtgcggcagagcaccaggaactacccccaggtcaacgtgatcaacttcaccacctcttggagcgacggcctggccctgaacgccctgatccacagccacaggcccgacctgttcgactggaacagcgtggtgtgccagcagagcgccacccagaggctggaacacgccttcaacatcgccagataccagctgggcatcgagaagctgctggatcccgaggacgtggacaccacctaccccgacaagaaaagcatcctcatgtacatcaccagcctgttccaggtgctgccccagcaggtgtccatcgaggccatccaggaagtggagatgctgcccaggccccccaaggtcaccaaagaggaacacttccagctgcaccaccagatgcactacagccagcagatcaccgtgagcctggcccagggctacgagaggaccagcagccccaagcccaggttcaagagctacgcctacacccaggccgcctacgtgaccacctccgaccccaccaggtcccccttccccagccagcatctcgaagcccccgaggacaagagcttcggcagcagcctgatggaaagcgaggtgaacctggacagataccagaccgccctggaagaagtgctgtcttggctgctgtccgccgaggacaccctgcaggcccagggcgagatcagcaacgacgtggaggtcgtgaaggaccagttccacacccacgagggctacatgatggacctgaccgcccaccagggcagagtgggcaacatcctgcagctgggcagcaagctgatcggcaccggcaagctgtccgaggacgaggaaaccgaggtgcaggaacagatgaacctgctgaacagcagatgggagtgcctgagggtggccagcatggaaaagcagagcaacctgcacagggtgctgatggatctgcagaaccagaagctcaaagagctgaacgactggctgaccaagaccgaggaaaggacccggaagatggaagaggaacccctgggccccgatctcgaagatctgaagaggcaggtgcagcagcacaaggtgctgcaggaagatctcgaacaggaacaggtccgggtcaacagcctgacccacatggtcgtggtggtggacgagagcagcggcgaccacgccaccgctgccctggaagagcagctgaaggtgctgggcgacagatgggccaacatctgccggtggaccgaggacagatgggtcctcctgcaggaccagcccgacctggcccctggcctgacaaccatcggcgccagccccacccagaccgtgaccctggtgacccagcccgtggtgaccaaagagaccgccatcagcaagctggaaatgcccagctccctgatgctggaagtgcccacccacaggctcctccagcagttccccctggacctggaaaagttcctggcctggctgaccgaggccgagaccaccgccaacgtgctgcaggacgccaccaggaaagagaggctgctggaagatagcaagggcgtgaaagagctgatgaagcagtggcaggacctgcagggggagattgaggcccacaccgacgtgtaccacaacctggacgagaacagccagaaaatcctgagaagcctggaaggcagcgacgacgccgtgctgctgcagaggcggctggacaacatgaacttcaagtggagcgagctgaggaagaagagcctgaacatcaggtcccatctggaagccagcagcgaccagtggaagaggctgcacctgagcctgcaggaactgctcgtctggctgcagctgaaagacgacgagctgtccaggcaggcccccatcggcggcgacttccccgccgtgcagaaacagaacgacgtgcacagggccttcaagcgggagctgaaaaccaaagagcccgtgatcatgagcaccctggaaaccgtgaggatcttcctgaccgagcagcccctggaaggactggaaaagctgtaccaggaacccagagagctgccccccgaggaacgggcccagaacgtgaccaggctgctgagaaagcaggccgaggaagtgaacaccgagtgggagaagctgaacctgcactccgccgactggcagaggaagatcgacgagaccctggaaaggctccaggaactgcaggaagccaccgacgagctggacctgaagctgagacaggccgaggtgatcaagggcagctggcagcccgtgggcgacctgctgatcgactccctgcaggatcacctggaaaaagtgaaggccctgcggggcgagatcgcccccctgaaagagaacgtcagccacgtcaacgacctggccaggcagctgaccaccctgggcatccagctgtccccctacaacctgtccaccctggaagatctgaacacaaggtggaagctgctgcaggtggccgtggaggacagagtgaggcagctgcacgaggcccacagggacttcggccctgcctcccagcacttcctgagcaccagcgtgcagggcccctgggagagggccatctcccccaacaaggtgccctactacatcaaccacgagacccagaccacctgctgggaccaccctaagatgaccgagctgtaccagtccctggccgacctgaacaatgtgcggttcagcgcctaccggaccgccatgaagctgaggcggctgcagaaagccctgtgcctggatctgctgtccctgagcgccgcctgcgacgccctggaccagcacaacctgaagcagaacgaccagcccatggatatcctgcagatcatcaactgtctgaccaccatctacgacaggctggaacaggaacacaacaacctggtcaacgtgcccctgtgcgtggacatgtgcctgaactggctgctgaacgtgtacgacaccggcaggaccggccggatcagggtgctgtccttcaagaccggcatcatcagcctgtgcaaggcccacctggaagataagtaccgctacctgttcaagcaggtggccagctctaccggcttctgcgaccagcggaggctgggcctgctgctgcacgacagcatccagatcccccggcagctgggcgaggtggcctccttcggcggcagcaacatcgagcccagcgtgcggagctgcttccagttcgccaacaacaagcccgagatcgaggccgccctgttcctggactggatgcggctggaaccccagagcatggtctggctgcccgtgctgcacagagtggctgccgccgagaccgccaagcaccaggccaagtgcaacatctgcaaagagtgccccatcatcggcttcaggtacagaagcctgaagcacttcaactacgacatctgccagagctgtttcttcagcggcagggtggccaagggccacaaaatgcactaccccatggtggagtactgcacccccaccacctccggcgaggacgtgagggacttcgccaaggtgctgaagaataagttccggaccaagcggtacttcgccaaacaccccaggatgggctacctgcccgtgcagaccgtgctggaaggcgacaacatggaaacctgataacacgcgtcgactcgagaggcctaataaagagctcagatgcatcgatcagagtgtgttggttttttgtgtgagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 11 - protein, 736 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 12 - DNA, 4955 bp, Artificial Sequence
  Synthetic Construct
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtaccccactacgggtctaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagcggccgatccgccaccatgctttggtgggaggaagtggaggactgctacgagagagaggacgtgcagaagaaaaccttcaccaagtgggtgaacgcccagttcagcaagttcggcaagcagcacatcgagaacctgttcagcgacctgcaggatggcaggagactgctggacctgctggagggcctgaccggccagaagctgcccaaggagaagggcagcaccagagtgcacgccctgaacaacgtgaacaaggccctgagagtgctgcagaacaacaacgtggacctggtgaacatcggcagcaccgacatcgtggacggcaaccacaagctgaccctgggcctgatctggaacatcatcctgcactggcaggtgaagaacgtgatgaagaacatcatggccggcctgcagcagaccaacagcgagaagatcctgctgagctgggtgaggcagagcaccagaaactacccccaggtgaacgtgatcaacttcaccacctcctggagcgacggcctggccctgaacgccctgatccacagccacagacccgacctgttcgactggaacagcgtggtgtgtcagcagagcgccacccagagactggagcacgccttcaacatcgccagataccagctgggcatcgagaagctgctggaccccgaggacgtggacaccacctaccccgacaagaaaagcatcctcatgtacattaccagcctgttccaggtgctgccccagcaggtgtccatcgaggccatccaggaagtggaaatgctgcccaggccccccaaagtgaccaaggaggagcacttccagctgcaccaccagatgcactacagccagcagatcacagtgagcctggcccagggctatgagagaaccagcagccccaagcccagattcaagagctacgcctacacccaggccgcctacgtgaccacctccgaccccaccagaagccccttccccagccagcacctggaggcccccgaggacaagagcttcggcagcagcctgatggagagcgaagtgaacctggacagataccagaccgccctggaggaagtgctgtcctggctgctgagcgccgaggacaccctgcaggcccagggcgagatcagcaacgacgtggaagtggtgaaggaccagttccacacccacgagggctacatgatggatctgaccgcccaccagggcagagtgggcaatatcctgcagctgggcagcaagctgatcggcaccggcaagctgagcgaggacgaggagaccgaagtgcaggagcagatgaacctgctgaacagcagatgggagtgcctgagagtggccagcatggagaagcagagcaacctgcacagagtgctgatggacctgcagaaccagaagctgaaggagctgaacgactggctgaccaagaccgaggagcggaccagaaagatggaggaggagcccctgggccccgacctggaggacctgaagagacaggtgcagcagcacaaagtgctgcaggaggacctggagcaggagcaggtgcgcgtgaacagcctgacccacatggtggtggtcgtggacgagagcagcggcgaccacgccacagccgccctggaagagcagctgaaagtgctgggcgacagatgggccaatatttgtaggtggaccgaggacagatgggtgctgctgcaggaccagcccgacctggcccctggcctgaccaccatcggcgccagccccacccagaccgtgaccctggtgacccagcccgtggtgacaaaggagaccgccatcagcaagctggagatgcccagctccctgatgctggaagtgcccacccaccgcctgctccagcagttccccctggacctggagaagttcctggcctggctgaccgaggccgaaaccaccgccaatgtgctccaggacgccactagaaaggagaggctgctggaggacagcaagggcgtgaaagagctgatgaagcagtggcaggatctgcagggcgaaatcgaggcccacaccgacgtgtaccacaacctggacgagaacagccagaagattctgaggagcctggagggcagcgacgacgccgtcctgctccagaggaggctggacaacatgaacttcaagtggagcgagctgcggaagaagagcctgaacatccggagccacctggaagccagcagcgaccagtggaagagactgcacctgagcctgcaggagctgctggtgtggctgcagctgaaggacgacgagctgagcagacaggcccccatcggcggcgacttccccgccgtgcagaagcagaacgacgtgcaccgggccttcaagagggagctgaaaaccaaggaacccgtgatcatgagcaccctggagacagtgcggatcttcctgaccgagcagcccctggagggactggagaagctgtaccaggagcccagagagctgccccccgaggagagagcccagaacgtgaccaggctgctgagaaagcaggccgaggaagtgaataccgagtgggagaagctgaatctgcacagcgccgactggcagagaaagatcgacgagaccctggagagactccaggaactgcaggaagccaccgacgagctggacctgaagctgagacaggccgaagtgatcaagggcagctggcagcctgtgggcgatctgctgatcgactccctgcaggatcacctggagaaagtgaaggccctgcggggcgagatcgcccccctgaaggagaatgtgagccacgtgaacgacctggccagacagctgaccaccctgggcatccagctgagcccctacaacctgagcacactggaggatctgaacacccggtggaaactgctgcaggtggccgtggaggatagagtgaggcagctgcacgaagcccacagagacttcggccctgcctcccagcacttcctgagcaccagcgtgcagggcccctgggagagagccatctcccccaacaaagtgccctactacatcaaccacgagacccagaccacctgctgggaccaccctaagatgaccgagctgtatcagagcctggccgacctgaacaatgtgcggttcagcgcctacagaaccgccatgaagctgcggagactgcagaaggccctgtgcctggatctgctgagcctgagcgccgcctgcgacgccctggaccagcacaacctgaagcagaatgaccagcccatggacatcctgcagatcatcaactgcctgaccacaatctacgaccggctggaacaggagcacaacaacctggtgaatgtgcccctgtgcgtggacatgtgcctgaattggctgctgaacgtgtacgacaccggcaggaccggcagaatccgcgtgctgagcttcaagaccggcatcatcagcctgtgcaaggcccacctggaggataagtaccgctacctgttcaagcaggtggccagcagcaccggcttctgcgatcagaggagactgggcctgctgctgcacgatagcatccagatccctaggcagctgggcgaagtggccagctttggcggcagcaacatcgagccctctgtgaggagctgcttccagttcgccaacaacaagcccgagatcgaggccgccctgttcctggactggatgaggctggagcctcagagcatggtgtggctgcctgtgctgcacagagtggccgccgccgagaccgccaagcaccaggccaagtgcaatatctgcaaggagtgccccatcatcggcttccggtacaggagcctgaagcacttcaactacgacatctgccagagctgctttttcagcggcagagtggccaagggccacaaaatgcactaccccatggtggagtactgcacccccaccacctccggcgaggatgtgagagacttcgccaaagtgctgaagaataagttccggaccaagcggtactttgccaagcaccccaggatgggctacctgcccgtgcagaccgtgctggaaggcgacaacatggagacctgatgaggagctcgagaggcctaataaagagctcagatgcatcgatcagagtgtgttggttttttgtgtgagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 13 - DNA, 106 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtgg

SEQ ID NO: 14 - DNA, 113 bp, Artificial Sequence
  Synthetic Construct
cgcccgacgcccgggctttgcccgggcggcctcagtgagcgagcgagcgcgccagctggcgtaatagcgaagaggcccgcaccgatcgcccttcccaacagttgcgcagcctg

SEQ ID NO: 15 - protein, 1035 aa, Artificial Sequence
  Synthetic Construct
MPEQERQITAREGASRKILSKLSLPTRAWEPAMKKSFAFDNVGYEGGLDGLGPSSQPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 16 - DNA, 305 bp, Artificial Sequence
  Synthetic Construct
cgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaa

SEQ ID NO: 17 - DNA, 56 bp, Artificial Sequence
  Synthetic Construct
aataaagacctcttattttcattcatcaggtgtggttggtttttttgtgtgggggc

SEQ ID NO: 18 - protein, 736 aa, Artificial Sequence
  Synthetic Construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

(19) United States
(12) Patent Application Publication
(10) Pub. No.: US 2023/0399656 A1
(43) Pub. Date: Dec. 14, 2023

(54) Methods for Purification of AAV Vectors by Anion Exchange Chromatography

(71) Applicant: Pfizer Inc., New York, NY (US)

(72) Inventors: Alexander BERRILL, Ballwin, MO (US); William S. KISH, Holly Springs, NC (US); John R. LIGHTHOLDER, Franklin, MA (US); Matthew K. ROACH, Cary, NC (US); William B. WELLBORN, White River Junction, VT (US); Tamara ZEKOVIC, Cary, NC (US)

(21) Appl. No.: 18/250,252

(22) PCT Filed: Nov. 1, 2021
(86) PCT No.: PCT/IB2021/060095

§ 371 Date: Apr. 24, 2023

Related U.S. Application Data
(60) Provisional application No. 63/253,215, filed on Oct. 7, 2021.
(60) Provisional application No. 63/217,194, filed on Jun. 30, 2021.
(60) Provisional application No. 63/109,049, filed on Nov. 3, 2020.

(51) Int. Cl. C12N 15/86; B01D 15/36; B01D 15/42; B01D 15/16

(52) U.S. Cl. CPC C12N 15/86; B01D 15/363; B01D 15/424; B01D 15/166; C12N 2750/14151; C12N 2750/14143

(57) ABSTRACT

The present disclosure provides methods for purifying a recombinant AAV (rAAV) vector from a solution by anion-exchange chromatography (AEX) to produce an eluate enriched for full capsids and depleted of empty capsids.

### CROSS-REFERENCE TO RELATED APPLICATIONS

This application claims priority to U.S. Provisional Patent Application No. 63/253,215, filed Oct. 7, 2021, U.S. Provisional Patent Application No. 63/217,194, filed Jun. 30, 2021, and U.S. Provisional Patent Application No. 63/109,049, filed Nov. 3, 2020, the contents of each of which are incorporated herein by reference in their entirety.

### SEQUENCE LISTING

The instant application contains a Sequence Listing which has been submitted electronically in ASCII format and is hereby incorporated by reference in its entirety. Said ASCII copy, created Oct. 15, 2021, is named PC072555A_Sequence_Listing_ST25.txt and is 1,048,576 bytes in size.

### FIELD OF INVENTION

The present invention relates to the purification of AAV, and in particular recombinant AAV (rAAV) vectors by anion exchange chromatography.

### BACKGROUND

Gene therapy, using a recombinant AAV (rAAV) vector to deliver a therapeutic transgene, has the potential to treat a wide range of serious diseases for which no cure, and in many cases, limited treatment exists (Wang et al. (2019) Nature Reviews 18:358-378). Manufacturing of gene therapy vectors is complex and requires specialized methods to purify the therapeutic rAAV vector from host cell impurities, and from viral capsids that do not contain a complete vector genome encoding the therapeutic transgene. In addition to development of a purification method that produces a clinical grade rAAV vector composition of high purity and with a good safety and efficacy profile, the purification method must also be scalable to high volume rAAV production to meet patient needs.

Ultracentrifugation using a cesium chloride gradient sedimentation is a robust method for removal of host cell protein and DNA, as well as separation of viral capsids that are empty (i.e., that do not contain a vector genome), partially packaged (also referred to as “intermediate capsids” and which contain a partial vector genome and/or non-transgene-related DNA) or fully packaged vectors (also referred to as “full capsids” and which contain a complete vector genome) (Burnham et al. (2015) Hum. Gene Ther. Meth. 26:228-245). However, cesium chloride gradient purification is laborious, time consuming and not amenable to large scale manufacturing.

Ultracentrifugation using an iodixanol gradient is less labor intensive but generally results in vector yields of lower purity (Hermens et al. Hum. Gene Ther. (1999) Chromatographic methods including affinity and/or ion exchange chromatography have proven useful for large-scale production of clinical grade rAAV, including separation of empty viral capsids from full rAAV vectors.

Empty capsids are produced by the host cells that produce and package the recombinant vector genome in the viral capsid. An excess of empty capsids are produced relative to full vectors in most mammalian expression systems, and various systems generate 1-30% full vectors (Penaud-Budloo et al. Molecular Therapy, Methods & Clinical Dev (2018) 8:166-180). The production of empty capsids may be due to an imbalance in the ratio of plasmids encoding the transgene to that of the rep/cap genes. The presence of empty capsids in a drug product may cause an undesirable immune response and/or compete with the recombinant vectors for binding sites on target cells.

Certain anion-exchange chromatographic methods, employing acetate buffers and resins such as POROS™ 50 HQ and Q-Sepharose XL, have been used to separate empty capsids from rAAV2 vector pools by relying on the slightly less anionic character of the empty capsids as compared to full vectors (U.S. Pat. No. 7,261,544; Qu et al. (2007) J. Virol. Meth. 140(1):183-192). A similar approach used a combination of affinity and ion exchange chromatography (IEX) and a 10 mM to 300 mM Tris acetate gradient at pH 8 with POROS™ 50 HQ resin to enrich for full AAV vectors of various serotypes (Nass et al. (2018) Molec. Thera. Meth. & Clin. Dev. 9:33-46). Other studies have identified buffers and conditions useful for chromatographic separation of empty capsids from full AAV vectors. For instance, Urabe determined that AAV1 material could be diluted with a Tris-HCl buffer comprising MgCl₂ and glycerol for load on an anion exchange chromatography (AEX) column and that solutions comprising antichaotropic ions were effective elution buffers for separation of the empty AAV1 capsids from full vectors (Urabe et al. (2006) Molec. Ther. 13(4):823-828). Others have described dilution (e.g., 50-fold) of affinity chromatography eluates and the use of shallow gradient elution (e.g., 20 mM to 180 mM NaCl) from a monolithic support in AEX methods for the separation of empty capsids from full AAV vectors (US 2019-0002841; US 2019-0002842; US 2019-0002843; US 2018-0002844). However, these methods also employ a high pH (9.8 to 10.2) which can lead to deamidation and/or aggregation of rAAV vector and may lead to a decrease in the therapeutic potency.

Processes using a combination of methods including, for example and in no particular order, tangential flow filtration (TFF) of a host cell supernatant, precipitation of the capsid material (including rAAV vectors and empty capsids) using ammonium sulfate, AEX chromatography and size-exclusion chromatography have been developed to separate the rAAV from empty capsids (Tomono et al. (2018) Molec. Ther. Meth. Clin. Dev. 11:180-190).

There remains a need for methods for preparation of clinical grade rAAV vector (e.g., rAAV9) with optimal purity, potency and consistency. These methods include the separation of rAAV comprising a vector genome with therapeutic transgene from empty AAV capsid at a scale necessary to meet the clinical need for treatment of disease (e.g., Duchenne Muscular Dystrophy (DMD), Friedreich's Ataxia (FA)).

### SUMMARY

The present disclosure provides an improved AEX method of purification of rAAV vectors including, but not limited to the separation of full rAAV vectors (e.g., rAAV9 vectors) from empty capsids. Such purified full rAAV vectors are suitable for production of a drug product for administration to a human subject, such as a subject with DMD. The disclosure also provides a novel method of preparation of a chromatography eluate comprising rAAV vectors (e.g., from affinity chromatography) for further purification by AEX. The disclosure also provides methods for regenerating an AEX stationary phase that allow the stationary phase to be used in multiple chromatography runs while maintaining the integrity of the process (e.g., successful purification of rAAV vectors, the separation of full vectors from empty capsids) while reducing manufacturing costs.

Those skilled in the art will recognize, or be able to ascertain using no more than routine experimentation, many equivalents to the specific embodiments of the invention described herein. Such equivalents are intended to be encompassed by the following embodiments (E).

E1. A method of purifying an rAAV vector by AEX, the method comprising a step of:
i) loading a solution comprising the rAAV vector to be purified onto a stationary phase in a column;
ii) performing gradient elution of material from the stationary phase in the column wherein a percentage of a first gradient elution buffer is varied in a manner inversely proportional to variation in a percentage of a second gradient elution buffer;
iii) collecting at least one fraction of eluate from the column during the gradient elution beginning when the absorbance of a column flow-through reaches an absorbance threshold.
   E2. The method of E1, wherein loading the solution comprising the rAAV vector onto the column comprises application of 2.5×10¹⁵ to 3.0×10¹⁷ vector genome (VG)/L of column volume onto the column.
   E3. The method of E1 or E2, wherein loading comprises application of 8.0×10¹² to 2.0×10¹⁸ total VG to the column.
   E4. The method of any one of E1-E3, wherein loading comprises application of a diluted, and optionally filtered solution comprising 2.6×10¹² to 6.8×10¹³ VG/mL of column volume onto a column (e.g., a 6.4 L column) as measured by qPCR analysis of a transgene sequence within the vector genome.
   E5. The method of any one of E1-E4, wherein loading comprises application of a diluted, and optionally filtered solution comprising 5×10¹³ to 1.3×10¹⁴ VG/mL of column volume onto a column (e.g., a 1.3 L column) as measured by qPCR analysis of the ITR sequences within the vector genome.
   E6. The method of any one of E1-E5, wherein the rAAV vector is produced in a vessel, and wherein the volume of the vessel is about 1 L, about 50 L, about 100 L, about 250 L, about 500 L, about 1000 L, about 2000 L or greater.
   E7. The method of any one of E1-E6, wherein the vessel is a single use bioreactor (SUB).
   E8. The method of any one of E1-E7, wherein the solution comprising the rAAV vector is selected from the group consisting of an affinity eluate, a supernatant from a cell lysate and a post-harvest solution that has been diluted and optionally filtered prior to loading.
   E9. The method of any one of E1-E8, wherein the solution comprising the rAAV vector is an affinity eluate that has been diluted and optionally filtered prior to loading.
   E10. The method of E1-E9, wherein the solution has undergone at least one other purification or processing step.
   E11. The method of E10, wherein the at least one other purification or processing step is selected from the group consisting of cell lysis, flocculation, filtration, chromatography (e.g., affinity chromatography), dilution, pH adjustment, conductivity adjustment and a combination thereof.
   E12. The method of any one of E1-E12, wherein the solution comprising the rAAV vector is an affinity eluate resulting from affinity chromatography purification of a rAAV vector produced in a single use bioreaction (SUB) with a volume.
   E13. The method of E12, wherein the SUB has a volume of about 1L to about 2000L.
   E14. The method of any one of E1-13, wherein the stationary phase is an AEX stationary phase.
   E15. The method of any one of E1-E14, wherein the stationary phase is positively charged.
   E16. The method of any one of E1-E15, wherein the stationary phase is a polystyrenedivinylbenzene particle with covalently bound quaternized polyethyleneimine, and optionally wherein the stationary phase is POROS™ 50 HQ.
   E17. The method of any one of E1-E16, further comprising application of a load chase solution to the AEX stationary phase in the column, optionally after loading the solution comprising the rAAV vector onto the stationary phase.
   E18. The method of E17, wherein 1 to 15 column volumes (CV) of the load chase solution comprising a buffering agent (e.g., Tris, BIS-Tris propane, diethanolamine, diethylamine, tricine, triethanolamine and/or bicine) are applied to the stationary phase in the column.
   E19. The method of E17 or E18, wherein the load chase solution comprises about 10 mM to 30 mM (e.g., about 20 mM) Tris, pH 8-10.
   E20. The method of any one of E1-E19, further comprising pre-use flushing of a stationary phase in a column.
   E21. The method of E20, wherein the pre-use flushing of the stationary phase in the column precedes loading the solution comprising the rAAV vector onto the column.
   E22. The method of E20 or E21, wherein the pre-use flushing of the stationary phase in the column comprises application of water for injection to the stationary phase.
   E23. The method of any one of E1-E22, further comprising sanitizing the stationary phase in the column.
   E24. The method of E23, wherein sanitizing the stationary phase in the column precedes loading the solution comprising the rAAV vector onto the column.
   E25. The method of E23 or E24, wherein sanitizing the stationary phase in the column comprises application of a solution comprising NaOH to the stationary phase.
   E26. The method of any one of E23-E25, wherein sanitizing the stationary phase in the column comprises application of a solution comprising about 0.1 M to about 1.0 M (e.g., about 0.5 M) NaOH to the stationary phase.
   E27. The method of any one of E23-E26, wherein sanitizing the stationary phase in the column comprises application of about 5 CV to about 10 CV, or about 14.4 CV to about 17.6 CV of the solution comprising about 0.1 M to about 1.0 M NaOH to the stationary phase.
   E28. The method of any one of E23-E27, wherein sanitizing the stationary phase in the column is by upward flow.
   E29. The method of any one of E1-E28, further comprising regenerating the stationary phase in the column.
   E30. The method of E29, wherein regenerating the stationary phase in the column precedes loading the solution comprising the rAAV vector onto the column.
   E31. The method of E29 or E30, wherein regenerating the stationary phase in the column comprises application of a solution comprising a component selected from the group consisting of a salt, a buffering agent and a combination thereof to the stationary phase.
   E32. The method of any one of E29-E31, wherein regenerating the stationary phase in the column comprises application of a solution comprising about 1 M to about 3 M (e.g., about 2 M) NaCl, about 50 mM to about 150 mM (e.g., about 100 mM) Tris, pH 8 to 10 (e.g., about 9) to the stationary phase.
   E33. The method of any one of E29-E32, wherein regenerating the stationary phase in the column comprises application of a solution comprising about 50 mM to about 150 mM (e.g., about 100 mM) Tris, pH 9 to the stationary phase.
   E34. The method of any one of E29-E33, wherein regenerating the stationary phase in the column comprises application of 4.5 to 5.5 CV of the solution comprising about 100 mM Tris, pH 9 to the stationary phase.
   E35. The method of any one of E29-E34, wherein regenerating the stationary phase in the column is performed more than once.
   E36. The method of any one of E1-E35, further comprising equilibration of the stationary phase in the column.
   E37. The method of E36, wherein equilibration of the stationary phase in the column precedes or follows loading the solution comprising the rAAV vector onto the column.
   E38. The method of E36 or E37, wherein equilibration of the stationary phase in the column comprises application of an equilibration buffer comprising at least one component selected from the group consisting of a buffering agent, a salt, an amino acid, a detergent and a combination thereof, to the stationary phase.
   E39. The method of E38, wherein the buffering agent is selected from the group consisting of Tris, BIS-Tris propane, diethanolamine, diethylamine, tricine, triethanolamine and/or bicine and a combination thereof.
   E40. The method of E36 or E39, wherein the salt is selected from the group consisting of sodium chloride, sodium acetate, ammonium acetate, magnesium chloride, sodium sulfate and a combination thereof.
   E41. The method of any one of E38-E40, wherein the salt is sodium acetate.
   E42. The method of any one of E38-E41, wherein the amino acid is selected from the group consisting of histidine, arginine, glycine, citrulline and a combination thereof.
   E43. The method of any one of E38-E42, wherein the amino acid is histidine.
   E44. The method of E38-E43, wherein the detergent is selected from the group consisting of poloxamer 188 (P188), Triton X-100, polysorbate 80 (PS80), Brij-35, nonyl phenoxypolyethoxylethanol (NP-40) and a combination thereof.
   E45. The method of any one of E38-E44, wherein the detergent is P188.
   E46. The method of any one of E36-E45, wherein equilibration of the stationary phase in the column comprises application of an equilibration buffer comprising about 50 mM to about 150 mM (e.g., about 100 mM Tris), pH about 8 to 10 (e.g., about 9) to the stationary phase.
   E47. The method of any one of E36-E46, wherein equilibration of the stationary phase in the column comprises application of an equilibration buffer comprising about 50 mM to about 150 mM (e.g., about 100 mM Tris), about 250 mM to about 750 mM (e.g., about 500 mM) sodium acetate, about 0.005 to about 0.015% (e.g., about 0.01%) P188, pH about 8 to about 10 (e.g., about 8.9) to the stationary phase.
   E48. The method of any one of E36-E47, wherein equilibration of the stationary phase in the column comprises application of an equilibration buffer comprising about 100 mM to about 300 mM (e.g., about 200 mM) histidine, about 100 mM to about 300 mM (e.g., about 200 mM Tris), about 0.1% to about 1.0% (e.g., about P188, pH about 8 to about 10 (e.g., about 8.8) to the stationary phase.
   E49. The method of any one of E36-E48, wherein equilibration of the stationary phase in the column comprises application of an equilibration buffer comprising about 50 mM to about 150 mM (e.g., about 100 mM) Tris, 0.005% to about 0.015% (e.g., about 0.01%) P188, pH about 8 to about 10 (e.g., about 8.9) to the stationary phase.
   E50. The method of any one of E36-E49, wherein equilibration of the stationary phase in the column comprises application of greater than about 4.5 CV of an equilibration buffer to the stationary phase.
   E51. The method of any one of E36-E50, wherein equilibration of the stationary phase in the column comprises application of 4.5 to 5.5 CV of an equilibration buffer to the stationary phase.
   E52. The method of any one of E36-E51, wherein equilibration of the stationary phase in the column is performed more than once.
   E53. The method of any one of E36-E52, wherein at least one equilibration buffer is applied to the stationary phase prior to loading the solution comprising the rAAV vector onto the column; and wherein at least one equilibration buffer is applied to the stationary phase after loading the solution comprising the rAAV vector onto the column.
   E54. The method of any one of E1-E53, comprising performing gradient elution of material from the stationary phase in the column.
   E55. The method of E54, wherein the gradient elution comprises application of 10 to 60 CV of at least two different solutions (e.g., gradient elution buffers), or a mixture of the two, to the stationary phase, and wherein over the course of the gradient elution, a percentage of a first solution is varied in a manner inversely proportional to a percentage of a second solution.
   E56. The method of E54 or E55, wherein the at least two different solutions (e.g., a first gradient elution buffer, a second gradient elution buffer) comprise a component selected from the group consisting of a buffering agent, a salt, a detergent, and a combination thereof.
   E57. The method of E56, wherein the buffering agent is selected from the group consisting of Tris, BIS-Tris propane, diethanolamine, diethylamine, tricine, triethanolamine and/or bicine.
   E58. The method of E56 or E57, wherein the salt is selected from the group consisting of sodium chloride, sodium acetate, ammonium acetate, magnesium chloride, sodium sulfate and a combination thereof.
   E59. The method of any one of E56-E58, wherein the detergent is selected from the group consisting of poloxamer 188 (P188), Triton X-100, polysorbate 80 (PS80), Brij-35, nonyl phenoxypolyethoxylethanol (NP-40) and a combination thereof.
   E60. The method of any one of E55-E59, wherein the at least two different solutions have different pH, salt concentration, conductivity and/or modifier concentration.
   E61. The method of any one of E55-E60, wherein the first solution (e.g., buffer A) comprises about 50 mM to about 150 mM (e.g., about 100 mM) Tris, about 0.005% to about 0.015% (e.g., about 0.01%) P188, about pH 8.5 to 9.5 (e.g., about 8.9).
   E62. The method of any one of E55-E61, wherein the second solution (e.g., buffer B) comprises about 400 mM to about 600 mM (e.g., about 500 mM) sodium acetate, about 50 mM to about 150 mM (e.g., about 100 mM) Tris, about 0.005% to about 0.015% (e.g., about 0.01%) P188, about pH 8.5 to 9.5 (e.g., about 8.9).
   E63. The method of any one of E55-E62, wherein performing the gradient elution comprises application of about 10 to 60 CV, about 20 to 40 CV or about 20 to 24 CV of the at least two different solutions to the stationary phase.
   E64. The method of any one of E55-E63, wherein at the start of the gradient elution the percentage of the first solution (e.g., a first gradient elution buffer, buffer A) is 50% to 100% and at the end of the gradient elution the percentage of the second solution (e.g., second gradient elution buffer, buffer B) is 50% to 100%, and wherein optionally 10 to 60 CV of the first solution, the second solution or a mixture of both are applied to the stationary phase during the gradient elution.
   E65. The method of any one of E55-E64, wherein at the start of the gradient elution the percentage of the first solution (e.g., a first gradient elution buffer, buffer A) is 100% and at the end of the gradient elution the percentage of the second solution (e.g., second gradient elution buffer, buffer B) is 100%, and wherein optionally 10 to 60 CV of the first solution, the second solution or a mixture of both are applied to the stationary phase during the gradient elution.
   E66. The method of any one of E55-E65, wherein a concentration of a component of the first solution (e.g., first gradient elution buffer) or second solution (e.g., second gradient elution buffer) increases or decreases continuously during the gradient elution; wherein a rate of increase or decrease of a concentration of a component of the first solution or second solution is equivalent to a change in concentration of a component per total CV; and wherein the rate of change in concentration of a component over a gradient elution is about 10 mM/CV to 50 mM/CV.
   E67. The method of any one of E54-E66, wherein full capsids are eluted from the stationary phase in a first elution peak and/or in a first portion of a second elution peak of the gradient elution.
   E68. The method of any one of E54-E67, wherein empty capsids are recovered in an AEX column flow-through and/or in a last portion of a second elution peak of the gradient elution.
   E69. The method of any one of E1-E68, comprising performing a gradient hold.
   E70. The method of E69, wherein the gradient hold comprises application of 1 to 10 CV of a gradient hold solution comprising a component selected from the group consisting of a salt, a buffering agent, a detergent and a combination thereof to the stationary phase in the column.
   E71. The method of E69 or E70, wherein performing the gradient hold comprises application of 1 to 10 CV of a gradient hold solution comprising about 5 mM to about 1 M (e.g., about 500 mM) sodium acetate, 50 mM to 150 mM (e.g., about 100 mM) Tris, about 0.005% to about 0.015% (e.g., about 0.01%) P188, pH about 8.5 to 9.5 (e.g., about 8.9) to the AEX stationary phase in the column.
   E72. The method of any one of E1-E71, comprising performing a step elution (e.g., isocratic elution) from the stationary phase in the column.
   E73. The method of E72, wherein performing the step elution comprises application of at least 2, 3, 4, 5, 6, 7, 8, 9, 10 or more step elution solutions to the column stationary phase.
   E74. The method of E72 or E73, wherein the at least 2, 3, 4, 5, 6, 7, 8, 9, 10 or more step elution solutions each comprise a component selected from the group consisting of a buffering agent, a salt, a detergent and a combination thereof.
   E75. The method of any one of E72-E74, wherein the at least 2, 3, 4, 5, 6, 7, 8, 9, 10 or more step elution solutions each comprise about 10 mM to about 50 mM (e.g., about 20 mM) Tris and about 5 mM to about 600 mM sodium acetate, pH about 8 to 10 (e.g., about 8.9 to about 9.1).
   E76. The method of any one of E72-E75, wherein step elution solutions with increasing concentration of sodium acetate are sequentially applied to the column.
   E77. The method of any one of E72-E76, wherein the final step elution solution comprises about 20 mM Tris, about 500 mM sodium acetate, pH about 8.9 to about 9.1.
   E78. The method of any one of E1-E77, comprising collecting at least one fraction of eluate from the column to recover full rAAV capsids, optionally during a gradient elution.
   E79. The method of E78, wherein a volume of the at least one fraction of eluate is selected from the group consisting of ⅛ of a CV, ¼ of a CV, ⅓ of a CV, ½ of a CV, ¾ of a CV, 1 CV, 2 CV, 3 CV, 4 CV, 5 CV, 6 CV, 7 CV, 8 CV, 9 CV, 10 CV or more.
   E80. The method of E79, wherein the absorbance of the at least one fraction of eluate is measured at 280 nm, and wherein optionally, the threshold is ≥0.5 mAU/mm path length measured at 280 nm.
   E81. The method of E80, wherein the at least one fraction of eluate is collected when the A280 of the eluate is ≥0.5 mAU/mm path length.
   E82. The method of E80 or E81, wherein a volume of the at least one fraction of eluate is equivalent to ⅛ of a CV to 10 CV, e.g., ⅛ of a CV, ¼ of a CV, ⅓ of a CV, ½ of a CV, ¾ of a CV, 1 CV, 2 CV, 3 CV, 4 CV, 5 CV, 6 CV, 7 CV, 8 CV, 9 CV, 10 CV or more of a CV, and wherein optionally, the A260/A280 ratio of the at least one fraction of eluate is ≥ to 1.25.
   E83. The method of any one of E78-E82, wherein at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more fractions of eluate are collected.
   E84. The method of any one of E78-E83, further comprising adjusting a pH of the at least one fraction of eluate collected from the column, optionally during a gradient elution.
   E85. The method of E84, wherein adjusting the pH of the at least one fraction of eluate comprises i) addition of 14.3% to 15% eluate volume by weight of a solution comprising a buffering agent, pH about 3.5 to the at least one fraction of eluate or ii) collecting the at least one fraction of eluate into a vessel comprising about 0.01 CV to 0.1 CV (e.g., about 0.066 CV) of a solution comprising a buffering agent.
   E86. The method of E85, wherein the buffering agent is about 200 mM to about 300 mM (e.g., about 250 mM) sodium citrate.
   E87. The method of E84-E86, wherein the pH of the at least one fraction of eluate collected from the column is adjusted from an initial pH of about 8.5 to about 9.1 to a pH of about 6.8 to about 7.6.
   E88. The method of E84-E87, wherein the pH of the at least one fraction of eluate collected from the column is adjusted from an initial pH of about 8.5 to about 9.1 to a pH of about 7.0 to about 7.4.
   E89. The method of any one of E78-E88, further comprising measuring an absorbance of at least one fraction of eluate collected from the column, optionally during a gradient elution.
   E90. The method of E89, wherein the absorbance is measured at 260 nm (A260), 280 nm (A280) or at 260 nm and 280 nm, and optionally wherein an A260/A280 ratio is determined.
   E91. The method of E90, wherein the absorbance at 260 nm and 280 nm is measured by size exclusion chromatography (SEC).
   E92. The method of E90 or E91, wherein the A260/A280 ratio of at least one fraction of eluate is at least 0.5, at least 0.55, at least 0.6, at least 0.65, at least 0.70, at least 0.75, at least 0.80, at least 0.85, at least 0.90, at least 0.95. at least 1.0, at least, 1.05, least 1.10, at least 1.11, at least 1.12, at least 1.13, at least 1.14, at least 1.15, at least 1.16, at least 1.17, at least 1.18, at least 1.19, at least 1.20, at least 1.21, at least 1.22, at least 1.23, at least 1.24, at least 1.25, at least 1.26, at least 1.27, at least 1.28, at least 1.29, at least 1.30, at least 1.31, at least 1.32, at least 1.33, at least 1.34, at least 1.35, at least 1.36, at least 1.37, at least, 1.38, at least 1.39, at least 1.40 or is about 0.5 to about 2.0, about 0.5 to about 1.8, about 0.5 to about 1.6, about 0.5 to about 1.4, about 0.5 to about 1.2, about 0.5 to about 1.0, about 0.5 to about 0.8, about 0.6 to about 2.0, about 0.8 to about 1.8, about 0.8 to about 1.6, about 0.8 to about 1.4, about 1.0 to about 1.4, or about 1.0 to about 1.2, optionally as measured by SEC.
   E93. The method of any one of E90 to E92, wherein the A260/A280 ratio of at least one fraction of eluate is at least 1.25.
   E94. The method of any one of E78-E93, further comprising combining at least two fractions of eluate collected from the column, optionally during a gradient elution to form a pooled eluate comprising the rAAV vector.
   E95. The method of E94, wherein 2 to 50 fractions of eluate are combined to form a pooled eluate.
   E96. The method of E94 or E95, wherein the at least two fractions of eluate each have an A260/A280 ratio of ≥1.25.
   E97. The method of any one of E94-E96, further comprising measuring the absorbance of the pooled eluate, and wherein the A260/A280 of the pooled eluate is ≥1.25 (e.g., about 1.28 to 1.35).
   E98. The method of any one of E94-E97, wherein the pooled eluate has a pH of 6.8 to 7.6 (e.g., 7.0 to 7.4).
   E99. The method of any one of E78-E98, wherein full capsids comprise 20% to 60%, 20% to 70%, 20% to 80%, 20% to 90%, 20% to 95%, 20% to 98%, 20% to 99%, 20% to greater than 99%, 40% to 50%, 40% to 60%, 40% to 70%, 40% to 80% (e.g., 44%, 45%, 50%, 53%) of total capsids in the at least one fraction of eluate, or the pooled eluate, and optionally wherein the capsids are measured by analytical ultracentrifugation (AUC).
   E100. The method of any one of E78-E99, wherein full capsids comprise about 55% of total capsids (e.g., 55%+/−7%) in the at least one fraction of eluate, or the pooled eluate.
   E101. The method of any one of E78-E99, wherein full capsids comprise about 49% (e.g., 49%+/−2%) of total capsids in the at least one fraction of eluate, or the pooled eluate.
   E102. The method of any one of E78-E99, wherein full capsids comprise 52+/−7% of total capsids in the at least one fraction of eluate, or the pooled eluate.
   E103. The method of any one of E78-E99, wherein the at least one fraction of eluate, or the pooled eluate, comprises 48% to 62% full rAAV capsids of total capsids, and wherein the at least one fraction of eluate, or the pooled eluate, is generated from purification of a rAAV vector produced in a 250 L SUB.
   E104. The method of any one of E78-E99, wherein the at least one fraction of eluate, or the pooled eluate, comprises 47% to 51% full rAAV capsids, of total capsids, and wherein the at least one fraction of eluate, or the pooled eluate, is generated from purification of a rAAV vector produced in a 2000 L SUB.
   E105. The method of any one of E78-E99, wherein the at least one fraction of eluate, or the pooled eluate, comprises greater than 30% (e.g., 40% to 55%, 45% to 65%, 40% to greater than 99%) full capsids of total capsids and wherein the solution comprising the rAAV vector to be purified comprises less than 30% (e.g., 12% to 25%) full capsids of total capsids in the solution.
   E106. The method of any one of E78-E105, wherein empty capsids comprise 10% to 99%, 10% to 90%, 10% to 80%, 10% to 70%, 10% to 65%, 10% to 60%, 10% to 50%, 10% to 40%, 10% to 30%, 10% to 20%, 20% to 65%, 20% to 60%, 20% to 50%, 20% to 40%, or 18% to 29%, (e.g., ≤29%) of total capsids in the at least one fraction of eluate, or the pooled eluate, and optionally wherein the capsids are measured by analytical ultracentrifugation (AUC).
   E107. The method of any one of E78-E106, wherein empty capsids comprise 20%+/−7% (e.g., 21%) of total capsids in the at least one fraction of eluate, or the pooled eluate.
   E108. The method of any one of E78-E106, wherein the at least one fraction of eluate, or the pooled eluate, comprises 11% to 31% empty capsids of total capsids, and wherein the at least one fraction of eluate, or the pooled eluate, is generated from purification of a rAAV vector produced in a 250 L SUB.
   E109. The method of any one of E78-E106, wherein the at least one fraction of eluate, or the pooled eluate, comprises 18% to 22% empty capsids of total capsids, and wherein the at least one fraction of eluate, or the pooled eluate, is generated from purification of a rAAV vector produced in a 2000 L SUB.
   E110. The method of any one of E78-E106, wherein the at least one fraction of eluate or the pooled eluate comprises less than 30% empty capsids of the total capsids, and wherein the solution comprising the rAAV vector to be purified comprises 40% to 90% empty capsids of total capsids in the solution.
   E111. The method of any one of E78-E110, wherein intermediate capsids comprise 10% to 65%, 10% to 60%, 10% to 50%, 10% to 40%, 10% to 30%, 10% to 20%, 20% to 65%, 20% to 60%, 20% to 50%, 20% to 40%, or 18% to 22% of total capsids in the at least one fraction of eluate, or the pooled eluate, and optionally wherein the capsids are measured by analytical ultracentrifugation (AUC).
   E112. The method of any one of E78-E111, wherein intermediate capsids comprise 28%+/−5% of total capsids in the at least one fraction of eluate, or the pooled eluate.
   E113. The method of any one of E78-E111, wherein the at least one fraction of eluate, or the pooled eluate, comprises 21% to 27% intermediate capsids of total capsids, and wherein the at least one fraction of eluate or the pooled eluate is generated from purification of a rAAV vector produced in a 250 L SUB.
   E114. The method of any one of E78-E111, wherein the at least one fraction of eluate, or the pooled eluate, comprises 28% to 36% intermediate capsids of total capsids, and wherein the at least one fraction of eluate, or the pooled eluate, is generated from purification of a rAAV vector produced in a 2000 L SUB.
   E115. The method of any one of E78-E111, wherein the at least one fraction of eluate, or the pooled eluate, comprises 45% to 65% full rAAV capsids, 19% to 28% intermediate capsids and 10% to 37% empty capsids of total capsids, and wherein the at least one fraction of eluate or the pooled eluate is generated from purification of a rAAV vector produced in a 250 L SUB.
   E116. The method of E115, wherein full capsids comprise 55%+/−7% of total capsids in the at least one fraction of eluate or the pooled eluate.
   E117. The method of E115, wherein intermediate capsids comprise 24%+/−3% of total capsids in the at least one fraction of eluate or the pooled eluate.
   E118. The method of E115, wherein empty capsids comprise 21%+/−10% of total capsids in the at least one fraction of eluate or the pooled eluate.
   E119. The method of any one of E78-E118, wherein the at least one fraction of eluate, or the pooled eluate, comprises 45% to 52% full rAAV capsids, 27% to 37% intermediate capsids and/or 18% to 22% empty capsids of total capsids, and wherein the at least one fraction of eluate, or the pooled eluate, is generated from purification of a rAAV vector produced in a 2000 L SUB.
   E120. The method of E119, wherein full capsids comprise 49%+/−2% of total capsids in the at least one fraction of eluate or the pooled eluate.
   E121. The method of E119, wherein the intermediate capsids comprise 32%+1-4% of total capsids in the at least one fraction of eluate or the pooled eluate.
   E122. The method of E119, wherein the empty capsids comprise 20%+/−2% of total capsids in the at least one fraction of eluate or the pooled eluate.
   E123. The method of any one of E94-E122, wherein the pooled eluate is enriched for full capsids, and/or depleted of empty capsids, as compared to the solution loaded onto the column.
   E124. The method of any one of E78-E123, wherein a % VG step yield of the at least one fraction of eluate, or the pooled eluate, is 1% to 10%, 1 to 20%, 1% to 30%, 1% to 40%, 1% to 50%, 1% to 60%, 1% to 70%, 1% to 80%, 1% to 90%, 1% to 99%, 5% to 95%, 10% to 85%, 15% to 75%, 20% to 65%, 25% to 55%, 30% to 45%, 30% to 80%, 40% to 70% or 100%.
   E125. The method of E124, wherein the % VG step yield of the at least one fraction of eluate, or the pooled eluate, is 31% to 66% (e.g., 47%+/−11%).
   E126. The method of E124, wherein the % VG step yield of an at least one fraction of eluate or a pooled eluate produced in a 250 L SUB is 30% to 70% (e.g., 37% to 60%).
   E127. The method of E124, wherein the % VG step yield of the at least one fraction of eluate or the pooled eluate produced in a 250 L SUB is 45%+/−8%.
   E128. The method of E124, wherein the % VG step yield of an at least one fraction of eluate or a pooled eluate produced in a 2000 L SUB is 25% to 75% (e.g., 31% to 66%).
   E129. The method of E124, wherein the % VG step yield of the at least one fraction of eluate or the pooled eluate produced in a 2000 L SUB is 50%+/−13%.
   E130. The method of any one of E124-E129, wherein the % VG step yield of the at least one fraction of eluate, or the pooled eluate, is greater than the % VG step yield of an otherwise identical fraction of eluate, or pooled eluate, purified by ultracentrifugation and cation exchange chromatography.
   E131. The method of any one of E78-E130, wherein the A260/A280 (SEC) of the at least one fraction of eluate, or the pooled eluate, produced in a 250 L SUB is 1.29+/−0.03.
   E132. The method of any one of E78-E130, wherein the A260/A280 (SEC) of the at least one fraction of eluate, or the pooled eluate, produced in a 2000 L SUB is 1.30+/−0.01
   E133. The method of any one of E78-E132, wherein a % VG column yield of the at least one fraction of eluate, or the pooled eluate, is 1% to 10%, 1 to 20%, 1% to 30%, 1% to 40%, 1% to 50%, 1% to 60%, 1% to 70%, 1% to 80%, 1% to 90%, 1% to 99%, 5% to 95%, 10% to 85%, 15% to 75%, 20% to 65%, 25% to 55%, 30% to 45%, 30% to 80%, 40% to 70% or 100%.
   E134. The method of E133, wherein the % VG column yield of the at least one fraction of eluate, or the pooled eluate, is 20% to 100% (e.g., 63%+/−26%).
   E135. The method of E133, wherein the % VG column yield of an at least one fraction of eluate, or a pooled eluate, produced in a 250 L SUB is 40% to 100%.
   E136. The method of E133, wherein the % VG column yield of an at least one fraction of eluate, or a pooled eluate, produced in a 2000 L SUB is 10% to 70% (e.g., 20% to 61%).
   E137. The method of any one of E78-E136, wherein the at least one fraction of eluate, or pooled eluate, is further subjected to a method of filtration selected from the group consisting of viral filtration, ultrafiltration/diafiltration (UF/DF), filtration through a 0.2 μm filter and a combination thereof, to produce a drug substance.
   E138. The method of E137, wherein full capsids comprise 20% to 60%, 20% to 70%, 20% to 80%, 20% to 90%, 20% to 95%, 20% to 98%, 20% to 99%, 20% to greater than 99%, 40% to 50%, 40% to 60%, 40% to 70%, 40% to 80% (e.g., 44%, 45%, 50%, 53%) of total capsids in the drug substance, and optionally wherein the capsids are measured by analytical ultracentrifugation (AUC).
   E139. The method of E137 or E138, wherein the drug substance comprises 45% to 65% full rAAV capsids, of total capsids, and optionally wherein the drug substance is generated from purification of a rAAV vector produced in a 250 L SUB.
   E140. The method of any one of E137-E139, wherein full capsids comprise 52+/−7% of total capsids in the drug substance
   E141. The method of E137 or E138, wherein the drug substance comprises 45% to 52% full rAAV capsids, of total capsids, and wherein the drug substance is generated from purification of a rAAV vector produced in a 2000 L SUB.
   E142. The method of E137 or E138, wherein the drug substance comprises greater than 30% (e.g., 40% to 55%, 45% to 65%, 40% to greater than 99%) full capsids of total capsids in the drug substance, and wherein the solution comprising the rAAV vector to be purified comprises less than 30% (e.g., 12% to 25%) full capsids of total capsids in the solution.
   E143. The method of any one of E137-E142, wherein empty capsids comprise 10% to 99%, 10% to 90%, 10% to 80%, 10% to 70%, 10% to 65%, 10% to 60%, 10% to 50%, 10% to 40%, 10% to 30%, 10% to 20%, 20% to 65%, 20% to 60%, 20% to 50%, 20% to 40%, or 18% to 29%, (e.g., ≤29%) of total capsids in the drug substance, and optionally wherein the capsids are measured by analytical ultracentrifugation (AUC).
   E144. The method of any one of E143, wherein the drug substance comprises 10% to 37% empty capsids of total capsids, and optionally wherein the drug substance is generated from purification of a rAAV vector produced in a 250 L SUB.
   E145. The method of E143 or E144, wherein empty capsids comprise 20%+/−7% of total capsids in the drug substance.
   E146. The method of any one of E143, wherein the drug substance comprises 18% to 22% empty capsids of total capsids, and optionally wherein the drug substance is generated from purification of a rAAV vector produced in a 2000 L SUB.
   E147. The method of any one of E143-E146, wherein the drug substance comprises less than 30% empty capsids of the total capsids in the drug substance, and wherein the solution comprising the rAAV vector to be purified comprises 40% to 90% empty capsids of total capsids in the solution.
   E148. The method of any one of E137-E147, wherein intermediate capsids comprise 10% to 65%, 10% to 60%, 10% to 50%, 10% to 40%, 10% to 30%, 10% to 20%, 20% to 65%, 20% to 60%, 20% to 50%, 20% to 40%, or 18% to 22% of total capsids in the drug substance, and optionally wherein the capsids are measured by analytical ultracentrifugation (AUC).
   E149. The method of any one of E137-E148, wherein the drug substance comprises 19% to 37% intermediate capsids of total capsids, and optionally wherein the drug substance is generated from purification of a rAAV vector produced in a 250 L or 2000 L SUB.
   E150. The method of E148 or E149, wherein intermediate capsids comprise 28%+/−5% of total capsids in the drug substance.
   E151. The method of any one of E137-E150, wherein the drug substance comprises 45% to 65% full rAAV capsids, 19% to 28% intermediate capsids and 10% to 37% empty capsids of total capsids, and wherein the drug substance is generated from purification of a rAAV vector produced in a 250 L SUB.
   E152. The method of E151, wherein the full capsids comprise 55%+/−7% of total capsids.
   E153. The method of E151 or E152, wherein the intermediate capsids comprise 24%+/−3% of total capsids.
   E154. The method of any one of E151-E153, wherein the empty capsids comprise 21%+/−10% of total capsids.
   E155. The method of any one of E137-E150, wherein the drug substance comprises 45% to 52% full rAAV capsids, 27% to 37% intermediate capsids and/or 18% to 22% empty capsids of total capsids, and wherein the drug substance is generated from purification of a rAAV vector produced in a 2000 L SUB.
   E156. The method of E155, wherein the full capsids comprise 49%+/−2% of total capsids.
   E157. The method of E155 or E156, wherein the intermediate capsids comprise 32%+/−4% of total capsids.
   E158. The method of any one of E155-E157, wherein the empty capsids comprise 20%+/−2% of total capsids.
   E159. The method of any one of E137-E158, wherein the drug substance is depleted of host cell protein (HCP) as compared to the amount of HCP in the solution comprising the rAAV vector to be purified.
   E160. The method of any one of E137-E159, wherein the drug substance comprises an amount of HCP below a lowest level of quantification (LLOQ) as measured by ELISA.
   E161. The method of any one of E137-E160, wherein the drug substance comprises an amount of HCP that is below the LLOQ, as measured by ELISA, wherein the solution comprising the rAAV vector to be purified comprises about 1 to 500 pg HCP/1×10⁹ VG, and optionally wherein the solution is generated from affinity chromatography purification of a rAAV vector produced in a 250 L SUB.
   E162. The method of any one of E137-E161, wherein the drug substance comprises an amount of HCP that is below the LLOQ, as measured by ELISA, wherein the solution comprising the rAAV vector to be purified comprises about 100 to 1000 pg HCP/1×10⁹ VG, and optionally wherein the solution is generated from affinity chromatography purification of a rAAV vector produced in a 2000 L SUB.
   E163. The method of any one of E137-E162, wherein the purity of the drug substance is about 90%, about 91%, about 92%, about 93%, about 94%, about 95%, about 96%, about 97%, about 98%, about 99% or 100%, optionally, as measured by reverse phase HPLC.
   E164. The method of E163, wherein the purity of the drug substance is about 98.6+/−0.6% and optionally wherein the drug substance is generated from purification of a rAAV vector produced in a 250 L SUB.
   E165. The method of E164, wherein the purity of the drug substance is about 99.3+/−0.3% and optionally wherein the drug substance is generated from purification of a rAAV vector produced in a 2000 L SUB.
   E166. The method of any one of E137-E165, wherein the drug substances comprises about 0 to 10% HMMS, optionally, as measured by SEC.
   E167. The method of E166, wherein the drug substance comprises 2.6+/−0.8% HMMS and optionally wherein the drug substance is generated from purification of a rAAV vector produced in a 250 L SUB.
   E168. The method of E166, wherein the drug substance comprises 2.9+/−0.4% HMMS and optionally wherein the drug substance is generated from purification of a rAAV vector produced in a 2000 L SUB.
   E169. The method of E137-E168, wherein the drug substance comprises about 7.0 to 25 pg/1×10⁹ VG of residual HC-DNA.
   E170. The method of E169, wherein the drug substance comprises about 17.4+/−6.7 pg/1×10⁹ VG of HC-DNA and optionally wherein the drug substance is generated from purification of a rAAV vector produced in a 250 L SUB.
   E171. The method of E169, wherein the drug substance comprises about 9.3+/−1.2 pg/1×10⁹ VG of HC-DNA, and optionally wherein the drug substance is generated from purification of a rAAV vector produced in a 2000 L SUB.
   E172. The method of any one of E137-E171, wherein the drug substance has an A260/A280 of about 1.24 to 1.32, optionally, as measured by size exclusion chromatography (SEC).
   E173. The method of E172, wherein the drug substance has an A260/A280 of 1.24 to 1.32, optionally, as measured by SEC, and wherein the drug substance is generated from purification of a rAAV vector produced in a 250 L SUB.
   E174. The method of E172, wherein the drug substance has an A260/A280 of 1.28 to 1.31, optionally, as measured by SEC, and optionally wherein the drug substance is generated from purification of a rAAV vector produced in a 2000 L SUB.
   E175. The method of any one of E1-E174, wherein a volume of the column is 1.0 mL to 6.6 L.
   E176. The method of any one of E1-E175, wherein the volume of the column is about 1.0 mL, about 5.1 mL, about 6.67 mL, about 1.256 L, about 1.3 L, about 6.3 L, about 6.4 L, or about 6.6 L.
   E177. The method of any one of E1-E176, wherein the rAAV vector comprises a capsid protein from an AAV serotype selected from the group consisting of AAV1, AAV2, AAV3, AAV3A, AAV3B, AAV4, AAV5, AAV6, AAV7, AAV8, AAV9, AAV10, AAVrh10, AAVrh74, AAV12, AAV2i8, NP4, NP22, NP66, AAVDJ, AAVDJ/8, AAVDJ/9, AAVLK03, AAV1.1, AAV2.5, AAV6.1, AAV6.3.1, AAV9.45, RHM4-1 (SEQ ID NO:5 of WO 2015/013313), RHM15-1, RHM15-2, RHM15-3/RHM15-5, RHM15-4, RHM15-6, AAV hu.26, AAV1.1, AAV2.5, AAV6.1, AAV6.3.1, AAV9.45, AAV2i8, AAV29G, AAV2,8G9, AVV-LK03, AAV2-TT, AAV2-TT-S312N, AAV3B-S312N, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15.
   E178. The method of any one of E1-E177, wherein a purified rAAV vector is produced.
   E179. The method of E178, wherein the purified rAAV vector is a drug substance.
   E180. The method of any one of E137-E174 or E179, wherein the drug substance and a pharmaceutically acceptable excipient are combined to form a drug product.
   E181. The method of any one of E178-E180, wherein the purified rAAV vector, the drug substance and/or the drug product is suitable for administration to a subject to treat a disease, disorder or condition.
   E182. The method of E181, wherein the disease, disorder or condition is Duchenne muscular dystrophy (DMD).
   E183. The method of any one of E1-E182, wherein the rAAV vector comprises a vector genome comprising a modified nucleic acid encoding a human mini-dystrophin.
   E184. The method of E183, wherein the modified nucleic acid comprises or consists of the nucleic acid sequence of SEQ ID NO: 1.
   E185. The method of E183 or E184, wherein the modified nucleic acid encodes a human mini-dystrophin comprising or consisting of the amino acid sequence of SEQ ID NO:2.
   E186. The method of any one of E183-E185, wherein the vector genome comprises a muscle specific promoter and/or enhancer selected from the group consisting of a synthetic hybrid muscle-specific promoter hCK, a synthetic hybrid muscle-specific promoter hCKplus, and a synthetic muscle-specific enhancer and promoter.
   E187. The method of E186, wherein the synthetic hybrid muscle-specific promoter hCK comprises or consists of the nucleic acid sequence of SEQ ID NO:3.
   E188. The method of E186, wherein the synthetic hybrid muscle-specific promoter hCKplus comprises or consists of the nucleic acid sequence of SEQ ID NO:4.
   E189. The method of E186, wherein the synthetic muscle-specific enhancer and promoter comprises or consists of the nucleic acid sequence of SEQ ID NO:5.
   E190. The method of any one of E183-E189, wherein the vector genome comprises a polyadenylation (polyA) signal sequence.
   E191. The method of E190, wherein the polyA signal sequence comprises or consists of the nucleic acid sequence of SEQ ID NO:6.
   E192. The method of any one of E183-E191, wherein the vector genome comprises a transcription terminator sequence.
   E193. The method of E192, wherein the transcription terminator sequence comprises or consists of the nucleic acid sequence of SEQ ID NO:9.
   E194. The method of any one of E183-E193, wherein the vector genome comprises at least one ITR.
   E195. The method of E194, wherein the at least one ITR comprises or consists of a nucleic acid sequence selected from SEQ ID NO:7, SEQ ID NO:8, SEQ ID NO:13, SEQ ID NO:14 and a combination thereof.
   E196. The method of any one of E183-E195, wherein the vector genome comprises an expression cassette, and wherein the expression cassette comprises or consists of the nucleic acid sequence of SEQ ID NO:10.
   E197. The method of any one of E1-E196, wherein the rAAV vector comprises a VP1 polypeptide of AAV9.
   E198. The method of E197, wherein the VP1 polypeptide comprises or consists of the amino acid sequence of SEQ ID NO:11.
   E199. The method of any one of E1-E198, wherein the rAAV vector comprises an AAV9 capsid protein and a transgene comprising the nucleic acid sequence of SEQ ID NO:1.
   E200. The method of any one of E1-E199, further comprising preparing the solution comprising the rAAV vector for purification by AEX.
   E201. The method of E200, wherein the solution comprising the rAAV vector is selected from the group consisting of an affinity eluate, a supernatant from a cell lysate and a post-harvest solution that has been diluted and optionally filtered prior to loading.
   E202. The method of E200 or E201, wherein the solution comprising the rAAV vector is an affinity eluate that has been diluted and optionally filtered prior to loading.
   E203. The method of any one of E200-E202, wherein the solution has already undergone at least one purification and/or processing step.
   E204. The method of any one of E200-E203, wherein the solution comprising the rAAV vector is an eluate resulting from affinity chromatography purification of a rAAV vector.
   E205. The method of any one of E200-E204, wherein preparation comprises diluting the solution comprising the rAAV vector
   E206. The method of E205, wherein diluting the solution comprising the rAAV vector comprises diluting the solution about 2-fold, about 3-fold, about 4-fold, about about, 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about-19 fold, about 20-fold, about 25-fold to produce a diluted solution.
   E207. The method of E205 or E206, wherein diluting the solution comprising the rAAV vector comprises diluting the solution about 15-fold to produce a diluted solution.
   E208. The method of any one of E205-E207, wherein diluting the solution comprising the rAAV vector is performed in-line with the AEX column, and wherein a dilution solution is delivered through a first tubing to a Y-connector, and the solution comprising the rAAV vector is delivered through a second tubing to the Y-connector.
   E209. The method of E208, wherein the dilution solution is delivered at a flow rate of 1 to 5 mL/min, and wherein the solution comprising the rAAV vector is delivered at a flow rate of 0.1 to 2 mL/min, such that the solution is diluted about 15-fold.
   E210. The method of E208 or E209, wherein the dilution solution comprises at least one component selected from the group consisting of a buffering agent, an amino acid, a detergent and a combination thereof.
   E211. The method of E210, wherein the buffering agent is selected from the group consisting of Tris, BIS-Tris propane, diethanolamine, diethylamine, tricine, triethanolamine, bicine and a combination thereof.
   E212. The method of E210 or E211, wherein the amino acid is selected from the group consisting of histidine, arginine, glycine, citrulline and a combination thereof.
   E213. The method of any one of E210-E212, wherein the amino acid is histidine.
   E214. The method of any one of E210-E213, wherein the detergent is selected from the group consisting of poloxamer 188 (P188), Triton X-100, polysorbate 80 (PS80), Brij-35, nonyl phenoxypolyethoxylethanol (NP-40) and a combination thereof.
   E215. The method of any one of E210-E214, wherein the detergent is P188.
   E216. The method of any one of E208-E215, wherein the dilution solution comprises about 100 mM to about 300 mM (e.g., about 200 mM) histidine, about 100 mM to about 300 mM (e.g., about 200 mM) Tris, about 0.1% to about 1.0% (e.g., about 0.5%) P188, pH about 8.5 to about 9.5 (e.g., about 8.8).
   E217. The method of any one of E205-E216, wherein diluting the solution comprising the rAAV vector precedes loading the solution comprising the rAAV vector onto the column.
   E218. The method of any one of E205-E217, wherein pH of the solution comprising the rAAV vector after diluting is increased as compared to the pH of the solution before diluting.
   E219. The method of any one of E205-E218, wherein pH of the solution comprising the rAAV vector before diluting is 3.0 to 4.4 and the pH of the solution after diluting is 8.5 to 9.5.
   E220. The method of any one of E205-E219, wherein conductivity of the solution comprising the rAAV vector diluting is decreased as compared to conductivity of the solution before diluting.
   E221. The method of any one of E205-E220, wherein conductivity of the solution comprising the rAAV vector before diluting is 5.0 to 7.0 mS/cm (e.g., 5.5 to 6.5 mS/cm) and conductivity of the solution after diluting is 1.7 to 3.5 mS/cm.
   E222. The method of any one of E205-E221, further comprising filtering the diluted solution comprising the rAAV vector.
   E223. The method of E222, wherein filtering the diluted solution comprises filtration through a 0.2 μm filter.
   E224. The method of E222 or E223, wherein the filter is in-line with the column.
   E225. The method of any one of E222-E224, wherein diluting and filtering the solution comprising the rAAV vector precedes loading the solution comprising the rAAV vector onto the column.
   E226. The method of any one of E205-E225, wherein the percent vector genome (% VG) yield of the diluted, and optionally filtered solution comprising the rAAV vector is 60% to 100% as compared to the amount of VG present in the solution prior to dilution, and optionally filtration.
   E227. The method of any one of E205-E226, wherein a % VG dilution yield of the diluted solution comprising the rAAV vector is 88%+/−36%.
   E228. A method of preparing a solution comprising a rAAV vector for purification by AEX, the method comprising a step of:
i) diluting a first solution 2 to 25-fold (e.g., 15-fold) with a dilution solution; and optionally
ii) filtering the solution from step i) through a filter to produce the diluted, and optionally filtered solution; wherein the pH of the diluted, and optionally filtered solution is increased as compared to the pH of the first solution; and wherein the conductivity of the diluted, and optionally filtered solution is decreased as compared to the conductivity of the first solution.
   E229. The method of preparing a solution comprising a rAAV vector for purification by AEX of E228, wherein i) the pH of the diluted, and optionally filtered solution is 8.5 to 9.5; ii) the conductivity of the diluted, and optionally filtered solution is 1.7 to 3.3 mS/cm; and/or iii) a % VG dilution yield of the solution after dilution is 35% to 100%.
   E230. The method of preparing a solution comprising a rAAV vector for purification by AEX of E228 or E229, wherein the rAAV vector comprises an AAV9 capsid protein.
   E231. A method of purifying an rAAV vector by AEX, the method comprising a step of:
i) loading a solution comprising the rAAV vector to be purified onto an AEX stationary phase in a column;
ii) performing gradient elution of a material from the stationary phase in the column wherein a percentage of a first gradient elution buffer is varied in a manner inversely proportional to variation in a percentage of a second gradient elution buffer;
iii) collecting at least one fraction of eluate from the column during the gradient elution beginning when the absorbance of a column flow through reaches an absorbance threshold;
iv) measuring an absorbance of the at least one fraction of eluate collected from the column and determining an A260/A280 ratio.
   E232. A method of purifying an rAAV vector by AEX of E231, wherein the method further comprises combining at least two fractions of eluate collected from the column to form a pooled eluate comprising the rAAV vector.
   E233. The method of purifying a rAAV vector by AEX of E231 or E232, wherein the AEX stationary phase is POROS™ 50 HQ.
   E234. The method of purifying a rAAV vector by AEX of any one of E231-E233, wherein the solution is an affinity eluate has been diluted and filtered prior to loading onto the stationary phase.
   E235. The method of purifying a rAAV vector of any one of E231-E234, wherein the material eluted from the stationary phase comprises the rAAV vector to be purified.
   E236. The method of purifying a rAAV vector by AEX of any one of E231-E235, wherein a first gradient elution buffer comprises about 50 mM to about 150 mM (e.g., about 100 mM) Tris, about 0.005% to about 0.015% (e.g., about 0.01%) P188, about pH 8.5 to 9.5 (e.g., about 8.9).
   E237. The method of purifying a rAAV vector by AEX of any one of E231-E236, wherein a second gradient elution buffer comprises about 400 mM to about 600 mM (e.g., about 500 mM) sodium acetate, about 50 mM to about 150 mM (e.g., about 100 mM) Tris, about 0.005% to about 0.015% (e.g., about 0.01%) P188, about pH 8.5 to 9.5 (e.g., about 8.9).
   E238. The method of purifying a rAAV vector by AEX of any one of E231-E237 wherein at the start of the gradient elution the percentage of the first gradient elution buffer is 100% and at the end of the gradient elution the percentage of the second gradient elution buffer is 100%.
   E239. The method of purifying a rAAV vector by AEX of any one of E236-E238, wherein about 15 to about 25 (e.g., 20 CV) of the first gradient elution buffer, the second gradient elution buffer or a mixture of both are applied to the stationary phase during the gradient elution; wherein a concentration of the sodium acetate varies from 0 mM to 500 mM during the gradient elution such that the rate of change in concentration of the sodium acetate over the course of the gradient elution is about 25 mM/CV.
   E240. The method of purifying a rAAV vector by AEX of any one of E231-E239, wherein full capsids are eluted from the stationary phase in a first elution peak; wherein full capsids are eluted from the stationary phase in a first portion of a second elution peak and/or wherein empty capsids are recovered in an AEX column flow-through and/or in a last portion of a second elution peak.
   E241. The method of purifying a rAAV vector by AEX of any one of E231-E240, wherein the absorbance threshold is ≥0.5 mAU/mm path length measured at 280 nm.
   E242. The method of purifying a rAAV vector by AEX of any one of E231-E241, wherein a volume of the at least one fraction of eluate is equivalent to ≥⅓ of a CV.
   E243. The method of purifying a rAAV vector by AEX of any one of E231-E242, wherein the collecting at least one fraction of eluate comprises collecting at least 10 fractions of eluate.
   E244. The method of purifying a rAAV vector by AEX of any one of E231-E242, wherein the pH of the at least one fraction of eluate is adjusted to a pH of 6.8 to 7.6 (e.g., about pH 7.2).
   E245. The method of purifying a rAAV vector by AEX of any one of E231-E244, wherein the A260/A280 ratio of the at least one fraction of eluate, the at least two fractions of eluate and/or the pooled eluate is ≥1.25 (e.g., about 1.28 to 1.35).
   E246. The method of purifying a rAAV vector by AEX of any one of E231-E245, wherein the pooled eluate has a % VG column yield of 20% to 100% (e.g., 63+/−26%).
   E247. The method of purifying a rAAV vector by AEX of any one of E231-E246, wherein the pooled eluate has a % VG step yield of 31% to 66% (e.g., 47+/−11%).
   E248. The method of purifying a rAAV vector by AEX of any one of E231-E247, wherein at least one fraction of eluate and/or pooled eluate is enriched for full capsids, and/or depleted of empty capsids, as compared to the diluted and filtered affinity eluate loaded onto the column.
   E249. The method of purifying a rAAV vector by AEX of any one of E231-E248, wherein a purified rAAV vector is produced.
   E250. The method of purifying a rAAV vector by AEX of any one of E231-E249, further comprising filtering the pooled eluate by a method selected from the group consisting of viral filtration, ultrafiltration/diafiltration (UF/DF), filtration through a 0.2 μm filter and a combination thereof, to produce a drug substance.
   E251. The method of purifying a rAAV vector by AEX of E250, wherein the drug substance comprises 45% to 65% (e.g., 52+/−7%) full capsids of the total capsids.
   E252. The method of purifying a rAAV vector by AEX of E250 or E251, wherein the drug substance comprises 19% to 37% (e.g., 28+/−5%) intermediate capsids of total capsids.
   E253. The method of purifying a rAAV vector by AEX of any one of E250-E252, wherein the drug substance comprises 10% to 37% (e.g., 20+/−7%) empty capsids of total capsids.
   E254. The method of purifying a rAAV vector by AEX of any one of E231-E253, wherein the rAAV vector comprises an AAV9 capsid protein.
   E255. A method of purifying a rAAV vector by AEX, the method comprising a step of:
i) loading an affinity eluate comprising the rAAV vector to be purified onto an AEX stationary phase (e.g., POROS™ 50 HQ) in a column, wherein the eluate has been
   a) diluted about 14.4 to 15.5 fold (e.g., about 15 fold) with a buffer comprising about 200 mM histidine, about 200 mM Tris, about 0.5% P188, pH 8.7 to 9.0, and optionally
   b) filtered through a 0.2 μm filter prior to loading onto the stationary phase;
ii) performing gradient elution of a material from the stationary phase in the column wherein about 20 CV of a first gradient elution buffer (e.g., 100 mM Tris, 0.01% P188, pH 8.9), a second gradient elution buffer (e.g., 500 mM sodium acetate, 100 mM Tris, 0.01% P188, pH 8.9) or a mixture of both are applied to the stationary phase; wherein a concentration of the sodium acetate is varied from 0 mM to 500 mM such that the rate of change in concentration of the sodium acetate over the course of the gradient elution is about 25 mM/CV;
iii) collecting about 10 fractions of eluate from the column during the gradient elution when the A280 of the eluate is >0.5 mAU/mm path length, and wherein a volume of the at least one fraction of eluate is equivalent to ≥⅓ of a CV;
iv) adjusting the pH of the about 10 fractions of eluate from the column to a pH of 6.8 to 7.6 by addition of 14.3% to 15% (eluate volume weight) of a solution comprising about 250 mM sodium citrate, pH 3.5;
v) measuring an absorbance of the about 10 fractions of eluate collected from the column and determining an A260/A280 ratio; and/or
vi) combining at least two fractions of eluate collected from the column to form a pooled eluate,
wherein the A260/A280 of each one of the at least two fractions of eluate is ≥1.25;
wherein the pooled eluate is enriched for full capsids, and/or depleted of empty capsids, as compared to the diluted, and optionally filtered affinity eluate loaded onto the column; and
wherein a purified rAAV vector is produced.
   E256. A method of purifying a rAAV vector by AEX of E255, wherein the material eluted from the stationary phase comprises the rAAV vector to be purified.
   E257. A method of purifying a rAAV vector by AEX, the method comprising a step of:
i) pre-use flushing comprising application of about 5 CV of water for injection to an AEX stationary phase in a column;
ii) sanitizing comprising application of about 16 CV of a solution comprising about 0.5 M NaOH to the AEX stationary phase in the column, optionally by upward flow;
iii) regenerating comprising application of about 5 CV of a solution comprising about 2 M NaCl, 100 mM Tris, pH 9 to the AEX stationary phase in the column;
iv) equilibration comprising application of about 5 CV of a solution comprising about 100 mM Tris, pH 9 to the AEX stationary phase in the column;
v) equilibration comprising application of about 5 CV of an equilibration buffer comprising about 100 mM Tris, 500 mM sodium acetate, 0.01% P188, pH 8.9 to the AEX stationary phase in the column;
vi) equilibration comprising application of about 5 CV of an equilibration buffer comprising about 200 mM histidine, 200 mM Tris, 0.5% P188, pH 8.8 to the AEX stationary phase in the column;
vii) loading an affinity eluate comprising the rAAV vector to be purified onto the AEX stationary phase in the column, optionally wherein the eluate has been
   a) diluted about 15 fold with a buffer comprising about 200 mM histidine, 200 mM Tris, 0.5% P188, pH 8.7 to 9.0, and optionally
   b) filtered through an 0.2 μm filter prior to loading onto the stationary phase;
viii) equilibration comprising application of about 5 CV of an equilibration buffer comprising about 100 mM Tris, 0.01% P188, pH 8.9 to the AEX stationary phase in the column;
ix) performing gradient elution of a material from the stationary phase in the column wherein about 20 CV of a first gradient elution buffer (e.g., 100 mM Tris, P188, pH 8.9), a second gradient elution buffer (e.g., 500 mM sodium acetate, 100 mM Tris, 0.01% P188, pH 8.9) or a mixture of both are applied to the stationary phase; wherein a concentration of the sodium acetate is varied from 0 mM to 500 mM such that the rate of change in concentration of the sodium acetate over the course of the gradient elution is about 25 mM/CV;
x) collecting about 10 fractions of eluate from the column during a gradient elution when the A280 of the of the eluate is ≥0.5 mAU/mm path length; and wherein a volume of the about 10 fractions of eluate is about ≥⅓ of a CV;
xi) adjusting a pH of the about 10 fractions of eluate to a pH of 6.8 to 7.6 by addition of 14.3% to 15% (eluate volume weight) of a solution comprising about 250 mM sodium citrate, pH 3.5;
xii) measuring of an absorbance of the about 10 factions of eluate collected from the column and determining an A260/A280 ratio; and/or
xiii) combining at least two fractions of eluate collected from the column to form a pooled eluate, wherein an A260/A280 of each of the at least two fractions of eluate is ≥1.25;
wherein the pooled eluate is depleted of empty capsids, and/or enriched for full capsids as compared to the affinity eluate and/or the diluted, and optionally filtered affinity eluate;
wherein at least one of steps i) to ix) is performed at a linear velocity of 270 to 330 cm/hr (e.g., about 300 cm/hr), a flow rate of 1.5 to 2.0 L/min (e.g., about 1.8 L/min) or about 314 mL/min through a 1.3 L column and/or a residence time of about 3.5 to 4.5 min/CV (e.g., 4 min/CV); and/or wherein a purified rAAV vector is produced.
   E258. A method of purifying a rAAV vector by AEX of E257, wherein the material eluted from the stationary phase comprises the rAAV vector to be purified.
   E259. A method of preparing a solution comprising a rAAV vector for purification by AEX, the method comprising a step of:
i) diluting an affinity eluate about 15-fold with a dilution solution comprising about 200 mM histidine, 200 mM Tris, 0.5% P188, pH 8.8; and
ii) filtering the affinity eluate from step i) through a 0.2 μm filter to produce a diluted and filtered affinity eluate; wherein the pH of the diluted and filtered affinity eluate is increased (e.g., to about 8.5 to 9.5) as compared to the pH of the affinity eluate; and wherein the conductivity of the diluted and filtered affinity eluate is decreased (e.g., to about 1.7 mS/cm to 3.3 mS/cm) as compared to the conductivity of the affinity eluate.
   E260. The method of preparing a solution comprising a rAAV vector for purification by AEX of E259, wherein the diluted, and optionally filtered affinity eluate is loaded on an AEX stationary phase.
   E261. The method of preparing a solution comprising a rAAV vector for purification by AEX of E259 or E260, wherein the affinity eluate is generated from affinity chromatography-based purification of a rAAV vector produced in a vessel with a volume of 250 L or 2000 L.
   E262. The method of preparing a solution comprising a rAAV vector for purification by AEX of any one of E259-E2561, wherein a % VG dilution yield of the affinity eluate after dilution is 88%+/−36%.
   E263. A method of preparing a stationary phase for use in a method of purifying a rAAV vector by AEX, the method comprising a step of:
i) pre-use flushing comprising application of ≥4.5 CV (e.g., about 5 CV) of water for injection to AEX stationary phase in a column;
ii) sanitizing comprising application of about 14.4 to 17.6 CV (e.g., about 16 CV) of a solution comprising about 0.1 M to 1.0 M (e.g., about 0.5 M) NaOH to the AEX stationary phase in the column, optionally by upward flow; and/or
iii) regenerating comprising application of about 4.5 to 5.5 CV (e.g., about CV) of a solution comprising about 1 M to 3 M NaCl, 50 mM to 150 mM Tris, pH 8.5 to 9.5 to the AEX stationary phase in the column; optionally wherein at least one of steps i)-iii) is performed at a linear velocity of 270 to 330 cm/hr (e.g., about 300 cm/hr), a flow rate of 1.5 to 2.0 L/min (e.g., about 1.8 L/min) and/or a residence time of 3.5 to 4.5 min/CV (e.g., about 4 min/CV).
   E264. The method of preparing a stationary phase for use in a method of purifying a rAAV vector by AEX of E263, further comprising equilibration comprising application of about 5 CV of one or more of the solutions comprising i) about 100 mM Tris, pH 9, ii) about 100 mM Tris, 500 mM sodium acetate, 0.01% P188, pH 8.9 and iii) about 200 mM histidine, 200 mM Tris, 0.5% P188, pH 8.8 to the AEX stationary phase in the column.
   E265. The method of preparing a stationary phase for use in a method of purifying a rAAV vector by AEX of E263 or E264, wherein at least one step is performed prior to loading a solution comprising the rAAV vector to be purified onto the column.
   E266. A purified rAAV vector prepared by a method of any one of E1-E227 or E231-E258.
   E267. A purified rAAV vector prepared by a method comprising a step of:
i) loading a solution comprising the rAAV vector to be purified onto an AEX stationary phase in a column;
ii) performing gradient elution of a material from the stationary phase in the column wherein about 20 CV of a first gradient elution buffer (e.g., 100 mM Tris, P188, pH 8.9), a second gradient elution buffer (e.g., 500 mM sodium acetate, 100 mM Tris, 0.01% P188, pH 8.9) or a mixture of both are applied to the stationary phase; wherein a concentration of a salt is varied from 0 mM to 500 mM such that the rate of change in concentration of the salt over the course of the gradient elution is about 25 mM/CV;
iii) collecting at least one (e.g., about 10) fraction of eluate from the column during a chromatography step (e.g., a gradient elution) when the absorbance of a column flow-through reaches an absorbance threshold (e.g., A280 is >0.5 mAU/mm path length);
iv) measuring an absorbance of the at least one fraction of eluate collected from the column and determining an A260/A280 ratio; and/or
v) combining at least two fractions of eluate collected from the column to form a pooled eluate comprising the purified rAAV vector.
   E268. The purified rAAV vector prepared by the method of E267, wherein the material eluted from the stationary phase comprises the rAAV vector to be purified.
   E269. The purified rAAV vector prepared by the method of E267-E268, wherein the salt is sodium acetate.
   E270. The purified rAAV vector prepared by the method of any one of E267-E269, wherein the solution is an affinity eluate that has been a) diluted about 14.4 to fold (e.g., about 15 fold) with a buffer comprising about 200 mM histidine, 200 mM Tris, 0.5% P188, pH 8.7 to 9.0, and optionally b) filtered through a 0.2 μm filter prior to loading onto the stationary phase.
   E271. The purified rAAV vector prepared by the method of any one of E267-E270, the method further comprising adjusting the pH of the at least one (e.g., about 10) fraction of eluate from the column to a pH of 6.8 to 7.6 by addition of 14.3% to 15% (eluate volume weight) of a solution comprising about 250 mM sodium citrate, pH 3.5.
   E272. The purified rAAV vector of any one of E267-E271, wherein the A260/A280 of each one of the at least two fractions of eluate is ≥1.25; and wherein the pooled eluate is enriched for full capsids, and/or depleted of empty capsids, as compared to the diluted, and optionally filtered affinity eluate loaded onto the column; and wherein the purified rAAV vector is produced.
   E273. The purified rAAV vector of any one of E267-E272, wherein the rAAV vector comprises an AAV9 capsid protein.
   E274. A purified rAAV vector prepared by a method comprising a step of:
i) loading a solution comprising the rAAV vector to be purified onto an AEX stationary phase in a column;
ii) performing gradient elution of a material from the stationary phase in the column wherein a percentage of a first gradient elution buffer is varied in a manner inversely proportional to variation in a percentage of a second gradient elution buffer; iii) collecting at least one fraction of eluate from the column during a chromatography step beginning when the absorbance of a column-flow through reaches an absorbance threshold;
iv) measuring an absorbance of the at least one fraction of eluate collected from the column and determining an A260/A280 ratio; and/or
v) combining at least two fractions of eluate collected from the column to form a pooled eluate comprising the purified rAAV vector.
   E275. The purified rAAV vector prepared by the method of E274, wherein the material eluted from the stationary phase comprises the rAAV vector to be purified
   E276. The purified rAAV vector prepared by the method of E274 or E275, wherein the rAAV vector comprises an AAV9 capsid protein.
   E277. The purified rAAV vector prepared by the method of any one of E274-E276, wherein the method further comprised filtering the pooled eluate by a method selected from the group consisting of viral filtration, ultrafiltration/diafiltration (UF/DF), filtration through a 0.2 μm filter and a combination thereof, to produce a drug substance.
   E278. The purified rAAV vector prepared by the method of E277, wherein the drug substance is used to make a drug product suitable for administration to a human subject to treat a disease, disorder or condition.
   E279. The purified rAAV vector prepared by the method of E278, wherein the disease, disorder or condition is DMD.
   E280. A solution comprising a rAAV vector for purification by AEX prepared by a method comprising a step of:
i) diluting a first solution (e.g., an affinity eluate) 2 to 25-fold (e.g., about with a buffer comprising 200 mM histidine, 200 mM Tris, 0.5% P188, pH 8.8;
and optionally
ii) filtering the first solution from step i) through a 0.2 μm filter to produce a diluted and optionally filtered solution; wherein the pH of the diluted and optionally filtered solution is increased as compared to the pH of the first solution; and wherein the conductivity of the diluted and optionally filtered solution is decreased as compared to the conductivity of the first solution.
   E281. The solution comprising a rAAV vector for purification by AEX of E280, wherein the first solution is an affinity eluate.
   E282. The solution comprising a rAAV vector for purification by AEX of E281, wherein the affinity eluate is generated from affinity purification of a rAAV vector produced in a vessel (e.g., a single use bioreactor) with a volume of 250 L or 2000 L.
   E283. The solution comprising a rAAV vector for purification by AEX of any one of E280-E282, wherein the pH of the diluted and optionally filtered solution is 8.5 to 9.5.
   E284. The solution comprising a rAAV vector for purification by AEX of any one of E280-E283, wherein the conductivity of the diluted and optionally filtered solution is 1.7 to 3.3 mS/cm.
   E285. The solution comprising a rAAV vector for purification by AEX of any one of E280-E284, wherein a % VG dilution yield of the diluted and optionally filtered solution is 88%+/−36%.
   E286. A solution comprising a rAAV vector for purification by AEX prepared by a method comprising a step of:
i) diluting a first solution (e.g., an affinity eluate) 2 to 25-fold (e.g., 15-fold) with a dilution solution comprising histidine, Tris, and P188; and optionally
ii) filtering the diluted first solution from step i) through a 0.2 μm filter to produce a diluted, and optionally filtered solution; wherein the pH of the diluted, and optionally filtered solution is increased as compared to the pH of the first solution; and wherein the conductivity of the diluted, and optionally filtered solution is decreased as compared to the conductivity of the first solution.
   E287. The solution comprising a rAAV vector for purification by AEX prepared by a method of E280, wherein the dilution solution comprises about 100 mM to 300 mM (e.g., about 200 mM) histidine, 100 mM to 300 mM (about 200 mM) Tris, 0.1% to 1.0% (about 0.5%) P188, pH 8.5 to 9.5.
   E288. The solution comprising a rAAV vector for purification by AEX prepared by a method of E280 or E281, wherein the pH the first solution is 3.0 to 4.4 prior to diluting, and optionally filtering, and the pH of the first solution after diluting, and optionally filtering, is 8.5 to 9.5 or 8.7 to 9.0 (e.g., 8.8, 9.0).
   E289. The solution comprising a rAAV vector for purification by AEX prepared by a method of any one of E280-E282, wherein conductivity of the first solution is 5.0 to 7.0 mS/cm (e.g., 5.5 to 6.5 mS/cm) prior to the step of diluting, and optionally filtering, and the conductivity of the first solution after the step of diluting, and optionally filtering, is 1.7 to 3.5 mS/cm, 1.8 to 2.8 mS/cm, or 2.2 to 2.6 mS/cm.
   E290. The solution comprising a rAAV vector for purification by AEX prepared by a method of any one of E280-E283, wherein the % VG dilution yield of the diluted and optionally filtered first solution is 35% to 100%.
   E291. The solution comprising a rAAV vector for purification by AEX prepared by a method of any one of E280-E28, wherein the rAAV vector comprises an AAV9 or an AAV3B capsid protein, and optionally, wherein the diluted and optionally filtered solution is loaded on an AEX stationary phase.
   E292. A method of regenerating an AEX stationary phase, the method comprising a step of:
i) post-use sanitizing of the stationary phase comprising application of 14.4 to 17.6 CV (e.g., about 16 CV) of a solution comprising about 0.1 M to 1.0 M (e.g., about 0.5 M) NaOH to the stationary phase, optionally by upward flow;
ii) regenerating the stationary phase comprising application of 4.5 to 5.5 CV (e.g., about 5 CV) of a solution comprising about 1 M to 3 M (e.g., about 2 M) NaCl, 50 mM to 150 mM (e.g., about 100 mM) Tris, pH 8.5 to 9.5 (e.g., about pH 9) to the stationary phase;
iii) equilibration of the stationary phase comprising application of 4.5 to 5.5 CV (e.g., about 5 CV) of a solution comprising about 50 mM to 150 mM (e.g., about 100 mM) Tris, pH 8.5 to 9.5 (e.g., about pH 9) to the stationary phase;
iv) post-use flushing of the stationary phase comprising application of ≥4.5 (e.g., about 5 CV) of water for injection to the stationary phase; and/or
v) applying a storage solution to the stationary phase comprising application 2.7 to 3.3 CV (e.g., about 3 CV) of a solution comprising about 17.5% ethanol to the stationary phase, optionally wherein at least one of steps i)-v) is performed at a linear velocity of 270 to 330 cm/hr (e.g., about 300 cm/hr), a flow rate of 1.5 to 2.0 L/min (e.g., about 1.8 L/min) through a 6.0 to 6.6 L (e.g., 6.4 L) column, or about 314 mL/min through a 1.3 L column, and/or a residence time of 3.5 to 4.5 min/CV (e.g., about 4 min/CV).
   E293. The method of regenerating an AEX stationary phase of E292, wherein any one of steps i)-v) step follows a chromatography elution step of a method of purifying a rAAV vector by AEX.
   E294. A regenerated AEX stationary phase prepared by a method comprising a step of:
i) post-use sanitizing of the stationary phase comprising application of 14.4 to 17.6 CV (e.g., about 16 CV) of a solution comprising about 0.1 M to 1.0 M (e.g., about 0.5 M) NaOH to the stationary phase, optionally by upward flow;
ii) regenerating the stationary phase comprising application of 4.5 to 5.5 CV (e.g., about 5 CV) of a solution comprising about 1 M to 3 M NaCl (e.g., about 2 M) NaCl, about 50 mM to 150 mM (e.g., about 100 mM) Tris, pH 8.5 to 9.5 (e.g., about pH 9) to the stationary phase;
iii) equilibration of the stationary phase comprising application of 4.5 to 5.5 CV (e.g., about 5 CV) of a solution comprising about 50 mM to 150 mM (e.g., about 100 mM) Tris, pH 8.5 to 9.5 (e.g., pH 9) to the stationary phase;
iv) post-use flushing of the stationary phase comprising application of ≥4.5 (e.g., about 5 CV) of water for injection to the stationary phase; and/or
v) applying a storage solution to the stationary phase comprising application of 2.7 to 3.3 CV (e.g., about 3 CV) of a solution comprising about 17% to 17.5% ethanol to the stationary phase; optionally
wherein at least one of steps i)-v) step is performed at a linear velocity of 270 to 330 cm/hr (e.g., about 300 cm/hr), a flow rate of 1.5 to 2.0 L/min (e.g., about 1.8 L/min) through a 6.0 to 6.6 L (e.g., 6.4 L) column, or about 314 mL/min through a 1.3 L column, and/or a residence time of 3.5 to 4.5 min/CV (e.g., about 4 min/CV).
   E295. The regenerated AEX stationary phase of E288, wherein the regenerated AEX stationary phase is used for purification of a rAAV vector.
   E296. A pharmaceutical composition comprising a purified rAAV vector made by the method of any one of E1-E227 or E231-E258.
   E297. A pharmaceutical composition comprising a purified rAAV vector of E267-E279.
   E298. Use of a rAAV vector purified according to the method of any one of E1-E227 or E231-E258 in the manufacture of a medicament for treating and/or preventing a disease, disorder or condition.
   E299. The use of E298, wherein the disease, disorder or condition is DMD.
   E300. The method of any one of E1-E182, wherein the rAAV vector comprises a vector genome comprising a modified nucleic acid encoding a deleted cooper-transporting ATPase2 (ATP7B) protein.
   E301. The method of E300, wherein the modified nucleic acid comprises a nucleic acid sequence encoding a deleted cooper-transporting ATPase2 (ATP7B) protein comprising or consisting of the amino acid sequence of SEQ ID NO:15.
   E302. The method of E300 or E301, wherein the deleted copper-transporting APTase2 comprises a deletion of Heavy-Metal-Associated sited HMA 1, HMA 2, HMA 3 and HMA 4.
   E303. The method of any one of E300-E302, wherein the vector genome further comprises an α1-antitrypsin promoter, a polyadenylation (polyA) signal sequence, a 5′ ITR and a 3′ ITR.
   E304. The method of E303, wherein the α1-antitrypsin promoter comprises or consists of the nucleic acid sequence of SEQ ID NO:16.
   E305. The method of E303, wherein the polyA signal sequence comprises or consists of the nucleic acid sequence of SEQ ID NO:17.
   E306. The method of E303, wherein the 5′ ITR and the 3′ ITR are AAV2 serotype ITRs.
   E307. The method of any one of E1-E182, wherein the rAAV vector comprises a VP1 polypeptide of AAV3B.
   E308. The method of E307, wherein the VP1 polypeptide comprises or consists of the amino acid sequence of SEQ ID NO:18.
   E309. A method of purifying an rAAV vector by AEX, the method comprising a step of:
i) loading a solution comprising the rAAV vector to be purified onto an AEX stationary phase in a column;
ii) performing gradient elution of a material from the stationary phase in the column wherein a percentage of a first gradient elution buffer is varied in a manner inversely proportional to variation in a percentage of a second gradient elution buffer; wherein at the start of the gradient elution the percentage of the first gradient elution buffer is about 75% to about 100% and at the end of the gradient elution the percentage of the second gradient elution buffer is about 60% to about 100%; and wherein the percentage of the second elution buffer increases at a rate of about 2% to 5% per CV over the gradient elution;
iii) collecting at least one fraction of eluate from the column when performing the gradient elution beginning when the percentage of the second gradient elution buffer is about 30% to about 35%,
and wherein the at least one fraction of eluate comprises the rAAV vector to be purified.
   E310. The method of purifying an rAAV vector by AEX of E309, wherein the solution comprising the rAAV vector is an affinity eluate that has been diluted about fold with a buffer comprising histidine, Tris and P188.
   E311. The method of purifying an rAAV vector by AEX of E309 or E310, wherein the first gradient elution buffer comprises 50 mM to 150 mM (e.g., about 100 mM) Tris, 0.005% to 0.015% (e.g., about 0.01%) P188, pH 8.5 to 9.5 (e.g., 8.9) and/or the second gradient elution buffer comprises 400 mM to 600 mM (e.g., about 500 mM) sodium acetate, 50 mM to 150 mM (e.g., about 100 mM) Tris, 0.005% to (e.g., about 0.01%) P188, pH 8.5 to 9.5 (e.g., pH 8.9).
   E312. The method of purifying an rAAV vector by AEX of any one of E309-E311, wherein collecting at least one fraction of eluate from the column comprises collecting the at least one fraction of eluate into a vessel comprising about 0.01 CV to 0.1 CV (e.g., about 0.066 CV) of a solution comprising 200 mM to 300 mM (e.g., about 250 mM) sodium citrate, pH 3.0 to 4.0 (e.g., about 3.5).
   E313. The method of purifying a rAAV vector by AEX of any one of E310-E312, wherein the at least one fraction of eluate is enriched for full capsids, and/or depleted of empty capsids, as compared to the affinity eluate; optionally wherein the rAAV vector is a rAAV3B vector; and optionally wherein the AEX stationary phase is POROS™ 50 HQ.
   E314. The method of purifying a rAAV vector by AEX of any one of E309-E313, wherein the collecting at least one fraction of eluate from the column when performing the gradient elution ends when the percentage of the second gradient elution buffer is about 50% to about 55%.
   E315. A method of purifying an rAAV vector by AEX, the method comprising a step of: i) loading a solution comprising the rAAV vector to be purified onto an AEX stationary phase in a column;
ii) performing gradient elution of a material from the stationary phase in the column wherein a percentage of a first gradient elution buffer is varied in a manner inversely proportional to variation in a percentage of a second gradient elution buffer; and
iii) collecting at least one fraction of eluate from the column during the gradient elution beginning when the absorbance of a column flow-through reaches an absorbance threshold, and wherein the at least one fraction of eluate comprises the rAAV vector to be purified.
   E316. The method of purifying a rAAV vector by AEX of E315, wherein the method further comprises measuring an absorbance of the at least one fraction of eluate collected from the column and determining an A260/A280 ratio.
   E317. The method of purifying a rAAV vector by AEX of E315 or E316, wherein the solution comprising the rAAV vector to be purified is diluted about 2-fold to 25-fold (e.g., 15-fold) with a dilution solution comprising histidine, Tris and P188, and optionally filtered prior to application to the stationary phase.
   E318. The method of purifying a rAAV vector by AEX of any one of E315-E317, wherein the solution is an affinity eluate.
   E319. The method of purifying a rAAV vector by AEX of E315-E318, wherein a pH of the diluted, and optionally filtered affinity eluate is increased as compared to a pH of the solution; and wherein a conductivity of the diluted, and optionally filtered affinity eluate is decreased as compared to a conductivity of the solution.
   E320. The method of purifying a rAAV vector by AEX of any one of E315-E319, wherein the first gradient elution buffer comprises about 50 mM to about 150 mM Tris, about 0.005% to about 0.015% P188 and has a pH of about pH 8.5 to 9.5; wherein the second gradient elution buffer comprises about 400 mM to about 600 mM sodium acetate, about 50 mM to about 150 mM Tris, about 0.005% to about P188 and has a pH of about pH 8.5 to 9.5; and wherein 10 to 60 column volumes (CV) (e.g., about 20 CV, about 37.5 CV) of the first gradient elution buffer, the second gradient elution buffer or a mixture of both are applied to the stationary phase during the gradient elution.
   E321. The method of purifying a rAAV vector by AEX of any one of E315-E320, wherein at a start of the gradient elution the percentage of the first gradient elution buffer is 50%-100% and at an end of the gradient elution the percentage of the second gradient elution buffer is 50%-100% and wherein the percentage of the second elution buffer increases at a rate of about 2% to 5% per CV over the gradient elution.
   E322. The method of purifying a rAAV vector by AEX of any one of E315-E321, wherein a concentration of sodium acetate of the first gradient elution buffer, the second gradient elution buffer or the mixture of both increases continuously during the gradient elution; and wherein the concentration of the sodium acetate increases at a rate of about 10 mM/CV to 50 mM/CV (e.g., about 10 mM/CV, about 25 mM/CV) over the gradient elution.
   E323. The method of purifying a rAAV vector by AEX of any one of E315-E322, wherein full capsids are eluted from the stationary phase in a first elution peak and/or in a first portion of a second elution peak during the gradient elution.
   E324. The method of purifying a rAAV vector by AEX of any one of E315-E323, wherein empty capsids are recovered in the column flow-through, in a first elution peak and/or in a last portion of a second elution peak during the gradient elution.
   E325. The method of purifying a rAAV vector by AEX of any one of E315-E324, wherein an absorbance of the at least one fraction of eluate is measured at 280 nm, and wherein optionally, an absorbance threshold is ≥0.5 mAU/mm path length measured at 280 nm.
   E326. The method of purifying a rAAV vector by AEX of any one of E315-E325, wherein a volume of the at least one fraction of eluate is equivalent to ⅛ of a CV to 10 CV, e.g., ⅛ of a CV, ¼ of a CV, ⅓ of a CV, ½ of a CV, ¾ of a CV, 1 CV, 2 CV, 3 CV, 4 CV, 5 CV, 6 CV, 7 CV, 8 CV, 9 CV, 10 CV or more of a CV, and optionally, wherein an A260/A280 ratio of the at least one fraction of eluate is ≥ to 1.25.
   E327. The method of purifying a rAAV vector by AEX of any one of E315-E326, wherein at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more, fractions of eluate are collected.
   E328. The method of purifying a rAAV vector by AEX of any one of E315-E327, wherein the method further comprises combining at least two fractions of eluate collected from the column, each having an A260/A280 ratio of ≥0.98 or ≥1.0 to form a pooled eluate comprising the rAAV vector.
   E329. The method of purifying a rAAV vector by AEX of E328, wherein the pooled eluate has a % VG column yield of 20% to 100% (e.g., 63+/−26%), a % VG step yield of 31% to 66% (e.g., 47+/−11%) and/or an A260/A280 ratio of ≥1.0.
   E330. The method of purifying a rAAV vector by AEX of E328 or E329, wherein the pooled eluate is enriched for full capsids, and/or depleted of empty capsids, as compared to the solution loaded onto the column.
   E331. The method of purifying a rAAV vector by AEX of any one of E315-E330, wherein a purified rAAV vector is produced.
   E332. The method of purifying a rAAV vector by AEX of any one of E328-E331, further comprising filtering the pooled eluate by a method selected from the group consisting of viral filtration, ultrafiltration/diafiltration (UF/DF), filtration through a 0.2 μm filter and a combination thereof, to produce a drug substance.
   E333. The method of purifying a rAAV vector by AEX of E332, wherein the drug substance comprises: i) 45% to 65% (e.g., 52+/−7%) full capsids of total capsids; ii) 19% to 37% (e.g., 28+/−5%) intermediate capsids of total capsids; and/or iii) 10% to 37% (e.g., 20+/−7%) empty capsids of total capsids.
   E334. The method of purifying a rAAV vector by AEX of E332-E333, wherein the drug substance is enriched for full capsids, and/or depleted of empty capsids, as compared to the solution loaded onto the column.
   E335. The method of purifying a rAAV vector by AEX of any one of E315-E334, wherein the rAAV vector comprises an AAV capsid protein from an AAV serotype selected from the group consisting of AAV1, AAV2, AAV3, AAV3A, AAV3B, AAV4, AAV5, AAV6, AAV7, AAV8, AAV9, AAV10, AAVrh10, AAVrh74, AAV12, AAV2i8, NP4, NP22, NP66, AAVDJ, AAVDJ/8, AAVDJ/9, AAVLK03, AAV1.1, AAV2.5, AAV6.1, AAV6.3.1, AAV9.45, RHM4-1 (SEQ ID NO:5 of WO 2015/013313), RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15.
   E336. The method of purifying a rAAV vector by AEX of any one of E315-E335, wherein the rAAV vector comprises an AAV9 capsid protein and a transgene comprising the nucleic acid of SEQ ID NO:1.
   E337. The method of purifying a rAAV vector by AEX of any one of E315-E336, wherein the rAAV vector comprises an AAV3B capsid protein and a transgene comprising a nucleic acid encoding the amino acid sequence of SEQ ID NO:15.
   E338. A method of preparing a solution comprising a rAAV vector for purification by AEX, the method comprising a step of:
i) diluting a first solution 2 to 25-fold (e.g., 15-fold) with a dilution solution comprising histidine, Tris and P188; and optionally
ii) filtering the first solution from step i) through a filter to produce a diluted, and optionally filtered solution;
   wherein the pH of the diluted, and optionally filtered solution is increased as compared to the pH of the first solution; and wherein the conductivity of the diluted, and optionally filtered solution is decreased as compared to the conductivity of the first solution.
   E339. The method of preparing a solution comprising a rAAV vector for purification by AEX of E338, wherein the first solution comprising the rAAV vector is selected from the group consisting of an affinity eluate, a supernatant from a cell lysate and a post-harvest solution.
   E340. The method of preparing a solution comprising a rAAV vector for purification by AEX of E338 or E339, wherein the dilution solution comprises about 100 mM to 300 mM (e.g., about 200 mM) histidine, 100 mM to 300 mM (e.g., about 200 mM) Tris, 0.1% to 1.0% (e.g., about 0.5%) P188 and has a pH of pH 8.5 to 9.5.
   E341. The method of preparing a solution comprising a rAAV vector for purification by AEX of any one of E338-E340, wherein i) a pH of the diluted, and optionally filtered solution is 8.5 to 9.5; ii) a conductivity of the diluted, and optionally filtered solution is 1.7 to 3.3 mS/cm; and/or iii) a % VG dilution yield of the diluted solution is 35% to 100%.
   E342. The method of preparing a solution comprising a rAAV vector for purification by AEX of any one of E338-E341, wherein the rAAV vector comprises an AAV9 capsid protein or an AAV3B capsid protein.
   E343. A purified rAAV vector prepared by a method comprising a step of:
i) loading a solution comprising the rAAV vector to be purified onto an AEX stationary phase in a column;
ii) performing gradient elution of a material from the stationary phase in the column wherein a first gradient elution buffer, a second gradient elution buffer or a mixture of both are applied to the stationary phase and a concentration of a salt is varied from 0 mM to 500 mM such that the rate of increase in concentration of the salt over the course of the gradient elution is about 10 mM/CV to 50 mM/CV (e.g., about 25 mM/CV);
iii) collecting at least one fraction of eluate from the column during gradient elution beginning when absorbance of a column flow-through reaches an absorbance threshold; and/or
vi) measuring an absorbance of the at least one fraction of eluate collected from the column and determining an A260/A280 ratio.
   E344. The purified rAAV vector prepared by the method of E343, wherein the method further comprises combining at least two fractions of eluate collected from the column when the A260/A280 ratio is ≥1.0 to form a pooled eluate comprising the purified rAAV vector.
   E345. The purified rAAV vector prepared by the method of E343 or E344, wherein the salt is sodium acetate.
   E346. The purified rAAV vector prepared by the method of any one of E343-E345, wherein the rAAV vector comprises an AAV9 capsid protein or an AAV3B capsid protein.
   E347. The purified rAAV vector prepared by the method of any one of E343-E346, wherein the solution comprising the rAAV vector is an affinity eluate that has been diluted and optionally filtered prior to loading onto the stationary phase.
   E348. The purified rAAV vector prepared by the method of any one of E343-E347, wherein the material eluted from the stationary phase comprises the rAAV vector.
   E349. The purified rAAV vector prepared by the method of any one of E344-E348, wherein the method further comprises filtering the pooled eluate by a method selected from the group consisting of viral filtration, ultrafiltration/diafiltration (UF/DF), filtration through a 0.2 μm filter and a combination thereof, to produce a drug substance.
   E350. The purified rAAV vector prepared by the method of E349, wherein the drug substance is used to make a drug product suitable for administration to a human subject to treat a disease, disorder or condition.
   E351. The purified rAAV vector prepared by the method of E350, wherein the disease, disorder or condition is DMD or Wilson's disease, and optionally wherein the rAAV vector comprises a nucleic acid encoding the amino acid sequence of SEQ ID NO:2 or SEQ ID NO:15.

### BRIEF DESCRIPTION OF DRAWINGS

FIG. 1 depicts the elution phases of exemplary AEX chromatograms generated using four elution salts on a 1 mL POROS™ 50 HQ column. A₂₆₀ trace is shown by a dashed line, while A₂₈₀ and conductivity traces are shown by solid lines. Solid bars indicate elution fractions that were used to form pools.

FIG. 2 depicts exemplary SEC A₂₆₀/A₂₈₀ of AEX elution fractions generated using four elution salts on a 1 mL POROS™ 50 HQ column.

FIG. 3 depicts an exemplary AEX chromatogram generated using a sodium acetate 9-step wash and elution carried out on a 5.1 mL POROS™ 50 HQ column. A₂₆₀ trace is shown by a dashed line, while A₂₈₀ and conductivity traces are shown by solid lines. Wash (W), elution (E), strip, and regeneration (Regen.) fractions are indicated consistent with Table 5 and Table 6.

FIG. 4A depicts an exemplary AEX chromatogram generated using a sodium acetate step elution run with a 600 cm/hr elution, 5.1×10¹³ vector genome/mL resin challenge (VG/mL resin, as measured by qPCR of the ITR sequences), and 57 mM Sodium acetate wash carried out on a 5.1 mL POROS™ 50 HQ column. A₂₆₀ trace is shown by a dashed line, while A₂₈₀ and conductivity traces are shown by solid lines. FIG. 4B depicts a magnified view of the chromatogram at the wash, elution and strip phases of the AEX run.

FIG. 5 depicts an exemplary method of in-line mixing of an AAV9 affinity eluate with 100 mM Tris, pH 9 to generate an AEX load (also referred to herein as a diluted affinity eluate). Fluids were delivered to the Y-connector with peristaltic pumps.

FIG. 6 depicts exemplary pH, conductivity, Z-Average, and aggregation (given as + or −) of an AAV9 affinity eluate diluted with 100 mM Tris, pH 9.

FIG. 7A and FIG. 7B depict exemplary % vector genome (VG) yield for affinity eluates diluted 5, 9, or 25-fold with 200 mM histidine, 200 mM Tris, X % (w/v) P188, pH 8.8, where X is 0.01%, 0.05%, 0.2%, and 0.5%, followed by filtration. A contour plot of % VG yield (post dilution and filtration) as a function of conductivity (controlled by dilution factor) and P188 concentration is depicted in FIG. 7A. One way ANOVA analyses of % VG yield (post dilution and filtration) as a function of P188 concentration or conductivity are depicted in FIG. 7B. Data is also presented in Table 13.

FIG. 8A depicts an exemplary chromatogram generated using the optimized AEX process. FIG. 8B depicts a zoom-in of AEX sodium acetate gradient elution, with fractions numbered 1-14, consistent with Table 15. A₂₆₀ trace is given in dashed line, while A₂₈₀ and conductivity traces are given in solid lines.

FIG. 9 depicts exemplary SEC A₂₆₀/A₂₈₀ values of chromatographic fractions generated using the optimized AEX method on 0%, 20%, 40%, 60%, 80%, and 100% Null affinity pools. Flow-through is abbreviated as F/T.

FIG. 10 depicts the elution phase of an exemplary 250 L SUB AEX chromatogram from Batch 250L-4, run on a 10 cm inner diameter (ID)×16 cm bed height (BH), 1.3 L POROS™ 50 HQ column. A₂₆₀ trace is shown by a dashed line, while A₂₈₀ and conductivity traces are shown by solid lines.

FIG. 11 depicts the elution phase of an exemplary 2000 L SUB Scale AEX chromatogram, batch 2000 L-4, run on a 20 cm ID×20.5 cm BH, 6.4 L POROS™ 50 HQ column. A₂₆₀ trace is shown by a dashed line, while A₂₈₀ and conductivity traces are shown by solid lines.

FIG. 12 depicts and exemplary chromatogram using the optimized AEX process for the purification of an AAV3B vector. A₂₆₀ trace is shown by a solid line, while A₂₈₀ trace is shown by a dashed line.

### DESCRIPTION

### 1. Definitions

Unless otherwise defined, all technical and scientific terms used herein have the meaning commonly understood by one of ordinary skill in the art to which this invention belongs. The terminology used herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. The following terms have the meanings given:

As used herein, the term “about,” or “approximately” refers to a measurable value such as an amount of the biological activity, length of a polynucleotide or polypeptide sequence, content of G and C nucleotides, codon adaptation index, number of CpG dinucleotides, dose, time, temperature, and the like, and is meant to encompass variations of 25%, 20%, 19%, 18%, 17%, 16%, 15%, 14%, 13%, 12%, 11%, 10%, 9%, 8%, 7%, 6%, 5%, 4%, 3%, 2%, 0.5% or even 0.1%, in either direction (greater than or less than) of the specified amount unless otherwise stated, otherwise evident from the context, or except where such number would exceed 100% of a possible value.

As used herein, the term “and/or” refers to and encompasses any and all possible combinations of one or more of the associated listed items, as well as the lack of combinations when interpreted in the alternative (“or”).

As used herein, the terms “adeno-associated virus” and/or “AAV” refer to a parvovirus with a linear single-stranded DNA genome and variants thereof. The term covers all subtypes and both naturally occurring and recombinant forms, except where required otherwise.

The canonical AAV wild-type genome comprises 4681 bases (Berns and Bohenzky (1987) Advances in Virus Research 32:243-307) and includes terminal repeat sequences (e.g., inverted terminal repeats (ITRs)) at each end which function in cis as origins of DNA replication and as packaging signals for the virus. The genome includes two large open reading frames, known as AAV replication (“AAV rep” or “rep”) and capsid (“AAV cap” or “cap”) genes, respectively. AAV rep and cap may also be referred to herein as AAV “packaging genes.” These genes code for the viral proteins involved in replication and packaging of the viral genome.

In wild type AAV, three capsid genes VP1, VP2 and VP3 overlap each other within a single open reading frame and alternative splicing leads to production of VP1, VP2 and VP3 capsid proteins (Grieger and Samulski (2005) J. Virol. 79(15):9933-9944). A single P40 promoter allows all three capsid proteins to be expressed at a ratio of about 1:1:10 for VP1, VP2, VP3, respectively, which complements AAV capsid production. More specifically, VP1 is the full-length protein, with VP2 and VP3 being increasingly shortened due to increasing truncation of the N-terminus. A well-known example is the capsid of AAV9 as described in U.S. Pat. No. 7,906,111, wherein VP1 comprises amino acid residues 1 to 736 of SEQ ID NO:123, VP2 comprises amino acid residues 138 to 736 of SEQ ID NO:123, and VP3 comprises amino acid residues 203 to 736 of SEQ ID NO:123. As used herein, the term “AAV Cap” or “cap” refers to AAV capsid proteins VP1, VP2 and/or VP3, and variants and analogs thereof. A second open reading frame of the capsid gene encodes an assembly factor, called assembly-activating protein (AAP), which is essential for the capsid assembly process (Sonntag et al. (2011) J. Virol.

At least four viral proteins are synthesized from the AAV rep gene—Rep 78, Rep 68, Rep 52 and Rep 40—named according to their apparent molecular weights. As used herein, “AAV rep” or “rep” means AAV replication proteins Rep 78, Rep 68, Rep 52 and/or Rep 40, as well as variants and analogs thereof. As used herein, rep and cap refer to both wild type and recombinant (e.g., modified chimeric, and the like) rep and cap genes as well as the polypeptides they encode. In some embodiments, a nucleic acid encoding a rep will comprise nucleotides from more than one AAV serotype. For instance, a nucleic acid encoding a rep protein may comprise nucleotides from an AAV2 serotype and nucleotides from an AAV3 serotype (Rabinowitz et al. (2002) J. Virology 76(2):791-801).

As used herein the terms “recombinant adeno-associated virus vector,” “rAAV” and/or “rAAV vector” refer to an AAV capsid comprising a vector genome. The vector genome comprises a polynucleotide sequence that is not, at least in part, derived from a naturally-occurring AAV (e.g., a heterologous polynucleotide not present in wild type AAV), and the rep and/or cap genes of the wild type AAV genome have been removed from the vector genome. Where the rep and/or cap genes of the AAV have been removed (and/or ITRs from an AAV have been added or remain), the nucleic acid within the AAV is referred to as the “vector genome.”

Therefore, the term rAAV vector encompasses both a rAAV viral particle that comprises a capsid but does not comprise a complete AAV genome; instead the recombinant viral particle can comprise a heterologous, i.e., not originally present in the capsid, nucleic acid, hereinafter referred to as a vector genome. Thus, a “rAAV vector genome” (or “vector genome”) refers to a heterologous polynucleotide sequence (including at least one ITR) that may, but need not, be contained within an AAV capsid. A rAAV vector genome may be double-stranded (dsAAV), single-stranded (ssAAV) or self-complementary (scAAV). Typically, a vector genome comprises a heterologous (to the original AAV from which it is derived) nucleic acid often encoding a therapeutic transgene, a gene editing nucleic acid, and the like.

As used herein, the terms “rAAV vector,” “rAAV viral particle” and/or “rAAV vector particle” refer to an AAV capsid comprised of at least one AAV capsid protein (though typically all of the capsid proteins, e.g., VP1, VP2 and VP3, or variant thereof, of a AAV are present) and containing a vector genome comprising a heterologous nucleic acid sequence. These terms are to be distinguished from an “AAV viral particle” or “AAV virus” that is not recombinant wherein the capsid contains a virus genome encoding rep and cap genes and which AAV virus is capable of replicating if present in a cell also comprising a helper virus, such as an adenovirus and/or herpes simplex virus, and/or required helper genes therefrom. Thus, production of a rAAV vector particle necessarily includes production of a recombinant vector genome using recombinant DNA technologies, as such, which vector genome is contained within a capsid to form a rAAV vector, rAAV viral particle, or a rAAV vector particle.

The genomic sequences of various serotypes of AAV, as well as the sequences of the inverted terminal repeats (ITRs), rep proteins, and capsid subunits are known in the art. Such sequences may be found in the literature or in public databases such as GenBank. See, e.g., GenBank Accession Numbers NC_002077 (AAV1), AF063497 (AAV1), NC_001401 (AAV2), AF043303 (AAV2), NC_001729 (AAV3), AF028705.1 (AAV3B), NC_001829 (AAV 4), U89790 (AAV4), NC_006152 (AAV5), AF028704 (AAV6), AF513851 (AAV7), AF513852 (AAV8), NC_006261 (AAV8), AY530579 (AAV9), AY631965 (AAV10), AY631966 (AAV11), and DQ813647 (AAV12); the disclosures of which are incorporated by reference herein. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99: 11854; Moris et al. (2004) Virology 33:375-383; international patent publications WO 00/28061, WO 99/61601, WO 98/11244; WO 2013/063379, WO 2014/194132, WO 2015/121501; and U.S. Pat. Nos. 6,156,303 and 7,906,111.

As used herein, the term “associated with” refers to with one another, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example, by means of hydrogen bonds, van der Waals interaction, hydrophobic interactions, magnetism, and a combination thereof.

As used herein, the term “coding sequence” or “nucleic acid encoding” refers to a nucleic acid sequence which encodes a protein or polypeptide and denotes a sequence which is transcribed (in the case of DNA) and translated (in the case of mRNA) into a polypeptide in vitro or in vivo when placed under the control of (operably linked to) appropriate regulatory sequences. The boundaries of a coding sequence are generally determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences.

As used herein, the term “chimeric” refers to a viral capsid or particle, with capsid or particle sequences from different parvoviruses, preferably different AAV serotypes, as described in Rabinowitz et al., U.S. Pat. No. 6,491,907, the disclosure of which is incorporated in its entirety herein by reference. See also Rabinowitz et al. (2004) J. Virol. 78(9):4421-4432. In some embodiments, a chimeric viral capsid is an AAV2.5 capsid which has the sequence of the AAV2 capsid with the following mutations: 263 Q to A; 265 insertion T; 705 N to A; 708 V to A; and 716 T to N. The nucleotide sequence encoding such capsid is defined as SEQ ID NO: 15 as described in WO 2006/066066. Other preferred chimeric AAV capsids include, but are not limited to, AAV2i8 described in WO 2010/093784, AAV2G9 and AAV8G9 described in WO 2014/144229, and AAV9.45 (Pulicherla et al. (2011) Molecular Therapy 19(6):1070-1078), AAV-NP4, NP22 and NP66, AAV-LK0 through AAV-LK019 described in WO 2013/029030, RHM4-1 and RHM15_1 through RHM5_6 described in WO 2015/013313, AAVDJ, AAVDJ/8, AAVDJ/9 described in WO 2007/120542.

As used herein, the term “eluate” refers to fluid exiting from a chromatography stationary phase (e.g., a monolith, membrane, resin, media) (e.g., “eluting from the stationary phase”) comprised of mobile phase and material that passed through the stationary phase or was displaced from the stationary phase. In some embodiments, a stationary phase includes, for example, a monolith, a membrane, a resin or a media. The mobile phase may be a solution that has been loaded onto a column and has flowed through the column (i.e., “flow-through fraction”); an equilibration solution (e.g. an equilibration buffer); an isocratic elution solution; a gradient elution solution; a solution for regenerating a stationary phase; a solution for sanitizing a stationary phase; a solution for washing; and combinations thereof.

As used herein, the term “flanked,” refers to a sequence that is flanked by other elements and indicates the presence of one or more flanking elements upstream and/or downstream, i.e., 5′ and/or 3′, relative to the sequence. The term “flanked” is not intended to indicate that the sequences are necessarily contiguous. For example, there may be intervening sequences between a nucleic acid encoding a transgene and a flanking element. A sequence (e.g., a transgene) that is “flanked” by two other elements (e.g., ITRs), indicates that one element is located 5′ to the sequence and the other is located 3′ to the sequence; however, there may be intervening sequences there between.

As used herein, the term “flocculation” refers to the process by which fine particulates are caused to clump together into a floc. The fine particles may include proteins, nucleic acids, cellular fragments resulting from lysis of host cells. In some embodiments, a floc that forms in a liquid phase may float to the top of the liquid (creaming), settle to the bottom (sedimentation) of the liquid or be filtered from the liquid phase.

As used herein, the term “fragment” refers to a material or entity that has a structure that includes a discrete portion of the whole but lacks one or more moieties found in the whole. In some embodiments, a fragment consists of a discrete portion. In some embodiments, a fragment consists of or comprises a characteristic structural element or moiety found in the whole. In some embodiments, a polymer fragment comprises, or consists of, at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., amino acid residues, nucleotides) found in the whole polymer.

rAAV vectors are referred to as “full,” a “full capsid,” “full vector” or a “fully packaged vector” when the capsid contains a complete, or essentially complete, vector genome, including a transgene. During production of rAAV vectors by host cells, vectors may be produced that have less packaged nucleic acid than the full capsids and contain, for example a partial or truncated vector genome. These vectors are referred to as “intermediates,” an “intermediate capsid,” a “partial” or a “partially packaged vector.” An intermediate capsid may also be a capsid with an intermediate sedimentation rate, that is a sedimentation rate between that of full capsids and empty capsids, when analyzed by analytical ultracentrifugation. Host cells may also produce viral capsids that do not contain any detectable nucleic acid material. These capsids are referred to as “empty(s),” or “empty capsids.” Full capsids may be distinguished from empty capsids based on A260/A280 ratios determined by SEC-HPLC, whereby the A260/A280 ratios have been previously calibrated against capsids (i.e., full, intermediate and empty) isolated by analytical ultracentrifugation. Other methods known in the art for the characterization of capsids include CryoTEM, capillary isoelectric focusing and charge detection mass spectrometry. Calculated isoelectric points of ˜6.2 and ˜5.8 for empty and full AAV9 capsids, respectively have been reported (Venkatakrishnan et al., J. Virology (2013) 87.9:4974-4984).”

As used herein, the term “null capsid” refers to a capsid produced intentionally to lack a vector genome. Such null a capsid may be produced by transfection of a host cell with a rep/cap and a helper plasmid, but not a plasmid that comprises the transgene cassette sequence, also known as a vector plasmid.

As used herein, the term “functional” refers to a biological molecule in a form in which it exhibits a property and/or activity by which it is characterized. A biological molecule may have two functions (i.e., bifunctional) or many functions (i.e., multifunctional).

As used herein, the term “gene” refers to a polynucleotide containing at least one open reading frame that is capable of encoding a particular polypeptide or protein after being transcribed and translated. “Gene transfer” or “gene delivery” refers to methods or systems for reliably inserting foreign DNA into host cells. Such methods can result in transient expression of non-integrated transferred DNA, extrachromosomal replication and expression of transferred replicons (e.g. episomes), and/or integration of transferred genetic material into the genomic DNA of host cells.

As used herein, the term “gradient elution” refers to application of a mixture of at least two different solutions with different pH, conductivity and/or modifier concentration to a chromatography stationary phase (including e.g., monolith, media, resin, membrane) that are gradually changed over the course of the elution. A gradient elution may be linear or non-linear. In contrast, during an isocratic elution, the chromatography mobile phase composition is constant, and during a “step elution,” the chromatography mobile phase composition changes in a stepwise manner. Over the course of the gradient elution, a percentage of a first solution is continuously varied in a manner inversely proportional to a percentage of a second solution. For example, at the start of a gradient elution, the percentage of gradient elution buffer A (e.g., a first gradient elution buffer) in the mixture is 100% and the percentage of gradient elution buffer B (e.g., a second gradient elution buffer) in the mixture is 0% such that a continuously varying gradient in the pH, conductivity and/or modifier concentration (increasing or decreasing, depending on the embodiment) is created as the solutions are mixed and flow through the stationary phase. In some embodiments, a concentration of a salt, such as sodium acetate, will change at a constant rate over the volume of a linear gradient. For example, for a 1 mL column with a 20 mL linear gradient (i.e., 20 CV), operating at a constant flow rate of 1 mL/minute, the salt concentration will change at a rate of 5% per minute. In some embodiments, rAAV capsids (e.g., full, intermediate, empty) are bound to a stationary phase during loading of a solution comprising the rAAV capsid to be purified onto an AEX stationary phase. During a gradient elution, as a percentage of buffer B increases, such that the concentration of a salt increases (e.g., Sodium acetate) full rAAV vectors are preferentially released (eluted) from the stationary phase, and empty capsids are preferentially retained on the stationary phase. Empty capsids are released in greater amounts as the percentage of buffer B further increases. Elution of full rAAV vector from the stationary phase can be monitored during gradient elution by measuring A260 and A280 of the eluate, such that an increase in the ratio of A260/A280 is indicative of an increase in the percentage of full rAAV vector in the eluate, and conversely, a decrease in the A260/A280 ratio is indicative of a decrease in the percentage of full rAAV vector and an increase in the percentage of empty capsids. In some embodiments, an absorbance of at least one fraction of eluate is measured using a method such as analytical size exclusion chromatography (SEC) in a high performance liquid chromatography (HPLC) system, on-line UV trace, off-line UV methods, etc., and wherein the absorbance is measured at one or more wavelengths (e.g., 260 nm and/or 280 nm).

As used herein, the term “heterologous” refers to a nucleic acid inserted into a vector (e.g., rAAV vector) for purposes of vector mediated transfer/delivery of the nucleic acid into a cell. Heterologous nucleic acids are typically distinct from the vector (e.g., AAV) nucleic acid, that is, the heterologous nucleic acid is non-native with respect to the viral (e.g., AAV) nucleic acid. Once transferred or delivered into a cell, a heterologous nucleic acid, contained within a vector, can be expressed (e.g., transcribed and translated if appropriate). Alternatively, a transferred or delivered heterologous nucleic acid in a cell, contained within the vector, need not be expressed. Although the term “heterologous” is not always used herein in reference to a nucleic acid, reference to a nucleic acid even in the absence of the modifier “heterologous” is intended to include a heterologous nucleic acid. For example, a heterologous nucleic acid would be a nucleic acid encoding a dystrophin polypeptide, or a fragment thereof, for example a codon optimized mini-dystrophin transgene described in WO 2017/221145, and incorporated herein by reference, for use in the treatment of Duchenne muscular dystrophy.

A further exemplary heterologous nucleic acid comprises a wild-type coding sequence, or a fragment thereof (e.g., truncated, internal deletion), of one of the following genes, and may or may not be codon-optimized:

| | | | | | |
|---|---|---|---|---|---|
| ABCA7 | COL17A1 | GBA | IDUA | PCSK9 | SGSH |
| ABCD1 | COL4A | GBE1 | IL2RG | PDE6C | SH3TC2 |
| ACAN | COL4A3 | GDAP1 | IMPDH1 | PDE6H | SLC25A13 |
| ADA | COL4A4 | GJB1 | ITGB2 | PINK1 | SLC25A15 |
| ADA2 | COL7A1 | GLA | ITGB4 | PKLR | SLC26A2 |
| ADAM10 | CPS1 | GLB1 | JAG1 | PMP22 | SMN |
| AGL | CRB1 | GLB1 | KDM6A | PON1 | SMPD1 |
| AIPL1 | CRX | GNAT2 | KMT2D | PPT1 | SNCA |
| APOB | CTNS | GNE | LAMA3 | PRKN | SORD |
| APOE4 | CTSD | GRN | LAMB3 | PRPF31 | SPATA7 |
| APP | CTSF | GRN | LAMC2 | PRPF8 | SPINK5 |
| ARG1 | CYBA | GRS | LAMP2 | PRPH2 | TGM1 |
| ARSA | CYBB | GUCA1B | LCA5 | PSEN1 | TPP1 |
| ARSB | CYP21A2 | GUCY2D | LDLR | PSEN2 | TULP1 |
| ASL | DDC | GYG1 | LPL | PYGL | UGT1A1 |
| ASS1 | DMD | HBA1 | LRAT | PYGM | VCP |
| ATF6 | DMPK | HBA2 | LRRK2 | RD3 | VEGF |
| ATP7B | DYSF | HBB | MFN2 | RDH12 | VEGFA |
| C9orf72 | F12 | HEXA | MPZ | RHO | VPS13C |
| CEP290 | F8 | HEXB | MTM1 | RP1 | VPS35 |
| CFTR | F9 | HGD | NAGLU | RPE65 | WAS |
| CHM | FANCA | HGH | NAGS | RPGR | WIPF1 |
| CHMP2B | FBLN5 | HGSNAT | NCF1 | RPGRIP1 | XPNPEP2 |
| CLN2 | FGF-1 | HINT1 | NCF2 | RS1 | BAG3 |
| CLN3 | FGFR2 | HMBS | NCF4 | SCL37A4 | ATP8B1 |
| CLN5 | FGFR3 | HNRNPA1 | NOTCH2 | SCN1A | ABCB11 |
| CLN6 | FXN | HNRNPA2B1 | OAT | SERPINA1 | ABCB4 |
| CNBP | G6PC | HTRA1 | OTC | SERPING1 | |
| CNGA3 | GAA | HTT | PAH | SGCA | |
| CNGB3 | GALNS | IDS | PARK7 | SGCG | |

As used herein, the term “homologous,” or “homology,” refers to two or more reference entities (e.g., nucleotide or polypeptide sequences) that share at least partial identity over a given region or portion. For example, when an amino acid position in two peptides is occupied by identical amino acids, the peptides are homologous at that position. Notably, a homologous peptide will retain activity or function associated with the unmodified or reference peptide and the modified peptide will generally have an amino acid sequence “substantially homologous” with the amino acid sequence of the unmodified sequence. When referring to a polypeptide, nucleic acid or fragment thereof, “substantial homology” or “substantial similarity,” means that when optimally aligned with appropriate insertions or deletions with another polypeptide, nucleic acid (or its complementary strand) or fragment thereof, there is sequence identity in at least about 95% to 99% of the sequence. The extent of homology (identity) between two sequences can be ascertained using computer program or mathematical algorithm. Such algorithms that calculate percent sequence homology (or identity) generally account for sequence gaps and mismatches over the comparison region or area. Exemplary programs and algorithms are provided below.

As used herein, the terms “host cell,” “host cell line,” and “host cell culture” are used interchangeably and refer to a cell into which an exogenous nucleic acid has been introduced, and includes the progeny of such a cell. A host cell includes a “transfectant,” “transformant,” “transformed cell,” and “transduced cell,” which includes the primary transfected, transformed or transduced cell, and progeny derived therefrom, without regard to the number of passages. In some embodiments, a host cell is a packaging cell for production of a rAAV vector.

As used herein, the term “host cell DNA” or “HCDNA” refers to residual DNA, derived from a host cell culture which produced a rAAV vector, and present in a chromatography fraction (e.g., an affinity eluate, an AEX eluate, a wash) or a chromatography load (e.g., an affinity load, an AEX load). Host cell DNA may be measured by methods know in the art such as qPCR to detect a sequence unique to the host cells. General DNA concentrations may be estimated using fluorescence dyes (e.g. PicoGreen® or SYBR® Green), absorbance measurement (e.g. at 260 nm, or 254 nm) or electrophoretic techniques (e.g. agarose gel electrophoresis, or capillary electrophoresis). An amount of HCDNA present in an eluate may be expressed relative to the amount of vg present in the eluate, for example, ng HCDNA/1×10¹⁴ vg or pg HCDNA/1×10⁹ vg. An amount of HCDNA present in an eluate may be expressed relative to the amount of vg present in a volume of eluate, for example, pg HCDNA/mL eluate.

As used herein, the term “host cell protein” or “HCP” refers to residual protein, derived from a host cell culture which produced a rAAV vector, present in a chromatography fraction (e.g., an affinity eluate, an AEX eluate, a wash) or a chromatography load (e.g., an affinity load, an AEX load). Host cell protein may be measured by methods known in the art, such as ELISA. Host cell protein can be semi-quantitatively measured by various electrophoretic staining methods (e.g., silver stain SDS-PAGE, SYPRO® Ruby stain SDS-PAGE, and/or Western blot). An amount of HCP present in an eluate may be expressed relative to the amount of vg present, for example, ng HCP/1×10¹⁴ vg or pg HCP/1×10⁹ vg.

As used herein, the term “identity” or “identical to” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, 99% or more identical.

Calculation of the percent identity of two nucleic acid or polypeptide sequences, for example, can be performed by aligning two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of a reference sequence. Nucleotides at corresponding positions are then compared. When a position in a first sequence is occupied by the same residue (e.g., nucleotide or amino acid) as the corresponding position in a second sequence, then the molecules are identical at that position. The percent identity between two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm.

To determine percent identity, or homology, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA. Other techniques for alignment are described in Methods in Enzymology, vol. 266: Computer Methods for Macromolecular Sequence Analysis (1996), ed. Doolittle, Academic Press, Inc. Of particular interest are alignment programs that permit gaps in the sequence. Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Also of interest is the BestFit program using the local homology algorithm of Smith and Waterman (1981, Advances in Applied Mathematics 2: 482-489) to determine sequence identity. The gap generation penalty will generally range from 1 to 5, usually 2 to 4 and in some embodiments will be 3. The gap extension penalty will generally range from about 0.01 to 0.20 and in some instances will be 0.10. The program has default parameters determined by the sequences inputted to be compared. Preferably, the sequence identity is determined using the default parameters determined by the program. This program is available also from Genetics Computing Group (GCG) package, from Madison, WI, USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

As used herein, the term “impurity” refers to any molecule other than the full rAAV vector being purified that is also present in a solution comprising the rAAV vector being purified. Impurities include empty capsids, intermediate capsids, biological macromolecules such as DNA, RNA, non-AAV proteins (e.g., host cell proteins), AAV aggregates, damaged AAV capsids, molecules that are part of an absorbent used for chromatography that may leach into a sample during prior purification steps, endotoxins, cell debris and chemicals from cell culture, including media components, plasmid DNA from transfection, an adventitious agent, bacteria and viruses.

As used herein, the terms “inverted terminal repeat,” “ITR,” “terminal repeat,” and “TR” refer to palindromic terminal repeat sequences at or near the ends of the AAV virus genome, comprising mostly complementary, symmetrically arranged sequences. These ITRs can fold over to form T-shaped hairpin structures that function as primers during initiation of DNA replication. They are also needed for viral genome integration into host genome, for the rescue from the host genome; and for the encapsidation of viral nucleic acid into mature virions. The ITRs are required in cis for vector genome replication and its packaging into viral particles. “5′ ITR” refer to the ITR at the 5′ end of the AAV genome and/or 5′ to a recombinant transgene. “3′ ITR” refers to the ITR at the 3′ end of the AAV genome and/or 3′ to a recombinant transgene. Wild-type ITRs are approximately 145 bp in length. A modified, or recombinant ITR, may comprise a fragment or portion of a wild-type AAV ITR sequence. One of ordinary skill in the art will appreciate that during successive rounds of DNA replication ITR sequences may swap such that the 5′ ITR becomes the 3′ ITR, and vice versa. In some embodiments, at least one ITR is present at the 5′ and/or 3′ end of a recombinant vector genome such that the vector genome can be packaged into a capsid to produce a rAAV vector (also referred to herein as “rAAV vector particle” or “rAAV viral particle”) comprising the vector genome.

The ITRs are required in cis for vector genome replication and its packaging into viral particles. “5′ ITR” refer to the ITR at the 5′ end of the AAV genome and/or 5′ to a recombinant transgene. “3′ ITR” refers to the ITR at the 3′ end of the AAV genome and/or 3′ to a recombinant transgene. Wild-type ITRs are approximately 145 bp in length. A modified, or recombinant ITR, may comprise a fragment or portion of a wild-type AAV ITR sequence. One of ordinary skill in the art will appreciate that during successive rounds of DNA replication ITR sequences may swap such that the 5′ ITR becomes the 3′ ITR, and vice versa.

As used herein, the term “isolated” refers to a substance or composition that is 1) designed, produced, prepared, and or manufactured by the hand of man and/or 2) separated from at least one of the components with which it was associated when initially produced (whether in nature and/or in an experimental setting). Generally, isolated compositions are substantially free of one or more materials with which they normally associate with in nature, for example, one or more protein, nucleic acid, lipid, carbohydrate and/or cell membrane. The term “isolated” does not exclude man-made combinations, for example, a recombinant nucleic acid, a recombinant vector genome (e.g., rAAV vector genome), a rAAV vector particle (e.g., such as, but not limited to, a rAAV vector particle comprising an AAV9 capsid) that packages, e.g., encapsidates, a vector genome and a pharmaceutical formulation. The term “isolated” also does not exclude alternative physical forms of the composition, such as hybrids/chimeras, multimers/oligomers, modifications (e.g., phosphorylation, glycosylation, lipidation), variants or derivatized forms, or forms expressed in host cells that are man-made.

Isolated substances or compositions may be separated from about 10%, about 20%, about 30%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure,” after having been combined with certain other components such as, for example, one or more carriers or excipients (e.g., buffer, solvent, water, etc.); in such embodiments, percent isolation or purity of the substance is calculated without including such carriers or excipients.

As used herein, the term “load chase” refers to a solution applied to a column after the load or load solution (as defined, infra) has been applied. A load chase serves to complete application of the load or load solution and to remove unbound material from the column.

As used herein, the terms “load” or “load solution” refer to any material (e.g., a solution) containing a product of interest (e.g., a full rAAV vector) that is loaded onto a chromatography stationary phase. In some embodiments, a “load solution” is exposed to a chromatography stationary phase. In some embodiments, a load solution is an affinity eluate. In some embodiments, a load solution is a diluted, and optionally filtered affinity eluate.

As used herein, the terms “stationary phase” or “chromatography stationary phase” are used to refer to any substance that can be used for separation of a product from another substance (e.g., an impurity). In some embodiments, a chromatography stationary phase is a resin, a media, a membrane, a membrane adsorber, or a monolith. In some embodiments, a chromatography stationary phase is a media that binds to AAV capsids under certain conditions. In some embodiments, a chromatography stationary phase is an ion exchange media (e.g., an anion exchange media, a cation exchange media). In some embodiments, a chromatography stationary phase is POROS™ 50 HQ.

As used herein, the term “modifier,” or “mobile phase modifier” is a component of the mobile phase that modifies the mobile phase in order to alter the chromatography. Such altering of the chromatography results in, for example, the removal, or washing off of, impurities from the stationary phase, or elution of a product or material of interest from the stationary phase (e.g., a rAAV vector). Examples of “modifiers” include a salt, a detergent, an amino acid (e.g., arginine, histidine, citrulline, glycine), an organic solvent (e.g., ethanol, ethylene glycol), a chaotropic agent (e.g., urea), or a displacer (also referred to as a selective elution agent).

As used herein, the terms “nucleic acid sequence,” “nucleotide sequence,” and “polynucleotide” refer interchangeably to any molecule composed of or comprising monomeric nucleotides connected by phosphodiester linkages. A nucleic acid may be an oligonucleotide or a polynucleotide. Nucleic acid sequences are presented herein in the direction from the 5′ to the 3′ direction. A nucleic acid sequence (i.e., a polynucleotide) of the present disclosure can be a deoxyribonucleic acid (DNA) molecule or ribonucleic acid (RNA) molecule and refers to all forms of a nucleic acid such as, double stranded molecules, single stranded molecules, small or short hairpin RNA (shRNA), micro RNA, small or short interfering RNA (siRNA), trans-splicing RNA, antisense RNA, messenger RNA, transfer RNA, ribosomal RNA.

Where a polynucleotide is a DNA molecule, that molecule can be a gene, a cDNA, an antisense molecule or a fragment of any of the foregoing molecules. Nucleotides are indicated herein by a single letter code: adenine (A), guanine (G), thymine (T), cytosine (C), inosine (I) and uracil (U). A nucleotide sequence may be chemically modified or artificial. Nucleotide sequences include peptide nucleic acids (PNA), morpholinos and locked nucleic acids (LNA), as well as glycol nucleic acids (GNA) and threose nucleic acids (TNA). Each of these sequences is distinguished from naturally-occurring DNA or RNA by changes to the backbone of the molecule. Also, phosphorothioate nucleotides may be used. Other deoxynucleotide analogs include methylphosphonates, phosphoramidates, phosphorodithioates, N3′-P5′-phosphoramidates, and oligoribonucleotide phosphorothioates and their 2′-O-allyl analogs and 2′-O-methylribonucleotide methylphosphonates which may be used in a nucleotide sequence of the disclosure.

As used here, the term “nucleic acid construct,” refers to a non-naturally occurring nucleic acid molecule resulting from the use of recombinant DNA technology (e.g., a recombinant nucleic acid). A nucleic acid construct is a nucleic acid molecule, either single or double stranded, which has been modified to contain segments of nucleic acid sequences, which are combined and arranged in a manner not found in nature. A nucleic acid construct may be a “vector” (e.g., a plasmid, a rAAV vector genome, an expression vector, etc.), that is, a nucleic acid molecule designed to deliver exogenously created DNA into a host cell.

As used herein, the term “operably linked” refers to a linkage of nucleic acid sequence (or polypeptide) elements in a functional relationship. A nucleic acid is operably linked when it is placed into a functional relationship with another nucleic acid sequence. For instance, a promoter or other transcription regulatory sequence (e.g., an enhancer) is operably linked to a coding sequence if it affects the transcription of the coding sequence. In some embodiments, operably linked means that nucleic acid sequences being linked are contiguous. In some embodiments, operably linked does not mean that nucleic acid sequences are contiguously linked, rather intervening sequences are between those nucleic acid sequences that are linked.

As used herein, the term “percent vector genome (VG) dilution yield” or “% VG dilution yield” refers to the amount of VG present in a diluted affinity pool (also referred to herein as a diluted affinity eluate) as a percentage of the amount of VG present in the affinity pool (also referred to herein as an affinity eluate) prior to dilution. For instance, % VG dilution yield=((amount of VG in diluted affinity pool)/(amount of VG in affinity pool))*100.

As used herein, the term “percent VG column yield” or “% VG column yield” refers to the amount of vector genomes (VG) present in a pooled eluate collected from an AEX column (i.e., an AEX pool) as a percentage of the amount of VG present in an affinity eluate that has been diluted only, or diluted and filtered.

In some embodiments, an affinity eluate comprising a rAAV vector to be purified has been diluted only and is referred to as a “diluted affinity pool.” Optionally, the rAAV vector to be purified is harvested from a 250 L or 2000 L vessel (e.g., a single use bioreactor (SUB)). For instance, % VG column yield=((amount of VG in AEX pool)/(amount of VG in diluted affinity pool))*100.

In some embodiments, an affinity eluate comprising a rAAV vector to be purified has been diluted and filtered and is referred to as an “AEX load.” Optionally, the rAAV vector to be purified is harvested from a small scale (e.g., less than 250 L) vessel (e.g., bioreactor). For instance, % VG column yield=((amount of VG in AEX pool)/(amount of VG in diluted and filtered affinity pool))*100.

As used herein, the term “percent VG step yield” or “% VG step yield” refers to the amount of VG in a pooled eluate collected from an AEX column (i.e., an AEX pool) as a percentage of the amount of VG present in the affinity pool (also referred to herein as an affinity eluate) prior to dilution or filtration. For instance, % VG step yield=((amount of VG in AEX pool)/(amount of VG in affinity pool))*100.

As used herein, the term “pharmaceutically acceptable” and “physiologically acceptable” refers to a biologically acceptable formulation, gaseous, liquid or solid, or mixture thereof, which is suitable for one or more routes of administration, in vivo delivery or contact.

As used herein, the terms “polypeptide,” “protein,” “peptide” or “encoded by a nucleic acid sequence” (i.e., encode by a polynucleotide sequence, encoded by a nucleotide sequence) refer to full-length native sequences, as with naturally occurring proteins, as well as functional subsequences, modified forms or sequence variants so long as the subsequence, modified form or variant retains some degree of functionality of the native full-length protein. In methods and uses of the disclosure, such polypeptides, proteins and peptides encoded by the nucleic acid sequences can be but are not required to be identical to the endogenous protein that is defective, or whose expression is insufficient, or deficient in a subject treated with gene therapy.

As used herein, the term “recombinant,” refers to a vector, polynucleotide (e.g., a recombinant nucleic acid), polypeptide or cell that is the product of various combinations of cloning, restriction or ligation steps (e.g., relating to a polynucleotide or polypeptide comprised therein), and/or other procedure that results in a construct that is distinct from a product found in nature. A recombinant virus or vector (e.g., rAAV vector) comprises a vector genome comprising a recombinant nucleic acid (e.g., a nucleic acid comprising a transgene and one or more regulatory elements). The terms respectively include replicates of the original polynucleotide construct and progeny of the original virus construct.

As used herein, the term “step elution” refers to application of a solution with a defined pH, conductivity, and/or modifier concentration to a chromatography stationary phase (including e.g., monolith, media, resin, membrane). A series of step elutions (e.g., with increasing conductivity or salt concentration) can be conducted to optimize separations. Each step elution solution has a defined composition that does not change during its application. Over the course of the step elution, as the series of solutions (e.g., a load chase, a pH stabilization solution, a wash buffer, an elution buffer) are applied to the stationary phase, the pH, conductivity and/or modifier concentration is increased, or decreased, relative to a preceding solution in the series. For example, at the start of a step elution series, the concentration of a modifier (e.g., a salt, e.g., sodium acetate) in the first solution is low, e.g., 0 to 10 mM, e.g., about 1 mM, about 2 mM, about 3 mM, about 4 mM, about 5 mM, about 6 mM, about 7 mM, about 8 mM, about 9 mM, about 10 mM). In each subsequent solution in the series, the concentration of the salt is increased, such that over the course of 2 to 20 solutions, the concentration of the salt is increased to, for example, 50 mM to 300 mM (e.g., about 50 mM, about 60 mM, about 70 mM, about 80 mM, about 90 mM, about 100 mM, about 120 mM, about 140 mM, about 160 mM, about 180 mM, about 200 mM). The salt concentration in the series of 2 to 20 (or more) solutions is not necessarily varied in equal or proportional increments.

In some embodiments, a step elution comprises 2 to 20 solutions, 2 to 10 solutions, 10 to 20 solutions, for example 2, 3, 4, 5,6, 7, 8, 19, 10, 11, 12, 13, 14, 15, 16, 17, 18, 19, 20 or more solutions. In some embodiments, rAAV capsids (e.g., full, intermediate, empty) are bound to a stationary phase during loading of a solution comprising the rAAV capsid onto an AEX stationary phase. During a step elution, as a pH, conductivity and/or modifier concentration is varied, full rAAV vectors are preferentially released (eluted) from the stationary phase, and empty capsids are preferentially retained on the stationary phase. Empty capsids are released in greater amounts as the concentration of modifier (e.g., salt) increases. Elution of full rAAV vector from the stationary phase can be monitored during step elution by measuring A260 and A280 of the eluate, such that an increase in the ratio of A260/A280 is indicative of an increase in the percentage of full rAAV vector in the eluate, and conversely, a decrease in the A260/A280 ratio is indicative of a decrease in the percentage of full rAAV vector and an increase in the percentage of empty capsids. In some embodiments, an absorbance of at least one fraction of eluate is measured using a method such as analytical size exclusion chromatography (SEC) in a high performance liquid chromatography (HPLC) system, on-line UV trace, off-line UV methods, etc., and wherein the absorbance is measured at one or more wavelengths (e.g., 260 nm and/or 280 nm).

As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments, a human subject is an adult, adolescent, or pediatric subject. In some embodiments, a subject is suffering from a disease, disorder or condition, e.g., a disease, disorder or condition that can be treated as provided herein. In some embodiments, a subject is suffering from a disease, disorder or condition associated with deficient or dysfunctional dystrophin, e.g., Duchenne muscular dystrophy. In some embodiments, a subject is susceptible to a disease, disorder, or condition. In some embodiments, a susceptible subject is predisposed to and/or shows an increased risk (as compared to the average risk observed in a reference subject or population) of developing a disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a human patient. In some embodiments, a subject is an individual to whom diagnosis and/or therapy is and/or has been administered (e.g., gene therapy for Duchenne muscular dystrophy). In some embodiments, a subject is a human patient with Duchenne muscular dystrophy.

Disease, disorders and conditions that can be treated using a rAAV vector purified according to the methods set forth herein include, for example a metabolic disease or disorder (e.g., Fabry disease, Gaucher disease, phenylketonuria, glycogen storage disease); a urea cycle disease or disorder (e.g., ornithine transcarbamylase deficiency); a lysosomal storage disease or disorder (e.g., metachromatic leukodystrophy, mucopolysaccharidosis); a liver disease or disorder (e.g., progressive familial intrahepatic cholestasis type 1-3); a blood disease or disorder (Hemophilia A, Hemophilia B, a thalassemia); a cancer (e.g., a carcinoma, a sarcoma, a blood cancer); a genetic disease or disorder (e.g., cystic fibrosis); or an infectious disease (e.g., HIV).

Diseases, disorders and conditions that can be treated using a rAAV vector purified according to the methods set forth herein include, for example: 21-hydroxylase-deficient congenital adrenal hyperplasia, achondrogenesis Type 1B, achondroplasia, achromatopsia, acid sphingomyelinase deficiency (Niemann-Pick disease type A or B), acute intermittent porphyria, adenosine deaminase 2 deficiency, adenosine deaminase deficiency (e.g., severe combined immunodeficiency, X-linked), adrenoleukodystrophy (e.g., X-linked), age-related macular degeneration (e.g., neovascular, wet), Alagille syndrome, alkaptonuria, alpha-1 antitrypsin deficiency, alpha-thalassemia, Alport syndrome, Alzheimer disease, Apert syndrome, arginase deficiency, argininosuccinate lyase (ASL) deficiency, argininosuccinate synthase (ASS1) deficiency (citrullinemia type 1), aromatic L-amino acid decarboxylase deficiency, autosomal recessive congenital ichthyosis, Becker muscular dystrophy, beta-thalassemia, carbamoylphosphatase synthetase I deficiency, ceroid lipofuscinosis, Charcot-Marie-Tooth neuropathy, choroideremia, chronic granulomatous disease, citrin deficiency, Crigler-Najjar syndrome type 1 and 2, critical limb ischemia, cystic fibrosis, cystinosis, Danon disease, diabetic macular retinopathy, dominant inherited short stature, Dravet syndrome, Duchenne muscular dystrophy, dysferlinopathy (e.g., Miyoshi myopathy, limb-girdle muscular dystrophy 2B), dystrophic epidermolysis bullosa, Fabry disease, familial hypercholesterolemia, familial lipoprotein lipase deficiency, Fanconia anemia (e.g., Fanconia anemia A), Friedreich's ataxia, frontotemporal dementia, Gaucher disease, glycogen storage disease type 1 A and 1B (Von Gierke's disease), glycogen storage disease type III, glycogen storage disease type IV, glycogen storage disease type V, glycogen storage disease type VI, glycogen storage disease type XV, GM1 gangliosidosis, gyrate atrophy, hemophilia A, hemophilia B, hereditary angiodema, types I-III, Huntington's disease, inclusion body myositis, junctional epidermolysis bullosa, Kabuki Syndrome, Leber congenital amaurosis, leukocyte adhesion defect type 1, limb girdle muscular dystrophy, limb girdle muscular dystrophy type 2C (gamma-sarcoglycanopathy), limb girdle muscular dystrophy type 2D, metachromatic leukodystrophy, mucopolysaccharidosis Type I, mucopolysaccharidosis type II (Hunter syndrome), mucopolysaccharidosis type IIIA, mucopolysaccharidosis type IIIB, mucopolysaccharidosis type IIIC, mucopolysaccharidosis type IIID, mucopolysaccharidosis type IVA (Morquio A syndrome), mucopolysaccharidosis type IVB (Morquio B syndrome), mucopolysaccharidosis type VI (Maroteaux-Lamy), myotonic dystrophy type 1, myotonic dystrophy type 2, N-acetylglutamate synthase (NAGS) deficiency, Netherton syndrome, neuronal ceroid lipofuscinosis, ornithine translocase deficiency, ornithine transcarbamylase deficiency disease, Parkinson's disease, phenylketonuria, Pompe, progressive familial intrahepatic cholestasis type 1-3, progressive myofibrillar myopathy, pyruvate kinase deficiency, retinitis pigmentosa, RPE65-related Leber congenital amaurosis, Sandhoff disease, sickle cell disease, spinal muscular atrophy, Tay Sachs disease, Wilson disease, Wiskott-Aldrich syndrome, Wiskott-Aldrich syndrome 2, X-linked adrenoleukodystrophy, X-linked chronic granulomatous disease, X-linked myotubular myopathy, X-linked retinitis pigmentosa, X-linked retinoschisis and X-linked severe combined immunodeficiency.

As used herein, the term “substantially” refers to the qualitative condition of exhibition of total or near-total extent or degree of a characteristic or property of interest. One of ordinary skill in the art will understand that biological and chemical phenomena rarely, if ever, go to completion and/or proceed to completeness or achieve or an absolute result. The term “substantially” is therefore used herein to capture the potential lack of completeness inherent in many biological and chemical phenomena.

As used herein, the term “therapeutic polypeptide” is a peptide, polypeptide or protein (e.g., enzyme, structural protein, transmembrane protein, transport protein) that may alleviate or reduce symptoms that result from an absence or defect in a protein in a target cell (e.g., an isolated cell) or organism (e.g., a subject). A therapeutic polypeptide or protein encoded by a transgene is one that confers a benefit to a subject, e.g., to correct a genetic defect, to correct a deficiency in a gene related to expression or function. Similarly, a “therapeutic transgene” is the transgene that encodes the therapeutic polypeptide. In some embodiments, a therapeutic polypeptide, expressed in a host cell, is an enzyme expressed from a transgene (i.e., an exogenous nucleic acid that has been introduced into the host cell). In some embodiments, a therapeutic polypeptide is a dystrophin protein, or fragment thereof, expressed from a therapeutic transgene transduced into a muscle cell (e.g., a skeletal muscle cell).

As used herein, the term “therapeutically effective amount” refers to an amount that produces the desired therapeutic effect for which it is administered. In some embodiments, the term refers to an amount that is sufficient, when administered to a population suffering from or susceptible to a disease, disorder or condition in accordance with a therapeutic dosing regimen, to treat the disease, disorder or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment.

As used herein, the term “transgene” is used to mean any heterologous polynucleotide for delivery to and/or expression in a host cell, target cell or organism (e.g., a subject). Such “transgene” may be delivered to a host cell, target cell or organism using a vector (e.g., rAAV vector). A transgene may be operably linked to a control sequence, such as a promoter. It will be appreciated by those of skill in the art that expression control sequences can be selected based on an ability to promote expression of the transgene in a host cell, target cell or organism. Generally, a transgene may be operably linked to an endogenous promoter associated with the transgene in nature, but more typically, the transgene is operably linked to a promoter with which the transgene is not associated in nature. An example of a transgene is a nucleic acid encoding a therapeutic polypeptide, for example an dystrophin polypeptide or fragment thereof, and an exemplary promoter is one not operable linked to a nucleotide encoding dystrophin in nature. Such a non-endogenous promoter can include a CBh promoter or a muscle specific promoter, among many others known in the art.

A nucleic acid of interest can be introduced into a host cell by a wide variety of techniques that are well-known in the art, including transfection and transduction.

“Transfection” is generally known as a technique for introducing an exogenous nucleic acid into a cell without the use of a viral vector. As used herein, the term “transfection” refers to transfer of a recombinant nucleic acid (e.g., an expression plasmid) into a cell (e.g., a host cell) without use of a viral vector. A cell into which a recombinant nucleic acid has been introduced is referred to as a “transfected cell.” A transfected cell may be a host cell (e.g., a CHO cell, Prol10 cell, HEK293 cell) comprising an expression plasmid/vector for producing a recombinant AAV vector. In some embodiments, a transfected cell (e.g., a packing cell) may comprise a plasmid comprising a transgene (e.g., an dystrophin transgene), a plasmid comprising an AAV rep gene and an AAV cap gene and a plasmid comprising a helper gene. Many transfection techniques are known in the art, which include, but are not limited to, electroporation, calcium phosphate precipitation, microinjection, cationic or anionic liposomes, and liposomes in combination with a nuclear localization signal.

As used herein, the term “transduction” refers to transfer of a nucleic acid (e.g., a vector genome) by a viral vector (e.g., rAAV vector) to a cell (e.g., a target cell, e.g., a muscle cell). In some embodiments, a gene therapy for Duchenne muscular dystrophy includes transducing a vector genome comprising a modified nucleic acid encoding dystrophin, or a fragment thereof, into a muscle cell. A cell into which a transgene has been introduced by a virus or a viral vector is referred to as a “transduced cell.” In some embodiments, a transduced cell is an isolated cell and transduction occurs ex vivo. In some embodiments, a transduced cell is a cell within an organism (e.g., a subject) and transduction occurs in vivo. A transduced cell may be a target cell of an organism which has been transduced by a recombinant AAV vector such that the target cell of the organism expresses a polynucleotide (e.g., a transgene, e.g., a modified nucleic acid encoding dystrophin, or a fragment thereof).

Cells that may be transduced include a cell of any tissue or organ type, or any origin (e.g., mesoderm, ectoderm or endoderm). Non-limiting examples of cells include liver (e.g., hepatocytes, sinusoidal endothelial cells), pancreas (e.g., beta islet cells, exocrine), lung, central or peripheral nervous system, such as brain (e.g., neural or ependymal cells, oligodendrocytes) or spine, kidney, eye (e.g., retinal), spleen, skin, thymus, testes, lung, diaphragm, heart (cardiac), muscle or psoas, or gut (e.g., endocrine), adipose tissue (white, brown or beige), muscle (e.g., fibroblasts, myocytes), synoviocytes, chondrocytes, osteoclasts, epithelial cells, endothelial cells, salivary gland cells, inner ear nervous cells or hematopoietic (e.g., blood or lymph) cells. Additional examples include stem cells, such as pluripotent or multipotent progenitor cells that develop or differentiate into liver (e.g., hepatocytes, sinusoidal endothelial cells), pancreas (e.g., beta islet cells, exocrine cells), lung, central or peripheral nervous system, such as brain (e.g., neural or ependymal cells, oligodendrocytes) or spine, kidney, eye (e.g., retinal), spleen, skin, thymus, testes, lung, diaphragm, heart (cardiac), muscle or psoas, or gut (e.g., endocrine), adipose tissue (white, brown or beige), muscle (e.g., fibroblast, myocytes), synoviocytes, chondrocytes, osteoclasts, epithelial cells, endothelial cells, salivary gland cells, inner ear nervous cells or hematopoietic (e.g., blood or lymph) cells.

In some embodiments, particular areas of a tissue or organ (e.g., muscle) may be transduced by a rAAV vector (e.g., a rAAV vector with a dystrophin, or portion of dystrophin, transgene) that is administered to the tissue or organ. In some embodiments, a muscle cell is transduced with a rAAV comprising a dystrophin transgene. In some embodiments, a skeletal muscle cell is transduced with a rAAV comprising a dystrophin transgene. In some embodiments, a cardiac muscle cell is transduced with a rAAV comprising a dystrophin transgene.

As used herein, the term “vector” refers to a plasmid, virus (e.g., a rAAV), cosmid, or other vehicle that can be manipulated by insertion or incorporation of a nucleic acid (e.g., a recombinant nucleic acid). A vector can be used for various purposes including, e.g., genetic manipulation (e.g., cloning vector), to introduce/transfer a nucleic acid into a cell, to transcribe or translate an inserted nucleic acid in a cell. In some embodiments a vector nucleic acid sequence contains at least an origin of replication for propagation in a cell. In some embodiments, a vector nucleic acid includes a heterologous nucleic acid sequence, an expression control element(s) (e.g., promoter, enhancer), a selectable marker (e.g., antibiotic resistance), a poly-adenosine (polyA) sequence and/or an ITR. In some embodiments, when delivered to a host cell, the nucleic acid sequence is propagated. In some embodiments, when delivered to a host cell, either in vitro or in vivo, the cell expresses the polypeptide encoded by the heterologous nucleic acid sequence. In some embodiments, when delivered to a host cell, the nucleic acid sequence, or a portion of the nucleic acid sequence is packaged into a capsid. A host cell may be an isolated cell or a cell within a host organism. In addition to a nucleic acid sequence (e.g., transgene) which encodes a polypeptide or protein, additional sequences (e.g., regulatory sequences) may be present within the same vector (i.e., in cis to the gene) and flank the gene. In some embodiments, regulatory sequences may be present on a separate (e.g., a second) vector which acts in trans to regulate the expression of the gene. Plasmid vectors may be referred to herein as “expression vectors.”

As used herein, the term “vector genome” refers to a nucleic acid that is packaged/encapsidated in an AAV capsid to form a rAAV vector. Typically, a vector genome includes a heterologous polynucleotide sequence (e.g., a transgene, regulatory elements, etc.) and at least one ITR. In cases where a recombinant plasmid is used to construct or manufacture a recombinant vector (e.g., rAAV vector), the vector genome does not include the entire plasmid but rather only the sequence intended for delivery by the viral vector. This non-vector genome portion of the recombinant plasmid is referred to as the “plasmid backbone,” which is important for cloning. selection and amplification of the plasmid, a process that is needed for propagation of recombinant viral vector production, but which is not itself packaged or encapsidated into a rAAV vector. Typically, the heterologous sequence to be packaged into the capsid is flanked by the ITRs such that when cleaved from the plasmid backbone, it is packaged into the capsid.

As used herein, the term “viral vector” generally refers to a viral particle that functions as a nucleic acid delivery vehicle and which comprises a vector genome (e.g., comprising a transgene which has replaced the wild type rep and cap) packaged within the viral particle (i.e., capsid) and includes, for example, lenti- and parvo-viruses, including AAV serotypes and variants (e.g., rAAV vectors). As noted elsewhere herein, a recombinant viral vector does not comprise a virus genome with a rep and/or a cap gene; rather, these sequences have been removed to provide capacity for the vector genome to carry a transgene of interest.

The present disclosure provides methods for purification of rAAV vectors (e.g., full rAAV vectors) from host cell harvests. In particular, the disclosure provides methods for purification of rAAV vectors (e.g., full rAAV vectors) from other nucleic acids and proteins (including empty capsids) produced by the host cell. Furthermore, the disclosure provides methods for the separation of empty capsids from full rAAV vectors (e.g., rAAV vectors comprising a vector genome). Each of these aspects of the disclosure is discussed further in the ensuing sections.

2. AAV and rAAV Vectors

AAV

As discussed supra, “adeno-associated virus” and/or “AAV” refer to parvoviruses with a linear single-stranded DNA genome and variants thereof. The term covers all subtypes and both naturally occurring and recombinant forms, except where required otherwise. Parvoviruses, including AAV, are useful as gene therapy vectors as they can penetrate a cell and introduce a nucleic acid (e.g., transgene) into the nucleus. In some embodiments, the introduced nucleic acid (e.g., rAAV vector genome) forms circular concatemers that persist as episomes in the nucleus of transduced cells. In some embodiments, a transgene is inserted in specific sites in the host cell genome, for example at a site on human chromosome 19. Site-specific integration, as opposed to random integration, is believed to likely result in a predictable long-term expression profile. The insertion site of AAV into the human genome is referred to as AAVS1. Once introduced into a cell, polypeptides encoded by the nucleic acid can be expressed by the cell. Because AAV is not associated with any pathogenic disease in humans, a nucleic acid delivered by AAV can be used to express a therapeutic polypeptide for the treatment of a disease, disorder and/or condition in a human subject.

Multiple serotypes of AAV exist in nature with at least fifteen wild type serotypes having been identified from humans thus far (i.e., AAV1-AAV15). Naturally occurring and variant serotypes are distinguished by having a protein capsid that is serologically distinct from other AAV serotypes. AAV type 1 (AAV1), AAV type 2 (AAV2), AAV type 3 (AAV3) including AAV type 3A (AAV3A) and AAV type 3B (AAV3B), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 12 (AAV12), AAVrh10, AAVrh74 (see WO 2016/210170), avian AAV, bovine AAV, canine AAV, equine AAV, primate AAV, non-primate AAV, and ovine AAV, and recombinantly produced variants (e.g., capsid variants with insertions, deletions and substitutions, etc.), such as variants referred to as AAV type 2i8 (AAV2i8), NP4, NP22, NP66, AAVDJ, AAVDJ/8, AAVDJ/9, AAVLK03, RHM4-1, among many others. AAV variants isolated from human CD34+ cell include AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15 (Smith et al. (2014) Molecular Therapy 22(9):1625-1634).

“Primate AAV” refers to AAV that infect primates, “non-primate AAV” refers to AAV that infect non-primate mammals, “bovine AAV” refers to AAV that infect bovine mammals, and so on. Serotype distinctiveness is determined on the basis of the lack of cross-reactivity between antibodies to one AAV as compared to another AAV. Such cross-reactivity differences are usually due to differences in capsid protein sequences and antigenic determinants (e.g., due to VP1, VP2, and/or VP3 sequence differences of AAV serotypes). However, some naturally occurring AAV or man-made AAV mutants (e.g., recombinant AAV) may not exhibit serological difference with any of the currently known serotypes. These viruses may then be considered a subgroup of the corresponding type, or more simply a variant AAV. Thus, as used herein, the term “serotype” refers to both serologically distinct viruses, e.g., AAV, as well as viruses, e.g., AAV, that are not serologically distinct but that may be within a subgroup or a variant of a given serotype.

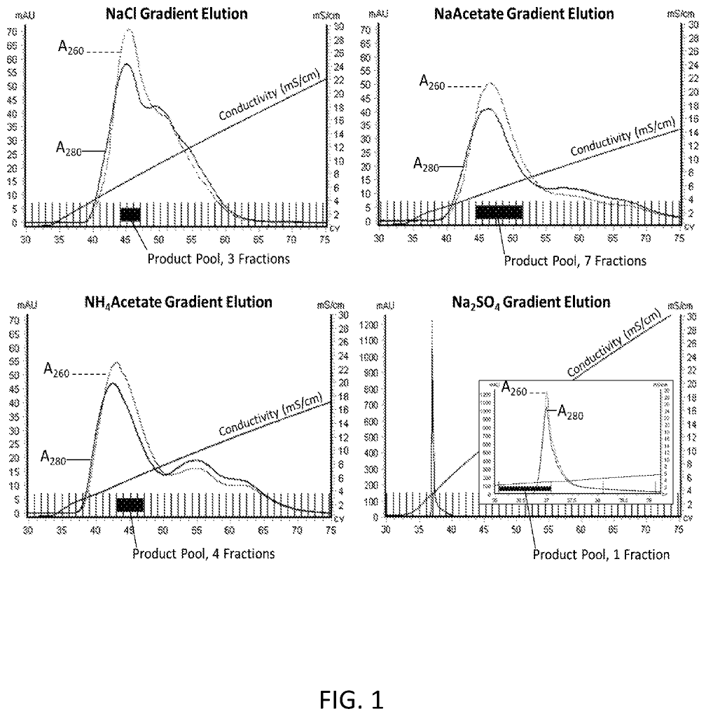

A comprehensive list and alignment of amino acid sequences of capsids of known AAV serotypes is provided by Marsic et al. (2014) Molecular Therapy 22(11):1900-1909, especially at supplementary FIG. 1.

Genomic sequences of various serotypes of AAV, as well as sequences of the native terminal repeats (ITRs), rep proteins, and capsid subunits are known in the art. Such sequences may be found in the literature or in public databases such as GenBank. See, e.g., GenBank Accession Numbers NC_002077 (AAV1), AF063497 (AAV1), NC_001401 (AAV2), AF043303 (AAV2), NC_001729 (AAV3), AF028705.1 (AAV3B), NC_001829 (AAV4), U89790 (AAV4), NC_006152 (AAV5), AF028704 (AAV6), AF513851 (AAV7), AF513852 (AAV8), NC_006261 (AAV8), AY530579 (AAV9), AY631965 (AAV10), AY631966 (AAV11), and DQ813647 (AAV12); the disclosures of which are incorporated by reference herein. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99: 11854; Moris et al. (2004) Virology 33:375-383; international patent publications WO 00/28061, WO 99/61601, WO 98/11244; WO 2013/063379; WO 2014/194132; WO 2015/121501, and U.S. Pat. Nos. 6,156,303 and 7,906,111. For illustrative purposes only, wild type AAV2 comprises a small (20-25 nm) icosahedral virus capsid of AAV composed of three proteins (VP1, VP2, and VP3; a total of 60 capsid proteins compose the AAV capsid) with overlapping sequences. The proteins VP1 (735 aa; Genbank Accession No. AAC03780), VP2 (598 aa; Genbank Accession No. AAC03778) and VP3 (533 aa; Genbank Accession No. AAC03779) exist in about a 1:1:10 ratio in the capsid. That is, for AAVs, VP1 is the full length protein and VP2 and VP3 are progressively shorter versions of VP1, with increasing truncation of the N-terminus relative to VP1. In one embodiment, of the method disclosed herein, a rAAV vector comprises an AAV9 VP1 comprising the amino acid sequence of SEQ ID NO:11.

Recombinant AAV (rAAV)

As discussed supra, a “recombinant adeno-associated virus” or “rAAV” is distinguished from a wild-type AAV by replacement of all or part of the viral genome with a non-native sequence. Incorporation of a non-native sequence within the virus defines the viral vector as a “recombinant” vector, and hence a “rAAV vector.” A rAAV vector can include a heterologous polynucleotide (e.g., human codon-optimized gene encoding human mini-dystrophin, e.g., SEQ ID NO:1) encoding a desired protein or polypeptide (e.g., a dystrophin polypeptide, or fragment thereof, e.g., SEQ ID NO:2). A recombinant vector sequence may be encapsidated or packaged into an AAV capsid and referred to as an “rAAV vector,” an “rAAV vector particle,” “rAAV viral particle” or simply a “rAAV.”

The present disclosure provides for methods of purifying a rAAV vector comprising a polynucleotide sequence not of AAV origin (e.g., a polynucleotide heterologous to AAV). The heterologous polynucleotide may be flanked by at least one, and sometimes by two, AAV terminal repeat sequences (e.g., inverted terminal repeats (ITRs)). The heterologous polynucleotide flanked by ITRs, also referred to herein as a “vector genome,” typically encodes a polypeptide of interest, or a gene of interest (“GOI”), such as a target for therapeutic treatment (e.g., a nucleic acid encoding dystrophin, or a fragment thereof, for the treatment of Duchenne muscular dystrophy). Delivery or administration of a rAAV vector to a subject (e.g. a patient) provides encoded proteins and peptides to the subject. Thus, a rAAV vector can be used to transfer/deliver a heterologous polynucleotide for expression for, e.g., treating a variety of diseases, disorders and conditions.

rAAV vector genomes generally retain 145 base ITRs in cis to the heterologous nucleic acid sequence that replaces the viral rep and cap genes. Such ITRs are necessary to produce a recombinant AAV vector; however, modified AAV ITRs and non-AAV terminal repeats including partially or completely synthetic sequences can also serve this purpose. ITRs form hairpin structures and function to, for example, serve as primers for host-cell-mediated synthesis of the complementary DNA strand after infection. ITRs also play a role in viral packaging, integration, etc. ITRs are the only AAV viral elements which are required in cis for AAV genome replication and packaging into rAAV vectors. A rAAV vector genome optionally comprises two ITRs which are generally at the 5′ and 3′ ends of the vector genome comprising a heterologous sequence (e.g., a transgene encoding a gene of interest, or a nucleic acid sequence of interest including, but not limited to, an antisense, and siRNA, a CRISPR molecule, among many others). A 5′ and a 3′ ITR may both comprise the same sequence, or each may comprise a different sequence. An AAV ITR may be from any AAV including by not limited to serotypes 1, 2, 3, 4, 5, 6, 7, 8, 9, 10 or 11 or any other AAV. An ITR is a sequence which mediates AAV genome replication and packaging.

A rAAV vector of the disclosure may comprise an ITR from an AAV serotype (e.g., wild-type AAV2, a fragment or variant thereof) that differs from the serotype of the capsid (e.g., AAV9 or other). Such a rAAV vector comprising at least one ITR from one serotype, but comprising an AAV capsid protein from a different serotype, may be referred to as a hybrid viral vector (see U.S. Pat. No. 7,172,893). An AAV ITR may include the entire wild type ITR sequence, or be a variant, fragment, or modification thereof, but will retain functionality.

In some embodiments, a heterologous polypeptide comprises an ITR (e.g., an ITR from AAV2, but can comprise an ITR from any wild type AAV serotype, or a variant thereof) positioned at the left and right ends (i.e., 5′ and 3′ termini, respectively) of a vector genome. In some embodiments, a left (e.g., 5′) ITR comprises or consists of the nucleic acid sequence of SEQ ID NO:7 or SEQ ID NO:8. In some embodiments, a left (e.g., 5′) ITR comprises a nucleic acid sequence that is about 80%, about 85%, about 90%, about 95%, about 98%, about 99% or 100% identical to SEQ ID NO:7 or SEQ ID NO:8. In some embodiments, a right (e.g., 3′) ITR comprises or consists of a nucleic acid sequence of SEQ ID NO:7 or SEQ ID NO:8. In some embodiments, a right (e.g., 3′) ITR comprises a nucleic acid sequence that is about 80%, about 85%, about 90%, about 95%, about 98%, about 99% or 100% identical to SEQ ID NO:7 or SEQ ID NO:8. Each ITR is in cis with but may be separated from each other, or other elements in the vector genome, by a nucleic acid sequence of variable length, such as a recombinant nucleic acid comprising a modified nucleic acid encoding dystrophin, or a fragment thereof, and regulatory elements. In some embodiments, ITRs are AAV2 ITRs, or variants thereof, and flank a dystrophin transgene. In some embodiments, a rAAV comprises a dystrophin transgene (e.g., comprising the nucleic acid sequence of SEQ ID NO:1) flanked by AAV2 ITRs (e.g., ITRs having the sequence as set forth in SEQ ID NO:7 or SEQ ID NO:8).

In some embodiments, a rAAV vector genome is linear, single-stranded and flanked by AAV ITRs. Prior to transcription and translation of the heterologous gene, a single stranded DNA genome of approximately 4700 nucleotides must be converted to a double-stranded form by DNA polymerases (e.g., DNA polymerases within the transduced cell) using the free 3′-OH of one of the self-priming ITRs to initiate second-strand synthesis. In some embodiments, full length-single stranded vector genomes (i.e., sense and anti-sense) anneal to generate a full length-double stranded vector genome. This may occur when multiple rAAV vectors carrying genomes of opposite polarity (i.e., sense or anti-sense) simultaneously transduce the same cell. Regardless of how they are produced, once double-stranded vector genomes are formed, the cell can transcribe and translate the double-stranded DNA and express the heterologous gene.

The efficiency of transgene expression from a rAAV vector can be hindered by the need to convert a single stranded rAAV genome (ssAAV) into double-stranded DNA prior to expression. This step is circumvented by using a self-complementary AAV genome (scAAV) that can package an inverted repeat genome that can fold into double-stranded DNA without the need for DNA synthesis or base-pairing between multiple vector genomes (McCarty, (2008) Molec. Therapy 16(10):1648-1656; McCarty et al., (2001) Gene Therapy 8:1248-1254; McCarty et al., (2003) Gene Therapy 10:2112-2118). A limitation of a scAAV vector is that size of the unique transgene, regulatory elements and IRTs to be package in the capsid is about half the size (i.e., ˜2,500 nucleotides of which 2,200 nucleotides may be a transgene and regulatory elements, plus two copies of the ˜145 nucleotide ITRs) of a ssAAV vector genome (i.e., ˜4,900 nucleotides including two ITRs).

scAAV vector genomes are made by deleting the terminal resolution site (TRS) from one rAAV ITR of the expression plasmid, thereby preventing initiation of replication from that end (see U.S. Pat. No. 8,784,799). AAV replication within a host cell is initiated at the wild type ITR of the genome and continues through the mutant ITR without terminal resolution and then back across the genome to create a dimer. The dimer is a self-complementary genome with a mutant ITR in the middle, and wild-type ITRs at each end. In some embodiments, a mutant ITR with a deleted TRS is at the 5′ end of the vector genome. In some embodiments, a mutant ITR with a deleted TRS is at the 3′ end of the vector genome. In some embodiments, a mutant ITR comprises the nucleic acid sequence of SEQ ID NO:13 or SEQ ID NO:14.

Without wishing to be bound by theory, while the two halves of a scAAV genome are complementary, it is unlikely that there is substantial base pairing within the capsid as many of the bases are in contact with amino acid residues of the inner capsid shell and the phosphate backbone is sequestered toward the center (McCarty, Molec. Therapy (2008) 16(10):1648-1656). It likely that upon uncoating, the two halves of the scAAV genome anneal to form a dsDNA hairpin molecule, with a covalently closed ITR at one end and two open-ended ITRs on the other. The ITRs flank a double-stranded region encoding, among other things, the transgene, and regulatory elements in cis thereto.

A viral capsid of a rAAV vector may be from a wild type AAV or a variant AAV such as AAV1, AAV2, AAV3, AAV3A, AAV3B, AAV4, AAV5, AAV6, AAV7, AAV8, AAV9, AAV10, AAVrh10, AAVrh74 (see WO2016/210170), AAV12, AAV2i8, AAV1.1, AAV2.5, AAV6.1, AAV6.3.1, AAV9.45, RHM4-1 (SEQ ID NO:5 of WO 2015/013313), RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAV avian AAV, bovine AAV, canine AAV, equine AAV, primate AAV, non-primate AAV, snake AAV, goat AAV, shrimp AAV, ovine AAV and variants thereof (see, e.g., Fields et al., VIROLOGY, volume 2, chapter 69 (4th ed., Lippincott-Raven Publishers). Capsids may be derived from a number of AAV serotypes disclosed in U.S. Pat. No. 7,906,111; Gao et al. (2004) J. Virol. 78:6381; Morris et al. (2004) Virol. 33:375; WO 2013/063379; WO 2014/194132; and include true type AAV (AAV-TT) variants disclosed in WO 2015/121501, and RHM4-1, RHM15-1 through RHM15-6, and variants thereof, disclosed in WO 2015/013313. Capsids may also be derived from AAV variants isolated from human CD34+ cell include AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15 (Smith et al. (2014) Molecular Therapy 22(9):1625-1634). One skilled in the art would know there are likely other AAV variants not yet identified that perform the same or similar function. A full complement of AAV cap proteins includes VP1, VP2, and VP3. The ORF comprising nucleotide sequences encoding AAV VP capsid proteins may comprise less than a full complement AAV Cap proteins or the full complement of AAV cap proteins may be provided.

In another embodiment, the present disclosure provides for the use of ancestral AAV vectors for use in therapeutic in vivo gene therapy. Specifically, in silico-derived sequences may be synthesized de novo and characterized for biological activities. Prediction and synthesis of ancestral sequences, in addition to assembly into a rAAV vector, may be accomplished using methods described in WO 2015/054653, the contents of which are incorporated by reference herein. Notably, rAAV vectors assembled from ancestral viral sequences may exhibit reduced susceptibility to pre-existing immunity in human populations as compared to contemporary viruses or portions thereof.

In some embodiments, a rAAV vector comprising a capsid protein encoded by a nucleotide sequence derived from more than one AAV serotype (e.g., wild type AAV serotypes, variant AAV serotypes) is referred to as a “chimeric vector” or “chimeric capsid” (See U.S. Pat. No. 6,491,907, the entire disclosure of which is incorporated herein by reference). In some embodiments, a chimeric capsid protein is encoded by a nucleic acid sequence derived from 2, 3, 4, 5, 6, 7, 8, 9, 10 or more AAV serotypes. In some embodiments, a recombinant AAV vector includes a capsid sequence derived from e.g., AAV1, AAV2, AAV3, AAV3A, AAV3B, AAV4, AAV5, AAV6, AAV7, AAV8, AAV9, AAV10, AAV11, AAVrh74, AAVrh10, AAV2i8, or variant thereof, resulting in a chimeric capsid protein comprising a combination of amino acids from any of the foregoing AAV serotypes (see, Rabinowitz et al. (2002) J. Virology 76(2):791-801). Alternatively, a chimeric capsid can comprise a mixture of a VP1 from one serotype, a VP2 from a different serotype, a VP3 from yet a different serotype, and a combination thereof. For example, a chimeric virus capsid may include an AAV1 cap protein or subunit and at least one AAV2 cap protein or subunit. A chimeric capsid can, for example include an AAV capsid with one or more B19 cap subunits, e.g., an AAV cap protein or subunit can be replaced by a B19 cap protein or subunit. For example, in one embodiment, a VP3 subunit of an AAV capsid can be replaced by a VP2 subunit of B19.

In some embodiments, chimeric vectors have been engineered to exhibit altered tropism or tropism for a particular tissue or cell type. The term “tropism” refers to preferential entry of the virus into certain cell or tissue types and/or preferential interaction with the cell surface that facilitates entry into certain cell or tissue types. AAV tropism is generally determined by the specific interaction between distinct viral capsid proteins and their cognate cellular receptors (Lykken et al. (2018) J. Neurodev. Disord. 10:16). Preferably, once a virus or viral vector has entered a cell, sequences (e.g., heterologous sequences such as a transgene) carried by the vector genome (e.g., a rAAV vector genome) are expressed.

A “tropism profile” refers to a pattern of transduction of one or more target cells, tissues and/or organs. For example, an AAV capsid may have a tropism profile characterized by efficient transduction of muscle cells with only low transduction of, for example, brain cells.

### 3. Recombinant Nucleic Acids

Methods of the present disclosure include purification of a rAAV vector comprising a recombinant nucleic acid including modified nucleic acids as well as plasmids and vector genomes that comprise a modified nucleic acid. A recombinant nucleic acid, plasmid or vector genome may comprise regulatory sequences to modulate propagation (e.g., of a plasmid) and/or control expression of a modified nucleic acid (e.g., a transgene). Recombinant nucleic acids may also be provided as a component of a viral vector (e.g., a rAAV vector). Generally, a viral vector includes a vector genome comprising a recombinant nucleic acid packaged in a capsid.

Modified Nucleic Acids

A modified, or variant form, of a gene, nucleic acid or polynucleotide (e.g., a transgene) refers to a nucleic acid that deviates from a reference sequence. A reference sequence may be a naturally occurring, wild type sequence (e.g., a gene) and may include naturally occurring variants (e.g., splice variants, alternative reading frames). Those skilled in the art will be aware that reference sequences can be found in publicly available databases such as GenBank (ncbi.nlm.nih.gov/genbank). Modified/variant nucleic acids may have substantially the same, greater or lesser activity, function or expression as compared to a reference sequence. Preferably, a modified, or variant nucleic acid, as used interchangeably herein, exhibits improved protein expression, e.g., a protein encoded thereby is expressed at a detectably greater level in a cell compared with the level of expression of a protein provided by an endogenous gene (e.g., a wild type gene, a mutant gene) in an otherwise identical cell. In some embodiments, a modified, or variant nucleic acid, as used interchangeably herein, exhibits improved protein expression, e.g., a protein encoded thereby is expressed at a detectably greater level in a cell compared with the level of expression of a protein provided by an endogenous gene comprising a mutation in an otherwise identical cell.

Modifications to nucleic acids include one or more nucleotide substitutions (e.g., substitution of 1-3, 3-5, 5-10, 10-15, 15-20, 20-25, 25-30, 30-40, 40-50, 50-100 or more nucleotides), additions (e.g., insertion of 1-3, 3-5, 5-10, 10-15, 15-20, 20-25, 25-30, 30-40, 40-50, 50-100 or more nucleotides), deletions (e.g., deletion of 1-3, 3-5, 5-10, 10-15, 15-20, 20-25, 25-30, 30-40, 40-50, 50-100 or more nucleotides, deletion of a motif, domain, fragment, etc.) of a reference sequence. A modified nucleic acid may be about 50%, about 60%, about 70%, about 80%, about 85%, about 90%, about 92%, about 93%, about 94%, about 95%, about 96% about 97% about 98% or about 99% identical to a reference sequence.

A modified nucleic acid may encode a polypeptide with about 50%, about 60%, about 70%, about 80%, about 85%, about 90%, about 95%, about 98%, about 99% or 100% identity to a reference polypeptide. In some embodiments, a modified nucleic acid encodes a polypeptide with 100% identify to a reference polypeptide.

In some embodiments, a modified nucleic acid (e.g., transgene) encodes a wild-type protein. Such modified nucleic acid may be codon optimized. “Optimized” or “codon-optimized,” as referred to interchangeably herein, refers to a coding sequence that has been optimized relative to a wild type coding sequence or reference sequence (e.g., a coding sequence for a mini-dystrophin polypeptide, e.g., SEQ ID NO:2, a coding sequence for a deleted copper transporting ATPase polypeptide, e.g., SEQ ID NO:15) to increase expression of the polypeptide, e.g., by minimizing usage of rare codons, decreasing the number of CpG dinucleotides, removing cryptic splice donor or acceptor sites, removing Kozak sequences, removing ribosomal entry sites, and the like. In some embodiments, a level of expression of a protein from a codon-optimized sequence is increased as compared to a level of expression of a protein from a wild type gene in an otherwise identical cell. In some embodiments, a level of expression of a protein from a codon-optimized sequence is not increased (e.g., expression is substantially similar) as compared to a level of expression of a protein from a wild-type gene in an otherwise identical cell. In some embodiments, a level of expression of a protein from a codon-optimized sequence is increased as compared to a level of expression of a protein from a mutant gene in an otherwise identical cell.

Examples of modifications include elimination of one or more cis-acting motifs and introduction of one or more Kozak sequences. In some embodiments, one or more cis-acting motifs are eliminated and one or more Kozak sequences are introduced.

Examples of cis-acting motifs that may be eliminated include internal TATA-boxes; chi-sites; ribosomal entry sites; ARE, INS, and/or CRS sequence elements; repeat sequences and/or RNA secondary structures; (cryptic) splice donor and/or acceptor sites, branch points; and restriction sites.

In some embodiments, a modified nucleic acid encodes a modified or variant polypeptide. A modified polypeptide (e.g., a codon optimized mini-dystrophin) encoded by a modified nucleic acid may retain all or a part of the function or activity of a polypeptide encoded by a wild type coding or reference sequence. In some embodiments, a modified polypeptide has one or more non-conservative or conservative amino acid changes. In some embodiments, certain domains that have been demonstrated to play a limited or no role in a function of a polypeptide are not present in a modified polypeptide (e.g., certain binding domains) (e.g., WO 2016/097219). Modified nucleic acids present in rAAV vectors may comprise fewer nucleotides than the wild type coding, or reference sequence, due to the packaging capacity of a rAAV capsid (e.g., shortened minidystrophin transgene, see WO 2001/83695; a B-domain deleted human Factor VIII transgene, see WO 2017/074526 all of which are incorporated herein by reference), and also include shortened transgenes that are both truncated and codon-optimized (e.g., a codon optimized mini-dystrophin transgene described in WO 2017/221145; deleted copper transporting ATPase2 with deletion of metal binding sites (MBS) 1-4, see WO 2016/097219 and WO 2016/097218 all of which are incorporated herein by reference). In some embodiments, a polypeptide encoded by a modified nucleic acid has less than, the same, or greater, but at least a part of, a function or activity of a polypeptide encoded by a reference sequence.

Modified nucleic acids may have a modified GC content (e.g., the number of G and C nucleotides present in a nucleic acid sequence), a modified (e.g., increased or decreased) CpG dinucleotide content and/or a modified (e.g., increased or decreased) codon adaptation index (CAI) relative to a reference and/or wild-type sequence. See, e.g., WO 2017/077451 (discussing various considerations well-known in the art for codon-optimization of nucleic acid sequences of interest, including publicly available software for analyzing nucleic acid sequences for optimization). As used herein, modified refers to a decrease or an increase in a particular value, amount or effect.

In some embodiments, a GC content of a modified nucleic acid sequence of the present disclosure is increased relative to a reference and/or a wild-type gene or coding sequence. The GC content of a modified nucleic acid is at least 5%, at least 6%, at least 7%, at least 8%, at least 9%, at least 10%, at least 12%, at least 14%, at least 15%, at least 17%, at least 20%, at least 30%, at least 40%, at least 50%, at least 60%, at least 70% greater than GC content of a wild type coding sequence. In some embodiments, GC content is expressed as a percentage of G (guanine) and C (cytosine) nucleotides in the sequence.

In some embodiments, a codon adaptation index of a modified nucleic acid sequence of the present disclosure is at least 0.74, at least 0.76, at least 0.77, at least 0.80, at least 0.85, at least 0.86, at least 0.87, at least 0.90, at least 0.95 or at least 0.98.

In some embodiments, a modified nucleic acid sequence of the present disclosure has a reduced level of CpG dinucleotides, that being a reduction of about 10%, 20%, 30%, 50% or more, as compared to a wild type or reference nucleic acid sequence. In some embodiments, a modified nucleic acid has 1-5 fewer, 5-10 fewer, 10-15 fewer, 15-20 fewer, 20-25 fewer, 25-30 fewer, 30-40 fewer, 40-45 fewer or 45-50 fewer, or even fewer di-nucleotides than a reference sequence (e.g., a wild type sequence).

It is known that methylation of CpG dinucleotides plays an important role in the regulation of gene expression in eukaryotes. Specifically, methylation of CpG dinucleotides in eukaryotes essentially serves to silence gene expression through interfering with the transcriptional machinery. As such, because of the gene silencing evoked by methylation of CpG motifs, nucleic acids and vectors having a reduced number of CpG dinucleotides will provide for high and longer-lasting transgene expression level.

Modified nucleic acid sequences may include flanking restriction sites to facilitate subcloning into an expression vector. Many such restriction sites are well known in the art, and include, but are not limited to Aval, Swal, ApaL1 and Xmal.

The present disclosure includes a modified nucleic acid of SEQ ID NO:1 which encodes a functionally active fragment of the dystrophin polypeptide. A “functionally active” or “functional dystrophin polypeptide” indicates that the fragment provides the same or similar biological function and/or activity as a full-length dystrophin polypeptide. That is, the fragment provides the same function including, but not limited to, as a structural protein of myofilaments of a muscle fiber. The biological activity of a functional fragment of dystrophin encompasses reversing or preventing the neuromuscular phenotype associated with Duchenne muscular dystrophy.

Thus, one embodiment of the invention relates to a method of purifying a rAAV vector comprising a modified nucleic acid encoding a mini-dystrophin protein, the nucleic acid comprising, consisting essentially of, or consisting of the nucleic acid sequence of SEQ ID NO:1 or a sequence at least about 90% identical thereto. In some embodiments, the nucleic acid is at least about 90%, 91%, 92%, 93%, 94%, 95%, 96%, 97%, 98%, or 99% identical to the nucleic acid sequence of SEQ ID NO:1. In certain embodiments, the nucleic acid has a length that is within the capacity of a viral vector, e.g., a parvovirus vector, e.g., a rAAV vector. In some embodiments, the nucleic acid is about 5000, 4900, 4800, 4700, 4600, 4500, 4400, 4300, 4200, 4100, or about 4000 nucleotides, or fewer.

In some embodiments, a nucleic acid encodes a mini-dystrophin protein comprising, consisting essentially of, or consisting of the amino acid sequence of SEQ ID NO:2 or a sequence at least about 90%, 91%, 92%, 93%, 94%, 95%, 96%, 97%, 98%, or 99% identical to the amino acid sequence of SEQ ID NO:2.

In some embodiments, a nucleic acid encodes a deleted copper transporting ATPase 2 protein comprising, consisting essentially of, or consisting of the amino acid sequence of SEQ ID NO:15 or a sequence at least about 90%, 91%, 92%, 93%, 94%, 95%, 96%, 97%, 98%, or 99% identical to the amino acid sequence of SEQ ID NO:15.

In some embodiments, a nucleic acid (e.g., SEQ ID NO:1) is part of a recombinant nucleic acid for production of dystrophin protein. The recombinant nucleic acid may further comprise regulatory elements useful for increasing expression of dystrophin. In some embodiments, a nucleic acid is part of a recombinant nucleic acid for production of cooper transporting ATPase 2 protein. The recombinant nucleic acid may further comprise regulatory elements useful for increasing expression of copper transporting ATPase 2.

Regulatory Elements

Methods of the present disclosure include purification of a rAAV vector comprising a recombinant nucleic acid including a modified nucleic acid encoding a polypeptide (e.g., mini-dystrophin) and various regulatory or control elements. Typically, regulatory elements are nucleic acid sequence(s) that influence expression of an operably linked polynucleotide. The precise nature of regulatory elements useful for gene expression will vary from organism to organism and from cell type to cell type including, for example, a promoter, enhancer, intron etc., with the intent to facilitate proper heterologous polynucleotide transcription and translation. Regulatory control can be affected at the level of transcription, translation, splicing, message stability, etc. Typically, a regulatory control element that modulates transcription is juxtaposed near the 5′ end of the transcribed polynucleotide (i.e., upstream). Regulatory control elements may also be located at the 3′ end of the transcribed sequence (i.e., downstream) or within the transcript (e.g., in an intron). Regulatory control elements can be located at a distance away from the transcribed sequence (e.g., 1 to 100, 100 to 500, 500 to 1000, 1000 to 5000, 5000 to 10,000 or more nucleotides). However, due to the length of an AAV vector genome, regulatory control elements are typically within 1 to 1000 nucleotides from the polynucleotide.

Promoter

As used herein, the term “promoter,” such as a “eukaryotic promoter,” refers to a nucleotide sequence that initiates transcription of a particular gene, or one or more coding sequences (e.g., an mini-dystrophin coding sequence), in eukaryotic cells (e.g., a muscle cell). A promoter can work with other regulatory elements or regions to direct the level of transcription of the gene or coding sequence(s). These regulatory elements include, for example, transcription binding sites, repressor and activator protein binding sites, and other nucleotide sequences known to act directly or indirectly to regulate the amount of transcription from the promoter, including, for example, attenuators, enhances and silencers. The promoter is most often located on the same strand and near the transcription start site, 5′ of the gene or coding sequence to which it is operably linked. A promoter is generally 100-1000 nucleotides in length. A promoter typically increases gene expression relative to expression of the same gene in the absence of a promoter.

As used herein, a “core promoter” or “minimal promoter” refers to the minimal portion of a promoter sequence required to properly initiate transcription. It may include any of the following: a transcription start site, a binding site for RNA polymerase and a general transcription factor binding site. A promoter may also comprise a proximal promoter sequence (5′ of a core promoter) that contains other primary regulatory elements (e.g., enhancer, silencer, boundary element, insulator) as well as a distal promoter sequence (3′ of a core promoter). In some embodiments, a core or minimal promoter is an α1-antitrypsin core or minimal promoter, optionally comprising or consisting of the nucleic acid of SEQ ID NO:16.

Examples of a suitable promoter include adenoviral promoters, such as the adenoviral major late promoter; heterologous promoters, such as the cytomegalovirus (CMV) promoter; the respiratory syncytial virus promoter; the Rous Sarcoma Virus (RSV) promoter; the albumin promoter; inducible promoters, such as the Mouse Mammary Tumor Virus (MMTV) promoter; the metallothionein promoter; heat shock promoters; the α-1-antitrypsin promoter; the hepatitis B surface antigen promoter; the transferrin promoter; the apolipoprotein A-1 promoter; chicken β-actin (CBA) promoter; the elongation factor 1a (EF1a) promoter; the hybrid form of the CBA promoter (CBh promoter); the CAG promoter (cytomegalovirus early enhancer element and promoter, the first exon, and the first intron of chicken beta-actin gene and the splice acceptor of the rabbit beta-globin gene) (Alexopoulou et al. (2008) BioMed. Central Cell Biol. 9:2); a creatine kinase promoter; and the human dystrophin gene promoter.

In some embodiments, the promoter is a creatinine kinase promoter, e.g., a promoter comprising, consisting essentially of, or consisting of the nucleotide sequence of SEQ ID NO:3 or SEQ ID NO:4.

In some embodiments of the present disclosure, a eukaryotic promoter sequence (e.g., a creatine kinase promoter) is operably linked to a modified nucleic acid encoding e.g., mini-dystrophin or a deleted copper transporting ATPase2. In some embodiments, a promoter comprising the nucleic acid sequence of SEQ ID NO:3 or SEQ ID NO:6 (e.g., a creatine kinase promoter) is operably linked to a modified nucleic acid encoding mini-dystrophin. In some embodiments, a promoter comprising a nucleic acid sequence at least 80%, at least 85%, at least 90%, at least 95%, at least 98%, at least 99% or 100% identical to the nucleic acid sequence of SEQ ID NO:3 or SEQ ID NO:4 is operably linked to a nucleic acid comprising the nucleic acid sequence of SEQ ID NO:1. In some embodiments, a promoter comprising the nucleic acid sequence of SEQ ID NO:16 (e.g., an α1-antitrypsin promoter) is operably linked to a modified nucleic acid encoding a deleted copper transporting ATPase 2 with deletion of MBS 1-4 (e.g., SEQ ID NO:15). In some embodiments, a promoter comprising a nucleic acid sequence at least 80%, at least 85%, at least 90%, at least 95%, at least 98%, at least 99% or 100% identical to the nucleic acid sequence of SEQ ID NO:16 is operably linked to a nucleic acid comprising the amino acid sequence of SEQ ID NO:15.

In some embodiments, a promoter comprising a nucleic acid sequence at least 95% identical to the nucleic acid sequence of SEQ ID NO:3 or SEQ ID NO:4 is operably linked to a nucleic acid sequence at least 95% identical to the nucleic acid sequence of SEQ ID NO:1 and induces expression of a polypeptide encoded by the nucleic acid sequence of SEQ ID NO:1 in muscle cells.

A promoter may be constitutive, tissue-specific or regulated. Constitutive promoters are those which cause an operably linked gene to be expressed essentially at all times. In some embodiments, a constitutive promoter is active in most eukaryotic tissues under most physiological and developmental conditions.

Regulated promoters are those which can be activated or deactivated. Regulated promoters include inducible promoters, which are usually “off,” but which may be induced to turn “on,” and “repressible” promoters, which are usually “on,” but may be turned “off.” Many different regulators are known, including temperature, hormones, cytokines, heavy metals and regulatory proteins. The distinctions are not absolute; a constitutive promoter may often be regulated to some degree. In some cases, an endogenous pathway may be utilized to provide regulation of the transgene expression, e.g., using a promoter that is naturally downregulated when the pathological condition improves.

A tissue-specific promoter is a promoter that is active in only specific types of tissues, cells or organs. Typically, a tissue-specific promoter is recognized by transcriptional activator elements that are specific to a particular tissue, cell and/or organ. For example, a tissue-specific promoter may be more active in one or several particular tissues (e.g., two, three or four) than in other tissues. In some embodiments, expression of a gene modulated by a tissue-specific promoter is much higher in the tissue for which the promoter is specific than in other tissues. In some embodiments, there may be little, or substantially no activity, of the promoter in any tissue other than the one for which it is specific. A promoter may be a tissue-specific promoter, such as the mouse albumin promoter, or the transthyretin promoter (TTR), which are active in liver cells. Other examples of tissue specific promoters include promoters from genes encoding skeletal α-actin, myosin light chain 2A, dystrophin, muscle creatine kinase which induce expression in skeletal muscle (Li et al. (1999) Nat. Biotech. 17:241-245). Liver specific expression may be induced using promoters from the albumin gene (Miyatake et al. (1997) J. Virol. 71:5124-5132), hepatitis B. virus core promoter (Sandig, et al. (1996) Gene Ther. 3:1002-1009) and alpha-fetoprotein (Arbuthnot et al., (1996) Hum. Gene. Ther. 7:1503-1514).

Enhancer

In another aspect, a modified nucleic acid encoding a therapeutic polypeptide further comprises an enhancer to increase expression of the therapeutic polypeptide. Typically, an enhancer element is located upstream of a promoter element but may also be located downstream or within another sequence (e.g., a transgene). An enhancer may be located 100 nucleotides, 200 nucleotides, 300 nucleotides or more upstream or downstream of a modified nucleic acid. An enhancer typically increases expression of a modified nucleic acid (e.g., encoding a therapeutic polypeptide) beyond the increased expression provided by a promoter element alone.

Many enhancers are known in the art, including, but not limited to, the cytomegalovirus major immediate-early enhancer. More specifically, the cytomegalovirus (CMV) MIE promoter comprises three regions: the modulator, the unique region and the enhancer (Isomura and Stinski (2003) J. Virol. 77(6):3602-3614). The CMV enhancer region can be combined with another promoter, or a portion thereof, to form a hybrid promoter to further increase expression of a nucleic acid operably linked thereto. For example, a chicken β-actin (CBA) promoter, or a portion thereof, can be combined with a CMV promoter/enhancer, or a portion thereof, to make a version of CBA termed the “CBh” promoter, which stands for chicken beta-actin hybrid promoter, as described in Gray et al. (2011, Human Gene Therapy 22:1143-1153). Like promoters, enhancers may be constitutive, tissue-specific or regulated.

In some embodiments of the present disclosure, a regulatory element comprises a hybrid enhancer and promoter, such as a synthetic hybrid enhancer and promoter derived from the creatine kinase (CK) gene which serves as a muscle specific transcription regulatory element (hCK) and which is operably linked to a modified nucleic acid encoding mini-dystrophin. In some embodiments, a synthetic hybrid enhancer and promoter comprising the nucleic acid sequence of SEQ ID NO:5 is operably linked to a modified nucleic acid encoding mini-dystrophin. In some embodiments, a synthetic hybrid enhancer and promoter derived from the creatine kinase (CK) gene comprising a nucleic acid sequence at least 80%, at least 85%, at least 90%, at least 95%, at least 98%, at least 99% or 100% identical to the nucleic acid sequence of SEQ ID NO:5 is operably linked to a nucleic acid comprising the nucleic acid sequence of SEQ ID NO:1.

In some embodiments, a synthetic hybrid enhancer and promoter derived from the creatine kinase (CK) gene comprising a nucleic acid sequence at least 95% identical to the nucleic acid sequence of SEQ ID NO:5 is operably linked to a nucleic acid sequence at least 95% identical to the nucleic acid sequence of SEQ ID NO:1 and induces expression of a polypeptide encoded by the nucleic acid sequence of SEQ ID NO:1 in muscle cells.

Fillers, Spacers and Stuffers

As disclosed herein, a recombinant nucleic acid intended for use in a rAAV vector may include an additional nucleic acid element to adjust the length of the nucleic acid to near, or at the normal size (e.g., approximately 4.7 to 4.9 kilobases), of the viral genomic sequence acceptable for AAV packaging into a rAAV vector (Grieger and Samulski (2005) J. Virol. 79(15):9933-9944). Such a sequence may be referred to interchangeably as filler, spacer or stuffer. In some embodiments, filler DNA is an untranslated (non-protein coding) segment of nucleic acid. In some embodiments, a filler or stuffer polynucleotide sequence is a sequence between about 1-10, 10-20, 20-30, 30-40, 40-50, 50-60, 60-70, 70-80, 80-90-90-100, 100-150, 150-200, 200-250, 250-300, 300-400, 400-500, 500-750, 750-1000, 1000-1500, 1500-2000, 2000-3000 or more in length.

AAV vectors typically accept inserts of DNA having a size ranging from about 4 kb to about 5.2 kb or about 4.1 to 4.9 kb for optimal packaging of the nucleic acid into the AAV capsid. In some embodiments, a rAAV vector comprises a vector genome having a total length between about 3.0 kb to about 3.5 kb, about 3.5 kb to about 4.0 kb, about 4.0 kb to about 4.5 kb, about 4.5 kb to about 5.0 kb or about 5.0 kb to about 5.2 kb. In some embodiments, a rAAV vector comprises a vector genome having a total length of about 4.5 kb. In some embodiments, a rAAV vector comprises a vector genome that is self-complementary. While the total length of a self-complementary (sc) vector genome in a rAAV vector is equivalent to a single-stranded (ss) vector genome (i.e., from about 4 kb to about 5.2 kb), the nucleic acid sequence (i.e., comprising the transgene, regulatory elements and ITRs) encoding the sc vector genome must be only half as long as a nucleic acid sequence encoding a ss vector genome in order for the sc vector genome to be packaged in the capsid.

Introns and Exons

In some embodiments, a recombinant nucleic acid includes, for example, an intron, exon and/or a portion thereof. An intron may function as a filler or stuffer polynucleotide sequence to achieve an appropriate length for vector genome packaging into a rAAV vector. An intron and/or an exon sequence can also enhance expression of a polypeptide (e.g., a transgene) as compared to expression in the absence of the intron and/or exon element (Kurachi et al. (1995) J. Biol. Chem. 270 (10):576-5281; WO 2017/074526). Furthermore, filler/stuffer polynucleotide sequences (also referred to as “insulators”) are well known in the art and include, but are not limited to, those described in WO 2014/144486 and WO 2017/074526.

An intron element may be derived from the same gene as a heterologous polynucleotide, or derived from a completely different gene or other DNA sequence (e.g., chicken β-actin gene, minute virus of mice (MVM)). In some embodiments, a recombinant nucleic acid includes at least one element selected from an intron and an exon derived from a non-cognate gene (i.e., not derived from the modified nucleic acid, e.g., transgene).

Polyadenylation Signal Sequence (polyA)

Further regulatory elements may include a stop codon, a termination sequence, and a polyadenylation (polyA) signal sequence, such as, but not limited to a bovine growth hormone poly A signal sequence (BHG polyA). A polyA signal sequence drives efficient addition of a poly-adenosine “tail” at the 3′ end of a eukaryotic mRNA which guides termination of gene transcription (see, e.g., Goodwin and Rottman J. Biol. Chem. (1992) 267(23):16330-16334). A polyA signal acts as a signal for the endonucleolytic cleavage of the newly formed precursor mRNA at its 3′ end and for addition to this 3′ end of an RNA stretch consisting only of adenine bases. A polyA tail is important for the nuclear export, translation and stability of mRNA. In some embodiments, a poly A is a SV40 early polyadenylation signal, a SV40 late polyadenylation signal, an HSV thymidine kinase polyadenylation signal, a protamine gene polyadenylation signal, an adenovirus 5 E1b polyadenylation signal, a growth hormone polyadenylation signal, a PBGD polyadenylation signal or an in silico designed polyadenylation signal.

In some embodiments, and optionally in combination with one or more other regulatory elements described herein, a polyA signal sequence of a recombinant nucleic acid is a polyA signal that is capable of directing and effecting the endonucleolytic cleavage and polyadenylation of the precursor mRNA resulting from the transcription of a modified nucleic acid encoding e.g., mini-dystrophin (e.g., SEQ ID NO:2) or a deleted copper transporting ATPase 2 (e.g., SEQ ID NO:15). In some embodiments, a polyA sequence comprises or consists of the nucleic acid sequence of SEQ ID NO:6 or SEQ ID NO:17. In some embodiments, a polyA sequence comprises a nucleic acid sequence about 80%, about 85%, about 90%, about 95%, about 98%, about 99% or 100% identical to the nucleic acid sequence of SEQ ID NO:6 or SEQ ID NO:17. In some embodiments, a recombinant nucleic acid comprises at least one of: a promoter sequence (e.g., SEQ ID NO:3, SEQ ID NO:4), a hybrid enhancer and promoter (e.g., SEQ ID NO:5) and a polyA (SEQ ID NO:6) and modulates the expression of a heterologous polypeptide, optionally encoded by the nucleic acid sequence of SEQ ID NO:1. In some embodiments, a recombinant nucleic acid comprises at least one of: a promoter sequence (e.g., SEQ ID NO:16), and a polyA (SEQ ID NO:17) and modulates the expression of a heterologous polypeptide comprising the amino acid sequence of SEQ ID NO:15.

In some embodiments, a rAAV9 vector with tropism for muscle cells, contains a vector genome comprising AAV ITRs (e.g., AAV2 ITRs) and a recombinant nucleic acid comprising a modified (i.e., codon-optimized) nucleic acid encoding mini-dystrophin and at least one of the following regulatory elements: a promoter (e.g., a human CK promoter), a hybrid enhancer and a poly A signal sequence.

In some embodiments, a rAAV3B vector with tropism for liver cells, contains a vector genome comprising AAV ITRs (e.g., AAV2 ITRs) and a recombinant nucleic acid comprising a modified (i.e., codon-optimized) nucleic acid encoding a deleted copper transporting ATPase 2 (e.g., amino acid sequence of SEQ ID NO:15) and at least one of the following regulatory elements: a promoter (e.g., an α1-antitrypsin promoter, e.g., of nucleic acid sequence SEQ ID NO:16) and a poly A signal sequence (e.g., nucleic acid of SEQ ID NO:17).

In some embodiments, a rAAV 9 vector with tropism for muscle cells, contains a vector genome comprising AAV ITRs (e.g., SEQ ID NO:7, SEQ ID NO:8) and a recombinant nucleic acid comprising a modified (i.e., codon-optimized) nucleic acid (e.g., SEQ ID NO:1) encoding mini-dystrophin and at least one of the following regulatory elements: a promoter (e.g., SEQ ID NO:3 or SEQ ID NO:4), a hybrid enhancer and promoter (e.g., SEQ ID NO:5) and a poly A (e.g., SEQ ID NO:6).

### 4. Assembly of Viral Vectors

A viral vector (e.g., rAAV vector) carrying a transgene (e.g., encoding mini-dystrophin) is assembled from a polynucleotide encoding a transgene, suitable regulatory elements and elements necessary for production of viral proteins which mediate cell transduction. Examples of a viral vector include but are not limited to adenoviral, retroviral, lentiviral, herpesvirus and adeno-associated virus (AAV) vectors, and in particular rAAV vector (as discussed, supra).

A vector genome component of a rAAV vector produced according to the methods of the disclosure include at least one transgene, e.g., a codon optimized mini-dystrophin transgene and associated expression control sequences for controlling expression of the modified nucleic acid encoding dystrophin, or a fragment thereof.

In an exemplary non-limiting embodiment, a vector genome includes a portion of a parvovirus genome, such as an AAV genome with rep and cap deleted and/or replaced by a modified nucleic acid (e.g., transgene, e.g., a codon optimized mini-dystrophin transgene) and its associated expression control sequences. A modified nucleic acid encoding dystrophin, or a fragment thereof, is typically inserted adjacent to one or two (i.e., is flanked by) AAV ITRs or ITR elements adequate for viral replication (Xiao et al. (1997) J. Virol. 71(2): 941-948), in place of the nucleic acid encoding viral rep and cap proteins. Other regulatory sequences suitable for use in facilitating tissue-specific expression of a codon optimized mini-dystrophin transgene in the target cell (e.g., muscle cell) may also be included.

Packaging Cell

One skilled in the art would appreciate that a rAAV vector comprising a transgene, and lacking virus proteins needed for viral replication (e.g., cap and rep), cannot replicate since such proteins are necessary for virus replication and packaging. Cap and rep genes may be supplied to a cell (e.g., a host cell, e.g., a packaging cell) as part of a plasmid that is separate from a plasmid supplying the vector genome with the transgene.

“Packaging cell” or “producer cell” means a cell or cell line which may be transfected with a vector, plasmid or DNA construct, and provides in trans all the missing functions which are required for the complete replication and packaging of a viral vector. The required genes for rAAV vector assembly include the vector genome (e.g., a codon optimized mini-dystrophin transgene, regulatory elements, and ITRs), AAV rep gene, AAV cap gene, and certain helper genes from other viruses such as, e.g., adenovirus. One of ordinary skill would understand that the requisite genes for AAV production can be introduced into a packaging cell in various ways including, for example, transfection of one or more plasmids. However, in some embodiments, some genes (e.g., rep, cap, helper) may already be present in a packaging cell, either integrated into the genome or carried on an episome. In some embodiments, a packaging cell expresses, in a constitutive or inducible manner, one or more missing viral functions.

Any suitable packaging cell known in the art may be employed in the production of a packaged viral vector. Mammalian cells or insect cells are preferred. Examples of cells useful for the production of a packaging cell in the practice of the disclosure include, for example, human cell lines, such as PER.C6, WI38, MRC5, A549, HEK293 (which express functional adenoviral E1 under the control of a constitutive promoter), B-50 or any other HeLa cell, HepG2, Saos-2, HuH7, and HT1080 cell lines. Suitable non-human mammalian cell lines include, for example, VERO, COS-1, COS-7, MDCK, BHK21-F, HKCC or CHO cells.

In some embodiments, a packaging cell is capable of growing in suspension culture. In some embodiments, a packaging cell is capable of growing in serum-free media. For example, HEK293 cells are grow in suspension in serum free medium. In another embodiment, a packaging cell is a HEK293 cell as described in U.S. Pat. No. 9,441,206 and deposited as American Type Culture Collection (ATCC) No. PTA 13274. Numerous rAAV packaging cell lines are known in the art, including, but not limited to, those disclosed in WO 2002/46359.

A cell line for use as a packaging cell includes insect cell lines. Any insect cell which allows for replication of AAV and which can be maintained in culture can be used in accordance with the present disclosure. Examples include Spodoptera frugiperda, such as the Sf9 or Sf21 cell lines, Drosophila spp. cell lines, or mosquito cell lines, e.g., Aedes albopictus derived cell lines. A preferred cell line is the Spodoptera frugiperda Sf9 cell line. The following references are incorporated herein for their teachings concerning use of insect cells for expression of heterologous polypeptides, methods of introducing nucleic acids into such cells, and methods of maintaining such cells in culture: Methods in Molecular Biology, ed. Richard, Humana Press, NJ (1995); O'Reilly et al., Baculovirus Expression Vectors: A Laboratory Manual, Oxford Univ. Press (1994); Samulski et al. (1989) J. Virol. 63:3822-3828; Kajigaya et al. (1991) Proc. Nat'l. Acad. Sci. USA 88: 4646-4650; Ruffing et al. (1992) J. Virol. 66:6922-6930; Kimbauer et al. (1996) Virol. 219:37-44; Zhao et al. (2000) Virol. 272:382-393; and U.S. Pat. No. 6,204,059.

As a further alternative, viral vectors of the disclosure may be produced in insect cells using baculovirus vectors to deliver the rep/cap genes and rAAV template as described, for example, by Urabe et al. (2002) Human Gene Therapy 13:1935-1943. When using baculovirus production for AAV, in some embodiments, a vector genome is self-complementary. In some embodiments, a host cell is a baculovirus-infected cell (e.g., an insect cell) comprising, optionally, additional nucleic acids encoding baculovirus helper functions, thereby facilitating production of a viral capsid.

A packaging cell generally includes one or more viral vector functions along with helper functions and packaging functions sufficient to result in replication and packaging of the viral vector. These various functions may be supplied together, or separately, to the packaging cell using a genetic construct such as a plasmid or an amplicon, and they may exist extrachromosomally within the cell line, or integrated into the host cell's chromosomes. In some embodiments, a packaging cell is transfected with at least i) a plasmid comprising a vector genome comprising a transgene and AAV ITRs and further comprising at least one of the following regulatory elements: an enhancer, a promoter, an exon, an intron and a poly A and ii) a plasmid comprising a rep gene (e.g., AAV2 rep) and a cap gene (e.g., AAV9 or other cap).

In some embodiments, a host cell is supplied with one or more of the packaging or helper functions incorporated within, e.g., a host cell line with one or more vector functions incorporated extrachromosomally or integrated into the cell's chromosomal DNA.

Helper Function

AAV is a dependovirus in that it cannot replicate in a cell without co-infection of the cell by a helper virus. Helper functions include helper virus elements needed for establishing active infection of a packaging cell, which is required to initiate packaging of the viral vector. Helper viruses include, typically, adenovirus or herpes simplex virus. Adenovirus helper functions typically include adenovirus components adenovirus early region 1A (E1a), E1b, E2a, E4, and viral associated (VA) RNA. Helper functions (e.g., E1a, E1b, E2a, E4, and VA RNA) can be provided to a packaging cell by transfecting the cell with one or more nucleic acids encoding various helper elements. Alternatively, a host cell (e.g., a packaging cell) can comprise a nucleic acid encoding the helper protein. For instance, HEK293 cells were generated by transforming human cells with adenovirus 5 DNA and now express a number of adenoviral genes, including, but not limited to E1 and E3 (see, e.g., Graham et al. (1977) J. Gen. Virol. 36:59-72). Thus, those helper functions can be provided by the HEK 293 packaging cell without the need of supplying them to the cell by, e.g., a plasmid encoding them. In some embodiments, a packaging cell is transfected with at least i) a plasmid comprising a vector genome comprising a transgene and AAV ITRs and further comprising at least one of the following regulatory elements: an enhancer, a promoter, an exon, an intron and a poly A and ii) a plasmid comprising a rep gene (e.g., AAV2 rep) and a cap gene (e.g., AAV9 or other cap) and iii) a plasmid comprising a helper function.

Any method of introducing a nucleotide sequence carrying a helper function into a cellular host for replication and packaging may be employed, including but not limited to, electroporation, calcium phosphate precipitation, microinjection, cationic or anionic liposomes, a carrier molecule (e.g., polyethylenimine (PEI)) and liposomes in combination with a nuclear localization signal. In some embodiments, helper functions are provided by transfection using a virus vector, or by infection using a helper virus, standard methods for producing viral infection may be used.

The vector genome may be any suitable recombinant nucleic acid, such as a DNA or RNA construct and may be single stranded, double stranded, or duplexed (i.e., self-complementary as described in WO 2001/92551).

### 4. Production of Packaged Viral Vector

Viral vectors can be made by several methods known to skilled artisans (see, e.g., WO 2013/063379). An exemplary non-limiting method is described in Grieger, et al. (2015) Molecular Therapy 24(2):287-297, the contents of which are incorporated by reference herein for all purposes. Briefly, efficient transfection of HEK293 cells is used as a starting point, wherein an adherent HEK293 cell line from a qualified clinical master cell bank is used to grow in animal component-free suspension conditions in shaker flasks and WAVE bioreactors that allow for rapid and scalable rAAV production. Using a triple transfection method (e.g., WO 96/40240), a HEK293 cell line suspension can generate greater than 1×10⁵ vector genome containing particles (VG)/cell, or greater than 1×10¹⁴ VG/L of cell culture, when harvested 48 hours post-transfection. More specifically, triple transfection refers a method whereby a packaging cell is transfected with three plasmids: one plasmid encodes the AAV rep and cap (e.g., AAV9 cap) genes, another plasmid encodes various helper functions (e.g., adenovirus or HSV proteins such as E1a, E1b, E2a, E4, and VA RNA, and another plasmid encodes a transgene (e.g., dystrophin, or a fragment thereof) and various elements to control expression of the transgene.

Single-stranded vector genomes are packaged into capsids as the plus strand or minus strand in about equal proportions. In some embodiments of a rAAV vector, a vector genome is in the plus strand polarity (i.e., the sense or coding sequence of the DNA strand). In some embodiments a rAAV vector, a vector is in the minus strand polarity (i.e., the antisense or template DNA strand). Given the nucleotide sequence of a plus strand in its 5′ to 3′ orientation, the nucleotide sequence of a minus strand in its 5′to 3′ orientation can be determined as the reverse-complement of the nucleotide sequence of the plus strand.

To achieve the desired yields, a number of variables are optimized such as selection of a compatible serum-free suspension media that supports both growth and transfection, selection of a transfection reagent, transfection conditions and cell density.

A rAAV vector may be purified by methods standard in the art such as by column chromatography or cesium chloride gradients. Methods for purifying rAAV vectors are known in the art and include methods described in Clark et al. (1999) Human Gene Therapy 10(6):1031-1039; Schenpp and Clark (2002) Methods Mol. Med. 69:427-443; U.S. Pat. No. 6,566,118 and WO 98/09657.

After rAAV vectors of the present disclosure have been produced and purified according to methods disclosed herein, they can be titered (e.g., the amount of rAAV vector in a sample can be quantified) to prepare compositions for administration to subjects, such as human subjects with Duchenne muscular dystrophy. rAAV vector titering can be accomplished using methods know in the art.

In some embodiments, the number of viral particles, including particles containing a vector genome and “empty” capsids that do not contain a vector genome, can be determined by electron microscopy, e.g., transmission electron microscopy (TEM). Such a TEM-based method can provide the number of vector particles (or virus particles in the case of wild type AAV) in a sample. In some embodiments, the amount of particles, containing a vector genome (full capsids), and “empty” capsids that do not contain a vector genome, can be determined by charge detection mass spectrometry, analytical ultracentrifugation (AUC), and/or measurement of absorbance at 260 nm and 280 nm to determine A260/A280 ratio.

In some embodiments, rAAV vector genomes can be titered using quantitative PCR (qPCR) using primers against any sequence in the vector genome, for example ITR sequences (e.g., SEQ ID NO:7 or SEQ ID NO:8), and/or sequences in the transgene (or regulatory elements). By performing qPCR in parallel on dilutions of a standard of known concentration, such as a plasmid containing the sequence of the vector genome, a standard curve can be generated permitting the concentration of the rAAV vector to be calculated as the number of vector genomes (VG) per unit volume such as microliters or milliliters. By comparing the number of vector particles as measured by, e.g., SEC or ELISA, to the number of vector genomes in a sample, the percentage of empty capsids can be estimated. Because the vector genome contains the therapeutic transgene, vg/kg or vg/ml of a vector sample may be more indicative of the therapeutic amount of the vector that a subject will receive than the number of vector particles, some of which may be empty and not contain a vector genome. Once the concentration of rAAV vector genomes in the stock solution is determined, it can be diluted into or dialyzed against suitable buffers for use in preparing a composition (e.g., a drug substance) for administration to subjects (e.g., subjects with Duchenne muscular dystrophy).

5. rAAV Vector Purification by Anion-Exchange Chromatography (AEX)

A novel, universal purification strategy, based on ion exchange chromatography methods, may be used to generate high purity rAAV vector preparations of various AAV serotypes and/or from chimeric capsids (e.g., AAV1, AAV2, AAV3 including AAV3A and AAV3B, AAV4, AAV5, AAV6, AAV7, AAV8, AAV9, AAV10, AAV12, AAVrh10, AAVrh74, avian AAV, bovine AAV, canine AAV, equine AAV, primate AAV, non-primate AAV, and ovine AAV, and recombinantly produced variants (e.g., capsid variants with insertions, deletions and substitutions, etc.), such as variants referred to as AAV2i8, NP4, NP22, NP66, AAVDJ, AAVDJ/8, AAVDJ/9, AAVLK03, RHM4-1, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15). In some embodiments, this process can be completed in less than a week, result in high full to empty capsid ratios (up to 70% full capsids), provide step yields up to 70% and purity suitable for clinical use. In some embodiments, such a method is universal with respect to AAV serotype and/or chimerism of the capsid. Scalable manufacturing technology, as described herein, may be used to manufacture GMP clinical and commercial grade rAAV vectors to treat disease (e.g., DMD, Friedreich's Ataxia, Wilson Disease etc.).

Production of recombinant AAV vector (rAAV) for gene therapy requires purification of the rAAV vector from a host cell (e.g., host cell debris including but not limited to host cell DNA, RNA, proteins, lipids, membrane and organelles) that produced the vector, as well as removal of capsids that do not contain a complete vector genome (e.g., intermediate and/or empty capsids) and thus, do not comprise a therapeutic transgene.

Such purification methods generally comprise multiple steps including, for example, lysis of the host cell, precipitation of cellular protein and DNA, separation of the rAAV vector from host cell protein and nucleic acids, and separation of the rAAV vector from empty and intermediate capsids by column purification, low speed centrifugation, ultracentrifugation, normal flow filtration, ultrafiltration/diafiltration or any combination of these methods. Column purification may include, for example, ion exchange chromatography (e.g., anion, cation), affinity chromatography, size exclusion chromatography, multimodal chromatography, and/or hydrophobic interaction chromatography. Centrifugation methods may include, for example, ultracentrifugation or low speed centrifugation (e.g., for removal of solids and clarification). Filtration methods may include, for example, diafiltration, depth filtration, nominal filtration and/or absolute filtration.

AEX employs a positively charged stationary phase (e.g., a resin) to separate substances (e.g., AAV capsids, DNA, protein, high molar mass species, amino acids) based on charge differences of said substances, and is useful for separating rAAV capsids from impurities based on charge differences at moderately acidic to alkaline pH (e.g., greater than pH 6). AEX can also separate empty capsids from rAAV vectors containing a complete vector genome (i.e., full capsid) by relying on the charge differences of empty capsids as compared to full capsids.

Without wishing to be bound by theory, the tightness of binding between an AAV capsid and an AEX chromatography stationary phase is related to the strength of the negative charge of the capsid, including the charge contribution from any nucleic acid within the capsid, solution pH and solution conductivity (Qu, G. et al., J. Virological. Methods (2007) 140:183-192). In some embodiments, an AEX chromatography stationary phase is a resin comprising polystyrenedivinylbenzene particles modified with covalently bound quaternized polyethyleneimine, and optionally OH groups (e.g., POROS™ 50 HQ resin). Polystyrenedivinylbenzene particles may comprise pores of 500-10,000 Angstroms (Å).

In some embodiments, an AEX chromatography stationary phase is a resin comprising agarose particles with a cationic ligand (e.g. Capto Q ImpRes, Q Sepharose High Performance). In some embodiments, an AEX chromatography stationary phase is a resin selected from the group consisting of Capto Q, Capto Q XP, Q Sepharose XL, STREAMLINE Q XL, Capto HiRes Q, RESOURCE Q, SOURCE 15 Q, SOURCE 30 Q, Q Sepharose HP, Q Sepharose FF, Q Sepharose™ BB, POROS™ 20 HQ, POROS™ XQ, TOYOPEARL QAE-550C, TOYOPEARL Q-600C AR, TOYOPEARL GigaCap Q-650S, TOYOPEARL GigaCap Q-650M, TOYOPEARL SuperQ-650S, TOYOPEARL SuperQ-650M, TOYOPEARL SuperQ-650C, TSKgel SuperQ-5PW (20), TSKgel SuperQ-5PW (30), Q Ceramic HyperD F, ESHMUNO® Q, Fractogel® EMD TMAE (S), Fractogel® EMD TMAE (M), Fractogel® EMD TMAE Hicap (M), Fractogel® EMD TMAE (S), Fractogel® EMD TMAE (M), Fractogel® EMD TMAE Hicap (M), Nuvia Q, Nuvia HP-Q, UNOsphere Q, Macro-Prep High Q, Macro-Prep 25 Q, BioRad AG® 1-X2, WorkBeads™ 40Q, WorkBeads™ 100Q, Cellufine MAX Q-r, Cellufine MAX Q-h, Praesto™ Q65, Praesto™ Q90, Praesto™ Jetted Q35, BAKERBOND™ POLYQUAT, BAKERBOND™ POLYPEI, YMC—BioPro Q30, YMC—BioPro Q75, YMC—BioPro SmartSep 010, YMC—BioPro SmartSep Q30, DEAE Sepharose FF, ANX Sepharose 4 FF (high sub), POROS™ 50 PI, POROS™ 50 D, TOYOPEARL NH₂-750F, TOYOPEARL GigaCap DEAE-650M, TOYOPEARL DEAE-650S, TOYOPEARL DEAE-650M, TOYOPEARL DEAE-650C, TSKgel DEAE-5PW (20), TSKgel DEAE-5PW (30), Ceramic HyperD DEAE, Hypercel Star AX, Fractogel® EMD DEAE (M), Fractogel® EMD DMAE (M) Resin, Macro-Prep DEAE, WorkBeads™ 40 DEAE, Cellufine MAX DEAE, DEAE PuraBead HF and WorkBeads™ 40 TREN.

In some embodiments, an AEX chromatography stationary phase is a monolith comprising porous poly-methacrylate with a cationic ligand (e.g. ClMmultus™ QA). In some embodiments, an AEX chromatography stationary phase is a membrane adsorber comprising polyethersulfone with a cationic ligand (e.g. Mustang Q, Mustang E, Sartobind® Q, Sartobind STIC® PA).

In some embodiments, a rAAV vector can be purified by AEX from a solution exiting from an affinity chromatography stationary phase (e.g., “eluting from the stationary phase”) comprised of mobile phase and material such as rAAV vector or capsid that passed through the stationary phase or was displaced from the stationary phase. This solution may be referred to as an affinity eluate or an “affinity pool.”

In some embodiments, a rAAV vector can be purified by AEX from a “supernatant from a cell lysate” (also known as a “clarified lysate”), which, as used herein, refers to a solution collected following sedimentation of lysed host cells from a host cell culture.

In some embodiments, a rAAV vector can be purified by AEX from a “post-harvest solution”, which, as used herein, refers to solution resulting from a cell lysis that has undergone flocculation, depth filtration and/or nominal filtration.

In some embodiments, a rAAV vector can be purified from a solution having undergone at least one other purification or processing step (e.g., cell lysis, flocculation, filtration, dilution, pH adjustment, chromatography). In some embodiments, an affinity eluate has been diluted, and optionally filtered prior to purification of the rAAV vector, such as prior to loading the affinity eluate onto the AEX column.

In some embodiments, a rAAV vector can be purified by AEX from an affinity eluate, optionally having undergone at least one other purification or processing step (e.g., cell lysis, flocculation, filtration, dilution, pH adjustment, chromatography). In some embodiments, a rAAV vector can be purified by AEX from a cell lysate, optionally having undergone at least one other purification or processing step (e.g., cell lysis, flocculation, filtration, dilution, pH adjustment, chromatography). In some embodiments, a rAAV vector can be purified by AEX from a post-harvest solution, optionally having undergone at least one other purification or processing step (e.g., cell lysis, flocculation, filtration, dilution, pH adjustment, chromatography).

As a solution comprising a substance to be purified (e.g., a rAAV vector) and impurities, flows through an AEX stationary phase, a substance that binds (e.g., negatively charged proteins such as an AAV capsid or rAAV vector) to a positively charged AEX stationary phase are retained within the stationary phase. Unbound substances pass through the column and are collected in a flow-through, and/or during a subsequent wash step. Bound substances may be eluted from the stationary phase by adjusting a salt concentration and/or pH within the column. For example, and without wishing to be bound by any particular theory of operation, a salt concentration of an elution buffer is gradually increased such that anions in the salt (e.g., acetate (C₂H₃O₂⁻), Cl⁻SO₄⁻²) compete with and displace (i.e., elute) a substance bound to the resin. In another embodiment, the pH of the solution within the column can be gradually decreased to decrease the negative charge of a bound substance and cause it to be released (i.e., eluted) from the stationary phase. Upon release from the stationary phase, a substance may be collected as a column eluate.

Without wishing to be bound by theory, separation of substances, such as a mixture of AAV capsids, or more specifically a mixture of a rAAV vector (i.e., a full capsid), an AAV capsid (e.g., an empty capsid, an intermediate capsid) and host cell proteins, will depend on the total charge difference of the substances. The charge composition of ionizable side groups will determine the total charge of a protein at a particular pH. At the isoelectric point (pl), the total charge on a protein is 0 and it will not bind to a matrix. If the pH is above the pl, a protein will have a negative charge and bind to an anion exchange column stationary phase.

An AEX protocol for separation of full rAAV vectors from empty capsids includes multiple steps, for example, pre-use flushing of a column media to displace storage solution, pre-use sanitizing of a column stationary phase, post-use sanitizing of a column stationary phase, equilibrating a column stationary phase, loading a solution (e.g., a diluted affinity eluate) comprising a rAAV vector onto a column stationary phase, eluting a substance to be purified from a stationary phase (e.g., by gradient elution, by step elution), applying a gradient hold to a column stationary phase, sanitizing a column stationary phase, regenerating a column stationary phase, applying a storage solution to a column stationary phase. One of skill in the art will understand that an AEX protocol for purification of rAAV vectors may comprise all, or only some of these steps. One of skill in the art will also understand that the order of these steps may vary, and that certain steps may be performed more than once, and not necessarily in sequence.

AEX Column Preparation

AEX methods of the disclosure may be performed at various scales utilizing columns ranging in volume from 1.0 mL to 20 L. In some embodiments, an AEX method includes use of a column with a column volume (CV) of about 1.0 mL, about 5.1 mL, about 49 mL, about 52 mL, about 6.67 mL, about 1.256 L, about 1.3 L, about 6.0 L, about 6.1 L, about 6.2 L, about 6.3 L, about 6.4 L, about 6.5 L, about 6.6 L, about 6.7 L, about 6.8 L, about 6.9 L, or about 7.0 L. In some embodiments, an AEX method of the disclosure includes use of a column with a CV of 1.0 mL to 20 L, e.g., 1.0 ml to 10 mL, 30 mL to 70 mL, 10 mL to 100 mL, 100 mL to 1000 mL, 1 L to 1.5 L, 1.5 L to 2.0 L, 2.0 L to 5 L, 5 L to 7.5 L, 7.5 L to 10 L, 10 L to 15 L or 15 L to 20 L. In some embodiments, an AEX method of the disclosure includes use of a column with a CV of 1.0 mL to 10 L, 10 mL to 10 L, 100 mL to 20 L, 100 mL to 10 L, 1 L to 20 L, 1L to 10 L, 1 L to 5 L, 1 L to 2 L or 1 L to 1.5 L. In some embodiments, an AEX method of the disclosure includes use of a column with a CV of 6.0 L to 6.6 L (e.g., 6.4 L).

A volume of solution applied to a column to, for example, to equilibrate a stationary phase therein, is generally expressed in terms of a “column volume” (CV), with one CV equivalent to the volume of the column.

In some embodiments, an AEX chromatography stationary phase (also referred to herein as “resin” or “media”) of the disclosure is a polystyrenedivinylbenzene particle with covalently bound quaternized polyethyleneimine (e.g., POROS™ 50 HQ resin).

Generally, prior to application (i.e., loading) of a solution to be purified (e.g., an affinity chromatography eluate, also referred to herein as an “affinity eluate” or an “affinity pool”) to a column comprising a chromatography stationary phase, at least one solution is applied to the stationary phase to, for example, flush, sanitize, regenerate and/or equilibrate the stationary phase. In some embodiments, an “affinity eluate” or an “affinity pool” has been diluted, and optionally filtered prior to loading of the solution onto the AEX column.

As disclosed herein, a method of preparing an AEX stationary phase for use in a method of purifying a rAAV (e.g., rAAV9, rAAV3B or others) vector from a solution (e.g., an affinity eluate) by AEX comprises pre-use flushing of the AEX stationary phase in a column. In some embodiments, pre-use flushing of the AEX stationary phase is intended to displace a storage solution (e.g., a solution comprising ethanol) from the stationary phase. In some embodiments, pre-use flushing of a column precedes loading a solution comprising a rAAV vector to be purified onto the column. In some embodiments, pre-use flushing comprises application of water (e.g., water for injection) to AEX stationary phase in a column. In some embodiments, pre-use flushing comprises an upward flow of water. During upward flow of pre-use flushing, the flow direction is opposite that of chromatographic separation steps (e.g., loading, washing or eluting), such that the solution (e.g., water) flows from the bottom of the column to the top of the column, whereas during a chromatographic separation step (e.g., loading) the solution flows from the top of the column to the bottom of the column. In some embodiments, pre-use flushing comprises application of 1 to 10 column volumes (CV) (e.g., about 5 CV) of water to AEX stationary phase in a column, at a linear velocity of 10 cm/hr to 1000 cm/hr and/or a flow rate of 0.2 L/min to 3.0 L/min. In some embodiments, pre-use flushing comprises application of ≥4.5 CV (e.g., about 5 CV) of water for injection to AEX stationary phase in a column, at a linear velocity of 270 cm/hr to 330 cm/hr (e.g., about 300 cm/hr), a flow rate of 1.5 L/min to 2.0 L/min (e.g., about 1.8 L/min) and/or a residence time (i.e., a contact time) of 3.5 min/CV to 4.5 min/CV (e.g., about 4 min/CV).

A method of preparing an AEX stationary phase for use in a method of purifying a rAAV (e.g., rAAV9, rAAV3B or others) vector from a solution (e.g., an affinity eluate) by AEX comprises sanitizing the AEX stationary phase in a column. Sanitizing an AEX stationary phase serves to reduce the bioburden (including, but not limited to bacteria) and/or inactivate microbes and viruses within the column, and more generally to remove contaminants such as proteins, particulates, etc. In some embodiments, sanitizing precedes loading a solution comprising a rAAV vector to be purified onto a column. In some embodiments, sanitizing comprises application of a solution comprising NaOH, ethanol, acetic acid, phosphoric acid, guanidine HCl, urea, PAB (phosphoric add, acetic acid, benzyl alcohol), peracetic acid etc. to an AEX stationary phase in a column. In some embodiments, sanitizing comprises application of a solution comprising 0.1 M to 1.0 M, about 0.1 M to about 0.8 M, about 0.1 M to about 0.6 M, about 0.2 M to about 0.8 M, about 0.2 M to about 0.6 M or about 0.4 M to about 0.6 M (e.g., about 0.5 M) NaOH to AEX stationary phase in a column. In some embodiments, sanitizing comprises application a solution comprising about 0.5 M NaOH to AEX stationary phase in a column using an upward flow (i.e., that is the flow direction is opposite that of chromatographic separation steps, e.g., loading, washing or eluting). In some embodiments, sanitizing comprises application a solution comprising about 0.5 M NaOH to AEX stationary phase in a column using an downward flow (i.e., that is the flow direction is in the same direction as that of chromatographic separation steps, e.g., loading, washing or eluting). In some embodiments, sanitizing comprises application of 14.4 CV to 17.6 CV (e.g., about 16 CV) of a solution comprising about 0.5 M NaOH to AEX stationary phase in a column. In some embodiments, sanitizing comprises application of 5 CV to 10 CV (e.g., about 8 CV) of a solution comprising about 0.5 M NaOH to AEX stationary phase in a column. In some embodiments, sanitizing comprises application of 5 CV to 20 CV of a solution comprising about 0.5 M NaOH to an AEX stationary phase in a column at a linear velocity of 100 cm/hr to 1000 cm/hr and/or a flow rate of 0.2 L/min to 3.0 L/min. In some embodiments, sanitizing comprises application of 14.4 CV to 17.6 CV (e.g. about 16 CV) of a solution comprising about 0.5 M NaOH to AEX stationary phase in a column at a linear velocity of 270 cm/hr to 330 cm/hr (e.g., about 300 cm/hr), a flow rate of 1.5 L/min to 2.0 L/min (e.g., about 1.8 L/min) and/or a residence time (i.e., the amount of time per column volume that the solution is in contact with the stationary phase within the column, and also referred to herein as the contact time) of 3.5 min/CV to 4.5 min/CV (e.g., about 4 min/CV). In some embodiments, sanitizing comprises application of 5 CV to 10 CV (e.g. about 8 CV) of a solution comprising about 0.5 M NaOH to AEX stationary phase in a column at a linear velocity of 270 cm/hr to 330 cm/hr (e.g., about 298 cm/hr) and/or a residence time of 1.5 min/CV to 2.5 min/CV (e.g., about 2 min/CV).

A method of preparing an AEX stationary phase for use in a method of purifying a rAAV (e.g., rAAV9, rAAV3B or others) vector from a solution (e.g., an affinity eluate) by AEX comprises regenerating (also referred to herein as “a rinse”) an AEX stationary phase in a column. One of skill in the art will understand that regenerating an ion exchange stationary phase serves to replace ions taken up in the exchange process with the original ions that occupied the exchange sites. In some embodiments, regeneration can also refer to bringing back a stationary phase to its original state by, for example, the removal of impurities using a strong solvent. In some embodiments, regenerating precedes loading a solution comprising a rAAV vector to be purified onto a stationary phase. In some embodiments, regenerating may be performed on a stationary phase more than once.

In some embodiments, regenerating comprises application of a solution comprising a salt and/or a buffering agent, with a pH ranging from 8 to 10, to an AEX stationary phase in a column. In some embodiments, a salt is selected from the group consisting of sodium chloride (NaCl), sodium acetate (NaAcetate,CH₃COONa), ammonium acetate (NH₄Acetate), magnesium chloride (MgCl₂) or sodium sulfate (Na₂SO₄). In some embodiments, a concentration of a salt in a solution (e.g., NaCl) ranges from 1 M to 5 M (e.g., about 1 M to about 4.5 M, about 1 to about 4M, about 1 M to about 3.5 M, about 1M to about 3 M, about 1M to about 2.5 M or about 1.5 M to about 2.5 M. In some embodiments, a concentration of a salt in a solution (e.g., NaCl) is about 1 M, about 2 M, about 3 M, about 4 M or about 5 M. In some embodiments, regenerating comprises application of a solution comprising 1 M to 3 M (e.g., 2 M) NaCl to the stationary phase in the column.

In some embodiments, a buffering agent is selected from the group consisting of Tris (e.g., a mixture of Tris Base and Tris-HCl), BIS-Tris propane, and/or bicine. In some embodiments, the concentration of the buffering agent (e.g., Tris) in a solution ranges from 10 mM to 500 mM (e.g., about 10 mM to about 450 mM, about 10 mM to about 400 mM, about 10 mM to about 350 mM, about 10 mM to about 300 mM, about 10 mM to about 250 mM, about 10 mM to about 200 mM, about 10 mM to about 150 mM, or about 50 mM to about 150 mM. In some embodiments, the concentration of the buffering agent (e.g., Tris) in a solution is about 10 mM, about 20 mM about 50 mM, about 100 mM, about 150 mM, about 200 mM, about 300 mM, about 400 mM or about 500 mM. In some embodiments, regenerating comprises application of a solution comprising 50 mM to 150 mM (e.g., 100 mM) Tris to a stationary phase in a column.

In some embodiments, regenerating comprises application of a solution with a pH of about 7 to 11 (e.g., about 7.5 to 10.5, about 8 to 10, or about 7, 7.5, 8, 8,5, 9, 9.5, 10, 10.5 or 11) to a stationary phase in a column.

In some embodiments, regenerating comprises application of a solution comprising about 1 M to 3 M (e.g., about 2 M) NaCl, 50 mM to 150 mM (e.g., about 100 mM) Tris, pH 8.5 to 9.5 (e.g., about 9) to AEX stationary phase in a column. In some embodiments, regenerating comprises application of 4.5 to 5.5 CV (e.g., about 5 CV) of a solution comprising about 2 M NaCl, 100 mM Tris, pH 9 to AEX stationary phase in a column. In some embodiments, regenerating comprises application of 1 to 10 CV of a solution comprising about 2 M NaCl, 100 mM Tris, pH 9 to AEX stationary phase in a column at a linear velocity of 100 to 1000 cm/hr and/or a flow rate of 0.2 to 3.0 L/min. In some embodiments, regenerating comprises application of 4.5 to 5.5 (e.g., about 5) CV of a solution comprising about 2 M NaCl, 100 mM Tris, pH 9 to AEX stationary phase in a column at a linear velocity of 270 to 330 cm/hr (e.g., about 298 cm/hr, about 300 cm/hr), a flow rate of 1.5 to 2.0 L/min (e.g., about 1.8 L/min) and/or a residence time (i.e., a contact time) of 1.5 to 4.5 min/CV (e.g., about 2 min/CV, about 4 min/CV).

In some embodiments, the present disclosure provides a method of preparing an AEX stationary phase for use in a method of purifying a rAAV (e.g., rAAV9, rAAV3B or others) vector by AEX, the method comprising a step of: i) pre-use flushing comprising application of ≥4.5 CV (e.g., about 5 CV) of water for injection to AEX stationary phase in a column; ii) sanitizing comprising application of about 5 CV to 10 CV (e.g., about 8 CV) or about 14.4 to 17.6 CV (e.g., about 16 CV) of a solution comprising 0.1 M to 1.0 M (e.g., about 0.5 M NaOH) to the AEX stationary phase in the column, optionally by upward flow; and/or iii) regenerating comprising application of 4.5 to 5.5 CV (e.g., about 5 CV) of a solution comprising 1 M to 3 M (e.g., about 2 M) NaCl, 50 mM to 150 mM (e.g., about 100 mM) Tris, pH 8.5 to 9.5 (e.g., about 9) to the AEX stationary phase in the column; wherein at least one of steps i)-iii) is performed at a linear velocity of 270 cm/hr to 330 cm/hr (e.g., about 298 cm/hr, about 300 cm/hr), a flow rate of 1.5 L/min to 2.0 L/min (e.g., about 1.8 L/min) and/or a residence time of 1.5 min/CV to 4.5 min/CV (e.g., about 2 min/CV, about 4 min/CV); optionally wherein at least one step is performed prior to loading a solution comprising the rAAV vector to be purified onto the column; and optionally wherein the AEX stationary phase is POROS™ 50 HQ. One of ordinary skill will understand that the above steps may be performed in any order and may be performed more than once.

Equilibration

A method of preparing an AEX stationary phase for use in a method of purifying a rAAV (e.g., rAAV9, rAAV3B or others) vector from a solution (e.g., an affinity eluate) by AEX comprises equilibration of the AEX stationary phase in a column. In some embodiments, equilibration of an AEX stationary phase in a column serves to adjust the pH, conductivity, modifier (e.g., salt, detergent, amino acid etc.) concentration, or other condition, of the mobile and stationary phase such that some substances loaded onto the column will bind to the stationary phase, and others will flow through with the mobile phase. For example, conditions within the column may be adjusted by the application of a series of equilibration buffers to the column such that full rAAV vectors bind to the stationary phase, and at least a portion of the empty capsids do not bind. In some embodiments, AEX stationary phase in a column is equilibrated prior to application of a solution comprising a substance to be purified (e.g., a rAAV vector) to the column. In some embodiments, AEX stationary phase in a column is equilibrated by application of an equilibration buffer (e.g., a first equilibration buffer, a second equilibration buffer, a third equilibration buffer, a fourth equilibration buffer, etc.). An equilibration buffer may also be referred to herein as a “wash buffer,” a “post-sanitization rinse,” a “rinse,” or a “regeneration buffer.” Reference to an equilibration buffer as a first, second, third, fourth, etc. equilibration buffer does not necessarily imply the order in which the buffers are applied to a column.

In some embodiments, an equilibration buffer (e.g., a first equilibration buffer, a second equilibration buffer, a third equilibration buffer, a fourth equilibration buffer, etc.) comprises at least one component selected from the group consisting of a buffering agent, a salt, an amino acid, a detergent and/or a combination thereof. In some embodiments, a buffering agent is Tris (e.g., a mixture of Tris Base and Tris-HCl), BIS-Tris propane, diethanolamine, diethylamine, tricine, triethanolamine and/or bicine. One of ordinary skill in the art would understand that a Tris buffer with a desired pH can be prepared using Tris Base, Tris-HCl or both. In some embodiments, a salt is sodium chloride (NaCl), sodium acetate (NaAcetate (CH₃COONa)), ammonium acetate (NH₄Acetate), magnesium chloride (MgCl₂) or sodium sulfate (Na₂SO₄). In some embodiments, a salt is sodium acetate. In some embodiments, an amino acid is histidine, arginine, glycine or citrulline. In some embodiments, a detergent is poloxamer 188 (P188), Triton X-100, Polysorbate 80, Brij-35 or nonyl phenoxypolyethoxylethanol (NP-40).

In some embodiments, an equilibration buffer comprises 10 mM to 350 mM of a buffering agent selected from the group consisting of Tris (e.g., a mixture of Tris Base and Tris-HCl), BIS-Tris propane, diethanolamine, diethylamine, tricine, triethanolamine and bicine. In some embodiments, an equilibration buffer comprises 10 mM to 350 mM, 10 mM to 300 mM Tris, 10 mM to 250 mM Tris, 10 mM to 200 mM Tris, 10 mM to 150 mM Tris, 10 mM to 100 mM Tris or 10 mM to 50 mM Tris. In some embodiments, an equilibration buffer comprises 30 mM to 350 mM Tris, 30 mM to 300 mM Tris, 30 mM to 250 mM Tris, 30 mM to 2000 mM Tris, 30 mM to 150 mM Tris, 30 mM to 100 mM Tris. In some embodiments, an equilibration buffer comprises 50 mM to 300 mM Tris, 50 mM to 250 mM Tris, 50 mM to 200 mM Tris, 50 mM to 150 mM Tris. In some embodiments, an equilibration buffer comprises 100 mM to 350 mM Tris, 100 mM to 250 mM Tris or 100 mM to 150 mM Tris. In some embodiments, an equilibration buffer comprises about 10 mM Tris, about 20 mM Tris, about 30 mM Tris, about 40 mM Tris, about 50 mM Tris, about 60 mM Tris, about 70 mM Tris, about 80 mM Tris, about 90 mM Tris, about 100 mM Tris, about 110 mM Tris, about 120 mM Tris, about 130 mM Tris, about 140 mM Tris, about 150 mM Tris, about 160 mM Tris, about 170 mM Tris, about 180 mM Tris, about 190 mM Tris, about 200 mM Tris, about 220 mM Tris, about 240 mM Tris, about 250 mM Tris, about 275 mM Tris, about 300 mM Tris or about 350 mM Tris. In some embodiments, an equilibration buffer comprises about 20 mM Tris, 100 mM Tris or 200 mM Tris.

In some embodiments, an equilibration buffer comprises 1 mM to 1M salt, and preferably about 500 mM salt. In some embodiments, an equilibration buffer comprises about 10 mM to about 950 mM, about 10 mM to about 900 mM, about 10 mM to about 850 mM, about 10M to about 800 mM, 10 mM to about 750 mM, about 10 mM to about 700 mM, about 10 mM to about 650 mM, about 10 mM to about 600 mM, about 10 mM to about 550 mM, about 50 mM to about 750 mM, about 50 mM to about 700 mM, about 50 mM to about 650 mM, about 50 mM to about 600 mM, about 50 mM to about 550 mM, about 100 mM to about 600 mM, about 200 mM to about 600 mM, about 300 mM to about 600 mM or about 400 mM to about 600 mM salt. In some embodiments, an equilibration buffer comprises about 500 mM salt. In some embodiments, an equilibration buffer comprises a salt selected from the group consisting of sodium chloride (NaCl), sodium acetate (NaAcetate,CH₃COONa), ammonium acetate (NH₄Acetate), magnesium chloride (MgCl₂) or sodium sulfate (Na₂SO₄).

In some embodiments, an equilibration buffer comprises 5 mM to 1 M sodium acetate. In some embodiments, an equilibration buffer comprises about 10 mM to about 950 mM, about 10 mM to about 900 mM, about 10 mM to about 850 mM, about 10M to about 800 mM, 10 mM to about 750 mM, about 10 mM to about 700 mM, about 10 mM to about 650 mM, about 10 mM to about 600 mM, about 10 mM to about 550 mM, about 50 mM to about 750 mM, about 50 mM to about 700 mM, about 50 mM to about 650 mM, about 50 mM to about 600 mM, about 50 mM to about 550 mM, about 100 mM to about 600 mM, about 200 mM to about 600 mM, about 300 mM to about 600 mM, or about 400 mM to about 600 mM sodium acetate. In some embodiments, an equilibration buffer comprises about 5 mM, about 6 mM, about 7 mM, about 8 mM, about 9 mM, about 10 mM, about 20 mM, about 30 mM, about 40 mM, about 50 mM, about 60 mM, about 70 mM, about 80 mM, about 90 mM, about 100 mM, about 150 mM, about 200 mM, about 250 mM, about 300 mM, about 350 mM, about 400 mM, about 450 mM, about 500 mM, about 550 mM or about 600 mM sodium acetate. In some embodiments, an equilibration buffer comprises about 500 mM sodium acetate.

In some embodiments, an equilibration buffer comprises an amino acid, e.g., histidine, arginine, glycine or citrulline. In some embodiments, an equilibration buffer comprises about 50 mM, about 75 mM, about 100 mM, about 125 mM, about 150 mM, about 175 mM, about 200 mM, about 225 mM, about 250 mM, about 275 mM or about 300 mM of an amino acid (e.g., histidine, arginine, glycine or citrulline).

In some embodiments, an equilibration buffer comprises an amino acid, e.g., histidine or arginine. In some embodiments, an equilibration buffer comprises 100 mM to 300 mM of an amino acid (e.g., histidine arginine, glycine or citrulline). In some embodiments, an equilibration buffer comprises about 10 mM to about 600 mM, about 10 mM to about 550 mM, about 10 mM to about 500 mM, about 10 mM to about 450 mM about 10 mM to about 400 mM, about 10 mM to about 350 mM, about 10 mM to about 300 mM, about 50 mM to about 600 mM, about 50 mM to about 550 mM, about 50 mM to about 500 mM, about 50 mM to about 450 mM, about 50 mM to about 400 mM, about 50 mM to about 350 mM, about 50 mM, to about 300 mM, about 100 mM to about 600 mM, about 100 mM to about 500 mM, about 100 mM to about 400 mM, about 100 mM to about 300 mM salt, or about 150 mM to about 250 mM of an amino acid (e.g., histidine). In some embodiments, an equilibration buffer comprises about 200 mM histidine.

In some embodiments, an equilibration buffer comprises a detergent, e.g., P188, Triton X-100, Polysorbate 80, Brij-35 or NP-40. In some embodiments, an equilibration buffer comprises 0.005% to 1.0% of a detergent (e.g., P188). In some embodiments, an equilibration buffer comprises 0.005% to 0.015% of a detergent (e.g., P188). In some embodiments, an equilibration buffer comprises 0.1% to 1.0% of a detergent (e.g., P188). In some embodiments, an equilibration buffer comprises about 0.005% to about 1.0%, about 0.005% to about 0.5%, about 0.005% to about 0.1% about 0.005% to about 0.05%, about 0.007% to about 0.07%, 0.008% to about 0.05% or about 0.008% to about 0.03% of P188. In some embodiments, an equilibration buffer comprises about 0.01% to about 1.5%, about 0.01% to about 1.0%. about 0.01% to about 0.75%, about 0.05% to about 1.5%, about 0.05% to about 1.0%, about 0.05% to about 0.75%, about 0.1% to about 1.5%, about 0.1% to about 1.0%, about 0.1% to about 0.75%, or about 0.25% to about 0.75% P188.

In some embodiments, an equilibration buffer comprises about 0.005%, about 0.006%, about 0.007%, about 0.008%, about 0.009%, about 0.01%, about 0.02%, about 0.03% about 0.04%, about 0.05%, about 0.06%, about 0.07%, about 0.08%, about 0.09%, about 0.1%, about 0.15%, about 0.2%, about 0.25%, about 0.3%, about 0.35%, about 0.4%, about 0.5%, about 0.55%, about 0.6%, about 0.65%, about 0.7%, about 0.75%, about 0.8%, about 0.85%, about 0.9%, 0.95% or about 1.0% of a detergent (e.g., P188). In some embodiments, an equilibration buffer comprises about 0.01% P188. In some embodiments, an equilibration buffer comprises about 0.5% P188.

In some embodiments, an equilibration buffer has a pH of 8 to 10. In some embodiments, an equilibration buffer has a pH of 8.7 to 9.3. In some embodiments, an equilibration buffer has a pH of 8.7 to 9.0. In some embodiments, an equilibration buffer has a pH of about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5 or about 10.0. In some embodiments, an equilibration buffer has a pH of about 8.8. In some embodiments, an equilibration buffer has a pH of about 8.9. In some embodiments, an equilibration buffer has a pH of about 9.0.

In some embodiments, an equilibration buffer comprises 20 mM Tris, pH 9.0. In some embodiments, an equilibration buffer comprises 100 mM Tris, pH 9.

In some embodiments, an equilibration buffer comprises 20 mM Tris and 500 mM NaCl, pH 9.0+/−0.3. In some embodiments, an equilibration buffer comprises 20 mM Tris and 500 mM NH₄Acetate, pH 9.0+/−0.3. In some embodiments, an equilibration buffer comprises about 20 mM Tris, 500 mM sodium acetate, pH 9.0+/−0.3. In some embodiments, an equilibration buffer comprises 20 mM Tris and 500 mM Na₂SO₄, pH 9.0+/−0.3.

In some embodiments, an equilibration buffer comprises 20 mM Tris, 7 mM salt (e.g., NaCl, sodium acetate, ammonium acetate (NH₄Acetate), MgCl₂ and Na₂SO₄) pH 9.0. In some embodiments, an equilibration buffer comprises 20 mM Tris, 7 mM sodium acetate, pH 9.0. In some embodiments, an equilibration buffer comprises 20 mM Tris, 14 mM sodium acetate, pH 9.0. In some embodiments, an equilibration buffer comprises 20 mM Tris, 21 mM sodium acetate, pH 9.0. In some embodiments, an equilibration buffer comprises 20 mM Tris, 42 mM sodium acetate, pH 9.0. In some embodiments, an equilibration buffer comprises 20 mM Tris, 49 mM sodium acetate, pH 9.0. In some embodiments, an equilibration buffer comprises 20 mM Tris, 57 mM sodium acetate, pH 9.0. In some embodiments, an equilibration buffer comprises 20 mM Tris, 67 mM sodium acetate, pH 9.0.

In some embodiments, an equilibration buffer comprises 50 mM to 150 mM (e.g., about 100 mM) Tris, pH 8.5 to 9.5 (e.g., about 9). In some embodiments, an equilibration comprises 50 mM to 150 mM (e.g., about 100 mM) Tris, 400 mM to 600 mM (e.g., about 500) mM sodium acetate, 0.005% to 0.015% (e.g., about 0.01%) P188, pH 8.5 to 9.5 (e.g., about 8.9). In some embodiments, an equilibration buffer comprises 100 mM to 300 mM histidine (e.g., about 200 mM) histidine, 100 mM to 300 mM (e.g., about 200 mM) Tris, 0.1% to 1.0% (e.g., about 0.5%) P188, pH 8.5 to 9.5 (e.g., about 8.8). In some embodiments, an equilibration buffer comprises 50 mM to 150 mM (e.g., about 100 mM) Tris, 0.005% to 0.015% (e.g., about 0.01%) P188, pH 8.5 to 9.5 (e.g., about 8.9).

In some embodiments, an equilibration buffer (e.g., a first equilibration buffer) comprises 100 mM Tris, pH 9. In some embodiments, an equilibration buffer (e.g., a first or a second equilibration buffer) comprises 100 mM Tris, 500 mM sodium acetate, 0.01% P188, pH 8.9. In some embodiments, an equilibration buffer (e.g., a second or a third equilibration buffer) comprises 200 mM histidine, 200 mM Tris, 0.5% P188, pH 8.8. In some embodiments, an equilibration buffer (e.g., a third or a fourth equilibration buffer) comprises 100 mM Tris, 0.01% P188, pH 8.9.

In some embodiments, an equilibration buffer described above may be a first, second, third and fourth equilibration buffer. In some embodiments, a first, second, third or fourth equilibration buffer is applied to a column stationary phase in sequential order. In some embodiments, a solution (e.g., an affinity eluate) is applied to the column between application of two equilibration buffers. For example, a first, second and third equilibration buffer may be applied to a column, followed by application of an affinity eluate, which is followed by application of a fourth equilibration buffer. In another example, a first and second equilibration buffer are applied to a column, followed by application of an affinity eluate, which is followed by application of a third equilibration buffer.

In some embodiments, an amount of equilibration buffer applied to a column is 1 CV to 5 CV, 4 CV to 6 CV, 4 CV to 10 CV, 4 CV to 15 CV, 4 CV to 21 CV, 10 CV to 21 CV, 15 CV to 21 CV or 19 CV to 21 CV. In some embodiment, an amount of equilibration buffer applied to a column is ≥4.5 CV. In some embodiments, an amount of an equilibration buffer applied to a column is 4.5 CV to 5.5 CV. In some embodiments, an amount of equilibration buffer applied to a column is about 2 CV, about 5 CV or about 10 CV. In some embodiments, an amount of equilibration buffer applied to a column is about 5 CV. In some embodiments, an amount of equilibration buffer applied to a column is about 20 CV.

A solution, including but not limited to an equilibration buffer, applied to a column is set to flow through the stationary phase at a particular rate (e.g., cm/hr, mL/min) so that the solution within the column is in contact with the stationary phase, for a particular period of time (referred to herein as “residence time” or “contact time”). In some embodiments, a residence time of a solution in a column is 0.1 min/CV to 10 min/CV, e.g., 0.1 min/CV to 1 min/CV, 1 min/CV to 2 min/CV, 2 min/CV to 4 min/CV, 4 min/CV to 6 min/CV, 6 min/CV to 8 min/CV, or 8 min/CV to 10 min/CV. In some embodiments, a residence time of a solution in a column is 0.1 min/CV, about 0.5 min/CV, about 1.5 min/CV, about 2 min/CV, about 3 min/CV, about 3.6 min/CV or about 4 min/CV, about 5 min/CV, about 6 min/CV, about 7 min/CV, about 8 min/CV, about 9 min/CV or about 10 min/CV. In some embodiments, a residence time of a solution in a column is 1.5 to 4.5 min/CV. In some embodiments, a residence time of a solution in a column is 3.5 to 4.5 min/CV.

In some embodiments, a residence time of a solution in a column with a height of about 5 cm, a diameter of about 0.5 cm and a volume of about 1.0 mL is about 0.5 min/CV. In some embodiments, a residence time of a solution in a column with a height of about 15 cm, a diameter of about 0.66 cm and a volume of about 5.1 mL is about 0.5 min/CV, about 1.5 min/CV or about 4 min/CV. In some embodiments, a residence time of a solution in a column with a height of about 19.5 cm, a diameter of about 0.66 and a volume of about 6.67 mL is about 4 min/CV. In some embodiments, a residence time of a solution in a column with a height of about 10 cm, a diameter of about 2.5 cm and volume of about 49 mL is 1.5 min/CV to 2.5 min/CV (e.g., about 2 min/CV). In some embodiments, a residence time of a solution in a column with a height of about 16 cm, a diameter of about 10 cm and volume of about 1.256 L to a 1.3 L is 3.5 min/CV to 4.5 min/CV (e.g., about 4 min/CV). In some embodiments, a residence time of a solution in a column with a height of about 20.5 cm, a diameter of 20 cm and a volume of about 6.4 L is about 3.6 min/CV. In some embodiments, a residence time of a solution in an about 6.4 L column is 3.5 min/CV to 4.5 min/CV (e.g., about 4 min/CV). In some embodiments, a residence time of a solution, including but not limited to an equilibration buffer, in a 6.0 L to 6.6 L (e.g., 6.4 L) column comprising an AEX stationary phase is 3.5 min/CV to 4.5 min/CV (e.g., about 4 min/CV).

One of ordinary skill will understand that linear velocity (also referred to herein as “linear flow velocity” or “velocity”) of a solution through a column is related, at least in part, to a volume and or dimension of the column and the stationary phase therein. In some embodiments, a linear velocity of a solution, including but not limited to an equilibration buffer, through a stationary phase in a column is 100 cm/hr to 1800 cm/hr, e.g., 100 cm/hr to 200 cm/hr, 200 cm/hr to 400 cm/hr, 400 cm/hr to 600 cm/hr, 600 cm/hr to 800 cm/hr, 800 cm/hr to 1000 cm/hr, 1000 cm/hr to 1500 cm/hr, or 1500 cm/hr to 1800 cm/hr. In some embodiments, a linear velocity of a solution through a stationary phase in a column is about 100 cm/hr, about 240 cm/hr, about 298 cm/hr, about 300 cm/hr, about 600 cm/hr, about 611 cm/hr or about 1790 cm/hr.

In some embodiments, a linear velocity of a solution through a stationary phase in a column that is about 5 cm high with a diameter of about 0.5 cm and a volume of about 1.0 mL is about 611 cm/hr. In some embodiments, a linear velocity of a solution through a stationary phase in a column that is about 15 cm high with a diameter of about 0.66 cm and a volume of about 5.1 mL is about 600 cm/hr. In some embodiments, a linear velocity of a solution through a stationary phase in a column that is about 15 cm high with a diameter of about 0.66 cm and a volume of about 5.1 mL is about 1790 cm/hr. In some embodiments, a linear velocity of a solution through a stationary phase in a column that is about 10 cm high with a diameter of about 2.5 cm and a volume of about 49 mL is about 298 cm/hr. In some embodiments, a linear velocity of a solution through a stationary phase in a column that is about 16 cm high with a diameter of about 10 cm and a volume of about 1256 mL is about 240 cm/hr. In some embodiments, a linear velocity of a solution through a stationary phase in a column that is about 20.5 cm high with a diameter of about 20 cm and a volume of about 6.4 L is 270 cm/hr to 330 cm/hr (e.g., 300 cm/hr). In some embodiments, a linear velocity of a solution, including but not limited to an equilibration buffer, through AEX stationary phase in a 6.0 L to 6.6 L (e.g., 6.4 L) column is about 270 cm/hr to 330 cm/hr (e.g., about 300 cm/hr).

In some embodiments, a flow rate (i.e., a volumetric flow rate) of a solution, including but not limited to an equilibration buffer, through a stationary phase in a column is 1.0 mL/min to 3.0 L/min, e.g., 1.0 mL/min to 10 mL/min, 10 mL/min to 100 mL/min, 100 mL/min to 500 mL/min, 500 mL/min to 1000 mL/min, 1 mL/min to 1.5 L/min, 1 mL/min to 2 L/min or 2 mL/min to 3 L/min. In some embodiments, a flow rate of a solution through a stationary phase in a column is about 1 mL/min, about 1.28 mL/min, about 1.67 mL/min, about 314 mL/min, about 1.57 L/min, about 1.8 L/min, about 2 L/min, about 3 L/min.

In some embodiments, a flow rate of a solution through a stationary phase in a column with a height of about 15 cm, a diameter of about 0.66 and volume of about 5.1 mL is about 1.28 mL/min. In some embodiments, a flow rate of solution through a stationary phase in a column with a height of about 19.5 cm, a diameter of about 0.66 and volume of about 6.67 mL is about 1.67 mL/min. In some embodiments, a flow rate of a solution through a stationary phase in a column with a height of about 16 cm, a diameter of 10 cm and a volume of about 1256 mL is about 314 mL/min. In some embodiments, a flow rate of a solution through a stationary phase in a column with a height of about 20.5 cm, a diameter of about 20 cm and a volume of about 6.4 L is about 1.8 L/min. In some embodiments, a flow rate of a solution, including but not limited to an equilibration buffer, through an AEX stationary phase in a 6.0 L to 6.6 L (e.g., 6.4 L) column is 1.5 mL/min to 2.0 L/min (e.g., about 1.8 L/min).

A method of preparing an AEX stationary phase for use in a method of purifying a rAAV (e.g., rAAV9, rAAV3B or others) vector from a solution (e.g., an affinity eluate) by AEX comprises equilibrating the AEX stationary phase in a column. In some embodiments, equilibrating precedes loading a solution comprising a rAAV vector to be purified onto a column. In some embodiments, equilibrating follows loading a solution comprising a rAAV vector to be purified onto a column.

In some embodiments, equilibrating comprises application of an equilibration buffer comprising 50 mM to 150 mM (e.g., about 100 mM) Tris, pH 8.5 to 9.5 to an AEX stationary phase in a column. In some embodiments, equilibrating comprises application of 4.5 CV to 5.5 CV (e.g., about 5 CV) of an equilibration buffer comprising 100 mM Tris, pH 9 to a 6.0 L to 6.6 L (e.g., 6.4 L) column comprising AEX stationary phase at a linear velocity of 270 cm/hr to 330 cm/hr (e.g., about 300 cm/hr), a flow rate of 1.5 L/min to 2.0 L/min (e.g., about 1.8 L/min) and/or a residence time of 3.5 min/CV to 4.5 min/CV (e.g., about 4 min/CV).

In some embodiments, equilibrating comprises application of an equilibration buffer comprising 400 mM to 600 mM sodium acetate, 50 mM to 150 mM Tris and 0.005% to 0.015% P188, pH 8.5 to 9.5 to an AEX stationary phase in a column. In some embodiments, equilibration comprises application of 4.5 CV to 5.5 CV (e.g., about 5 CV) of an equilibration buffer comprising 100 mM Tris, 500 mM sodium acetate, 0.01% P188, pH 8.9 to a column comprising an AEX stationary phase at a linear velocity of 270 cm/hr to 330 cm/hr (e.g., about 298 cm/hr, about 300 cm/hr), a flow rate of 1.5 L/min to 2.0 L/min (e.g., about 1.8 L/min) and/or a residence time of 1.5 min/CV to 4.5 min/CV (e.g., about 2 min/CV, about 4 min/CV). In some embodiments, a column is 6.0 L to 6.6 L (e.g., 6.4 L). In some embodiments, a column is 30 mL to 70 mL (e.g., about 49 mL, about 52 mL).

In some embodiments, equilibrating comprises application of an equilibration buffer comprising 100 mM to 300 mM histidine, 100 mM to 300 mM Tris, and 0.0% to 1.0% P188, pH 8.5 to 9.5 to an AEX stationary phase in a column. In some embodiments, equilibrating comprises application of ≥4.5 CV (e.g., about 5 CV) of an equilibration buffer comprising 200 mM histidine, 200 mM Tris, 0.5% P188, pH 8.8 to a column comprising an AEX stationary phase at a linear velocity of 270 cm/hr to 330 cm/hr (e.g., about 298 cm/hr, about 300 cm/hr), a flow rate of 1.5 L/min to 2.0 L/min (1.8 L/min) and/or a residence time of 1.5 min/CV to 4.5 min/CV (e.g., about 2 min/CV, about 4 min/CV). In some embodiments, a column is 6.0 L to 6.6 L (e.g., 6.4 L). In some embodiments, a column is 30 mL to 70 mL (e.g., about 49 mL, about 52 mL).

In some embodiments, equilibration comprises application of an equilibration buffer comprising 50 mM to 150 mM Tris and 0.005% to 0.015% P188, pH 8.5 to 9.5 to an AEX stationary phase in a column. In some embodiments, equilibration comprises application of 4.5 CV to 5.5 CV (e.g., about 5 CV) of an equilibration buffer comprising 100 mM Tris, 0.01% P188, pH 8.9 to a column comprising an AEX stationary phase at a linear velocity of 270 cm/hr to 330 cm/hr (e.g., about 298 cm/hr, about 300 cm/hr), a flow rate of 1.5 L/min to 2.0 L/min (1.8 L/min) and/or a residence time of 1.5 min/CV to 4.5 min/CV (e.g., 2 min/CV, 4 min/CV). In some embodiments, a column is 6.0 L to 6.6 L (e.g., 6.4 L). In some embodiments, a column is 30 mL to 70 mL (e.g., about 49 mL, about 52 mL).

In some embodiments, the present disclosure provides a method preparing an AEX stationary phase for use in a method of purifying a rAAV (e.g., rAAV9, rAAV3B or others) vector by AEX, the method comprising a step of: i) pre-use flushing comprising application of ≥4.5 CV (e.g., about 5 CV) of water for injection to the AEX stationary phase in a column; ii) sanitizing comprising application of about 5 CV to 10 CV (e.g., about 8 CV) or about 14.4 to 17.6 CV (e.g., about 16 CV) of a solution comprising 0.1 M to 1.0 M (e.g., about 0.5 M) NaOH to the AEX stationary phase in the column, optionally, by upward flow; iii) regenerating comprising application of 4.5 CV to 5.5 CV (e.g., about 5 CV) of a solution comprising 1 M to 3 M (e.g., about 2 M) NaCl, 50 mM to 150 mM (e.g., about 100 mM Tris), pH 8.5 to 9.5 (e.g., about 9) to the AEX stationary phase in the column; iv) equilibration comprising application of 4.5 CV to 5.5 CV (e.g., about 5 CV) of a solution comprising 50 mM to 150 mM (e.g., about 100 mM) Tris, pH 8.5 to 9.5 (e.g., about 9) to the AEX stationary phase in the column; v) equilibration comprising application of 4.5 CV to 5.5 CV (e.g., about 5 CV) of an equilibration buffer comprising 50 mM to 150 mM (e.g., about 100 mM) Tris, 400 mM to 600 mM (e.g., about 500 mM) sodium acetate, 0.005% to 0.015% (e.g., about 0.01%) P188, pH 8.5 to 9.5 (e.g., about 8.9) to the AEX stationary phase in the column; vi) equilibration comprising application of ≥4.5 CV (e.g., about 5 CV) of an equilibration buffer comprising 100 mM to 300 mM (e.g., about 200 mM) histidine, 100 mM to 300 mM (e.g., about 200 mM) Tris, 0.1% to 1.0% (e.g., about 0.5%) P188, pH 8.5 to 9.5 (e.g., 8.8) to the AEX stationary phase in the column; and/or vii) equilibration comprising application of 4.5 CV to 5.5 CV (e.g., about 5 CV) of an equilibration buffer comprising 50 mM to 150 mM (e.g., about 100 mM) Tris, 0.005% to 0.015% (e.g., about 0.01%) P188, pH 8.5 to 9.5 (e.g., 8.9) to the AEX stationary phase in the column; optionally wherein at least one of steps i)-vii) is performed at a linear velocity of 270 cm/hr to 330 cm/hr (e.g., about 298 cm/hr, about 300 cm/hr), and/or a residence time of 1.5 min/CV to 4.5 min/CV (e.g., about 2 min/CV, about 4 min/CV); optionally wherein the AEX stationary phase is POROS™ 50 HQ; optionally wherein the rAAV vector is a rAAV9 vector or a rAAV3B vector, and optionally wherein a step (e.g., a load step) may occur between any step of equilibration. In some embodiments, at least one of steps i)-vii) is performed at a flow rate of 1.5 L/min to 2.0 L/min (e.g., about 1.8 L/min) through a 6 L to 6.6 L column (e.g., about 6.4 L), or about 314 mL/min through a 1.3 L column. One of ordinary skill will understand that the order of the above steps may be varied.

Dilution and Filtration

A method of purifying a rAAV (e.g., rAAV9, rAAV3B or others) vector from a solution (e.g., an affinity eluate) by AEX comprises preparation of the solution by diluting, and optionally filtering, the solution. A solution comprising a rAAV vector to be purified may be an affinity eluate, a supernatant from a cell lysate and/or a post-harvest solution having undergone at least one purification or processing step. A solution comprising a rAAV vector to be purified may be diluted, and optionally filtered prior to loading onto an AEX column in order to make the solution compatible with processing through the AEX column. In some embodiments, diluting, and optionally filtering, a solution comprising a rAAV vector to be purified results in a change in pH and/or conductivity of the solution. In some embodiments, a solution comprising a rAAV vector to be purified is an eluate resulting from affinity chromatography purification of a rAAV vector produced in a 1 L to 2000 L (or greater) single use bioreactor (SUB).

A method of preparing a solution comprising a rAAV vector for purification by AEX comprises i) diluting an affinity eluate, and optionally ii) filtering the affinity eluate from step i) to produce the diluted affinity eluate (also referred to herein as a “diluted affinity pool,” “load,” or “AEX load”). In some embodiments, pH of an affinity eluate after dilution, and optional filtration is increased as compared to pH of the affinity eluate before the dilution. In some embodiments, conductivity of an affinity eluate after dilution, and optional filtration is decreased as compared to conductivity of the affinity eluate before the dilution. In some embodiments, the diluted, and optionally filtered affinity eluate is loaded on an AEX stationary phase.

In some embodiments, an affinity eluate is generated from affinity purification of a rAAV vector produced in a vessel (e.g., a single use bioreactor (SUB)) with a volume of 1 mL to 2000 L, or greater than 2000 L. In some embodiments, an affinity eluate is generated from affinity chromatography purification of a rAAV vector produced in a vessel (e.g., a SUB) with a volume of about 1 mL, about 10 mL, about 50 mL, about 100 mL, about 250 mL, about 500 mL, about 750 mL, about 1 L, about 50 L, about 100 L, about 250 L, about 500 L, about 1000 L, about 2000 L or greater. In some embodiments, an affinity eluate is generated from affinity purification of a rAAV vector produced in a vessel (e.g., a SUB) with a volume of 1 mL to 100 mL, 100 mL to 500 mL, 500 mL to 750 mL, 750 mL, to 1 L, 1 L to 10 L, 10 L to 50 L, 50 L to 100 L, 100 L to 250 L, 250 L to 500 L, 500 L to 750 L, 750 L to 1000 L, 1000 L to 1500 L, 1500 L to 2000 L, 2000 L to 3500 L, 3500 L to 4000 L or 4500 L to 5000 L. In some embodiments, an affinity eluate is generated from affinity purification of a rAAV vector produced in a vessel (e.g., a SUB) with a volume of 1 mL to 5000 L, 100 mL to 5000 L, 100 mL to 4000 L, 100 mL to 2000 L, 100 mL to 1000 L, 1 L to 5000 L, 1 L to 4000 L, 1L to 2000 L, 1 L to 1000 L, 500 mL to 5000 L, 500 mL to 2000 L or 500 mL to 1000 L.

In some embodiments, diluting a solution comprising a rAAV vector to be purified (e.g., an affinity eluate) comprises diluting the solution about 2 to 25-fold or about 5 to 20-fold, or about 10 to 20-fold (e.g., about 5-fold, about, 6-fold, about 7-fold, about 8-fold, about 9-fold, about 10-fold, about 11-fold, about 12-fold, about 13-fold, about 14-fold, about 15-fold, about 20-fold, about 25-fold) to produce a diluted affinity eluate. In some embodiments, diluting a solution comprising a rAAV vector to be purified (e.g., an affinity eluate) comprises diluting the solution about 2-fold. In some embodiments, diluting a solution comprising a rAAV vector to be purified (e.g., an affinity eluate) comprises diluting the solution about 15-fold.

In some embodiments, diluting a solution comprising a rAAV vector to be purified (e.g., an affinity eluate) is performed “in-line” with the column, and wherein a dilution solution (diluent) is delivered through a first tubing to a Y-connector, and the solution comprising a rAAV vector to be purified is delivered through a second tubing to the Y-connector, and optionally wherein a static mixer is contained within a third tubing located after the Y-connector.

In some embodiments, diluting a solution comprising a rAAV vector to be purified (e.g., an affinity eluate) is performed “in-line” and directed into a holding vessel (e.g., a break tank). For example, a dilution solution (diluent) is delivered through a first tubing to a Y-connector, and a solution comprising a rAAV vector to be purified is delivered through a second tubing to the Y-connector, wherein the end of the Y-connector is connected to a holding vessel which is optionally, connected to a chromatography column (e.g., an AEX column).

In some embodiments, diluting comprises delivery of a dilution solution through a first tubing to a Y-connector at a flow rate of 1 to 5 mL/min (e.g., about 3.5 mL/min) and delivery of a solution comprising a rAAV vector to be purified (e.g., an affinity eluate) through a second tubing at a flow rate of 0.1 to 2 mL/min (e.g., about 0.25 mL/min).

In some embodiments, diluting comprises delivery of a dilution solution through a first tubing to a Y-connector at a flow rate of about 3.5 mL/min and delivery of an affinity eluate through a second tubing at a flow rate of about 0.25 mL/min, such that the affinity eluate is diluted about 15-fold.

In some embodiments, diluting comprises dilution of a solution comprising a rAAV vector to be purified (e.g., an affinity eluate) with a dilution solution comprising a buffering agent (Tris, BIS-Tris propane, diethanolamine, diethylamine, tricine, triethanolamine and/or bicine). In some embodiments, a solution comprising a rAAV vector to be purified (e.g., an affinity eluate) is diluted with a dilution solution comprising 10 mM to 500 mM buffering agent (e.g., Tris). In some embodiments, a dilution solution comprises about 10 mM to about 450 mM, about 10 mM to about 400 mM, about 10 mM to about 350 mM, about 10 mM to about 300 mM, about 50 mM to about 450 mM, about 50 mM to about 400 mM, about 50 mM, about 350 mM, about 50 mM to about 300 mM, about 100 mM to about 450 mM, about 100 mM to about 400 mM, about 100 mM to about 350 mM, about 100 mM to about 300 mM, or about 150 mM to about 250 mM Tris. In some embodiments, a dilution solution comprises about 200 mM Tris.

In some embodiments, a dilution solution comprises an amino acid, e.g., histidine, arginine, glycine or citrulline. In some embodiments, a dilution solution comprises about 50 mM, about 75 mM, about 100 mM, about 125 mM, about 150 mM, about 175 mM, about 200 mM, about 225 mM, about 250 mM, about 275 mM, about 300 mM, about 350 mM, about 400 mM, about 450 mM, about 500 mM, about 550 mM or about 600 mM of an amino acid (e.g., histidine, arginine, glycine or citrulline).

In some embodiments, dilution solution comprises an amino acid, e.g., histidine or arginine. In some embodiments, an dilution solution comprises 10 mM to 600 mM of an amino acid (e.g., histidine arginine, glycine or citrulline). In some embodiments, an equilibration buffer comprises about 10 mM to about 600 mM, about 10 mM to about 550 mM, about 10 mM to about 500 mM, about 10 mM to about 450 mM about 10 mM to about 400 mM, about 10 mM to about 350 mM, about 10 mM to about 300 mM, about 50 mM to about 600 mM, about 50 mM to about 550 mM, about 50 mM to about 500 mM, about 50 mM to about 450 mM, about 50 mM to about 400 mM, about 50 mM to about 350 mM, about 50 mM, to about 300 mM, about 100 mM to about 600 mM, about 100 mM to about 500 mM, about 100 mM to about 400 mM, about 100 mM to about 300 mM, or about 150 mM to about 250 mM of an amino acid (e.g., histidine). In some embodiments, a dilution solution comprises about 200 mM histidine.

In some embodiments, a dilution solution comprises a detergent, e.g., P188, Triton X-100, Polysorbate 80, Brij-35 or NP-40. In some embodiments, dilution solution comprises 0.005% to 1.5% detergent (e.g., P188). In some embodiments, a dilution solution comprises 0.1% to 1.0% detergent (e.g., P188). In some embodiments, a dilution solution comprises about 0.01% to about 1.5%, about 0.01% to about 1.0%. about 0.01% to about 0.75%, about 0.05% to about 1.5%, about 0.05% to about 1.0%, about 0.05% to about 0.75%, about 0.1% to about 1.5%, about 0.1% to about 1.0%, about 0.1% to about 0.75%, or about 0.25% to about 0.75% detergent (e.g., P188). In some embodiments, a dilution solution comprises about 0.5% P188.

In some embodiments, a dilution solution has a pH of 8 to 10. In some embodiments, a dilution solution has a pH of 8.5 to 9.5. In some embodiments, a dilution solution has a pH of 8.7 to 9.0. In some embodiments, a dilution solution has a pH of about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5 or about 10.0. In some embodiments, a dilution solution has a pH of about 8.8. In some embodiments, a dilution solution has a pH of about 8.9. In some embodiments, a dilution solution has a pH of about 9.0.

In some embodiments, diluting comprises dilution of a solution comprising a rAAV vector to be purified (e.g., an affinity eluate) with a buffer selected from the group consisting of 20 mM Tris, pH 9; 1 M Tris Base, pH 11; 100 mM Tris, pH 9; 100 mM Tris, 0.01% P188, pH 9; 100 mM Tris, 0.1% P188, pH 9; 100 mM Tris, 1.0% P188, pH 9; 1 M Tris, pH 9; 150 mM acetate, 100 mM glycine, 25 mM MgCl₂, pH 4.2; 5 mM Arginine, 2 mM MgCl₂, 0.1% P188, 100 mM Tris, pH 8.9; 50 mM arginine, 2 mM MgCl₂, 0.1% P188, 100 mM Tris, pH 9; 500 mM Arginine, 2 mM MgCl₂. 0.1% P188, 400 mM Tris, pH 9.1; 200 mM Glycine, 5 mM MgCl₂, 200 mM Tris, pH 8.9; 200 mM Histidine. 200 mM Tris, pH 8.9; 200 mM Histidine, 200 mM Tris, 5 mM MgCl₂, pH 8.9; 200 mM Histidine, 200 mM Tris, 5 mM MgCl₂, 5% Glycerol, pH 8.9; 200 mM Histidine, 250 mM Tris, 10 mM MgCl₂, 25% Glycerol, pH 8.9; 200 mM Histidine, 200 mM Tris, 5 mM MgCl₂, 5% Iodixanol pH 8.8; 200 mM Histidine, 200 mM Tris, 10 mM MgCl₂, 25% Iodixanol, pH 8.8; 200 mM Histidine, 200 mM Tris, 0.5% Triton X-100, pH 8.9; 200 mM histidine, 200 mM Tris, 0.5% P188, pH 8.8; and a combination thereof.

In some embodiments, diluting comprises dilution of a solution comprising a rAAV vector to be purified (e.g., an affinity eluate) with a buffer comprising about 20 mM Tris, pH 9, about 1 M Tris base, pH 11, or both. In some embodiments, diluting comprises dilution of a solution comprising a rAAV vector to be purified (e.g., an affinity eluate) 7 to 8 fold (e.g., about 7.1 fold) with a buffer comprising about 20 mM Tris, pH 9, about 1 M Tris Base, pH 11, or both.

In some embodiments, diluting comprises dilution of a solution comprising a rAAV vector to be purified (e.g., an affinity eluate) with a buffer comprising 100 mM to 300 mM (e.g., about 200 mM) histidine, 100 mM to 300 mM (e.g., about 200 mM) Tris, 0.1% to 1.0% (e.g., 0.5%) P188, pH 8.7 to 9.0. In some embodiments, diluting comprises dilution of a solution comprising a rAAV vector to be purified (e.g., an affinity eluate) 10 to 20 fold by weight (e.g., about 15 fold) with a buffer comprising about 200 mM histidine, 200 mM Tris, 0.5% P188, pH 8.7 to 9.0 (e.g., about 8.8). In some embodiments, diluting comprises dilution of an affinity eluate comprising a rAAV vector to be purified 14.4 to 15.5 fold by weight (e.g., about 15 fold) with a buffer comprising about 200 mM histidine, 200 mM Tris, 0.5% P188, pH 8.7 to 9.0 (e.g., about pH 8.8), and thereby forming a diluted affinity eluate.

In some embodiments, prior to dilution of a solution comprising a rAAV vector to be purified (e.g., an affinity eluate) the solution is spiked with 20 mM MgCl₂ so that the concentration of MgCl₂ in the diluted solution is about 1.7 mM. In some embodiments, MgCl₂ stabilizes the rAAV vectors in a solution.

In some embodiments, filtering comprises filtration of a solution comprising a rAAV vector to be purified (e.g., an affinity eluate, a diluted affinity eluate) prior to loading the solution onto an AEX column. In some embodiments, prior filtering, a filter is pre-wet with water for injection and/or a dilution solution. In some embodiments, filtering comprises filtration of a solution comprising a rAAV vector to be purified (e.g., an affinity eluate, a diluted affinity eluate) through a filter which collects aggregates, such as nucleic acid or protein aggregates, or other high molecular mass species, but allows AAV capsids to flow through. In some embodiments, a filter is an 0.1 μm to 0.45 μm filter (e.g., a 0.2 μm polyethersulfone (PES) filter or a 0.45 μm PES filter). In some embodiments, filtering comprises filtration of a diluted affinity eluate comprising a rAAV vector to be purified through an 0.2 μm filter prior to loading onto an AEX column. A filter used to filter a solution comprising a rAAV vector to be purified (e.g., an affinity eluate, a diluted affinity eluate) may be separate from the column, or may be in-line with the column or chromatography apparatus (also referred to as a chromatography skid).

In some embodiments, filtering comprises filtration of a diluted affinity eluate comprising a rAAV vector to be purified through an in-line 0.2 μm filter before loading the eluate onto an AEX column.

In some embodiments, pH of a solution comprising a rAAV vector to be purified (e.g., an affinity eluate) is 3.0 to 4.4 prior to diluting, and optionally filtering, and pH of the solution comprising a rAAV vector to be purified (e.g., an affinity eluate) after diluting, and optionally filtering, is 8.5 to 9.5, 8.7 to 9.0 or ≥8.6 (e.g., about pH 8.8, pH 9.0).

In some embodiments, conductivity of a solution comprising a rAAV vector to be purified (e.g., an affinity eluate) is 5.0 mS/cm to 7.0 mS/cm (e.g., about 5.5 mS/cm to 6.5 mS/cm) prior to diluting, and optionally filtering, and conductivity of the solution comprising a rAAV vector to be purified (e.g., an affinity eluate) after diluting, and optionally filtering, is 1.7 mS/cm to 3.5 mS/cm, 1.8 mS/cm to 2.8 mS/cm, 2.2 mS/cm to 2.6 mS/cm or ≤2.5 mS/cm. In some embodiments, conductivity of an affinity eluate after diluting, and optionally filtering, is about 1.8 mS/cm to about 2.8 mS/cm. In some embodiments, conductivity of an affinity eluate following dilution, and optionally filtering, is about 2.3+/−0.5 mS/cm.

As used herein, the term “percent VG dilution yield” or “% VG dilution yield” refers to the amount of VG present in a diluted affinity pool (also referred to herein as a diluted affinity eluate) as a percentage of the amount of VG present in the affinity pool (also referred to herein as an affinity eluate) prior to dilution. For instance, % VG dilution yield=((amount of VG in diluted affinity pool)/(amount of VG in affinity pool))*100.

In some embodiments, a percentage of VG recovered in a diluted, and optionally filtered solution (% VG dilution yield) comprising a rAAV vector to be purified (e.g., an affinity eluate) is 60% to 100% of the VG present in a solution (e.g., an affinity eluate) prior to diluting, and optionally filtering. In some embodiments, a % VG yield of a diluted, and optionally filtered solution comprising a rAAV vector to be purified (e.g., an affinity eluate) is 60% to 70%, 70% to 80%, 80% to 90%, 90% to 100% of the VG present in a solution (e.g., an affinity eluate) prior to diluting, and optionally filtering. In some embodiments, a % VG yield of a diluted, and optionally filtered solution comprising a rAAV vector to be purified (e.g., an affinity eluate) is about 60%, about 65%, about 70%, about 75%, about 80%, about 85%, about 90%, about 95%, about 98%, about 99% or about 100% of the VG present in a solution prior to diluting, and optionally filtering.

In some embodiments of diluting an affinity eluate according to methods disclosed herein results in % VG dilution yield of 88%+/−36%. In some embodiments of diluting an affinity eluate according to methods disclosed herein results in % VG dilution yield of 120%+/−12%. Diluting an affinity eluate resulting from affinity chromatography purification of a rAAV vector produced in a 250 L SUB results in a % VG dilution yield of 35% to 100% (e.g., 41% to 92%). Diluting an affinity eluate resulting from affinity chromatography purification of a rAAV vector produced in a 2000 L SUB results in a % VG dilution yield of 70% to >100% (e.g., 88% to 154%).

In some embodiments, a Z-average (given in units of nm, and determined by dynamic light scattering (DLS) of a diluted and, optionally filtered solution comprising a rAAV vector to be purified (e.g., an affinity eluate) is measured. A Z-average measures the level of aggregation of rAAV capsids present in a solution. In some embodiments, a Z-average of a diluted and, optionally filtered solution comprising a rAAV vector to be purified is about 15 nm to 40 nm, 15 nm to 20 nm, 20 nm to 30 nm or 30 nm to 40 nm. In some embodiments, a Z-average of a diluted and, optionally filtered solution comprising a rAAV vector to be purified is about 15 nm, about 16 nm, about 17 nm, about 18 nm, about 19 nm, about 20 nm, 21 nm, about 22 nm, about 23 nm, about 24 nm, about 25 nm, about 26 nm, about 27 nm, about 28 nm, about 29 nm, about 30 nm, about 35 nm or about 40 nm.

A method of purifying a rAAV (e.g., rAAV9, rAAV3B or others) vector from a solution (e.g., an affinity eluate) by AEX comprises diluting the solution by 14 to 16 fold (e.g., about 15 fold) with a buffer comprising 100 mM to 300 mM (e.g., about 200 mM) histidine, 100 mM to 300 mM (e.g., about 200 mM) Tris, 0.1% to 1.0% (e.g., about 0.5%) P188, pH 8.7 to 9.0 (e.g., about pH 8.8); and optionally comprises filtering comprising filtration of the diluted solution through a 0.1 μm to 0.45 μm (e.g., about 0.2 μm) filter, and wherein the diluted, and optionally filtered solution has a pH of about 8.6 to 9.0 (e.g., about pH 8.9) and a conductivity of 1.8 mS/cm to 2.8 mS/cm.

A method of preparing an affinity eluate comprising a rAAV vector for purification by AEX chromatography, as disclosed herein, comprises i) diluting the affinity eluate 2 to 25-fold (e.g., about 15-fold) with a buffer comprising 200 mM histidine, 200 mM Tris, 0.5% P188, pH 8.8; and ii) optionally filtering the affinity eluate from step i) through a 0.2 μm filter to produce the diluted affinity eluate; wherein the pH of the diluted affinity eluate is increased as compared to the pH of the affinity eluate; wherein the conductivity of the diluted affinity eluate is decreased as compared to the conductivity of the affinity eluate; optionally wherein the rAAV vector is an AAV9 vector or an AAV3B vector; and optionally wherein the affinity eluate is produced by affinity purification of a rAAV vector produced in a vessel (e.g., a SUB) with a volume of 250 L or 2000 L.

Load

A method of purifying a rAAV (e.g., rAAV9, rAAV3B or others) vector by AEX disclosed herein comprises loading a solution comprising a substance to be purified (e.g., a rAAV vector) onto an AEX stationary phase in a column. Loading may be performed by gravity feeding the load onto the column or pumping the load onto the chromatography column. In some embodiments, a solution comprising a rAAV vector to be purified by AEX is selected from the group consisting of an affinity eluate, a supernatant from a cell lysate and a post-harvest solution, each having undergone at least one other purification or processing step (e.g., cell lysis, flocculation, filtration, dilution, pH adjustment, chromatography). A solution comprising a rAAV vector to be purified may be diluted, filtered and/or pH adjusted prior to loading the solution onto an AEX column in order to make the solution compatible with processing through the AEX column. In some embodiments, a solution comprising a rAAV vector to be purified is an eluate resulting from affinity chromatography purification of a rAAV vector produced in a 100 L to 500 L (e.g., about 250 L), 1000 L to 3000 L (e.g., about 2000 L) or larger vessel (e.g., a single use bioreactor (SUB)), and wherein the eluate has been diluted and filtered.

In some embodiments, loading comprises application of a diluted, and optionally filtered solution (e.g., an affinity eluate) comprising about 2.0×10¹² vector genomes (VG)/mL to 2.0×10¹⁵ VG/mL, e.g., 2.0×10¹² VG/mL to 2.0×10¹³ VG/mL, 2.0×10¹³ VG/mL to 2.0×10¹⁴ VG/mL, 1.0×10¹⁴ VG/mL to 3.0×10¹⁴ VG/mL, 2.0×10¹⁴ VG/mL to 2.0×10¹⁵ VG/mL, or more of column volume (also referred to as a “column challenge VG/mL resin”) onto an AEX column, as measured by qPCR analysis of a sequence within the vector genome. In some embodiments, loading comprises application of a diluted solution (e.g., an affinity eluate) comprising 6.3×10¹³ to 9.4×10¹³ VG/mL of column volume onto an about 30 mL to 70 mL AEX column as measured by qPCR analysis of a transgene sequence within the vector genome (e.g., wherein the transgene is an ATP7B transgene). In some embodiments, loading comprises application of a diluted and, optionally filtered solution (e.g., an affinity eluate) comprising 5×10¹³ to 1.3×10¹⁴ VG/mL of column volume onto an about 1.3 L AEX column as measured by qPCR analysis of ITR sequences within the vector genome. In some embodiments, loading comprises application of a diluted and, optionally filtered solution (e.g., an affinity eluate) comprising 2.6×10¹² to 6.8×10¹³ VG/mL of column volume onto an about 6.4 L AEX column as measured by qPCR analysis of a transgene sequence within the vector genome.

In some embodiments, loading comprises application of a diluted and, optionally filtered solution (e.g., an affinity eluate) comprising 2.5×10¹⁵ VG/L to 2.5×10¹⁶ VG/L, 2.5×10¹⁶ VG/L to 2.5×10¹⁷ VG/L, 2.5×10¹⁵ VG/L to 3.0×10¹⁷ VG/L or more of column volume onto an AEX column.

In some embodiments, loading comprises application of a diluted and, optionally filtered solution (e.g., an affinity eluate) comprising 8.0×10¹² total VG to 2.0×10¹⁸ total VG, e.g., 8.0×10¹² total VG to 8.0×10¹³ total VG, 8.0×10¹³ to 8.0×10¹⁴ total VG, 8.0×10¹⁴ total VG to 8.0×10¹⁵ total VG, 8.0×10¹⁵ total VG to 8.0×10¹⁶ total VG, 8.0×10¹⁶ total VG to 8.0×10¹⁷ total VG, 8.0×10¹⁷ total VG to 2.0×10¹⁸ total VG, or more onto an AEX column. In one embodiment, loading comprises application of a diluted and, optionally filtered solution (e.g., an affinity eluate) comprising ≤15×10¹⁶ VG/L of column volume onto an AEX column, and optionally wherein the VG are measured by quantitative polymerase chain reaction (qPCR) analysis of the transgene.

When a solution comprising a rAAV vector to be purified (e.g., an affinity eluate) is loaded onto a column, the solution flows through the column stationary phase at a particular rate (e.g., cm/hr, mL/min) and is in contact with the stationary phase for a particular period of time (i.e., residence time).

In some embodiments, a residence time of a solution comprising a rAAV vector loaded onto a column is 0.1 min/CV to 5 min/CV, e.g., 0.1 min/CV to 1.0 min/CV, 1.0 min/CV to 2 min/CV, 2 min/CV to 3 min/CV, 3 min/CV to 4 min/CV, 4 min/CV to 5 min/CV or more. In some embodiments, a residence time of a solution comprising a rAAV vector loaded onto a column is about 0.5 min/CV. In some embodiments, a residence time of a solution comprising a rAAV vector loaded onto a column is about 1.5 min/CV. In some embodiments, a residence time of a solution comprising a rAAV vector loaded onto a column is about 2.0 min/CV. In some embodiments, a residence time of a solution comprising a rAAV vector loaded onto a column is 3.5 min/CV to 4.5 min/CV. In some embodiments, a residence time of a diluted and/or filtered affinity eluate comprising a rAAV vector loaded on a 6.0 L to 6.6 L (e.g., about 6.4 L) AEX column is 3.0 min/CV to 5.0 min/CV (e.g., about 4 min/CV).

In some embodiments, a linear velocity of a solution comprising a rAAV vector loaded onto a column is 100 cm/hr to 1800 cm/hr, e.g., 100 cm/hr to 200 cm/hr, 200 cm/hr to 400 cm/hr, 400 cm/hr to 600 cm/hr, 600 cm/hr to 800 cm/hr, 800 cm/hr to 1000 cm/hr, 1000 cm/hr to 1500 cm/hr, 1500 cm/hr to 1800 cm/hr. In some embodiments, a linear velocity of a solution comprising a rAAV vector loaded onto a column is 270 cm/hr to 330 cm/hr (e.g. about 298 cm/hr, about 300 cm/hr). In some embodiments, a linear velocity of a solution comprising a rAAV vector loaded onto a column is about 300 cm/hr, about 600 cm/hr, about 611 cm/hr or about 1790 cm/hr. In some embodiments, a linear velocity of a diluted, and optionally filtered affinity eluate comprising a rAAV vector loaded on a 6.0 L to 6.6 L (e.g., about 6.4 L) AEX column is 270 cm/hr to 330 cm/hr (e.g., about 300 cm/hr).

In some embodiments, a flow rate of a solution comprising a rAAV vector loaded onto a column is 1.0 mL/min to 3.0 L/min, e.g., 1.0 mL/min to 10 mL/min, 10 mL/min to 100 mL/min, 100 mL/min to 500 mL/min, 500 mL/min to 1000 mL/min, 1 mL/min to 1.5 L/min, 1 mL/min to 2 L/min, 2 mL/min to 3 L/min. In some embodiments, a flow rate of a solution comprising a rAAV vector loaded onto a column is about 1.28 mL/min. In some embodiments, a flow rate of a solution comprising a rAAV vector loaded onto a column is about 314 mL/min. In some embodiments, a flow rate of a solution comprising a rAAV vector through stationary phase in a column is 1.5 L/min to 2.0 L/min. In some embodiments, a flow rate of a solution comprising a rAAV vector loaded onto a column is about 1.8 L/min. In some embodiments, a flow rate of a diluted and/or filtered affinity eluate comprising a rAAV vector loaded on a 6.0 L to 6.6 L (e.g., about 6.4 L) column is 1.5 L/min to 2.0 L/min (e.g., about 1.8 L/min).

In some embodiments, a method of purifying a rAAV vector (e.g., rAAV9, rAAV3B or others) from an affinity eluate comprises i) diluting the affinity eluate with a buffer comprising a detergent (e.g., P188), an amino acid (e.g., histidine) and a buffer (e.g., Tris); ii) optionally filtering the diluted affinity eluate; and iii) loading the diluted, and optionally filtered affinity eluate onto a column comprising an AEX stationary phase wherein the AEX stationary phase has been flushed, sanitized, rinsed and/or equilibrated prior to loading, and optionally wherein the AEX stationary phase is POROS™ 50 HQ.

In some embodiments, a method of purifying a rAAV vector (e.g., rAAV9, rAAV3B or others) from an affinity eluate comprises i) diluting the affinity eluate 14.4 to 15.5 fold (e.g., about 15 fold) with a buffer comprising about 100 mM to 300 mM (e.g., about 200 mM) histidine, 100 mM to 300 mM (e.g., about 200 mM) Tris, 1.0% to 1.5% (e.g., about 0.5%) P188, pH 8.7 to 9.0; ii) optionally filtering the diluted affinity eluate through an in-line 0.1 to 0.45 μm (e.g., about 0.2 μm) filter; and iii) loading the diluted and filtered affinity eluate onto a column comprising an AEX stationary phase; optionally wherein at least one step is performed at a linear velocity of 270 cm/hr to 330 cm/hr (e.g., about 298 cm/hr, about 300 cm/hr), a flow rate of 1.5 L/min to 2.0 L/min (e.g., about 1.8 L/min) through column and/or a residence time of 1.5 min/CV to 4.5 min/CV (e.g., about 2 min/CV, about 4 min/CV) and, optionally wherein the AEX stationary phase is POROS™ 50 HQ. In some embodiments, a column is a 6.0 L to 6.6 L (e.g., about 6.4 L) column,

In some embodiments, the present disclosure provides a method of purifying a rAAV (e.g., rAAV9, rAAV3B or others) vector by AEX, the method comprising a step of: i) pre-use flushing comprising application of ≥4.5 CV (e.g., about 5 CV) of water for injection to an AEX stationary phase in a column; ii) sanitizing comprising application of 5 CV to 10 CV (e.g., about 8 CV) or 14.4 to 17.6 CV (e.g., about 16 CV) of a solution comprising 0.1 M to 1.0 M (e.g., about 0.5 M) NaOH to the AEX stationary phase in the column, optionally by upward flow; iii) regenerating comprising application of 4.5 CV to 5.5 CV (e.g., about 5 CV) of a solution comprising 1 M to 3 M (e.g., about 2 M) NaCl), 50 mM to 150 mM (e.g., about 100 mM) Tris, pH 8.5 to 9.5 (e.g., about 9) to the AEX stationary phase in the column; iv) equilibration comprising application of 4.5 CV to 5.5 CV (e.g., about 5 CV) of a solution comprising 50 mM to 150 mM (e.g., about 100 mM) Tris, pH 8.5 to 9.5 (e.g., about 9) to the AEX stationary phase in the column; v) equilibration comprising application of 4.5 CV to 5.5 CV (e.g., about 5 CV) of an equilibration buffer comprising 50 mM to 150 mM (e.g., about 100 mM) Tris, 400 mM to 600 mM (e.g., about 500 mM) sodium acetate, 0.005% to 0.015% (e.g., about 0.01%) P188, pH 8.5 to 9.0 (e.g., about 8.9) to the AEX stationary phase in the column; vi) equilibration comprising application of ≥4.5 CV (e.g., about 5 CV) of an equilibration buffer comprising 100 mM to 300 mM (e.g., about 200 mM) histidine, 100 mM to 300 mM (e.g., about 200 mM) Tris, 0.1% to 1.0% (e.g., about 0.5%) P188, pH 8.5 to 95. (e.g., about 8.8) to the AEX stationary phase in the column; vii) loading the affinity eluate to an AEX stationary phase in the column, optionally wherein the eluate has been a) diluted about 14.4 to 15.5 fold (e.g., about 15 fold) with a buffer comprising 100 mM to 300 mM (e.g., about 200 mM) histidine, 100 mM to 300 mM (e.g., about 200 mM) Tris, 0.1% to 1.0% (e.g., about 0.5%) P188, pH 8.7 to 9.0, and optionally b) filtered through an in-line 0.1 μm to 0.45 μm (e.g., about 0.2 μm) filter prior to application to the stationary phase; and/or viii) equilibration comprising application of 4.5 CV to 5.5 CV (e.g., about 5 CV) of an equilibration buffer comprising 50 mM to 150 mM (e.g., about 100 mM) Tris, 0.005% to 0.015% (e.g., about 0.01%) P188, pH 8.5 to 9.5 (e.g., about 8.9) to the AEX stationary phase in the column; optionally wherein at least one of steps i)-viii) is performed at a linear velocity of 270 cm/hr to 330 cm/hr (e.g., about 298 cm/hr, about 300 cm/hr), and/or a residence time of 1.5 min/CV to 4.5 min/CV (e.g., about 2 min/CV, about 4 min/CV); optionally wherein the rAAV vector is a rAAV9 or rAAV3B vector; and optionally wherein the AEX stationary phase is POROS™ 50 HQ. In some embodiments, at least one of steps i)-vii) is performed at a flow rate of 1.5 L/min to 2.0 L/min (e.g., about 1.8 L/min) through a 6 L to 6.6 L column (e.g., about 6.4 L), or about 314 mL/min through a 1.3 L column. One of ordinary skill will understand that the order of the above steps may be varied.

Load Chase

A method of purifying a rAAV vector (e.g., rAAV9, rAAV3B or others) from a solution (e.g., an affinity eluate) comprises application of a load chase solution to a column stationary phase following application of the solution comprising the rAAV vector. A load chase serves to complete application of the load or load solution and to remove unbound material from the column. In some embodiments, a load chase serves to remove unbound material from the column. In some embodiments, a load chase solution comprises 5 mM to 50 mM (e.g., about 20 mM) Tris, pH 8.5 to 9.5 (e.g., about 9). In some embodiments, 9 to 11 CV (e.g., about 10 CV) of a load chase solution is applied to the column stationary phase. In some embodiments, a load chase solution is applied to the column stationary phase at velocity of 200 cm/hr to 2000 cm/hr (e.g., about 1800 cm/hr) and/or with a residence time of 0.5 minutes/CV. In some embodiments, 9 CV to 11 CV (e.g., about 10 CV) of a load chase solution comprising 20 mM Tris, pH 9 is applied to AEX stationary phase in a column, optionally at a velocity of 200 cm/hr to 2000 cm/hr (e.g., about 1800 cm/hr) and/or with a residence time of about 0.5 minutes/CV.

Gradient Elution

A method of purifying a rAAV vector (e.g., rAAV9, rAAV3B or others) from a solution (e.g., an affinity eluate) comprises recovery of full, intermediate and/or empty capsids by gradient elution. Gradient elution may comprise use of at least 2 different solutions (e.g., gradient elution buffers) with different pH, conductivity, and/or modifier concentration. Over the course of a gradient elution, a percentage of a first solution is varied in a manner inversely proportional to variation of a percentage of a second solution such that a gradient in the pH, conductivity, and/or modifier concentration is created as the solutions are mixed and flow through the column stationary phase. For example, at the start of a gradient elution, a percentage of a first solution (e.g., a first gradient elution buffer, buffer A) is 100% and a percentage of a second solution (e.g., a second gradient elution, buffer B) is 0% and at the end of the gradient elution the percentage of the first solution is 0% and the percentage of the second solution is 100%. In another embodiment, at the start of a gradient elution, a percentage of a first solution (e.g., a first gradient elution buffer, buffer A) is 100% and a percentage of a second solution (e.g., a second gradient elution, buffer B) is 0% and at the end of the gradient elution the percentage of the first solution is 25% and the percentage of the second solution is 75%. One of ordinary skill would understand that the percentage of each solution at the start of the gradient and at the end of the gradient can be anywhere between 0% and 100%. For instance, in some embodiments, at the start of the elution, at the end of the elution, or at any point over the course of the elution, a percentage of a first gradient elution buffer relative to a second gradient elution buffer is about 100%/0%, about 99%/1%, about 98%/2%, about 97%3%, about 96%/4%, about 95%/5%, about 90%10%, about 80%20%, about 75%/25%, about 70%/30%, about 60%/40%, about 50%/50%, about 40%/60%, about 30%/70%, about 25%/75%, about 20%/80%, about 10%/90%, about 5%/95%, about 4%/96%, about 3%/97%, about 2%/98%, about 1%/99% or about 0%/100%.

In some embodiments, at the start of the elution, at the end of the elution, or at any point over the course of the elution, a percentage of a first gradient elution buffer relative to a percentage of a second gradient elution buffer is about 100% to 90%/0% to 10%, 90% to 80%/10% to 20%, 80% to 70%/20% to 30%, 70% to 60%/30% to 40%, 60% to 50%/40% to 50%, 50% to 40%/50% to 60%, 40% to 30%/60% to 70%, 30% to 20%/70% to 80%, 20% to 10%/80% to 90%, 10% to 0%/90% to 100%.

In some embodiments, over the course of application of 10 to 60 CV of a solution to the column, a percentage of buffer A (e.g., a first gradient elution buffer) is decreased, and a percentage of buffer B (e.g., a second gradient elution buffer) is increased such that at the end of the gradient elution, the percentage of gradient elution buffer A is 0%, and the percentage of gradient elution buffer B is 100%. In some embodiments, over the course of application of about 20 CV of a solution to the column, the percentage of buffer A (e.g., a first gradient elution buffer) is decreased, and the percentage of buffer B (e.g., a second gradient elution buffer) is increased such that the rate of increase of Buffer B is about 5% of buffer B per CV and such that the final percentage of buffer B in the solution is 100%. In some embodiments, over the course of application of about 37.5 CV of a solution to the column, the percentage of buffer A (e.g., a first gradient elution buffer) is decreased, and the percentage of buffer B (e.g., a second gradient elution buffer) is increased such that the rate of increase of Buffer B is about 2% of buffer B per CV, and such that the final percentage of buffer B in the solution is 75%.

In some embodiments, over the course of application of 10 to 60 CV of a solution to the column, the percentage of buffer A (e.g., a first elution buffer) is increased, and the percentage of buffer B (e.g., a second elution buffer) is decreased such that at the end of the gradient elution, the percentage of gradient elution buffer A is 100%, and the percentage of gradient elution buffer B is 0%. One of skill in the art with recognize that a gradient elution may be run to different percentages of buffer (e.g., from 0% to 75% buffer B, corresponding to 100% to 25% buffer A; from 0% to 50% buffer B, corresponding to 100% to 50% buffer A).

In some embodiments, a method of purifying a rAAV vector by AEX of the disclosure comprises performing gradient elution of a material from a stationary phase in a column wherein a concentration of a component of a first gradient elution buffer or a second gradient elution buffer increases or decreases continuously during the gradient elution. In some embodiments, a material eluted from the stationary phase comprises a rAAV vector to be purified. A rate of increase or decrease of a concentration of a component of a first gradient elution buffer or a second gradient elution buffer may be equivalent to a change in concentration of the component per total CV. In some embodiments, a rate of increase of a concentration of sodium acetate during a gradient elution is equivalent to a change in concentration of the sodium acetate per total CV applied to a stationary phase during the elution. In some embodiments, a change in concentration of a component is relative to a concentration of the component at the start of a elution as compared to a concentration of the component at the end of the elution. For example, a concentration of a component (e.g., a salt such as sodium acetate) at the start of a gradient elution is 0 mM to 100 mM, and the concentration of the component at the end of the elution is 100 mM to 1 M. In some embodiments, a concentration of a salt (e.g., sodium acetate) at the start of a gradient elution is 0 mM and the concentration of the salt at the end of the gradient elution is 400 mM to 600 mM (e.g., about 500 mM). In some embodiments, a change in a concentration of a component is 2 mM to 1 M from the start of a gradient to the end of a gradient elution, over the course of 2 CV to 100 CV of elution buffer. In some embodiments, a change in concentration of a salt is from about 0 mM to about 500 mM from the start a gradient to the end of a gradient elution over the course of 10 CV to 60 CV, 10 CV to 50 CV, 10 CV to 40 CV, 10 CV to 30 CV or 15 CV to 25 CV (e.g., 20 CV) of elution buffer, such that when the elution gradient comprises 20 CV of solution, the rate of change of sodium acetate concentration is about 500 mM per 20 CV, or 25 mM/CV. In some embodiments, a change in concentration of a salt is from about 0 mM to about 375 mM from the start a gradient to the end of a gradient elution over the course of 10 CV to 60 CV, 10 CV to 50 CV, 10 CV to 40 CV, 10 CV to 30 CV or 15 CV to 25 CV (e.g., 37.5 CV) of elution buffer, such that when the elution gradient comprises 37.5 CV of solution, the rate of change of concentration of sodium acetate is about 375 mM per 37.5 CV, or 10 mM/CV.

In some embodiments, during a gradient elution, a concentration of sodium acetate of a first gradient elution buffer, a second gradient elution buffer or a mixture of both increases continuously during the gradient elution; wherein a rate of increase of the sodium acetate is equivalent to a change in concentration of the sodium acetate per total CV applied to the stationary phase; and wherein the rate of change in concentration of the sodium acetate over the gradient elution is about 5 mM/CV to 15 mM/CV, 10 mM/CV to 50 mM/CV, 10 mM/CV to 40 mM/CV, 10 mM to 30 mM/CV or 20 mM/CV to 30 mM/CV (e.g., about 10 mM/CV, about 25 mM/CV).

In some embodiments, a change in concentration of a component over a gradient elution is about 1 mM/CV to 1 M/CV, e.g., 1 mM/CV to 10 mM/CV, 1 mM/CV to 25 mM/CV, 5 mM/CV to 15 mM/CV, 10 mM/CV to 50 mM/CV, 50 mM/CV to 100 mM/CV, 100 mM/CV to 500 mM/CV, 500 mM/CV to 1 M/CV, 1 mM/CV to 750 mM/CV, 1 mM/CV to 500 mM/CV, 1 mM/CV to 100 mM/CV, 10 mM/CV to 750 mM/CV or 50 mM/CV to 500 mM/CV.

In some embodiments, over the course of a gradient elution, a concentration of a salt in the gradient solution may vary. In some embodiments, over the course of a gradient elution, a concentration of a salt (e.g., sodium chloride, sodium acetate, ammonium acetate, magnesium chloride, sodium sulfate and a combination thereof) in the gradient solution may increase or decrease. For example, at the start of a gradient elution, a concentration of a salt in the gradient solution may be 0 mM to 100 mM, and increase to 50 mM to 1 M, e.g., 50 mM to 100 mM, 100 mM to 150 mM, 150 mM to 200 mM, 200 mM to 250 mM, 250 mM to 300 mM, 300 mM to 400 mM, 400 mM to 500 mM, 500 mM to 600 mM, 600 mM to 700 mM, 700 mM to 800 mM, 800 mM to 900 mM, 900 mM to 1 M, 50 mM to 750 mM, 50 mM to 500 mM, 50 mM to 400 mM, 50 mM to 200 mM, 100 mM to 1 M, 100 mM to 750 mM, 100 mM to 500 mM, 100 mM to 400 mM or 100 mM to 200 mM over the course of the gradient elution. In a further example, at the start of a gradient elution, a concentration of salt in the gradient solution may be 50 mM to 1 M, e.g., 50 mM to 100 mM, 100 mM to 150 mM, 150 mM to 200 mM, 200 mM to 250 mM, 250 mM to 300 mM, 300 mM to 400 mM, 400 mM to 500 mM, 500 mM to 600 mM, 600 mM to 700 mM, 700 mM to 800 mM, 800 mM to 900 mM, 900 mM to 1 M, 50 mM to 750 mM, 50 mM to 500 mM, 50 mM to 400 mM, 50 mM to 200 mM, 100 mM to 1 M, 100 mM to 750 mM, 100 mM to 500 mM, 100 mM to 400 mM or 100 mM to 200 mM and decrease to 0 mM to 100 mM over the course of the gradient elution. In some embodiments, at the start of a gradient elution a concentration of sodium acetate in the gradient solution is about 0 mM and, at the end of the gradient elution the concentration of sodium acetate is about 500 mM. In some embodiments, at the start of a gradient elution a concentration of sodium acetate in the gradient elution solution is about 0 mM and, at the end of the gradient elution the concentration of sodium acetate in the gradient elution solution is about 375 mM.

In some embodiments, over the course of a gradient elution, a pH of the gradient solution may vary. In some embodiments, over the course of a gradient elution, a pH of the gradient solution may increase or may decrease. In some embodiments, at the start of a gradient elution, a pH of the gradient solution may be between 7.0 and 11.0 (e.g., 7.0 to 7.5, 7.5 to 8.0, 8.0 to 8.5, 8.5 to 9.0, 9.0 to 9.5, 10.0 to 10.5, 10.5 to 11, 7.5 to 10.5, 8.0 to 10.0, 8.5 to 9.5 or 8.0 to 9.0). In some embodiments, at the end of a gradient elution, a pH of the gradient solution may be between 7.0 and 11.0 (e.g., 7.0 to 7.5, 7.5 to 8.0, 8.0 to 8.5, 8.5 to 9.0, 9.0 to 9.5, 10.0 to 10.5, 10.5 to 11, 7.5 to 10.5, 8.0 to 10.0, 8.5 to 9.5 or 8.0 to 9.0).

In some embodiments, over the course of a gradient elution, a conductivity of the gradient solution may vary. In some embodiments, over the course of a gradient elution, a conductivity of the gradient solution may increase or may decrease. In some embodiments, at the start of a gradient elution, a conductivity of the gradient solution may be between 1.0 mS/cm and 2.5 mS/cm, e.g., 1.2 mS/cm and 2.0 mS/cm. In some embodiments, at the end of a gradient elution, a conductivity of the gradient solution may be between 20 mS/cm and 35 mS/cm, e.g., 27 mS/cm and 33 mS/cm. In some embodiments, at the start of a gradient elution a conductivity of the gradient solution is about 1.6 mS/cm and at the end of the gradient elution the conductivity of the gradient solution is about 30 mS/cm.

In some embodiments, over the course of a gradient elution, a concentration of a buffer in the gradient solution may vary. In some embodiments, over the course of a gradient elution, a concentration of a buffer (e.g., Tris (e.g., a mixture of Tris Base and Tris-HCl), BIS-Tris propane, diethanolamine, diethylamine, tricine, triethanolamine and/or bicine of) in the gradient solution may increase or decrease. For example, at the start of a gradient elution a concentration of a buffer in the gradient solution may range from 10 mM to 500 mM, e.g., from 10 mM to 400 mM, from 10 mM to 300 mM, from 10 mM to 200 mM, from 10 mM to 50 mM, from 50 mM to 100 mM, from 50 mM to 150 mM, from 100 mM to 200 mM, from 100 mM to 400 mM, from 200 mM to 300 mM, from 300 mM to 400 mM, from 400 mM to 500 mM, or more. At the end of a gradient elution a concentration of a buffer in the gradient solution may range from 10 mM to 500 mM, e.g., from 10 mM to 400 mM, from 10 mM to 300 mM, from 10 mM to 200 mM, from 10 mM to 50 mM, from 50 mM to 100 mM, from 50 mM to 150 mM, from 100 mM to 200 mM, from 100 mM to 400 mM, from 200 mM to 300 mM, from 300 mM to 400 mM, from 400 mM to 500 mM, or more.

In some embodiment, over the course of a gradient elution, a concentration of a detergent in the gradient solution may vary. In some embodiments, over the course of a gradient elution, a concentration of a detergent (e.g., poloxamer 188 (P188), Triton X-100, polysorbate 80 (PS80), Brij-35, nonyl phenoxypolyethoxylethanol (NP-40) and a combination thereof) in the gradient solution, may increase or decrease. For example, at the start of a gradient elution a concentration of a detergent (e.g., P188) in the gradient solution may range from 0.005% to 1.0%, e.g., from 0.005% to 0.01%, from 0.005% to 0.5%, from 0.01% to 1.0%, from 0.01% to 0.5%, from 0.01% to 0.02%, from 0.02% to 0.03%, from 0.03% to 0.04%, from 0.04% to 0.05%, from 0.05% to 0.06%, from 0.05% to 1.0%, from 0.05% to 0.5%, from 0.07% to 0.08%, from 0.08% to 0.09%, from 0.09% to 0.1%, from 0.1% to 0.5%, from 0.1% to 1.0%, from 0.5% to 1.0%.

In some embodiments, at the end of a gradient elution, a concentration of a detergent (e.g., P188) in the gradient solution may range from 0.005% to 1.0%, e.g., from 0.005% to 0.01%, from 0.005% to 0.5%, from 0.01% to 1.0%, from 0.01% to 0.5%, from 0.01% to 0.02%, from 0.02% to 0.03%, from 0.03% to 0.04%, from 0.04% to 0.05%, from 0.05% to 0.06%, from 0.05% to 1.0%, from 0.05% to 0.5%, from 0.07% to 0.08%, from 0.08% to 0.09%, from 0.09% to 0.1%, from 0.1% to 0.5 from 0.1% to 1.0%, from 0.5% to 1.0%.

Over the course of a gradient elution, while one or more aspects of the gradient solution (e.g., a salt concentration) may be varied, other aspects of the gradient, such as conductivity, pH, buffer concentration, detergent concentration etc., may remain constant. For example, pH of a gradient solution may range from 7.0 to 11.0, e.g., from 7.5 to 10.5, from 8.0 to 10.0, from 8.5 to 9.5 or from 8.0 to 9.0, from 7.0 to 7.5, from 7.5 to 8.0, from 8.0 to 8.5, from 8.5 to 9.0, from 9.0 to 9.5, from 9.5 to 10, from 10.0 to 10.5 or from 10.5 to 11.0, but be constant throughout the gradient elution (e.g., a pH of about 8.8, about 8.9, about 9). In some embodiments, a pH of a gradient elution solution is about 8.9.

In some embodiments, a concentration of a buffer, such as Tris, BIS-Tris propane, bicine and a combination thereof, in a gradient elution may range from 10 mM to 500 mM, e.g., from 10 mM to 30 mM, from 10 mM to 50 mM, from 50 mM to 100 mM, from 100 mM to 200 mM, from 200 mM to 300 mM, from 300 mM to 400 mM, from 400 mM to 500 mM, from 10 mM to 400 mM, from 10 mM to 300 mM, about 10 mM to 200 mM, about 50 mM to about 150 mM or more, but be constant throughout the gradient elution (e.g., about 20 mM, about 100 mM). In some embodiments, a concentration of a buffer, such as Tris, in a gradient elution is 50 mM to 150 mM. In some embodiments, a concentration of a buffer, such as Tris, in a gradient elution solution is about 100 mM.

In some embodiments, a concentration of a detergent, such as poloxamer 188 (P188), Triton X-100, polysorbate 80 (PS80), Brij-35, nonyl phenoxypolyethoxylethanol (NP-40) and a combination thereof, in a gradient elution may range from 0.005% to 0.01%, from 0.005% to 0.5%, from 0.01% to 1.0%, from 0.01% to 0.5%, from 0.01% to 0.02%, from 0.02% to 0.03%, from 0.03% to 0.04%, from 0.04% to 0.05%, from 0.05% to 0.06%, from 0.05% to 1.0%, from 0.05% to 0.5% from 0.07% to 0.08%, from 0.08% to 0.09%, from 0.09% to 0.1%, from 0.1% to 0.5%, from 0.1% to 1.0%, from 0.5% to 1.0% but be constant throughout the gradient elution. In some embodiments, a concentration of P188 during a gradient elution is 0.05% to 0.1%. In some embodiments, a concentration of P188 during a gradient elution is about 0.01%.

During a gradient elution, as conditions within the column change, for example, pH, conductivity, salt concentration and/or modifier concentration, substances loaded onto the column elute from the column at varying points during the gradient.

In some embodiments, AAV capsids (e.g., full, intermediate, empty) are bound to a stationary phase during loading a solution comprising the capsids to be purified. During a gradient elution, as a percentage of a gradient elution buffer increases, such that the concentration of a salt increases (e.g., sodium acetate), full rAAV vectors are preferentially released (eluted) from the stationary phase, and empty capsids are preferentially retained on the stationary phase. Empty capsids are released in greater amounts as the percentage of the gradient elution buffer further increases (along with the salt concentration). Empty capsids may also be recovered in an AEX column flow-through that is, the unbound fraction. In some embodiments, full and/or intermediate capsids are recovered in a first elution peak and in a portion of a second elution peak (e.g., the first ⅔s of a second elution peak) from an AEX column. Elution of full rAAV vector from the stationary phase can be monitored during a gradient elution by measuring an A260 and A280 of the eluate, such that an increase in the A260/A280 ratio is indicative of an increase in the presence of full rAAV vector in the eluate.

In some embodiments, performing a gradient elution comprises application of about 20 CV of a solution to a column, wherein the solution is buffer A, buffer B or a mixture of buffer A and buffer B and wherein at the start of the gradient elution, the solution is 100% buffer A and at the end of the step the solution is 100% B, such that a gradient between buffer A and buffer B is created over the course of the elution phase, optionally, wherein the rate of increase of buffer B is about 5% of buffer B per CV, and optionally, when buffer B comprises sodium acetate, the concentration of sodium acetate increases at a rate of 25 mM per CV.

In some embodiments, performing a gradient elution comprises application of about 37.5 CV of a solution to a column, wherein the solution is buffer A, buffer B or a mixture of buffer A and buffer B and wherein at the start of the gradient elution, the solution is 100% buffer A and at the end of the step the solution is 75% buffer B and 25% buffer A, such that a gradient between buffer A and buffer B is created over the course of the elution phase, optionally, wherein the rate of increase of buffer B is about 2% of buffer B per CV, and optionally, when buffer B comprises sodium acetate, the concentration of sodium acetate increase at a rate of 10 mM per CV.

In some embodiments, buffer A (e.g., a first gradient elution buffer) comprises 50 mM to 150 mM (e.g., about 100 mM) Tris, 0.005% to 0.015% (e.g., about 0.01%) P188, pH 8.5 to 9.5 (e.g., about 8.9). In some embodiments, buffer B (e.g., a second gradient elution buffer) comprises about 400 mM to 600 mM (e.g., about 500 mM) sodium acetate, 50 mM to 150 mM (e.g., about 100 mM) Tris, 0.005% to 0.015% (e.g., about 0.01%) P188, pH 8.5 to 9.5 (e.g., about 8.9).

In some embodiments, a gradient elution begins with application of 100% buffer A to the column and ends with application of 100% buffer B to the column over the course of 20 CV to 24 CV (e.g., about 20 CV), such that a gradient between buffer A and buffer B is created over the course of the elution phase, and wherein buffer A comprises about 100 mM Tris, 0.01% P188, pH 8.9 and buffer B comprises about 500 mM sodium acetate, 100 mM Tris, 0.01% P188, pH 8.9. In some embodiments, a gradient elution begins with application of 100% buffer A to the column and ends with application of 75% buffer B and 25% buffer A to the column over the course of 30 CV to 40 CV (e.g., about 37.5 CV), such that a gradient between buffer A and buffer B is created over the course of the elution phase, and wherein buffer A comprises about 100 mM Tris, 0.01% P188, pH 8.9 and buffer B comprises about 500 mM sodium acetate, 100 mM Tris, 0.01% P188, pH 8.9.

In some embodiments, a gradient elution buffer comprises 5 mM to 40 mM (e.g., about 20 mM) Tris, pH 9.0. In some embodiments, a gradient elution buffer comprises 5 mM to 40 mM (e.g., about 20 mM) Tris, 400 mM to 600 mM (e.g., about 500 mM) salt (e.g., NaCl, NaAcetate, NH₄Acetate and Na₂SO₄), pH 9.0. In some embodiments, a gradient elution buffer comprises about 20 mM Tris, 500 mM sodium acetate, pH 9.0.

In some embodiments, a residence time of a gradient elution buffer (e.g., buffer, A, buffer B or a mixture of buffer A and buffer B) in an AEX column is 0.1 min/CV to 15 min/CV, e.g., 0.1 min/CV to 1 min/CV, 1 min/CV to 2 min/CV, 1.5 min/CV to 2.5 min/CV, 2 min/CV to 4 min/CV, 4 min/CV to 6 min/CV, 6 min/CV to 8 min/CV, or 8 min/CV to 10 min/CV, 10 min/CV to 12 min/CV, 12 min/CV to 15 min/CV. In some embodiments, a residence time of a solution in a column is 0.1 min/CV, about 0.5 min/CV, about 1.5 min/CV, about 2.0 min/CV, about 2.5 min/CV, about 3 min/CV, about 3.6 min/CV or about 4 min/CV, about 5 min/CV, about 6 min/CV, about 7 min/CV, about 8 min/CV, about 9 min/CV or about 10 min/CV. In some embodiments, a residence time of a gradient elution buffer (e.g., buffer A, buffer B or a mixture of buffer A and buffer B) in a column is about 3.6 min/CV or 4 min/CV. In some embodiments, a residence time of a gradient elution buffer (e.g., buffer A, buffer B or a mixture of buffer A and buffer B) in a column is about 2.0 min/CV.

In some embodiments, a residence time of a gradient elution buffer (e.g., buffer A, buffer B or a mixture of buffer A and buffer B) in a column is 1.5 to 2.5 min/CV (e.g., about 2 min/CV). In some embodiments, a residence time of a gradient elution buffer (e.g., a buffer A, buffer B, or mixture of buffer A and buffer B) in a column is 3.5 to 4.5 min/CV (e.g., about 4 min/CV). In some embodiments, a residence time of a gradient elution buffer (e.g., buffer A, buffer B or a mixture of buffer A and buffer B) in a column is about 11 min/CV.

In some embodiments, a linear velocity of a gradient elution buffer (e.g., buffer A, buffer B or a mixture of buffer A and buffer B) through a stationary phase in a column is 50 to 1800 cm/hr, e.g., 50 cm/hr to 100 cm/hr, 100 cm/hr to 200 cm/hr, 200 cm/hr to 400 cm/hr, 400 cm/hr to 600 cm/hr, 600 cm/hr to 800 cm/hr, 800 cm/hr to 1000 cm/hr, 1000 cm/hr to 1500 cm/hr, or 1500 cm/hr to 1800 cm/hr. In some embodiments, a linear velocity of a gradient elution buffer (e.g., buffer A, buffer B or a mixture of buffer A and buffer B) through a stationary phase in a column is about 298 cm/hr or about 300 cm/hr. In some embodiments, a linear velocity of a gradient elution buffer (e.g., buffer A, buffer B or a mixture of buffer A and buffer B) through a stationary phase in a column is about 75 cm/hr, about 204 cm/hr, about 298 cm/hr, about 300 cm/hr, about 597 cm/hr, or about 600 cm/hr. In some embodiments, a linear velocity of a gradient elution buffer (e.g., a buffer A, buffer B or mixture of buffer A and buffer B) through an AEX stationary phase in a 6.0 L to 6.6 L (e.g., 6.4 L) column is about 270 cm/hr to 330 cm/hr (e.g., about 300 cm/hr).

In some embodiments, a flow rate of a gradient elution buffer (e.g., buffer A, buffer B or a mixture of buffer A and buffer B) through a stationary phase in a column is about 0.2 mL/min to 2.0 L/min e.g., 0.2 mL/min to 1 mL/min, 1.0 mL/min to 10 mL/min, 10 mL/min to 100 mL/min, 100 mL/min to 500 mL/min, 500 mL/min to 1 L/min, 1 L/min to 1.5 L/min, or 1 L/min to 2 L/min. In some embodiments, a flow rate of a gradient elution buffer (e.g., buffer A, buffer B or a mixture of buffer A and buffer B) through a stationary phase in a column is about 0.47 mL/min. In some embodiments, a flow rate of a gradient elution buffer (e.g., buffer A, buffer B or a mixture of buffer A and buffer B) through a stationary phase in a column is about 1.67 mL/min. In some embodiments, a flow rate of a gradient elution buffer (e.g., buffer A, buffer B or a mixture of buffer A and buffer B) through a stationary phase in a column is about 314 mL/min. In some embodiments, a flow rate of a gradient elution buffer (e.g., buffer A, buffer B or a mixture of buffer A and buffer B) through a stationary phase in a column is about 1.8 L/min. In some embodiments, a flow rate of a gradient elution buffer (e.g., buffer A, buffer B or a mixture of buffer A and buffer B) through a stationary phase in a column is 1.5 to 2.0 L/min. In some embodiments, a flow rate of a gradient elution buffer (e.g., buffer A, buffer B or a mixture of buffer A and buffer B) through an AEX stationary phase in a 1.3 L column is about 314 mL/min. In some embodiments, a flow rate of a gradient elution buffer (e.g., buffer A, buffer B or a mixture of buffer A and buffer B) through an AEX stationary phase in a 6.0 L to 6.6 L (e.g., 6.4 L) column is about 1.8 L/min.

In some embodiments, a method of purifying a rAAV vector (e.g., rAAV9, rAAV3B or others) from a solution (e.g., an affinity eluate) comprises application of a gradient elution buffer to a column comprising POROS™ 50 HQ stationary phase. In some embodiments, a method of purifying a rAAV vector (e.g., AAV9, AAV3B or others) from an affinity eluate comprises performing a gradient elution beginning with application of 100% buffer A (e.g., comprises 50 mM to 150 mM (e.g., about 100 mM) Tris, 0.005% to 0.015% (e.g., about 0.01%) P188, pH 8.5 to 9.5 (e.g., about 8.9)) and ending with application of 75% to 100% buffer B (e.g., 50 mM to 150 mM (e.g., about 100 mM) Tris, 0.005% to 0.015% (e.g., about 0.01%) P188, pH 8.5 to 9.5 (e.g., about 8.9)) over 15 to 40 CV (e.g., about 20 CV, about 37.5 CV) to a column comprising an AEX stationary phase, wherein the rate of change of the percentage of buffer B during the gradient elution is 2% buffer B/CV to 5% buffer B/CV. In some embodiments, the column is a 6.0 L to 6.6 L (e.g., 6.4 L) column.

In some embodiments, a method of purifying a rAAV vector (e.g., AAV9, AAV3B or others) from an affinity eluate comprises performing a gradient elution beginning with application of 100% of a first buffer comprising about 100 mM Tris, 0.1% P188, pH 8.9 and ending with application of 75% to 100% of a second buffer comprising 500 mM sodium acetate, 100 mM Tris, 0.01% P188, pH 8.9 over 15 CV to 40 CV (e.g., about 20 CV, about 37.5 CV) to a column comprising an AEX stationary phase at a linear velocity of 270 cm/hr to 330 cm/hr (e.g., about 298 cm/hr, about 300 cm/hr), a flow rate of 1.5 L/min to 2.0 L/min (e.g., about 1.8 L/min) and/or a residence time of 1.5 min/CV to 4.5 min/CV (e.g., 2 min/CV, 4 min/CV), such that a gradient between the first buffer and the second buffer is created over the course of the elution, and wherein the rate of change of the percentage of buffer B during the gradient elution is 2% buffer B/CV to 5% buffer B/CV. In some embodiments, the column is a 6.0 L to 6.6 L (e.g., 6.4 L) column.

In some embodiments, the present disclosure provides a method of purifying a rAAV (e.g., rAAV9, rAAV3B or others) vector by AEX, the method comprising a step of: i) pre-use flushing comprising application of ≥4.5 CV (e.g., about 5 CV) of water for injection to an AEX stationary phase in a column; ii) sanitizing comprising application of 5 CV to 10 CV (e.g., about 8 CV) or 14.4 to 17.6 CV (e.g., about 16 CV) of a solution comprising 0.1 M to 1.0 M (e.g., about 0.5 M) NaOH to the AEX stationary phase in the column, optionally by upward flow; iii) regenerating comprising application of 4.5 to 5.5 CV (e.g., about 5 CV) of a solution comprising 1 M to 3 M (e.g., about 2 M) NaCl, 50 mM to 150 mM (e.g., about 100) mM Tris, pH 8.5 to 95. (e.g., about 9) to the AEX stationary phase in the column; iv) equilibration comprising application of 4.5 to 5.5 CV (e.g., about 5 CV) of a solution comprising 50 mM to 150 mM (e.g., about 100 mM) Tris, pH 8.5 to 9.5 (e.g., about 9) to the AEX stationary phase in the column; v) equilibration comprising application of 4.5 to 5.5 CV (e.g., about 5 CV) of an equilibration buffer comprising 50 mM to 150 mM (e.g., about 100 mM) Tris, 400 mM to 600 mM (e.g., about 500 mM) sodium acetate, 0.005% to 0.015% (e.g., about 0.01%) P188, pH 8.5 to 9.5 (e.g., about 8.9) to the AEX stationary phase in the column; vi) equilibration comprising application of ≥4.5 CV (e.g., about 5 CV) of an equilibration buffer comprising 100 mM to 300 mM (e.g., about 200 mM) histidine, 100 mM to 300 mM (e.g., 200 mM) Tris, 0.1% to 1.0% (e.g., about 0.5%) P188, pH 8.5 to 9.5 (e.g., about 8.8) to the AEX stationary phase in the column; vii) loading an affinity eluate comprising the rAAV vector to be purified to the AEX stationary phase in the column, optionally wherein the eluate has been a) diluted about 14.4 to 15.5 fold (e.g., about 15 fold) with a buffer comprising 100 to 300 mM (e.g., about 200 mM) histidine, 100 to 300 mM (e.g., about 200 mM) Tris, 0.1% to 1.0% (e.g., about 0.5%) P188, pH 8.7 to 9.0, and optionally b) filtered through an in-line 0.2 μm filter prior to application to the AEX stationary phase; viii) equilibration comprising application of 4.5 to 5.5 CV (e.g., about 5 CV) of an equilibration buffer comprising 50 mM to 150 mM (e.g., about 100 mM) Tris, 0.005% to 0.015% (e.g., about 0.01%) P188, pH 8.5 to 9.5 (e.g., about 8.9) to the AEX stationary phase in the column; and/or ix) performing gradient elution of material from the stationary phase in the column beginning with application of 100% of a first buffer comprising 50 mM to 150 mM (e.g., about 100 mM) Tris, 0.005% to 0.015% (e.g., about 0.01%) P188, pH 8.5 to 9.0 (e.g., about 8.9) and ending with application of 100% of a second buffer comprising 400 mM to 600 mM (e.g., about 500 mM) sodium acetate, 50 mM to 150 mM (e.g., about 100 mM) Tris, 0.005% to 0.015% (e.g., about 0.01%) P188, pH 8.5 to 9.5 (e.g., pH 8.9) to the stationary phase over 20 CV to 24 CV (e.g., about 20 CV); optionally wherein at least one of steps i)-ix) is performed at a linear velocity of 270 cm/hr to 330 cm/hr (e.g., about 298 cm/hr, about 300 cm/hr), and/or a residence time of 1.5 min/CV to 4.5 min/CV (e.g., about 2 min/CV, about 4 min/CV); optionally wherein the rAAV vector is a rAAV9 or a rAAV3B vector; and optionally wherein the AEX stationary phase is POROS™ 50 HQ. In some embodiments, at least one of steps i)-ix) is performed at a flow rate of 1.5 L/min to 2.0 L/min (e.g., about 1.8 L/min) through a 6 L to 6.6 L column (e.g., about 6.4 L), or about 314 mL/min through a 1.3 L column. In some embodiments, material eluted from the stationary phase during gradient elution comprises a rAAV vector to be purified. One of ordinary skill will understand that the order of the above steps may be varied.

Gradient Hold

In some embodiments, a method of purifying a rAAV vector (e.g., rAAV9, rAAV3B or others) from a solution (e.g., an affinity eluate) comprises applying a gradient hold solution to a column comprising an AEX stationary phase (e.g., POROS™ 50 HQ) for an extended volume to ensure complete gradient formation, preferably following a gradient elution. In some embodiments, a gradient hold solution comprises at least one component selected from the group consisting of a salt, a buffer, a detergent, an amino acid and a combination thereof. In some embodiments, a gradient hold solution comprises a salt selected from the group consisting of sodium chloride, sodium acetate, ammonium acetate, magnesium chloride, sodium sulfate and a combination thereof. In some embodiments, a gradient hold solution comprises a buffer selected from the group consisting of Tris, BIS-Tris propane, bicine and a combination thereof. In some embodiments, a gradient hold solution comprises a detergent selected from the group consisting of as poloxamer 188 (P188), Triton X-100, polysorbate 80 (PS80), Brij-35, nonyl phenoxypolyethoxylethanol (NP-40) and a combination thereof. In some embodiments, a gradient hold solution comprises a salt, a buffer and a detergent. In some embodiments, a gradient hold solution comprises an amino acid selected from the group consisting of the amino acid is selected from the group consisting of histidine, arginine, glycine, citrulline and a combination thereof. In some embodiments a gradient hold solution comprises sodium acetate, Tris and P188.

In some embodiments, a gradient hold solution comprises 5 mM to 1 M (e.g., about 500 mM) sodium acetate, 1 mM to 1 M (e.g., about 100 mM) Tris, 0.005% to 0.015% (e.g., about 0.01%) P188, pH 8.5 to 9.5 (e.g., about 8.9). In some embodiments, 1 CV to 10 CV, e.g., 1 CV to 3 CV, 1 CV to 5 CV, 4.4 CV to 5.5 CV, 1 CV to 8 CV, or 5 CV to 10 CV of gradient hold solution are applied to a column stationary phase. In some embodiments, 4.5 CV to 5.5 CV (e.g., about 5 CV) of a gradient hold solution comprising about 500 mM sodium acetate, 100 mM Tris, 0.01% P188, pH 8.9 are applied to an AEX column stationary phase (e.g., POROS™ 50 HQ) at a linear velocity of 270 cm/hr to 330 cm/hr (e.g., about 300 cm/hr), a flow rate of 0.4 mL/min to 2.0 L/min (e.g., about 1.8 L/min) and/or a residence time of 3.5 to 11 min/CV.

Step Elution

A method of purifying a rAAV vector (e.g., rAAV9, rAAV3B or others) from a solution (e.g., an affinity eluate) comprises a step elution (also referred to as an “isocratic elution”). In some embodiments, a step elution comprises application of at least one step elution solution to a column stationary phase, however, more commonly multiple step elution solutions (e.g., 2, 3, 4, 5, 6, 7, 8, 9, 10, 11, 12, 13, 14, 15, 16, 17, 18, 19, 20 or more) are applied to a column stationary phase.

In some embodiments, a step elution solution comprises at least one component selected from the group consisting of a salt, a buffer, a detergent, an amino acid and a combination thereof. In some embodiments, a step elution solution comprises a salt selected from the group consisting of sodium chloride, sodium acetate, ammonium acetate, magnesium chloride, sodium sulfate and a combination thereof. In some embodiments, a step elution solution comprises a buffer selected from the group consisting of Tris, BIS-Tris propane, bicine and a combination thereof. In some embodiments, a step elution solution comprises an amino acid selected from the group consisting of the amino acid is selected from the group consisting of histidine, arginine, glycine, citrulline and a combination thereof. In some embodiments, a step solution comprises a detergent selected from the group consisting of as poloxamer 188 (P188), Triton X-100, polysorbate 80 (PS80), Brij-35, nonyl phenoxypolyethoxylethanol (NP-40) and a combination thereof. In some embodiments, a step elution solution comprises a salt, a buffer and a detergent. In some embodiments a step elution solution comprises sodium acetate and Tris.

In some embodiments, a concentration of a buffer (e.g., Tris) in a step elution solution is about 1 mM to 500 mM, e.g., 1 mM to 10 mM, 10 mM to 50 mM, 50 mM to 100 mM, 100 mM to 200 mM, 200 mM to 300 mM, 300 mM to 400 mM, 400 mM to 500 mM. In some embodiments, a concentration of Tris in a step elution solution is about 20 mM.

In some embodiments a concentration of a salt (e.g., sodium acetate) in a step elution solution is about 5 mM to 600 mM, e.g., 5 mM to 50 mM. 50 mM to 100 mM, 100 mM to 200 mM, 200 mM to 300 mM, 300 mM to 400 mM, 400 mM to 500 mM, 500 mM to 600 mM. In some embodiments, a concentration of sodium acetate in a step elution solution is about 64 mM, about 75 mM, about 85 mM, about 95 mM, about 100 mM, about 105 mM, about 109 mM, about 110 mM, about 150 mM, about 200 mM, about 300 mM, about 400 mM, about 500 mM or more.

In some embodiments, a step elution solution comprises 10 mM to 50 mM (e.g., about 20 mM) Tris, 5 to 600 mM salt, pH 8.9 to 9.1. In some embodiments, a salt is sodium acetate. In some embodiments, at least one step elution solution comprises a buffer selected from the group consisting of 20 mM Tris, 64 mM sodium acetate, pH 9.0; 20 mM Tris, 75 mM sodium acetate, pH 9.0; 20 mM Tris, 85 mM sodium acetate, pH 9.0; 20 mM Tris, 95 mM sodium acetate, pH 9.0; 20 mM Tris, 100 mM sodium acetate, pH 9.0; 20 mM Tris, 105 mM sodium acetate, pH 9.0; 20 mM Tris, 109 mM sodium acetate, pH 9.0; and 20 mM Tris, 500 mM sodium acetate, pH 9.0.

In some embodiments, 1 CV to 20 CV, e.g., 1 CV to 3 CV, 2 CV to 3 CV, 1 CV to 8 CV, 4 CV to 11 CV, 5 CV to 10 CV, 10 CV to 20 CV or 15 CV to 20 CV of at least one step elution solution are applied to a column stationary phase. In some embodiments, about 2.5 CV, about 5 CV, about 10 CV or about 20 CV of at least one step elution solution are applied to a column stationary phase.

In some embodiments, a step elution solution is applied to a column stationary phase at a linear velocity of 50 cm/hr to 2000 cm/hr (e.g., about 75 cm/hr, about 150 cm/hr, about 204 cm/hr, about 600 cm/hr, about 1800 cm/hr). In some embodiments, a residence time of a step elution solution within a column stationary phase is 1 min/CV to 15 min/CV (e.g., about 1.5 min/CV, about 6 min/CV, about 12 min/CV).

In some embodiments, at least 2, 3, 4, 5, 6, 7, 8, 9, 10 or more step elution solutions are applied to a stationary phase in a column. In some embodiments, 1 CV to 20 CV of at least one step elution solution (e.g., 2, 3, 4, 5, etc.) comprising 20 mM Tris, 5 to 600 mM salt (e.g., sodium acetate), pH 8.9 to 9.1 (e.g., pH 9.0) are applied to an AEX column (e.g., POROS™ 50 HQ) at a linear velocity of 50 cm/hr to 2000 cm/hr and a residence time of 1 min/CV to 15 min/CV.

In some embodiments, a step elution solution may also be a strip solution, and preferably applied to a column stationary phase as the final step elution step. A final step elution solution (i.e., a strip solution) may be applied to a column stationary phase to cause the release of a substance (e.g., a rAAV vector) from the stationary phase. In some embodiments, a final step elution solution may have a high salt concentration (e.g., >450 mM). In some embodiments, a final step elution solution may comprise 20 mM Tris, 500 mM salt (e.g., sodium acetate), pH 8.9 to 9.1.

A method of purifying a rAAV vector (e.g., rAAV9, rAAV3B or others) from a solution (e.g., an affinity eluate) comprises application of a strip solution to a column stationary phase, preferably following application of at least one step elution solution. In some embodiments, a strip solution comprises 20 mM Tris, 500 mM sodium acetate, pH 8.9 to 9.1.

Fraction Collection, Neutralization and Pooling

A method of purifying a rAAV vector (e.g., rAAV9, rAAV3B or others) from a solution (e.g., an affinity eluate) by AEX comprises collecting at least one fraction of eluate from an AEX column to recover and enrich for full capsids, optionally during a gradient elution. In some embodiments, full capsids are collected in a first elution peak and in a portion of a second elution peak (e.g., the first ⅔ of the second elution peak). Empty capsids may be recovered in an AEX column flow-through, that is, the unbound fraction. Empty capsids may also be recovered in an elution peak, though generally at a lower level as compared to the recovery in a column flow through. Intermediate capsids may be recovered with full capsids or empty capsids.

During an elution (e.g., a gradient elution) of an AEX method of purifying a rAAV vector, eluate from an AEX column may be collected in discrete fractions of a particular volume, and/or with a particular attribute (e.g., absorbance at a particular wavelength). For example, a volume of eluate such as 1 mL to 4 L, e.g., 1 mL to 10 mL, 1 mL to 3 L, 1 mL to 2 L, 1 mL to 1 L, 1 mL to 100 mL, 10 mL to 50 mL, 50 mL to 100 mL, 100 mL to 250 mL, 250 mL to 500 mL, 500 mL to 1 L, 1 L to 1.5 L, 1.5 L. to 2 L, 2 L to 3 L, 3 L to 4 L, or more (e.g., about 1 mL, 5 mL, 10 mL, 100 mL, 500 mL, 1 L, 2 L, 3 L, 4 L etc.) or specific CV equivalents such as ⅛ of a CV to 10 CV, e.g., ⅛ of a CV to 1 CV, 1 CV to 2 CV, 2 CV to 5 CV, 5 CV to 8 CV, 8 CV to 10 CV or more (e.g., ⅛ of a CV, ¼ of a CV, ⅓ of a CV, ½ of a CV, 1 CV, 2 CV, 3 CV, 4 CV, 5 CV, 6 CV, 7 CV, 8 CV, 9 CV or more) of eluate may be collected from an AEX column during a chromatography step (e.g., gradient elution). In some embodiments, a volume of eluate ≥⅓ CV may be collected from an AEX column during a chromatography step. In some embodiments, a volume of eluate of about ½ CV may be collected from an AEX column during a chromatography step. In some embodiments, collecting at least one fraction of eluate from an AEX column during a chromatography step (e.g., a gradient elution) comprises collecting the eluate when an absorbance (e.g., absorbance at 260 nm and/or 280 nm) of a column-flow through reaches an absorbance threshold (e.g., ≥0.5 mAU/mm path length, e.g., 10 mAU/mm path length). In some embodiments, collecting at least one fraction of eluate from an AEX column during a chromatography step (e.g., a gradient elution) comprises collecting the eluate when a gradient elution solution comprises a particular percentage of an elution buffer, for example when the gradient elution solution comprises about 30% to about 35% (e.g., about 32%) to about 50% to about 55% (e.g., about 52%) of the second elution buffer (e.g., buffer B). In some embodiments, a second elution buffer (e.g., buffer B) comprises 500 mM sodium acetate, 100 mM Tris, 0.01% P188, pH 8.9.

In some embodiments, an eluate is collected in multiple fractions (e.g., 5 fractions, 10 fractions, 20 fractions or more) of a particular volume (e.g., ⅓ CV, ½ CV). In some embodiments, an eluate is collected as a single fraction. In some embodiments, an eluate is collected in a single fraction when the A280 of the eluate is ≥0.5 mAU, and optionally collected for about 2.3 CV.

In some embodiments, collecting at least one fraction eluate from an AEX column comprises measuring an absorbance at 260 nm (A260) and/or absorbance at 280 nm (A280) of the eluate collected from the column, optionally during a gradient elution. In some embodiments, measuring an absorbance (e.g., at A260 or A280) of an AEX eluate is performed in-line with collecting the at least one fraction eluate. In some embodiments, when an eluate collected from an AEX column during a chromatography elution(e.g., a gradient elution) has an A280 of 0.5 to 10 mAU/mm path length, at least one fraction of eluate is collected. In some embodiments, collecting eluate from an AEX column comprises collecting at least one fraction of eluate with a volume of ≥⅓ of a CV. In some embodiments, collecting at least one fraction of eluate (e.g., a first fraction of eluate) from an AEX column, optionally during a gradient elution, comprises collecting at least one fraction of eluate when the A280 of the eluate is ≥0.5 mAU/mm path length, and wherein a volume of the at least one fraction of eluate is ≥⅓ of a CV.

In some embodiments, one to 25 fractions, e.g., 1 to 5 fractions, 5 to 10 fraction, 10 to 15 fractions, 15 to 20 fractions or 20 to 25 fractions of eluate are collected from an AEX column, optionally during a gradient elution. In some embodiments, at least one, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more, fractions of eluate are collected from an AEX column. In some embodiments, at least 10 fractions of eluate, each with a volume of ≥⅓ of a CV, are collected from an AEX column, optionally during a gradient elution. In some embodiments, at least 20 fractions of eluate, each with a volume of about ½ of a CV, are collected from an AEX column, optionally during a gradient elution.

In some embodiments, a method of purifying a rAAV vector (e.g., rAAV9, rAAV3B or others) from an affinity eluate by AEX comprises collecting the first of about 10 fractions of eluate from an AEX column, optionally during a gradient elution, when the A280 of the eluate is >0.5 mAU/mm path length, and wherein each fraction has a volume of ≥⅓ of a CV.

In some embodiments, a method of purifying a rAAV vector (e.g., rAAV3B or others) from an affinity eluate by AEX comprises collecting the first of about 20 fractions of eluate from an AEX column, optionally during a gradient elution, when a percentage of a second elution buffer (e.g., buffer B) of the gradient elution solution is about 30% to about 35% (e.g., about 32%) and continuing the collecting until the percentage of a second elution buffer (e.g., buffer B) is about 50% to 55% (e.g., about 52%) of the gradient elution solution, and wherein each fraction has a volume of about ½ of a CV.

In some embodiments, a method purifying a rAAV vector (e.g., rAAV9, rAAV3B or others) from an solution (e.g., an affinity eluate) by AEX comprises adjusting a pH of at least one fraction of eluate collected from an AEX column, optionally during a gradient elution. In some embodiments, adjusting a pH of at least one fraction of eluate is referred to as a neutralization step. In some embodiments, a pH of at least one fraction of eluate collected from an AEX column is pH 8.5 to 9.1 prior to pH adjustment. In some embodiments, a pH of at least one fraction of eluate is adjusted to a pH of 6.8 to 7.6 (e.g., about pH 7.2). In some embodiments, a pH of at least one fraction of eluate is adjusted to a pH of 7.5 to 7.7 (e.g., about pH 7.6).

In some embodiments, a pH of at least one fraction of eluate collected from an AEX column is adjusted to a pH of 6.8 to 7.6 by addition of 14% to 16% (eluate volume weight) (e.g., 14.3% to 14.7%, 14.3% to 15%, 15% to 16%) of a solution comprising 50 mM to 500 mM, e.g., about 50 mM to 100 mM, 50 mM to 400 mM, 50 mM to 300 mM, 50 mM to 200 mM, 100 mM to 200 mM, 100 mM to 300 mM, 200 mM to 300 mM, 300 mM to 400 mM, or 400 mM to 500 mM sodium citrate, pH 3.0 to 4.0 (e.g., about 3.5). In some embodiments, adjusting a pH of at least one fraction of eluate collected from an AEX column, optionally during a gradient elution, comprises adjustment of the pH to 6.8 to 7.6 (e.g., about pH 7.2) by addition of an eluate volume weight of 14% to 16% (e.g., about 15%) eluate volume weight of a solution comprising about 250 mM sodium citrate, pH 3.5. In some embodiments, a pH of at least one fraction of eluate collected from an AEX column is adjusted by addition of a solution comprising about 50 mM citrate, pH 3.6.

In some embodiments, a pH of at least one fraction of eluate collected from an AEX column is adjusted to a pH of about 7.5 to 7.7 by collecting the at least one faction into a vessel comprising about 0.01 CV to 0.1 CV (e.g., about 0.066 CV) of a solution comprising 50 mM to 500 mM, e.g., about 50 mM to 100 mM, 50 mM to 400 mM, 50 mM to 300 mM, 50 mM to 200 mM, 100 mM to 200 mM, 100 mM to 300 mM, 200 mM to 300 mM, 300 mM to 400 mM, or 400 mM to 500 mM sodium citrate, pH 3.0 to 4.0 (e.g., about 3.5). In some embodiments, adjusting a pH of at least one fraction of eluate collected from an AEX column, optionally during a gradient elution, comprises adjustment of the pH to 7.5 to 7.7 (e.g., about pH 7.6) by collecting the at least one fraction into a vessel comprising about 0.01 CV to 0.1 CV (e.g., about 0.066 CV) of a solution comprising about 250 mM sodium citrate, pH 3.5.

In some embodiments, a method of purifying a rAAV vector (e.g., rAAV9, rAAV3B or others) from a solution (e.g., an affinity eluate) by AEX comprises measuring an absorbance of at least one fraction of eluate collected from an AEX column, optionally during a gradient elution. In some embodiments, an absorbance of at least one fraction of eluate is measured using analytical size exclusion chromatography (SEC) in a high performance liquid chromatography (HPLC) system, and measuring the absorbance at one or more wavelengths (e.g., 260 nm and/or 280 nm).

In some embodiments, measuring an absorbance of at least one fraction of eluate collected from an AEX column comprises measuring the absorbance at 260 nm (A260) and 280 nm (A280), and optionally determining an A260/A280 ratio (when measured by SEC, the measurement may be referred to as SEC A260/A280 or A260/A280 (SEC)). An A260/A280 ratio of at least one fraction of eluate collected from an AEX column is at least 0.5 to 2.0, e.g., at least 0.5 to 0.75, 0.75 to 1.0, 1.0 to 1.25, 1.25 to 1.5, 0.5 to 1.5, 1.5 to 2.0 or more. An A260/A280 ratio of at least one fraction of eluate collected from an AEX column is at least 0.5, at least 0.55, at least 0.6, at least 0.65, at least 0.7, at least 0.75, at least 0.8, at least 0.85, at least 0.9, at least 0.95, at least 1.0, at least 1.10, at least 1.11, at least 1.12, at least 1.13, at least 1.14, at least 1.15, at least 1.16, at least 1.17, at least 1.18, at least 1.19, at least 1.20, at least 1.21, at least 1.22, at least 1.23, at least 1.24, at least 1.25, at least 1.26, at least 1.27, at least 1.28, at least 1.29, at least 1.30, at least 1.31, at least 1.32, at least 1.33, at least 1.34, at least 1.35, at least 1.36, at least 1.37, at least, 1.38, at least 1.39, at least 1.40 or greater). In some embodiments, an A260/A280 ratio of at least one fraction of eluate collected from an AEX column, optionally during a gradient elution, is at least 1.25.

In some embodiments, a method of purifying a rAAV vector (e.g., rAAV9, rAAV3B or others) from a solution (e.g., an affinity eluate) by AEX comprises measuring a % of high molecular mass species (HMMS) of at least one fraction of eluate collected from an AEX column, optionally during a gradient elution. In some embodiments, a % of HMMS is measured by SEC. In some embodiments, a % HMMS of at least one fraction of eluate collected during AEX purification of rAAV vectors produced in a 250 L SUB ranges from 0% to 10% (e.g., 0% to 3.2%). In some embodiments, a % HMMS of at least one fraction of eluate collected during AEX purification of rAAV vectors produced in a 2000 L SUB ranges from 0.5% to 15% (e.g., 1.2% to 8.3%).

In some embodiments, a method of purifying a rAAV vector (e.g., rAAV9, rAAV3B or others) from a solution (e.g., an affinity eluate) by AEX comprises determining a % purity of at least one fraction of eluate collected from an AEX column, optionally during a gradient elution. In some embodiments, a % purity is determined by RP-HPLC. In some embodiments, a % purity of at least one fraction of eluate collected during AEX purification of rAAV vectors produced in a 250 L SUB ranges from 95% to 100% (e.g., 99.1% to 99.4%). In some embodiments, a % purity of at least one fraction of eluate collected during AEX purification of rAAV vectors produced in a 2000 L SUB ranges from 75% to 100% (e.g., 79.6% to 98.7%).

In some embodiments, a method of purifying a rAAV vector (e.g., rAAV9, rAAV3B or others) from a solution (e.g., an affinity eluate) by AEX comprises measuring an amount of host cell DNA (HC-DNA) of at least one fraction of eluate collected from an AEX column, optionally during a gradient elution. In some embodiments, an amount of HC-DNA is measured by qPCR. In some embodiments, an amount of HC-DNA of at least one fraction of eluate collected during AEX purification of rAAV vectors produced in a 250 L SUB ranges from 0.1 pg/1×10⁹ VG to 20 pg/1×10⁹ VG (e.g., 1.0 pg/1×10⁹ VG to 5.9 pg/1×10⁹ VG). In some embodiments, an amount of HC-DNA of at least one fraction of eluate collected during AEX purification of rAAV vectors produced in a 2000 L SUB ranges from 0.1 pg/1×10⁹ VG to 50 pg/1×10⁹ VG (e.g., 2.7 pg/1×10⁹ VG to 26.5 pg/1×10⁹ VG).

In some embodiments, a method of purifying a rAAV vector (e.g., rAAV9, rAAV3B or others) from a solution (e.g., an affinity eluate) by AEX comprises measuring an amount of host cell protein (HCP) of at least one fraction of eluate collected from an AEX column, optionally during a gradient elution. In some embodiments, an amount of HCP is measured by ELISA. In some embodiments, an amount of HCP of at least one fraction of eluate collected during AEX purification of rAAV vectors produced in a 250 L SUB ranges from an amount lower than the level of quantification (LLOQ) to 5.78 pg/1×10⁹ VG. In some embodiments, an amount of HCP of at least one fraction of eluate collected during AEX purification of rAAV vectors produced in a 2000 L SUB is LLOQ.

In some embodiments, a method of purifying a rAAV vector (e.g., rAAV9, rAAV3B or others) from a solution (e.g., an affinity eluate) comprises combining at least two fractions of eluate collected from an AEX column (e.g., during a gradient elution) to form a pooled eluate (also referred to herein as an “AEX pool”). In some embodiments, at least two fractions of eluate from an AEX column, each having an A260/A280 ratio (e.g., measured by SEC) of at least 0.5 to 2.0, e.g., at least 0.5 to 0.75, 0.75 to 1.0, 1.0 to 1.25, 1.25 to 1.5, 0.5 to 1.5, 1.5 to 2.0 or more. In some embodiments, at least two fractions of eluate from an AEX column, each having an A260/A280 ratio (e.g., measured by SEC) of at least 0.5, at least 0.55, at least 0.6, at least 0.65, at least 0.7, at least 0.75, at least 0.8, at least 0.85, at least 0.9, at least 0.95, at least 1.0, at least 1.10, at least 1.11, at least 1.12, at least 1.13, at least 1.14, at least 1.15, at least 1.16, at least 1.17, at least 1.18, at least 1.19, at least 1.20, at least 1.21, at least 1.22, at least 1.23, at least 1.24, at least 1.25, at least 1.26, at least 1.27, at least 1.28, at least 1.29, at least 1.30, at least 1.31, at least 1.32, at least 1.33, at least 1.34, at least 1.35, at least 1.36, at least 1.37, at least, 1.38, at least 1.39, at least 1.40 or greater), are combined to form a pooled eluate. In some embodiments, combining at least two fractions of eluate collected from an AEX column, optionally during a gradient elution, comprises combining at least two fractions of eluate, each having an A260/A280 ratio of ≥0.98 to form a pooled eluate. In some embodiments, combining at least two fractions of eluate collected from an AEX column, optionally during a gradient elution, comprises combining at least two fractions of eluate, each having an A260/A280 ratio of ≥1.0 to form a pooled eluate. In some embodiments, combining at least two fractions of eluate collected from an AEX column, optionally during a gradient elution, comprises combining at least two fractions of eluate, each having an A260/A280 ratio of ≥1.22 to form a pooled eluate. In some embodiments, combining at least two fractions of eluate collected from an AEX column, optionally during a gradient elution, comprises combining at least two fractions of eluate, each having an A260/A280 ratio of ≥1.24 to form a pooled eluate. In some embodiments, combining at least two fractions of eluate collected from an AEX column, optionally during a gradient elution, comprises combining at least two fractions of eluate, each having an A260/A280 ratio of ≥1.25 to form a pooled eluate.

In some embodiments, combining at least two fractions of eluate to form a pooled eluate comprises pooling 2 to 7, 2 to 10, 2 to 15, 2 to 20 or 2 to 50 fractions of eluate collected from an AEX column, optionally during a gradient elution. In some embodiments, an A260/A280 ratio of a pooled eluate is at least 0.5 to 2.0, e.g., at least 0.5 to 0.75, 0.75 to 1.0, 1.0 to 1.25, 1.25 to 1.5, 0.5 to 1.5, 1.5 to 2.0 or more. In some embodiments, an A260/A280 ratio of a pooled eluate is at least 0.5, at least 0.55, at least 0.6, at least 0.65, at least 0.7, at least 0.75, at least 0.8, at least 0.85, at least 0.9, at least 0.95, at least 1.0, at least 1.10, at least 1.11, at least 1.12, at least 1.13, at least 1.14, at least 1.15, at least 1.16, at least 1.17, at least 1.18, at least 1.19, at least 1.20, at least 1.21, at least 1.22, at least 1.23, at least 1.24, at least 1.25, at least 1.26, at least 1.27, at least 1.28, at least 1.29, at least 1.30, at least 1.31, at least 1.32, at least 1.33, at least 1.34, at least 1.35, at least 1.36, at least 1.37, at least, 1.38, at least 1.39, at least 1.40 or greater). In some embodiments, an A260/A280 ratio of a pooled eluate is >0.97. In some embodiments, an A260/A280 ratio of a pooled eluate is 0.97 to 1.03. In some embodiments, an A260/A280 ratio of a pooled eluate is 1.0 to 1.05. In some embodiments, an A260/A280 ratio of a pooled eluate is 1.20 to 1.40. In some embodiments, an A260/A280 ratio of a pooled eluate is ≥1.25, for instance about 1.28 to 1.35, and is enriched for full capsids as compared to the affinity eluate or diluted affinity eluate prior to purification by AEX.

In some embodiments, a pooled eluate comprises only a single fraction, for example, when only a single fraction meets a predetermined criterion, such as a A280 value or A260/A280 ratio. In some embodiments, a pooled eluate comprises only a single fraction, for example, when a single fraction is collected over the course of performing a gradient elution, starting at a particular point (e.g., when a particular A280 value is measured) and ending at a particular point (e.g., a particular A280 value is measured, a specific volume of eluate is collected).

In some embodiments, a pooled eluate has a pH of about 6.5 to 8, 6.8 to 7.6, about 6.8 to 7.8, 7.0 to 7.6, about 7.0 to 7.4 or about 7.0 to 7.2. In some embodiments a pooled eluate has a pH of about 6.8 to 7.6.

In some embodiments, a method of purifying a rAAV vector (e.g., rAAV9, rAAV3B or others) from an affinity eluate comprises i) collecting the first of at least one (e.g., about 10) fraction of eluate from an AEX column during a chromatography step (e.g., a gradient elution) when the A280 of the eluate is >0.5 mAU/mm path length, and wherein a volume of the at least one fraction of eluate is equivalent to ⅛ of a CV to 2 CV (e.g., about ⅓ of a CV); ii) adjusting the pH of the at least one (e.g., about 10) fraction of eluate from the column to a pH of 6.8 to 7.6 by addition of 14.3% to 15% (eluate volume weight) of a solution comprising about 200 mM to 300 mM (e.g., about 250 mM) sodium citrate, pH 3.0 to 4.0 (e.g., about 3.5); iii) measuring an absorbance of at least one fraction of eluate collected from the column and determining an A260/A280 ratio; and/or iv) combining at least two fractions of eluate collected from the column to form a pooled eluate, wherein an A260/A280 of each of the at least two fractions of eluate is ≥1.25; wherein an A260/A280 of the pooled eluate is ≥1.25, (e.g., about 1.28 to 1.35), and optionally wherein a pH of the at least one fraction of eluate or the pooled eluate is 6.8 to 7.6, and wherein the at least one fraction of eluate or the pooled eluate is enriched for full capsids, and/or depleted of empty capsids, as compared to an affinity eluate or a diluted, and optionally filtered affinity eluate prior to purification by AEX.

In some embodiments, a method of purifying a rAAV vector (e.g., rAAV9, rAAV3B or others) from an affinity eluate comprises i) collecting the first of at least one fraction (e.g., about 20) of eluate from an AEX column during a gradient elution step when a gradient elution solution comprises about 65% to about 70% (e.g., about 68%) of a first elution buffer (e.g., buffer A) comprising about 100 mM Tris, about 0.01% P188, pH 8.9 and about 30% to about 35% (e.g., about 32%) of a second elution buffer (e.g., buffer B) comprising about 500 mM sodium acetate, about 100 mM Tris, about 0.01% P188, pH 8.9 and continuing to collect all fractions of eluate until the percentage of the first elution buffer is about 45% to about 50% (e.g., about 48%) and the percentage of the second elution buffer is about 50% to about 55% (e.g., about 52%), and wherein a volume of the at least one fraction of eluate is equivalent to ⅛ of a CV to 2 CV (e.g., about ½ of a CV); ii) adjusting the pH of the at least one fraction (e.g., about 20) of eluate from the column to a pH of 7.5 to 7.7 by collecting the at least one fraction of eluate into a vessel comprising 0.01 CV to 0.1 CV (e.g., about 0.066 CV) of a solution comprising about 200 mM to 300 mM (e.g., about 250 mM) sodium citrate, pH 3.0 to 4.0 (e.g., about 3.5); iii) measuring an absorbance of at least one fraction of eluate collected from the column and determining an A260/A280 ratio; and/or iv) combining at least two fractions of eluate collected from the column to form a pooled eluate, wherein an A260/A280 of each of the at least two fractions of eluate is ≥0.98; wherein an A260/A280 of the pooled eluate is ≥1.0, and wherein the at least one fraction of eluate or the pooled eluate is enriched for full capsids, and/or depleted of empty capsids, as compared to an affinity eluate or a diluted affinity eluate prior to purification by AEX.

In some embodiments, the present disclosure provides a method of purifying an rAAV (e.g., rAAV9, rAAV3B or others) vector by AEX, the method comprising a step of: i) pre-use flushing comprising application of ≥4.5 CV (e.g., about 5 CV) of water for injection to an AEX stationary phase in a column; ii) sanitizing comprising application of 14.4 CV to 17.6 CV (e.g., about 16 CV) of a solution comprising 0.1 M to 1.0 M (e.g., about 0.5 M) NaOH to the AEX stationary phase in the column, optionally by upward flow; iii) regenerating comprising application of 4.5 CV to 5.5 CV (e.g., about 5 CV) of a solution comprising 1 M to 3 M (e.g., about 2 M) NaCl, 50 mM to 150 mM (e.g., about 100 mM) Tris, pH 8.5 to 9.5 (e.g., about pH 9) to the AEX stationary phase in the column; iv) equilibration comprising application of 4.5 CV to 5.5 CV (e.g., about 5 CV) of a solution comprising 50 mM to 150 mM (e.g., about 100 mM) Tris, pH 8.5 to 9.5 (e.g., about 9) to the AEX stationary phase in the column; v) equilibration comprising application of 4.5 CV to 5.5 CV (e.g., about 5 CV) of an equilibration buffer comprising 50 mM to 150 mM (e.g., about 100 mM) Tris, 400 mM to 600 mM (e.g., about 500 mM) sodium acetate, 0.005% to 0.015% (e.g., about 0.01%) P188, pH 8.5 to 9.5 (e.g., about 8.9) to the AEX stationary phase in the column; vi) equilibration comprising application of ≥4.5 CV (e.g., about 5 CV) of an equilibration buffer comprising 100 mM to 300 mM (e.g., about 200 mM) histidine, 100 mM to 300 mM (e.g., about 200 mM) Tris, 0.1% to 1.0% (e.g., about 0.5%) P188, pH 8.5 to 9.5 (e.g., about 8.8) to the AEX stationary phase in the column; vii) loading an affinity eluate comprising the rAAV vector to be purified to the AEX stationary phase in the column, optionally wherein the eluate has been a) diluted about 14.4 to 15.5 fold (e.g., about 15 fold) with a buffer comprising 100 mM to 300 mM (e.g., about 200 mM) histidine, 100 mM to 300 mM. (e.g., about 200 mM) Tris, 0.1% to 1.0% (e.g., about 0.5%) P188, pH 8.7 to 9.0, and optionally b) filtered through an in-line 0.2 μm filter prior to application to the stationary phase; viii) equilibration comprising application of 4.5 CV to 5.5 CV (e.g., about 5 CV) of an equilibration buffer comprising 50 mM to 150 mM (e.g., about 100 mM) Tris, 0.005% to 0.015% (e.g., about 0.01%) P188, pH 8.5 to 9.5 (e.g., about 8.9) to the AEX stationary phase in the column; ix) performing gradient elution of a material from the stationary phase in the column beginning with application of 100% of a first buffer comprising 50 mM to 150 mM (e.g., about 100 mM) Tris, 0.005% to 0.015% (e.g., about 0.01%) P188, pH 8.5 to 9.5 (e.g., 8.9) and ending with application of 100% of a second buffer comprising 400 mM to 600 mM (e.g., about 500 mM) sodium acetate, 50 mM to 150 mM (e.g., about 100 mM) Tris, 0.005% to 0.015% (e.g., about 0.01%) P188, pH 8.5 to 9.5 (e.g., pH 8.9) to the stationary phase over 20 CV to 24 CV (e.g., about 20 CV); x) collecting the first of at least one (e.g., about 10) fraction of eluate from the column during the gradient elution, when the A280 of the eluate has a ≥0.5 mAU/mm path length, and wherein a volume of the at least one fraction of eluate is equivalent to ⅛ to 2 CV (e.g., about ⅓ of a CV); xi) adjusting a pH of at least one (e.g., about 10) fraction of eluate from the column to a pH of 6.8 to 7.6, optionally, by addition of 14.3% to 15% (eluate volume weight) of a solution comprising 200 mM to 300 mM (e.g., about 250 mM) sodium citrate, pH 3.0 to 4.0 (e.g., about 3.5); xii) measuring an absorbance of at least one fraction of eluate collected from the column and determining an A260/A280 ratio (e.g., by SEC); and/or xiii) combining at least two fractions of eluate fraction collected from the column to form a pooled eluate, wherein an A260/A280 of the at least one fraction of eluate is ≥1.25, wherein an A260/A280 of the pooled eluate is ≥1.25, (e.g., about 1.28 to 1.35), and optionally wherein a pH of the at least one fraction of eluate or the pooled eluate is 6.8 to 7.6, and wherein the at least one fraction of eluate or the pooled eluate is enriched for full capsids, and/or depleted of empty capsids, as compared to the affinity eluate or the diluted, and optionally filtered affinity eluate; optionally wherein at least one of steps i) to ix) is performed at a linear velocity of 270 cm/hr to 330 cm/hr (e.g., about 300 cm/hr), a flow rate of 1.5 L/min to 2.0 L/min (e.g., about 1.8 L/min) through a 6.0 L to 6.6 L (e.g., about 6.4 L) column, or about 314 mL/min through a 1.3 L column, and/or a residence time of 3.5 min/CV to 4.5 min/CV (e.g., about 4 min/CV); optionally wherein the rAAV vector is an AAV9 vector; and optionally wherein the AEX stationary phase is POROS™ 50 HQ. In some embodiments, a material eluted from the stationary phase during gradient elution includes a rAAV vector to be purified.

In some embodiments, the present disclosure provides a method of purifying an rAAV (e.g., rAAV9, or AAV3B or others) vector by AEX, the method comprising a step of: i) sanitizing comprising application of 5 CV to 10 CV (e.g., about 8 CV) of a solution comprising 0.1 M to 1.0 M (e.g., about 0.5 M) NaOH to the AEX stationary phase in the column; ii) regenerating comprising application of 4.5 CV to 5.5 CV (e.g., about 5 CV) of a solution comprising 1 M to 3 M (e.g., about 2 M) NaCl, 50 mM to 150 mM (e.g., about 100 mM) Tris, pH 8.5 to 9.5 (e.g., about pH 9) to the AEX stationary phase in the column; iii) equilibration comprising application of 4.5 CV to 5.5 CV (e.g., about 5 CV) of an equilibration buffer comprising 50 mM to 150 mM (e.g., about 100 mM) Tris, 400 mM to 600 mM (e.g., about 500 mM) sodium acetate, 0.005% to 0.015% (e.g., about 0.01%) P188, pH 8.5 to 9.5 (e.g., about 8.9) to the AEX stationary phase in the column; iv) equilibration comprising application of ≥4.5 CV (e.g., about 5 CV) of an equilibration buffer comprising 100 mM to 300 mM (e.g., about 200 mM) histidine, 100 mM to 300 mM (e.g., about 200) mM Tris, 0.1% to 1.0% (e.g., about 0.5%) P188, pH 8.5 to 9.5 (e.g., about 8.8) to the AEX stationary phase in the column; v) loading an affinity eluate comprising the rAAV vector to be purified to the AEX stationary phase in the column, optionally wherein the eluate has been diluted about 14.4 to 15.5 fold (e.g., about 15 fold) with a buffer comprising 100 mM to 300 mM (e.g., about 200 mM) histidine, 100 mM to 300 mM. (e.g., about 200 mM) Tris, 0.1% to 1.0% (e.g., about 0.5%) P188, pH 8.7 to 9.0; vi) equilibration comprising application of 4.5 CV to 5.5 CV (e.g., about 5 CV) of an equilibration buffer comprising 50 mM to 150 mM (e.g., about 100 mM) Tris, 0.005% to 0.015% (e.g., about 0.01%) P188, pH 8.5 to 9.5 (e.g., about 8.9) to the AEX stationary phase in the column; vii) performing gradient elution of a material from the stationary phase in the column beginning with application of 100% of a first buffer comprising 50 mM to 150 mM (e.g., about 100 mM) Tris, 0.005% to 0.015% (e.g., about 0.01%) P188, pH 8.5 to 9.5 (e.g., 8.9) and ending with application of 75% of a second buffer comprising 400 mM to 600 mM (e.g., about 500 mM) sodium acetate, 50 mM to 150 mM (e.g., about 100 mM) Tris, 0.005% to 0.015% (e.g., about 0.01%) P188, pH 8.5 to 9.5 (e.g., pH 8.9) to the stationary phase over 20 to 40 CV (e.g., about 37.5 CV); viii) collecting the first of at least one fraction (e.g., about 20) of eluate from the column during the gradient elution, when the percentage of the first buffer is about 65% to about 70% (e.g., about 68%) and when the percentage of the second buffer is about 30% to 35% (e.g., about 32%) and continuing to collect all fractions of eluate until the percentage of the first buffer is about 45% to about 50% (e.g., about 48%) and the percentage of the second buffer is about 50% to 55% (e.g., about 52%), and wherein a volume of the at least one fraction of eluate is equivalent to ⅛ to 2 CV (e.g., about ½ of a CV); ix) adjusting a pH of at least one (e.g., about 20) fraction of eluate from the column to a pH of 7.5 to 7.7, optionally, by collecting the at least one fraction of eluate into a vessel comprising about 0.01 CV to 0.1 CV (e.g., about 0.066 CV) of a solution comprising 200 mM to 300 mM (e.g., about 250 mM) sodium citrate, pH 3.0 to 4.0 (e.g., about 3.5); x) measuring an absorbance of at least one fraction of eluate collected from the column and determining an A260/A280 ratio (e.g., by SEC); and/or xii) combining at least two fractions of eluate fraction collected from the column to form a pooled eluate, wherein an A260/A280 of the at least one fraction of eluate is ≥0.98, wherein an A260/A280 of the pooled eluate is ≥1.0, and wherein the at least one fraction of eluate or the pooled eluate is enriched for full capsids, and/or depleted of empty capsids, as compared to the affinity eluate or the filtered affinity eluate; optionally wherein at least one of steps i) to vii) is performed at a linear velocity of 270 cm/hr to 330 cm/hr (e.g., about 298 cm/hr) and/or a residence time of 1.5 min/CV to 4.5 min/CV (e.g., about 2 min/CV); optionally wherein the rAAV vector is a rAAV3B vector; and optionally wherein the AEX stationary phase is POROS™ 50 HQ. In some embodiments, a material eluted from the stationary phase during gradient elution includes a rAAV vector to be purified.

In some embodiments, the present disclosure provides a method of purifying an rAAV (e.g., rAAV9 or others) vector by AEX, the method comprising a step of: i) pre-use flushing comprising application of ≥4.5 CV (e.g., about 5 CV) of water for injection to an AEX stationary phase in a column; ii) sanitizing comprising application of 14.4 CV to 17.6 CV (e.g., about 16 CV) of a solution comprising 0.1 M to 1.0 M (e.g., about 0.5 M) NaOH to the AEX stationary phase in the column, optionally by upward flow; iii) regenerating comprising application of 4.5 CV to 5.5 CV (e.g., about 5 CV) of a solution comprising 1 M to 3 M (e.g., about 2 M) NaCl, 50 mM to 150 mM (e.g., about 100 mM) Tris, pH 8.5 to 9.5 (e.g., about pH 9) to the AEX stationary phase in the column; iv) equilibration comprising application of 4.5 CV to 5.5 CV (e.g., about 5 CV) of a solution comprising 50 mM to 150 mM (e.g., about 100 mM) Tris, pH 8.5 to 9.5 (e.g., about 9) to the AEX stationary phase in the column; v) equilibration comprising application of 4.5 CV to 5.5 CV (e.g., about 5 CV) of an equilibration buffer comprising 50 mM to 150 mM (e.g., about 100 mM) Tris, 400 mM to 600 mM (e.g., about 500 mM) sodium acetate, 0.005% to 0.015% (e.g., about 0.01%) P188, pH 8.5 to 9.5 (e.g., about 8.9) to the AEX stationary phase in the column; vi) equilibration comprising application of ≥4.5 CV (e.g., about 5 CV) of an equilibration buffer comprising 100 mM to 300 mM (e.g., about 200 mM) histidine, 100 mM to 300 mM (e.g., about 200 mM) Tris, 0.1% to 1.0% (e.g., about 0.5%) P188, pH 8.5 to 9.5 (e.g., about 8.8) to the AEX stationary phase in the column; vii) loading an affinity eluate comprising the rAAV vector to be purified to the AEX stationary phase in the column, optionally wherein the eluate has been a) diluted about 14.4 to 15.5 fold (e.g., about 15 fold) with a buffer comprising 100 mM to 300 mM (e.g., about 200 mM) histidine, 100 mM to 300 mM (e.g., about 200 mM) Tris, 0.1% to 1.0% (e.g., about 0.5%) P188, pH 8.7 to 9.0, and optionally b) filtered through an in-line 0.2 μm filter prior to application to the stationary phase; viii) equilibration comprising application of 4.5 CV to 5.5 CV (e.g., about 5 CV) of an equilibration buffer comprising 50 mM to 150 mM (e.g., about 100 mM) Tris, 0.005% to 0.015% (e.g., about 0.01%) P188, pH 8.5 to 9.5 (e.g., about 8.9) to the AEX stationary phase in the column; ix) performing gradient elution of a material from the stationary phase in the column beginning with application of 100% of a first buffer comprising 50 mM to 150 mM (e.g., about 100 mM) Tris, 0.005% to 0.15% (e.g., about 0.01%) P188, pH 8.5 to 9.5 (e.g., 8.9) and ending with application of 100% of a second buffer comprising 400 mM to 600 mM (e.g., about 500 mM) sodium acetate, 50 mM to 150 mM (e.g., about 100 mM) Tris, 0.005% to 0.015% (e.g., about P188, pH 8.5 to 9.5 (e.g., pH 8.9) to the stationary phase over 20 CV to 24 CV (e.g., about 20 CV); x) collecting a fraction of eluate from the column during the gradient elution, when the A280 of the eluate is ≥0.5 mAU/mm path length, and wherein a volume of the fraction of eluate is equivalent to ⅛ to 2 CV (e.g., about ⅓ of a CV); and/or xi) adjusting a pH of the fraction of eluate from the column to a pH of 6.8 to 7.6, optionally, by addition of 14.3% to 15% (eluate volume weight) of a solution comprising about 200 mM to 300 mM (e.g., about 250 mM) sodium citrate, pH 4.0 to 4.5 (e.g., about 3.5); and wherein the fraction of eluate is enriched for full capsids, and/or depleted of empty capsids, as compared to the affinity eluate or the diluted, and optionally filtered affinity eluate; optionally wherein at least one of steps i) to ix) is performed at a linear velocity of 270 cm/hr to 330 cm/hr (e.g., about 300 cm/hr), a flow rate of 1.5 L/min to 2.0 L/min (e.g., about 1.8 L/min) through a 6.0 L to 6.6 L (e.g., about 6.4 L) column, or about 314 mL/min through a 1.3 L column, and/or a residence time of 3.5 min/CV to 4.5 min/CV (e.g., about 4 min/CV); optionally wherein the rAAV vector is an AAV9 vector; and optionally wherein the AEX stationary phase is POROS™ 50 HQ. In some embodiments, a material eluted from the stationary phase during gradient elution includes a rAAV vector to be purified.

In some embodiments, the present disclosure provides a method of purifying an rAAV (e.g., rAAV9, AAV3B or others) vector by AEX, the method comprising a step of: i) sanitizing comprising application of 5 CV to 10 CV (e.g., about 8 CV) of a solution comprising 0.1 M to 1.0 M (e.g., about 0.5 M) NaOH to the AEX stationary phase in the column; ii) regenerating comprising application of 4.5 CV to 5.5 CV (e.g., about 5 CV) of a solution comprising 1 M to 3 M (e.g., about 2 M) NaCl, 50 mM to 150 mM (e.g., about 100 mM) Tris, pH 8.5 to 9.5 (e.g., about pH 9) to the AEX stationary phase in the column; iii) equilibration comprising application of 4.5 CV to 5.5 CV (e.g., about 5 CV) of a solution comprising 50 mM to 150 mM (e.g., 100 mM) Tris, pH 8.5 to 9.5 (e.g., about 9) to the AEX stationary phase in the column; iv) equilibration comprising application of 4.5 CV to 5.5 CV (e.g., about 5 CV) of an equilibration buffer comprising 50 mM to 150 mM (e.g., about 100 mM) Tris, 400 mM to 600 mM (e.g., about 500 mM) sodium acetate, 0.005% to 0.015% (e.g., about 0.01%) P188, pH 8.5 to 9.5 (e.g., about 8.9) to the AEX stationary phase in the column; v) equilibration comprising application of ≥4.5 CV (e.g., about 5 CV) of an equilibration buffer comprising 100 mM to 300 mM (e.g., about 200 mM) histidine, 100 mM to 300 mM (e.g., about 200 mM) Tris, 0.1% to 1.0% (e.g., about 0.5%) P188, pH 8.5 to 9.5 (e.g., about 8.8) to the AEX stationary phase in the column; vi) loading an affinity eluate comprising the rAAV vector to be purified to the AEX stationary phase in the column, optionally wherein the eluate has been diluted about 14.4 to 15.5 fold (e.g., about 15 fold) with a buffer comprising 100 mM to 300 mM (e.g., about 200 mM) histidine, 100 mM to 300 mM (e.g., about 200 mM) Tris, 0.1% to 1.0% (e.g., about 0.5%) P188, pH 8.7 to 9.0; vii) equilibration comprising application of 4.5 CV to 5.5 CV (e.g., about 5 CV) of an equilibration buffer comprising 50 mM to 150 mM (e.g., about 100 mM) Tris, 0.005% to 0.015% (e.g., about 0.01%) P188, pH 8.4 to 9.5 (e.g., about 8.9) to the AEX stationary phase in the column; viii) performing gradient elution of a material from the stationary phase in the column beginning with application of 100% of a first buffer comprising 50 mM to 150 mM (e.g., about 100 mM) Tris, 0.005% to 0.15% (e.g., about 0.01%) P188, pH 8.5 to 9.5 (e.g., 8.9) and ending with application of 75% of a second buffer comprising 400 mM to 600 mM (e.g., about 500 mM) sodium acetate, 50 mM to 150 mM (e.g., about 100 mM) Tris, 0.005% to 0.015% (e.g., about 0.01%) P188, pH 8.5 to 9.5 (e.g., pH 8.9) to the stationary phase over 20 CV to 40 CV (e.g., about 37.5 CV); ix) collecting a fraction of eluate from the column during the gradient elution when the percentage of the first buffer is about 65% to about 70% (e.g., about 68%) and when the percentage of the second buffer is about 30% to about 35% (e.g., about 32%) and continuing to collect all fractions of eluate until the percentage of the first buffer is about 45% to about 50% (e.g., about 48%) and the percentage of the second buffer is about 50% to 55% (e.g., about 52%), and wherein a volume of the fraction of eluate is equivalent to ⅛ to 2 CV (e.g., about ½ of a CV); and/or x) adjusting a pH of the fraction of eluate from the column to a pH of 7.5 to 7.7, optionally, by collecting the fraction of elute into a vessel comprising about 0.01 CV to 0.1 CV (e.g., about 0.066 CV) of a solution comprising 200 mM to 300 mM (e.g., about 250 mM) sodium citrate, pH 3.0 to 4.0 (e.g., about 3.5); and wherein the fraction of eluate is enriched for full capsids, and/or depleted of empty capsids, as compared to the affinity eluate or the diluted affinity eluate; optionally wherein at least one of steps i) to viii) is performed at a linear velocity of 270 cm/hr to 330 cm/hr (e.g., about 298 cm/hr) and/or a residence time of 1.5 min/CV to 4.5 min/CV (e.g., about 2 min/CV); optionally wherein the rAAV vector is an AAV3B vector; and optionally wherein the AEX stationary phase is POROS™ 50 HQ. In some embodiments, a material eluted from the stationary phase during gradient elution includes a rAAV vector to be purified.

Characterization of Pooled Eluate and Drug Substance

A method of purifying a rAAV vector (e.g., rAAV9, rAAV3B or others) from a solution (e.g., an affinity eluate) by AEX comprises collecting at least one fraction of eluate from an AEX column during an elution step (e.g., a gradient elution) and forming a pooled eluate enriched for full capsids as compared to a percentage of full capsids in the solution. A method of purifying a rAAV vector from a solution by AEX comprising collecting at least one fraction of eluate from the AEX column during an elution step and forming a pooled eluate further comprises filtering the pooled eluate by a method selected from the group consisting of viral filtration, ultrafiltration/diafiltration (UF/DF), filtration through a 0.2 μm filter and a combination thereof, to produce a drug substance. In some embodiments, quality attributes, including A260/A280 (e.g., as measured by SEC), percentages of full capsid, intermediate capsid and empty capsid, % purity, % HMMS, amount of HCP and/or amount of HC-DNA of a pooled eluate are not substantially different from the same quality attribute of a drug substance produced from the pooled eluate.

In some embodiments, the percentage of full capsids in an affinity eluate comprising an rAAV vector to be purified is less than 20% of total capsids. In some embodiments, a pooled eluate or drug substance prepared by methods disclosed herein is enriched for full capsids such that full capsids comprise 20% to 60%, 20% to 70%, 20% to 80%, 20% to 90%, 20% to 95%, 20% to 98%, 20% to 99%, 20% to greater than 99%, 40% to 50%, 40% to 60%, 40% to 70%, 40% to 80% (e.g., 44%, 45%, 50%, 53%) of total capsids in the pooled eluate or drug substance, and optionally wherein the capsids are measured by analytical ultracentrifugation (AUC) (Burnham B. et al. Human Gene Therapy Methods (2015) 26; 228-242). In some embodiments, a pooled eluate or drug substance prepared by methods disclosed herein is enriched for full capsids such that full capsids comprise 52+/−7% of total capsids in the pooled eluate or drug substance. In some embodiments, a method of purifying a rAAV vector from an affinity eluate comprises increasing the percentage of full capsids from less than 30% (e.g., 12% to 25%) in an affinity eluate to greater than 30% (e.g., 40% to 55%, 45% to 65%, 40% to greater than 99%) of total capsids in a pooled AEX eluate or drug substance.

In some embodiments, a pooled AEX eluate prepared by methods disclosed herein is enriched for full capsids such that full capsids comprise 22.9+/−2.9% of total capsids in the pooled eluate. In some embodiments, a method of purifying a rAAV vector from an affinity eluate comprises increasing the percentage of full capsids from less than 20% (e.g., 10% to 19%) in an affinity eluate to 20% or greater (e.g., 20% to 30%, 30% to 40%, 40% to 55%, 45% to 65%, 40% to greater than 99%) of total capsids in a pooled AEX eluate. In some embodiments, a method of purifying a rAAV vector from an affinity eluate comprises increasing the percentage of full capsids from 11.1±2.1 in an affinity eluate to 22.9±2.9% of total capsids in a pooled AEX eluate.

A method of purifying a rAAV vector (e.g., rAAV9, rAAV3B or others) from a solution (e.g., an affinity eluate) by AEX comprises collecting at least on fraction of eluate from an AEX column during an elution step (e.g., a gradient elution) and forming a pooled eluate with a depleted percentage of empty capsids as compared to the percentage of empty capsids in the solution, and wherein the pooled eluate is further subjected to a method of filtration selected from the group consisting of viral filtration, ultrafiltration/diafiltration (UF/DF), filtration through a 0.2 μm filter and a combination thereof, to produce a drug substance. In some embodiments, a percentage of empty capsids in an affinity eluate comprising an rAAV vector to be purified is 70% or greater of total capsids. In some embodiments, a pooled eluate or drug substance prepared by methods disclosed herein is depleted of empty capsids such that empty capsids comprise 10% to 65%, 10% to 60%, 10% to 50%, 10% to 40%, 10% to 30%, 10% to 20%, 20% to 65%, 20% to 60%, 20% to 50%, 20% to 40%, or 18% to 29%, (e.g., ≤29%) of total capsids in the pooled eluate or drug substance, and optionally wherein the capsids are measured by analytical ultracentrifugation (AUC). In some embodiments, a pooled eluate or drug substance prepared by methods disclosed herein is depleted of empty capsids such that empty capsids comprise 20%+/−7% of total capsids in the pooled eluate or drug substance. In some embodiments, a method of purifying a rAAV vector from an affinity eluate comprises reducing a percentage of empty capsids from 40% to 90% in an affinity eluate, to ≤30% of total capsids in a pooled AEX eluate or drug substance. In some embodiments, a method of purifying a rAAV vector from an affinity eluate comprises reducing a percentage of empty capsids from 79.7±2.5% in an affinity eluate, to 67.5±3.8% of total capsids in a pooled AEX eluate or drug substance.

A method of purifying a rAAV vector (e.g., rAAV9, AAV3B or others) from a solution (e.g., an affinity eluate) by AEX comprises collecting at least one fraction of eluate from an AEX column during an elution step (e.g., a gradient elution) and forming a pooled eluate comprising intermediate capsids, and wherein the pooled eluate is further subjected to a method of filtration selected from the group consisting of viral filtration, ultrafiltration/diafiltration (UF/DF), filtration through a 0.2 μm filter and a combination thereof, to produce a drug substance. In some embodiments, intermediate capsids comprise 10% to 65%, 10% to 60%, 10% to 50%, 10% to 40%, 10% to 30%, 10% to 20%, 20% to 65%, 20% to 60%, 20% to 50%, 20% to 40%, or 18% to 22% of total capsids in a pooled eluate or drug substance, and optionally wherein the capsids are measured by analytical ultracentrifugation (AUC). In some embodiments, intermediate capsids comprise 28%+/−5% of total capsids in a pooled eluate or drug substance. In some embodiments, intermediate capsids comprise 9.6%+/−1.4% of total capsids in a pooled eluate or drug substance.

A method of purifying a rAAV vector (e.g., rAAV9, rAAV3B or others) from a solution (e.g., an affinity eluate) by AEX comprises collecting at least one fraction of eluate from an AEX column during an elution step (e.g., a gradient elution) and forming a pooled eluate or drug substance that is enriched for full capsids and depleted of empty capsids as compared to the percentage of full capsids and empty capsids in the solution comprising the rAAV vector to be purified. In addition to full capsids and empty capsids, capsids which contain a partial vector genome (also referred to as a truncated, or fragmented vector genome) and/or non-transgene-related DNA (i.e., intermediate capsids) may, in certain non-limiting exemplary embodiments, make up the balance of capsid species in a pooled eluate (e.g., a pooled AEX eluate) or drug substance.

In some embodiments, a method of purifying a rAAV vector from an affinity eluate by AEX produces a pooled eluate or drug substance comprising about 53% full rAAV capsids, about 23% intermediate capsids and about 24% empty capsids of total capsids.

In some embodiments, a method of purifying a rAAV vector from an affinity eluate by AEX produces a pooled eluate or drug substance comprising about 44% full rAAV capsids, about 27% intermediate capsids and about 29% empty capsids of total capsids.

In some embodiments, a method of purifying a rAAV vector from an affinity eluate by AEX produces a pooled eluate or drug substance comprising 20% to >99% full rAAV capsids, 5% to 65% intermediate capsids and 10% to 65% empty capsids of total capsids.

In some embodiments, a method of purifying a rAAV vector from an affinity eluate by AEX produces a pooled eluate or drug substance comprising 45% to 65% full rAAV capsids, 19% to 28% intermediate capsids and 10% to 37% empty capsids. In some embodiments, the affinity eluate is generated from affinity chromatography purification of a rAAV vector produced in a vessel with a volume of 100 L to 500 L (e.g., about 250 L), optionally, wherein the vessel is a SUB.

In some embodiments, a pooled eluate or drug substance prepared by methods disclosed herein is enriched for full capsids such that full capsids comprise 55%+/−7% of total capsids in the pooled eluate or the drug substance. In some embodiments, the rAAV vector present in the pooled eluate or drug substance is produced in a vessel with a volume of 100 L to 500 L (e.g., about 250 L), optionally, wherein the vessel is SUB.

In some embodiments, a pooled eluate or drug substance prepared by methods disclosed herein comprises 24%+/−3% intermediate capsids of total capsids in the pooled eluate or the drug substance. In some embodiments, the rAAV vector present in the pooled eluate or drug substance is produced in a vessel with a volume of 100 L to 500 L (e.g., about 250 L), optionally, wherein the vessel is SUB.

In some embodiments, a pooled eluate or drug substance prepared by methods disclosed herein is depleted of empty capsids such that empty capsids comprise 21%+/−10% of total capsids in the pooled eluate or the drug substance. In some embodiments, the rAAV vector present in the pooled eluate or drug substance is produced in a vessel with a volume of 100 L to 500 L (e.g., about 250 L), optionally, wherein the vessel is SUB.

In some embodiments, a method of purifying a rAAV vector from an affinity eluate by AEX produces a pooled eluate or drug substance comprising 45% to 52% full rAAV capsids, 27% to 37% intermediate capsids and/or 18% to 22% empty capsids. In some embodiments, the affinity eluate is generated from affinity chromatography purification of a rAAV vector produced in a vessel of about 1000 L to 3000 L (e.g., about 2000 L), optionally, wherein the vessel is a SUB.

In some embodiments, a pooled eluate or drug substance prepared by methods disclosed herein is enriched for full capsids such that full capsids comprise 49%+/−2% of total capsids in the pooled eluate or the drug substance. In some embodiments, the rAAV vector present in the pooled eluate or drug substance is produced in a vessel with a volume of 1000 L to 3000 L (e.g., about 2000 L), optionally, wherein the vessel is SUB.

In some embodiments, a pooled eluate or drug substance prepared by methods disclosed herein comprises 32%+/−4% intermediate capsids of total capsids in the pooled eluate or the drug substance. In some embodiments, the rAAV vector present in the pooled eluate or drug substance is produced in a vessel with a volume of 1000 L to 3000 L (e.g., about 2000 L), optionally, wherein the vessel is SUB.

In some embodiments, a pooled eluate or drug substance prepared by methods disclosed herein is depleted of empty capsids such that empty capsids comprise 20%+/−2% of total capsids in the pooled eluate or the drug substance. In some embodiments, the rAAV vector present in the pooled eluate or drug substance is produced in a vessel with a volume of 1000 L to 3000 L (e.g., about 2000 L), optionally, wherein the vessel is SUB.

A method of purifying a rAAV vector (e.g., rAAV9, rAAV3B or others) from a solution (e.g., an affinity eluate) by AEX comprises collecting at least one fraction of eluate from an AEX column during an elution step (e.g., a gradient elution) and forming a pooled eluate, and optionally a drug substance, comprising rAAV vectors that may be quantified by quantitative polymerase chain reaction (qPCR) analysis of vector genomes (VG or vg). qPCR analysis may measure copies of ITR sequence, copies of transgene sequence and/or copies of any other nucleotide sequence present in an intact vector genome.

An amount of VG present in a pooled eluate from an AEX column may be expressed as a % VG column yield which refers to the amount of VG present in the pooled eluate collected from an AEX column (i.e., an AEX pool) as a percentage of the amount of VG present in the sample to be purified, e.g., affinity eluate, which in some embodiments has been diluted only, or diluted and filtered and applied to the AEX column.

A method of purifying a rAAV vector according to methods disclosed herein results in % VG column yield of 63%+/−26%. A method of purifying a rAAV vector according to methods disclosed herein results in % VG column yield of 1% to 10%, 1 to 20%, 1% to 30%, 1% to 40%, 1% to 50%, 1% to 60%, 1% to 70%, 1% to 80%, 1% to 90%, 1% to 99%, 5% to 95%, 10% to 85%, 15% to 75%, 20% to 65%, 25% to 55%, 30% to 45%, 30% to 80%, 35% to 65%, 40% to 70% or 100%.

In some embodiments, purification of rAAV vector produced in a 250 L SUB by methods disclosed herein results in a % VG column yield of 40% to 100%. In some embodiments, purification of a rAAV vector produced in a 2000 L SUB by methods disclosed herein results in a % VG column yield of 10% to 70% (e.g., 20% to 61%).

An amount of VG present in a pooled eluate from an AEX column may be expressed as a % VG step yield which refers to the amount of VG present in a pooled eluate collected from an AEX column (i.e., an AEX pool) as a percentage of the amount of VG present in an affinity eluate prior to dilution or filtration.

A method of purifying a rAAV vector according to methods disclosed herein results in % VG step yield of 47%+/−11%. A method of purifying a rAAV vector according to methods disclosed herein results in % VG step yield of 1% to 10%, 1 to 20%, 1% to 30%, 1% to 40%, 1% to 50%, 1% to 60%, 1% to 70%, 1% to 80%, 1% to 90%, 1% to 99%, 5% to 95%, 10% to 85%, 15% to 75%, 20% to 65%, 25% to 55%, 30% to 45%, 30% to 80%, 35% to 65%, 40% to 70% or 100%.

In some embodiments, purification of rAAV vector produced in a 250 L SUB by methods disclosed herein results in a % VG step yield of 30% to 70% (e.g., 37% to 60%). In some embodiments, purification of rAAV vector produced in a 250 L SUB by methods disclosed herein results in a % VG step yield of 45%+/−8%.

In some embodiments, purification of a rAAV vector produced in a 2000 L SUB by methods disclosed herein results in a % VG step yield of 50%+/−13%. In some embodiments, purification of a rAAV vector produced in a 2000 L SUB by methods disclosed herein results in a % VG step yield of 25% to 75% (e.g., 31% to 66%).

A method of purifying a rAAV vector (e.g., rAAV9, rAAV3B or others) from a solution (e.g., an affinity eluate) by AEX comprises collecting at least one fraction of eluate from an AEX column during an elution step (e.g., a gradient elution) and forming a pooled eluate, or drug substance, with a reduced amount of host cell protein (HCP) as compared to the amount of HCP in the solution. In some embodiments, a reduced amount of HCP in a pooled eluate, in at least one fraction of eluate, or in a drug substance, is lower than a level of quantification (LLOQ), as measured by ELISA. In some embodiments, a reduced amount of HCP in a pooled eluate, in at least one fraction of eluate, or in a drug substance, is 10 ng to 2000 ng/1×10⁹ VG, 50 ng to 200 ng/1×10⁹ VG, 100 ng to 1000 ng/1×10⁹ VG or 200 to 2000 ng/1×10⁹ VG.

In some embodiments, a method of purifying a rAAV vector (e.g., rAAV9, rAAV3b or others) from an affinity eluate by AEX comprises reducing an amount of HCP from 1 to 500 pg/1×10⁹ VG (e.g., about 50 pg/1×10⁹) in the affinity eluate, to an amount LLOQ in a pooled eluate, in at least one fraction of eluate, or in a drug substance, and wherein the rAAV vector is produced in a 250 L SUB.

In some embodiments, a method of purifying a rAAV vector (e.g., rAAV9, rAAV3B or others) from an affinity eluate by AEX comprises reducing an amount of HCP from 100 to 500 pg/1×10⁹ VG (e.g., about 330 pg/1×10⁹) in the affinity eluate, to an amount LLOQ in a pooled eluate, in at least one fraction of eluate, or in a drug substance, and wherein the rAAV vector is produced in a 2000 L SUB.

A method of purifying a rAAV vector (e.g., rAAV9, rAAV3B or others) from a solution (e.g., an affinity eluate) by AEX comprises collecting at least one fraction of eluate from an AEX column during an elution step (e.g., a gradient elution) and forming a pooled eluate, or drug substance, comprising the rAAV vector and wherein the purity of the rAAV vector is at least about 90%, about 91%, about 92%, about 93%, about 94%, about 95%, about 96%, about 97%, about 98%, about 99% or 100% as measured by, e.g., analytical reverse phase HPLC, capillary gel electrophoresis.

In some embodiments, purification of a rAAV vector produced in a 250 L SUB by methods disclosed herein results in a rAAV vector preparation (e.g., a drug substance) with a purity of 98.6%+/−0.6%. In some embodiments, purification of a rAAV vector produced in a 1000 L to 3000 L (e.g., about 2000 L) vessel (e.g., SUB) by methods disclosed herein results in a rAAV vector preparation (e.g., a drug substance) with a purity of 99.3%+/−0.3%.

A method of purifying a rAAV vector (e.g., rAAV9, rAAV3B or others) from a solution (e.g., an affinity eluate) by AEX comprises collecting at least one fraction of eluate from an AEX column during an elution step (e.g., a gradient elution) and forming a pooled eluate, or drug substance, with a percentage of HMMS of 0% to 10%. In some embodiments, a percentage of HMMS is measured by size exclusion chromatography (SEC). In some embodiments, purification of a rAAV vector produced in a 100 L to 300 L (e.g., about 250 L) vessel (e.g., SUB) by methods disclosed herein results in a rAAV vector preparation comprising 2.6%+/−0.8% HMMS as measured by SEC. In some embodiments, purification of a rAAV vector produced in a 2000 L SUB by methods disclosed herein results in a rAAV vector preparation comprising 2.9%+/−0.4% HMMS.

A method of purifying a rAAV vector (e.g., rAAV9, rAAV3B or others) from a solution (e.g., an affinity eluate) by AEX comprises collecting at least one fraction of eluate from an AEX column during an elution step (e.g., a gradient elution) and forming a pooled eluate, or drug substance, with about 7.0 to 25 pg residual HC-DNA/1×10⁹ VG. In some embodiments, an amount of HC-DNA is measured by qPCR. In some embodiments, purification of a rAAV vector produced in a 250 L SUB by methods disclosed herein results in a rAAV vector preparation (e.g., a pooled eluate, a drug substance) comprising 17.4+/−6.7 pg HC-DNA/1×10⁹ VG. In some embodiments, purification of a rAAV vector produced in a 2000 L SUB by methods disclosed herein results in a rAAV vector preparation (e.g., a pooled eluate, a drug substance) comprising 9.3+/−1.2 pg HC-DNA/1×10⁹ VG.

A method of purifying a rAAV vector (e.g., rAAV9, rAAV3B or others) from a solution (e.g., an affinity eluate) by AEX comprises collecting at least one fraction of eluate from an AEX column during an elution step (e.g., a gradient elution) and forming a pooled eluate, or drug substance, with an A260/A280 of about 1.24 to 1.32. In some embodiments, an A260/A280 is measured by size exclusion chromatography (SEC). In some embodiments, purification of a rAAV vector produced in a 250 L SUB by methods disclosed herein results in a rAAV vector preparation (e.g., a pooled eluate, a drug substance) with an A260/A280 of 1.24 to 1.32, as measured by SEC. In some embodiments, purification of a rAAV vector produced in a 2000 L SUB by methods disclosed herein results in a rAAV vector preparation (e.g., a pooled eluate, a drug substance) with an A260/A280 of 1.28 to 1.31, as measured by SEC.

A method of purifying a rAAV vector (e.g., rAAV9, rAAV3B or others) from a solution (e.g., an affinity eluate) by AEX comprises collecting at least one fraction of eluate from an AEX column during an elution step (e.g., a gradient elution) and forming a pooled eluate, wherein the pooled eluate is subjected to a method of filtration selected from the group consisting of viral filtration, ultrafiltration/diafiltration (UF/DF), filtration through a 0.2 μm filter, and a combination thereof, to produce a drug substance suitable for production of a therapeutic drug product. In some embodiments, the drug substance is suitable for administration to a human subject to treat a disease, disorder or condition (e.g., Duchenne muscular dystrophy). In some embodiments, the rAAV vector is an AAV9 vector.

AEX Stationary Phase Regeneration

Following elution (e.g., gradient elution) and collection of at least one fraction of eluate comprising a full rAAV capsid from an AEX column, additional steps may be performed to prepare the column stationary phase for further rAAV purification runs. Such steps may include, for example, sanitization, equilibration, regeneration, flush and/or storage. One of skill in the art will understand that one or more steps may be performed, in varying order and frequency.

A method of regenerating AEX stationary phase in a column for use in further rAAV purification runs comprises post-use sanitizing of the stationary phase. In some embodiments, post use sanitizing of the stationary phase follows an elution step (e.g., a gradient elution). In some embodiments, sanitizing comprises application of a solution comprising about 0.1 M to 1 M, about 0.2 M to 0.8 M, about 0.3 to about 0.7 M or about 0.4 M to about 0.6 M NaOH to AEX stationary phase in a column. In some embodiments, sanitizing comprises application of a solution comprising about 0.5 M NaOH to AEX stationary phase in a column. In some embodiments, post-use sanitizing comprises application of a solution comprising about 0.5 M NaOH to AEX stationary phase in a column and use of an upward flow. In some embodiments, post-use sanitizing comprises application of 14.4 to 17.6 CV (e.g., about 16 CV) of a solution comprising 0.5 M NaOH to AEX stationary phase in a column. In some embodiments, post-use sanitizing comprises application of 2 to 20 CV, 5 to 15 CV, 7 to 13 CV (e.g., about 5, about 7.5, about 10, about 16 CV, etc.) of a solution comprising about 0.5 M NaOH to AEX stationary phase in a column at a linear velocity of 50 to 2000 cm/hr, a flow rate of 0.2 to 3.0 L/min and/or a residence time of 2 to 15 min/CV. In some embodiments, post-use sanitizing comprises application of 14.4 to 17.6 CV (e.g. about 16 CV) of a solution comprising 0.5 M NaOH to AEX stationary phase in a column at a linear velocity of 270 to 330 cm/hr (e.g., about 300 cm/hr), a flow rate of 1.5 to 2.0 L/min (e.g., about 1.8 L/min) through a 6.0 to 6.6 L (e.g., about 6.4 L) column, or about 314 mL/min through a 1.3 L column, and/or a residence time of 3.5 to 4.5 min/CV (e.g., about 4 min/CV).

A method of regenerating a column stationary phase for further rAAV purification runs comprises regenerating the stationary phase (in some embodiments, such a step may be referred to as a “equilibration”). In some embodiments, regenerating a column stationary phase follows an elution step (e.g., a gradient elution). In some embodiments, regenerating comprises application of a solution comprising a salt (e.g., NaCl, sodium acetate, ammonium acetate (NH₄Acetate), MgCl₂ and Na₂SO₄) and buffering agent (e.g., Tris, BIS-Tris propane, diethanolamine, diethylamine, tricine, triethanolamine and/or bicine) to a stationary phase in a column. In some embodiments, regenerating comprises application of a solution comprising about 0.1 M to 5 M (e.g., 0.1 M to 4 M, 0.1 M to 3.5 M, 0.1 M to 3 M, 0.1 M to 2.5 M, 0.5 M to 4 M, 0.5 m to 3.5 M, 0.5 M to 3.0 M, 0.5 M to 2.5 M, 1 M to 4 M, 1M to 3.5 M, 1 M to 3 M, 1 M to 2.5 M or about 1.5 M to 2.5 M) of a salt to the stationary phase. In some embodiments, regenerating comprises application of a solution comprising about 1 mM to 500 mM (e.g., 1 mM to 450 mM, 1 mM to 400, 1 mM to 350 mM, 1 mM to 300 mM, 1 mM to 250 mM, 1 mM to 200 mM, 50 mM to 450 mM, 50 mM to 400 mM, 50 mM to 350 mM, 50 mM to 300 mM, 50 mM to 250 mM, 50 mM to 200 mM or 50 mM to 150 mM) of a buffering agent to the stationary phase.

In some embodiments, regenerating comprises application of a solution with a pH of about 7.0 and 11.0 (e.g., 7.5 to 10.5, 8.0 to 10.0, 8.5 to 9.5 or 8.0 to 9.0) to the stationary phase.

In some embodiments, regenerating comprises application of a solution comprising about 2 M NaCl, 100 mM Tris, pH 9 to AEX stationary phase in a column. In some embodiments, regenerating comprises application of a solution comprising 2 M NaCl, 25 mM Tris, pH 9 to AEX stationary phase in a column. In some embodiments, regeneration comprises application of 2 to 15 CV (e.g., about 5 CV, about 10 CV) of a solution (e.g., a regeneration solution) to AEX stationary phase in a column. In some embodiments, regeneration comprises application of 4.5 to 5.5 CV (e.g., about 5 CV) of a solution comprising 2 M NaCl, 100 mM Tris, pH 9 to AEX stationary phase in a column. In some embodiments, regeneration comprises application of 2 to 15 CV (e.g., about 5 CV, about 10 CV) of a solution comprising 2 M NaCl, 100 mM Tris, pH 9 to AEX stationary phase in a column at a linear velocity of 100 to 2000 cm/hr, a flow rate of 0.2 to 3.0 L/min and/or a residence time of 2 min/CV to 15 min/CV. In some embodiments, regenerating comprises application of 4.5 to 5.5 CV (e.g., about 5 CV) of a solution comprising 2 M NaCl, 100 mM Tris, pH 9, to AEX stationary phase in a column at a linear velocity of 270 to 330 cm/hr (e.g., about 300 cm/hr), a flow rate of 1.5 to 2.0 L/min (e.g., about 1.8 L/min) through a 6.0 to 6.6 L (e.g., about 6.4 L) column, or about 314 mL/min through a 1.3 L column, and/or a residence time of 3.5 to 4.5 min/CV (e.g., about 4 min/CV).

A method of regenerating a column stationary phase for further rAAV purification runs comprises equilibration of the stationary phase (in some embodiments, such a step may be referred to as a “regeneration step”). In some embodiments, equilibration of stationary phase in a column follows an elution step (e.g., a gradient elution). In some embodiments, equilibration of media in a column comprises application of a solution comprising about 100 mM Tris, pH 9 to AEX stationary phase in a column. In some embodiments, equilibration of a column comprises application of a solution comprising 20 mM Tris, pH 9 to AEX stationary phase in a column. In some embodiments, equilibration of a column comprises application of 2 to 15 CV (e.g., about 5 CV, 10 CV) of a solution (e.g., an equilibration solution) to AEX media in a column. In some embodiments, equilibration of a column comprises application of 4.5 to 5.5 CV (e.g., about 5 CV) of a solution comprising 100 mM Tris, pH 9 to AEX stationary phase in a column. In some embodiments, equilibration of a column comprises application of 2 to 15 CV (e.g., about 5 CV, about 10 CV) of a solution comprising 100 mM Tris, pH 9 to AEX stationary phase in a column at a linear velocity of 100 to 2000 cm/hr, a flow rate of to 3.0 L/min and/or a residence time of 2 min/CV to 15 min/CV. In some embodiments, equilibration of a column comprises application of 4.5 to 5.5 CV (e.g., about 5 CV) of a solution comprising 100 mM Tris, pH 9 to AEX stationary phase in a column at a linear velocity of 270 to 330 cm/hr (e.g., about 300 cm/hr), a flow rate of 1.5 to 2.0 L/min (e.g., about 1.8 L/min) through a 6.0 to 6.6 L (e.g., about 6.4 L) column, or about 314 mL/min through a 1.3 L column, and/or a residence time of 3.5 to 4.5 min/CV (e.g., about 4 min/CV).

A method of regenerating a column stationary phase for further rAAV purification runs comprises post-use flushing (i.e., flushed) of the stationary phase. In some embodiments, post-use flushing of a column follows an elution step (e.g., a gradient elution). In some embodiments, post-use flushing of a column comprises application of water for injection (e.g. purified water) to AEX stationary phase in a column. In some embodiments, post-use flushing of a column comprises application of ≥4.5 CV (e.g., about 5 CV) of water for injection to AEX stationary phase in the column. In some embodiments, post-use flushing of a column comprises application of 2 to 15 CV (e.g., about 5 CV, about 10 CV) of a solution comprising water for injection to AEX stationary phase in a column at a linear velocity of 100 to 2000 cm/hr, a flow rate of 0.2 to 3.0 L/min and/or a residence time of 2 min/CV to 15 min/CV. In some embodiments, post-use flushing of a column comprises application of 4.5 to 5.5 CV (e.g., about 5 CV) of water for injection to AEX stationary phase in a column at a linear velocity of 270 to 330 cm/hr (e.g., about 300 cm/hr), a flow rate of 1.5 to 2.0 L/min (e.g., about 1.8 L/min) through a 6.0 to 6.6 L (e.g., about 6.4 L) column, or about 314 mL/min through a 1.3 L column, and/or a residence time of 3.5 to 4.5 min/CV (e.g., about 4 min/CV).

A method of regenerating a column stationary phase for further rAAV purification runs comprises applying a storage solution to the stationary phase. In some embodiments, applying a storage solution to a column follows an elution step (e.g., a gradient elution). In some embodiments, a storage solution comprising 16% to 20% ethanol (e.g., about 17.5%) is applied to AEX stationary phase in a column. In some embodiments, 2 to 11 CV (e.g., about 3 CV, about 10 CV) of a storage buffer are applied to AEX stationary phase in a column. In some embodiments, 2.7 to 3.3 CV (e.g., about 3 CV) of a storage solution comprising 17.5% ethanol is applied to AEX stationary phase in a column. In some embodiments, 2 to 11 CV (e.g., about 3 CV) of a storage solution comprising 17.5% ethanol is applied to AEX stationary phase in a column at a linear velocity of 100 to 2000 cm/hr, a flow rate of to 3.0 L/min and/or a residence time of 2 min/CV to 15 min/CV. In some embodiments, applying a storage solution to a column comprises application of 2.7 to 3.3 CV (e.g., about 3 CV) of a solution comprising 17.5% ethanol to AEX stationary phase in a column, at a linear velocity of 270 to 330 cm/hr (e.g., about 300 cm/hr), a flow rate of 1.5 to 2.0 L/min (e.g., about 1.8 L/min) through a 6.0 to 6.6 L (e.g., 6.4 L) column, or about 314 mL/min through a 1.3 L column, and/or a residence time of 3.5 to 4.5 min/CV (e.g., about 4 min/CV).

A method of regenerating a column stationary phase for further rAAV purification runs, the method comprising a step of: i) post-use sanitizing comprising application of 14.4 to 17.6 CV (e.g. about 16 CV) of a solution comprising about 0.5 M NaOH to the stationary phase; ii) regenerating comprising application of 4.5 to 5.5 CV (e.g., about 5 CV) of a solution comprising about 2 M NaCl, 100 mM Tris, pH 9 to the stationary phase; iii) equilibration comprising application of 4.5 to 5.5 CV (e.g., about 5 CV) of a solution comprising about 100 mM Tris, pH 9 to the stationary phase; iv) post-use flushing comprising application of 4.5 to 5.5 CV (e.g., about 5 CV) of water for injection to the stationary phase; and/or v) applying a storage solution to the stationary phase comprising application of 2.7 to 3.3 CV (e.g., about 3 CV) of a storage solution comprising about 17.5% ethanol to the column; wherein at least one of steps i)-v) is performed at a linear velocity of 270 to 330 cm/hr (e.g., about 300 cm/hr), a flow rate of 1.5 to 2.0 L/min (e.g., about 1.8 L/min) through a 6.0 to 6.6 L (e.g., 6.4 L) column or about 314 mL/min through a 1.3 L column, and/or a residence time of 3.5 to 4.5 min/CV (e.g., about 4 min/CV) and wherein the stationary phase is AEX stationary phase, optionally POROS™ 50 HQ stationary phase.

A method of regenerating AEX stationary phase for further rAAV purification runs comprises application of an ethanol washout solution to the stationary phase prior to the first step of a method of purifying a rAAV vector (i.e., prior to sanitization, prior to equilibration, etc.). In some embodiments, an ethanol washout solution comprises about 20 mM Tris, pH 9. In some embodiments, application of an ethanol washout solution to the column stationary phase comprises application of 8 to 12 CV (e.g., about 10 CV) of a solution comprising about 20 mM Tris, pH 9 to AEX stationary phase. In some embodiments, application of an ethanol washout solution to AEX stationary phase comprises application of 8 to 12 CV (e.g., about 10 CV) of a solution comprising about 20 mM Tris, pH 9 to AEX stationary phase at a velocity of 100 to 1000 cm/hr (e.g., about 600 cm/hr) and/or with a residence time of 1 to 10 min/CV (e.g., about 1.5 min/CV).

### EQUIVALENTS

The foregoing written specification is considered to be sufficient to enable one skilled in the art to practice the disclosure. The foregoing description and Examples detail certain exemplary embodiments of the disclosure. It will be appreciated, however, that no matter how detailed the foregoing may appear in text, the disclosure may be practiced in many ways and the disclosure should be construed in accordance with the appended claims and any equivalents thereof.

All references cited herein, including patents, patent applications, papers, textbooks, and the like, and the references cited therein, to the extent that they are not already, are hereby incorporated herein by reference in their entirety.

### EXEMPLARY EMBODIMENTS

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1: Screening of Elution Salts to Enrich for Full AAV9 Vectors Via AEX Chromatography

Preparation of AEX Load for Elution Salt Screening

HEK 293 cells were grown in suspension culture and transfected with 3 plasmids to produce AAV9 vector per standard methods known in the art (Grieger et al. (2016) Molecular Therapy 24(2):287-297). HEK 293 cells were harvested, lysed, flocculated, and the resulting lysate was filtered. AAV9 vector was purified from the clarified lysate by affinity chromatography. An affinity column was equilibrated, loaded with clarified lysate, washed, and the purified AAV9 vector was eluted. The AAV9 vector affinity pool (also referred to as affinity eluate) was pH 4.4, with a conductivity of 5.3 mS/cm. The affinity pool was diluted 7.6-fold with 20 mM Tris, pH 9, adjusted to pH 9 with 1 M Tris base, pH 11, and filtered through a 0.2 μm filter. The resulting solution was pH 9, with a conductivity of 1.9 mS/cm, and was loaded on to AEX columns for elution salt screening studies.

Elution Salt Screening Via AEX Chromatography on a 1 mL POROS™ HQ Column

Four elution salts were studied for their ability to resolve AAV9 empty (i.e., an AAV capsid that does not contain a recombinant vector genome) and full capsids (i.e., an AAV capsid that contains a recombinant vector genome) during AEX chromatography. Consistent with Table 1, a POROS™ 50 HQ column (0.5 cm inner diameter, 5 cm bed height, 1 mL column volume) was equilibrated, loaded and washed. A 50 CV gradient was developed from 0-50% B buffer, followed by a 10 CV step elution, carried out with 100% B buffer. Four B buffers were employed, with compositions of 20 mM Tris, 500 mM salt, pH 9. The salts were one of NaCl, NaAcetate, NH₄Acetate, or Na₂SO₄.

TABLE 1
AEX chromatography methods for screening gradient elution
salts on 1 mL POROS ™ 50 HQ column.

| Column Bed | | | Load Challenge: | |
|---|---|---|---|---|
| Height: 5 cm | Column Cross Sectional Area: 0.2 cm² | | 9.3 × 10¹³ VG^{#}/mL resin | |
| Column | Column Volume: 1.0 mL | | Residence | |
| Diameter: 0.5 cm | | Column | Time | Velocity |
| Step | Solution | Volumes | (min/CV) | (cm/hr) |
| Equilibration 1 | 20 mM Tris, 500 mM salt*, pH 9 | 20 | 0.5 | 611 |
| Equilibration 2 | 20 mM Tris, pH 9 | 20 | 0.5 | 611 |
| Load | Affinity Pool diluted with 20 mM | | 0.5 | 611 |
| | Tris, pH 9 and adjusted to pH 9 | | | |
| | with 1M Tris Base, pH 11 | | | |
| Wash | 20 mM Tris, pH 9 | 10 | 0.5 | 611 |
| Gradient | A Buffer: 20 mM Tris, pH 9 | 50 | 1.5 | 204 |
| Elution | B Buffer: 20 mM Tris, 500 mM | | | |
| (0-50% B)/50 CV | salt*, pH 9 | | | |
| Step Elution | 20 mM Tris, 500 mM salt*, pH 9 | 10 | 1.5 | 204 |
| Regeneration | 2M NaCl, 25 mM Tris, pH 9 | 10 | 0.5 | 611 |
| Sanitization | 0.5M NaOH | 10 | 6 | 51 |
| Equilibration | 20 mM Tris, pH 9 | 10 | 0.5 | 611 |
| Storage | 20% Ethanol | 10 | 0.5 | 611 |

*Elution salts: NaCl, NaAcetate, NH₄Acetate, Na₂SO₄
^{#}vector genome (VG) was measured by qPCR analysis of ITR sequence.

The impact of the elution salts on the shape and A₂₆₀/A₂₈₀ ratios of elution peaks is shown by the chromatograms of FIG. 1. The A₂₆₀/A₂₈₀ ratio provides an estimation of the percentage of AAV capsids that contain a recombinant vector genome (% full), with higher ratios indicating higher % full (Sommer et al. Molecular Therapy (2003) 7(1):122-128). Elution via NaCl led to a main peak with a high A₂₆₀/A₂₈₀ (indicative of full vectors) and a closely joined shoulder with a low A₂₆₀/A₂₈₀ (indicative of empty capsids). Elution via NaAcetate and NH₄Acetate generated a main peak with high A₂₆₀/A₂₈₀, and two resolved peaks with low A₂₆₀/A₂₈₀. These results indicate that NaAcetate and NH₄Acetate resolved empty capsids from AAV9 vectors better than NaCl. Conversely, Na₂SO₄ eluted the AAV9 material in one sharp peak with moderate A₂₆₀/A₂₈₀, implying little separation of AAV9 vectors from empty capsid.

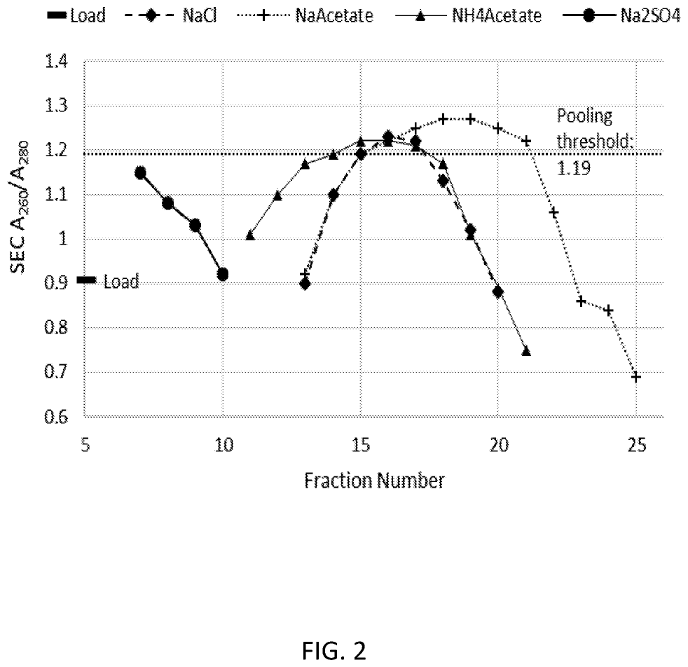

One milliliter eluate fractions were collected throughout the gradients and neutralized with 0.15 mL of 50 mM citrate, pH 3.6. Neutralized fractions were analyzed by HPLC-SEC A₂₆₀/A₂₈₀. During the HPLC-SEC method, absorbance was monitored at 214, 260, and 280 nm. The A₂₆₀/A₂₈₀ ratio provides an estimation of the % full of AAV capsids (Sommer et al. Molecular Therapy (2003) 7(1):122-128). The AAV9 signature SEC elution peaks at 260 and 280 nm were integrated and the ratio of these two values is reported as SEC A₂₆₀/A₂₈₀ as shown in FIG. 2. The NaAcetate gradient generated elution fractions with a maximum SEC A₂₆₀/A₂₈₀ of 1.27, higher than analogous maximum values for NaCl (1.23), NH₄Acetate (1.22), and Na₂SO₄ (1.15). Further, NaAcetate-based elution produced 7 contiguous eluate fractions with SEC A₂₆₀/A₂₈₀≥1.19, higher than analogous results for NaCl (3 fractions), NH₄Acetate (4 fractions) and Na₂SO₄ (0 fractions). Fractions with SEC A₂₆₀/A₂₈₀≥1.19 were pooled together, assayed by qPCR of the ITRs to determine vector genome (VG) yield, and analyzed by analytical ultracentrifugation (AUC) to determine distribution of empty, full, and intermediate (AAV capsids that have less packaged nucleic acid than full capsids and contain, for example, a partial, fragmented or a truncated vector genome and/or non-transgene-related DNA) capsids.

The elution salt screening studies demonstrated that NaAcetate outperformed NaCl, NH₄Acetate, and Na₂SO₄ in terms of VG yield, and % full of the recovered AAV9 vector as judged by SEC A₂₆₀/A₂₈₀ and AUC (Table 2). The NaAcetate AEX pool had an SEC A₂₆₀/A₂₈₀ of 1.24, slightly higher than analogous values obtained using NaCl (pool SEC A₂₆₀/A_{280=1.23}) and NH₄Acetate (SEC A₂₆₀/A₂₈₀=1.21), and significantly higher than the Na₂SO₄ pool (SEC A₂₆₀/A₂₈₀=1.15). AUC analysis on AEX pools implied that the NaAcetate gradient generated slightly higher % full (43%) than the NaCl gradient (˜38%), and significantly higher % Full than the Na₂SO₄ gradient (20%). Notably, the NaAcetate gradient elution reduced the percentage of empty capsids (% empty) from 75% in the AEX load to 29% in the AEX pool. To better delineate the performance of the elution salts, NaCl, NaAcetate, and NH₄Acetate were selected to be used on a 5.1 mL POROS™ 50 HQ column, described in the section below.

TABLE 2
Results from elution salt screening studies on a 1 mL POROS ™ 50 HQ column.

| | % VG | | | | | | |
|---|---|---|---|---|---|---|---|
| | Column | SEC-A₂₆₀/A₂₈₀ | | | AUC of AEX Pool | | |
| Sample/ | Yield | Max | Fractions ≥ | Pooled | % | % | % |
| Elution Salt | (qPCR) | Value | 1.19 | Product | full | inter. | empty |
| AEX Load | N/A | 0.91 | N/A | N/A | 12 | 13 | 75 |
| NaCl | 17 | 1.23 | 3 | 1.23 | ~38 | ~25 | ~38 |
| NaAcetate | 21 | 1.27 | 7 | 1.24 | 43 | 27 | 29 |
| NH₄Acetate | 12 | 1.22 | 4 | 1.21 | LLOQ | | |
| Na₂SO₄ | 22 | 1.15 | 0 | 1.15 | 20 | 30 | 50 |

% VG Column Yield was determined as (VG in AEX pool)/(VG in AEX load); and thus, did not account for losses incurred upon load preparation. AUC data on the NaCl gradient elution AEX pool was inconclusive and thus is reported here as an approximation (~).
LLOQ—Lower than the lower limit of quantitation.

Elution Salt Screening Via AEX Chromatography on a 5.1 mL POROS™ 50 HQ Column

Elution salts NaCl, NaAcetate, and NH₄Acetate were studied for their ability to resolve AAV9 empty capsids and full vectors during AEX chromatography. A POROS™ 50 HQ column (0.66 cm inner diameter, 15 cm bed height, 5.1 mL column volume) was equilibrated, loaded, washed, and eluted with NaCl, NaAcetate, or NH₄Acetate gradients (Table 3). One milliliter elution fractions were collected throughout the gradient, neutralized with 0.15 mL of 50 mM citrate, pH 3.6, and analyzed by HPLC-SEC A₂₆₀/A₂₈₀.

NaAcetate-based elution produced 20 contiguous fractions with SEC A₂₆₀/A₂₈₀>=1.19, higher than analogous results for NaCl (8 fractions) and NH₄Acetate (11 fractions). Fractions with SEC A₂₆₀/A₂₈₀≥1.19 were pooled together, assayed by qPCR of the ITRs to determine VG yield, and analyzed by analytical ultracentrifugation (AUC) to determine % full.

TABLE 3
AEX chromatography methods for screening elution salts performed
on a 5.1 mL POROS ™ 50 HQ column.

| Column Bed | | | Load Challenge: | |
|---|---|---|---|---|
| Height: 15 cm | Column Cross Sectional Area: 0.34 cm² | | 3 × 10¹³ VG/mL resin | |
| Column | Column Volume: 5.1 mL | | Residence | |
| Diameter: 0.66 cm | | Column | Time | Velocity |
| Step | Solution | Volumes | (min/CV) | (cm/hr) |
| Sanitization | 0.5M NaOH | 10 | 2.3 | 389 |
| Equilibration 1 | 20 mM Tris, pH 9 | 10 | 0.5 | 611 |
| Equilibration 2 | 20 mM Tris, 500 mM salt*, pH 9 | 20 | 0.5 | 1790 |
| Equilibration 3 | 20 mM Tris, pH 9 | 10 | 0.5 | 1790 |
| Load | Affinity Pool diluted with 20 mM | | 0.5 | 1790 |
| | Tris, pH 9 and 1M Tris Base, pH 11 | | | |
| Wash | 20 mM Tris, pH 9 | 10 | 0.5 | 1790 |
| Gradient Elution | A Buffer: 20 mM Tris, pH 9 | 50 | 1.5 | 597 |
| (0-50% B)/50 | B Buffer: 20 mM Tris, 500 mM | | | |
| CV | salt*, pH 9 | | | |
| Step Elution | 20 mM Tris, 500 mM salt*, pH 9 | 10 | 1.5 | 1790 |
| Regeneration | 2M NaCl, 25 mM Tris, pH 9 | 10 | 0.5 | 149 |
| Sanitization | 0.5M NaOH | 10 | 6 | 1790 |
| Equilibration | 20 mM Tris, pH 9 | 10 | 0.5 | 1790 |
| Storage | 20% Ethanol | 10 | 0.5 | 597 |

*Elution salts: NaCl, NaAcetate, NH₄Acetate

Elution salt screening studies demonstrated that NaAcetate outperformed NaCl and NH₄Acetate in terms of the % full of the recovered AAV9 vector as judged by SEC A₂₆₀/A₂₈₀ and AUC (Table 4). The NaAcetate AEX pool had an SEC A₂₆₀/A₂₈₀ of 1.26, higher than analogous values obtained using NaCl (pool SEC A₂₆₀/A₂₈₀=1.24) and NH₄Acetate (SEC A₂₆₀/A₂₈₀=1.19). AUC analysis on AEX pools implied that NaAcetate gradients generated slightly higher % full capsids (43%) than the NaCl gradient (39%) and the NH₄Acetate gradient (36%).

Collectively, the AEX runs carried out at 1 mL and 5.1 mL column volume scale showed that NaAcetate resolved empty capsids from full AAV9 vectors better than NaCl and NH₄Acetate. Therefore, NaAcetate was employed as the elution salt in further developed versions of the AEX process.

TABLE 4
Results from elution salt screening studies on
a 5.1 mL POROS ™ 50 HQ column.

| | % VG | | | | | |
|---|---|---|---|---|---|---|
| | Column | SEC-A₂₆₀/A₂₈₀ | | AUC of AEX Pool | | |
| Sample/ | Yield | Fractions ≥ | Pooled | % | % | % |
| Elution Salt | (qPCR) | 1.19 | Product | Full | Inter. | Empty |
| AEX Load | N/A | N/A | N/A | 12 | 13 | 75 |
| NaCl | 13 | 8 | 1.24 | 39 | 31 | 30 |
| NaAcetate | 10 | 20 | 1.26 | 43 | 27 | 30 |
| NH₄Acetate | 7 | 11 | 1.19 | 36 | 10 | 54 |

% VG Column Yield was determined as (VG in AEX pool)/(VG in AEX load); and thus, did not account for losses incurred upon load preparation.

### Example 2: Enrichment of Full AAV9 Vectors Via AEX Chromatography with a Sodium Acetate Step Elution

Based at least in part on the results in Example 1, NaAcetate was selected to study step elution operation of an AEX chromatography column for separation of AAV9 empty capsids from full vectors. Affinity eluate was generated as described in Example 1, diluted with 20 mM Tris, pH 9 and 1 M Tris Base, pH 11, and filtered through a 0.2 μm filter.

Screening of Optimal NaAcetate Step Elution Conditions

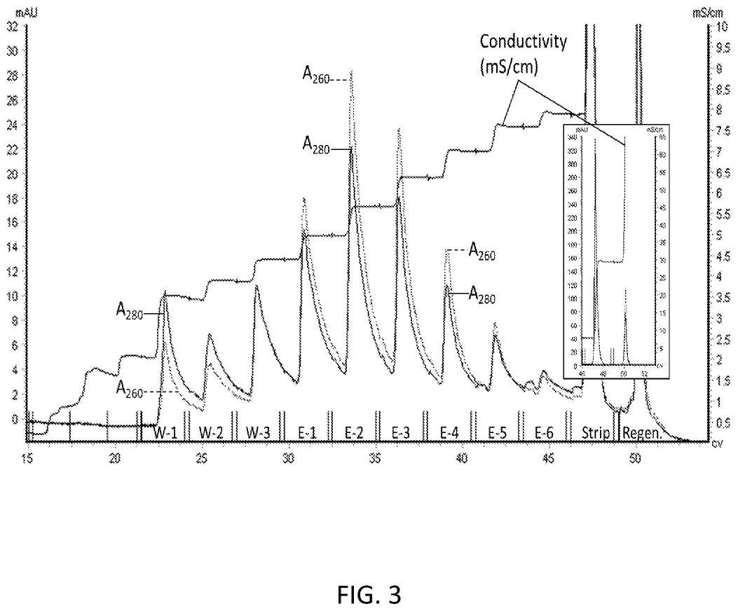

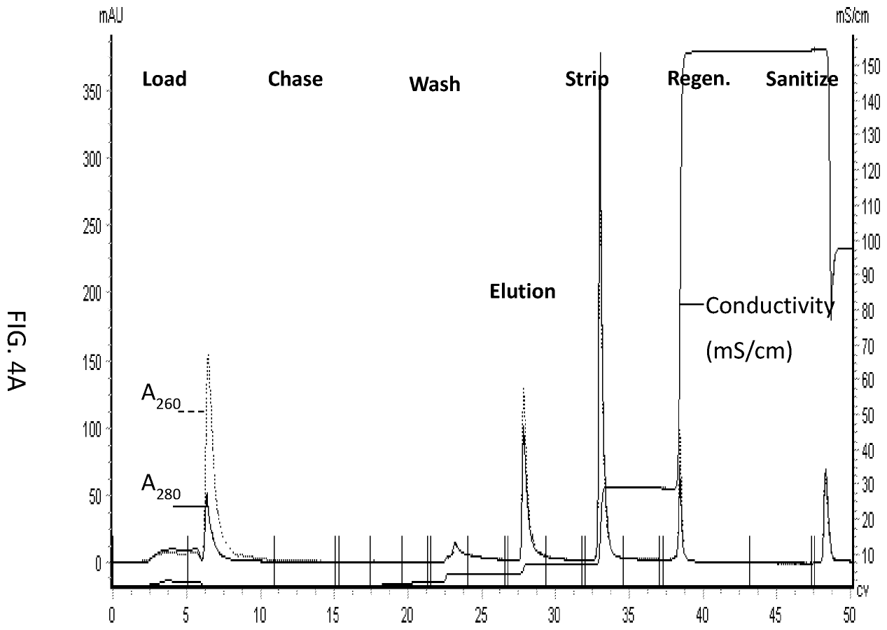

For screening purposes, a nine-step wash and elution AEX method was carried out with step increases in NaAcetate concentration in 20 mM Tris, pH 9. Consistent with Table 5, a POROS™ 50 HQ column (0.66 cm ID×15 cm BH; 5.1 mL CV) was equilibrated, loaded, washed, and eluted. Wash and elution buffers were formed in the FPLC system by mixing A Buffer: 20 mM Tris, pH 9, and B Buffer: 20 mM Tris, 140 mM NaAcetate, pH 9. Fractions were neutralized and assayed by SEC A₂₆₀/A₂₈₀, AUC, and qPCR of the ITRs. FIG. 3 depicts the 9-step chromatogram and shows a stark change in inline A₂₆₀/A₂₈₀ upon gradual increase in NaAcetate concentration of wash and elution buffers.

TABLE 5
AEX screening method employing 9-Step NaAcetate elution (3 washes and 6 elutions).

| Column Bed Height: 15 cm | | | Column Cross Sectional Area: 0.34 cm² | | Load Challenge: |
|---|---|---|---|---|---|
| Column Diameter: 0.66 cm | | | Column Volume: 5.1 mL | | 3.3 × 10¹³ VG/mL |
| | | Linear | Residence | | resin |
| | | Velocity | Time | | % of B |
| Step | CV | (cm/hr) | (min/CV) | Solution | Buffer* |
| Storage Washout | 10 | 600 | 1.5 | 20 mM Tris, pH 9 | 0 |
| Equilibration | 20 | 600 | 1.5 | 20 mM Tris, 500 mM NaAcetate, pH 9 | 0 |
| Equilibration | 5 | 600 | 1.5 | 20 mM Tris, 7 mM NaAcetate, pH 9 | 5 |
| Equilibration | 10 | 1800 | 0.5 | 20 mM Tris, pH 9 | 0 |
| Sample Loading | N/A | 1800 | 0.5 | Affinity Pool, diluted 7.1-fold with | N/A |
| | | | | 20 mM Tris, pH 9 and 1M Tris | |
| | | | | Base, pH 11 | |
| Load Chase | 10 | 1800 | 0.5 | 20 mM Tris, pH 9 | 0 |
| pH Stabilization | 2 | 600 | 1.5 | 20 mM Tris, 7 mM NaAcetate, pH 9 | 5 |
| pH Stabilization | 2 | 600 | 1.5 | 20 mM Tris, 14 mM NaAcetate, pH 9 | 10 |
| pH Stabilization | 2 | 600 | 1.5 | 20 mM Tris, 21 mM NaAcetate, pH 9 | 15 |
| Wash-1 | 2.5 | 600 | 1.5 | 20 mM Tris, 42 mM NaAcetate, pH 9 | 30 |
| Wash-2 | 2.5 | 600 | 1.5 | 20 mM Tris, 49 mM NaAcetate, pH 9 | 35 |
| Wash-3 | 2.5 | 600 | 1.5 | 20 mM Tris, 57 mM NaAcetate, pH 9 | 40 |
| Elution-1 | 2.5 | 600 | 1.5 | 20 mM Tris, 64 mM NaAcetate, pH 9 | 46 |
| Elution-2 | 2.5 | 600 | 1.5 | 20 mM Tris, 75 mM NaAcetate, pH 9 | 54 |
| Elution-3 | 2.5 | 600 | 1.5 | 20 mM Tris, 85 mM NaAcetate, pH 9 | 61 |
| Elution-4 | 2.5 | 600 | 1.5 | 20 mM Tris, 95 mM NaAcetate, pH 9 | 68 |
| Elution-5 | 2.5 | 600 | 1.5 | 20 mM Tris, 105 mM NaAcetate, pH 9 | 75 |
| Elution-6 | 2.5 | 600 | 1.5 | 20 mM Tris, 109 mM NaAcetate, pH 9 | 78 |
| Strip | 5 | 600 | 1.5 | 20 mM Tris, 500 mM NaAcetate, pH 9 | 0 |
| Regeneration | 10 | 1800 | 0.5 | 25 mM Tris, 2M NaCl, pH 9 | 0 |
| Sanitization | 10 | 150 | 6 | 0.5M NaOH | 0 |
| Equilibration | 10 | 1800 | 0.5 | 20 mM Tris pH 9 | 0 |
| Storage | 10 | 1800 | 0.5 | 20% Ethanol | 0 |

*Wash, pH stabilization, and elution buffers were made in the FPLC system by mixing A & B Buffers; A buffer: 20 mM Tris, pH 9; B buffer: 20 mM Tris, 140 mM NaAcetate, pH 9

Analytical results from the nine-step run show that empty AAV9 capsids can be resolved from full AAV9 vectors via NaAcetate step washes and elutions (Table 6). Wash 1 and 2 selectively removed bound empty capsids from the AEX column. Notably, wash 1 and 2 generated SEC A₂₆₀/A₂₈₀ values of 0.58 and 0.79 respectively, and a % empty (AUC) of 98% and 85%, respectively. Elution fractions 1-4 enriched for full AAV9 vector, with SEC A₂₆₀/A₂₈₀ values in the range of 1.27-1.30, and % full (AUC) in the range of 29-53%, considerably higher than the 12% full in the AEX load. Based on these findings, a step wash and elution method based on NaAcetate was designed and is provided below.

TABLE 6
Results from NaAcetate 9-step elution studies carried
out on a 5.1 mL POROS ™ 50 HQ column.

| Fraction | | Load | W-1 | W-2 | W-3 | E-1 | E-2 | E-3 | E-4 | E-5 | Strip |
|---|---|---|---|---|---|---|---|---|---|---|---|
| [NaAcetate] (mM) | | 20 | 42 | 49 | 57 | 64 | 75 | 85 | 95 | 105 | 500 |
| Conductivity | | 1.8 | 3.5 | 3.9 | 4.4 | 5.0 | 5.7 | 6.4 | 7.0 | 7.6 | 29.7 |
| (mS/cm) | | | | | | | | | | | |
| SEC A₂₆₀/A₂₈₀ | | 0.88 | 0.58 | 0.79 | 1.16 | 1.27 | 1.29 | 1.30 | LLOQ | LLOQ | 0.61 |
| AUC | % full | 12 | 1 | 1 | 3 | 29 | 53 | 51 | 30 | 10 | 2 |
| | % inter. | 13 | 1 | 14 | 48 | 42 | 26 | 18 | 9 | 3 | 9 |
| | % empty | 75 | 98 | 85 | 49 | 30 | 21 | 31 | 61 | 87 | 89 |

W—Wash step; E—Elution step.
LLOQ—lower than the limit of quantitation.

Enrichment of Full AAV9 Vectors Via a Step NaAcetate Wash, Elution Method

Based on the results above, AEX methods with various step NaAcetate wash and elution were tested for their ability to enrich for full AAV9 vector capsids. Consistent with Table 7, a POROS™ 50 HQ column (0.66 cm ID×15 cm BH, 5.1 mL CV) was equilibrated, washed and eluted. Wash, pH stabilization and elution buffers were made in the FPLC system by mixing A Buffer: 20 mM Tris, pH 9, and B buffer: 20 mM Tris, 140 mM NaAcetate, pH 9. Across the studied methods, multiple parameters were varied, namely elution linear velocity (75-600 cm/hr), load challenge (5.1×10¹³ to 1.1×10¹⁵ VG/mL resin), and concentration of NaAcetate in the wash step (57 mM or 68 mM).

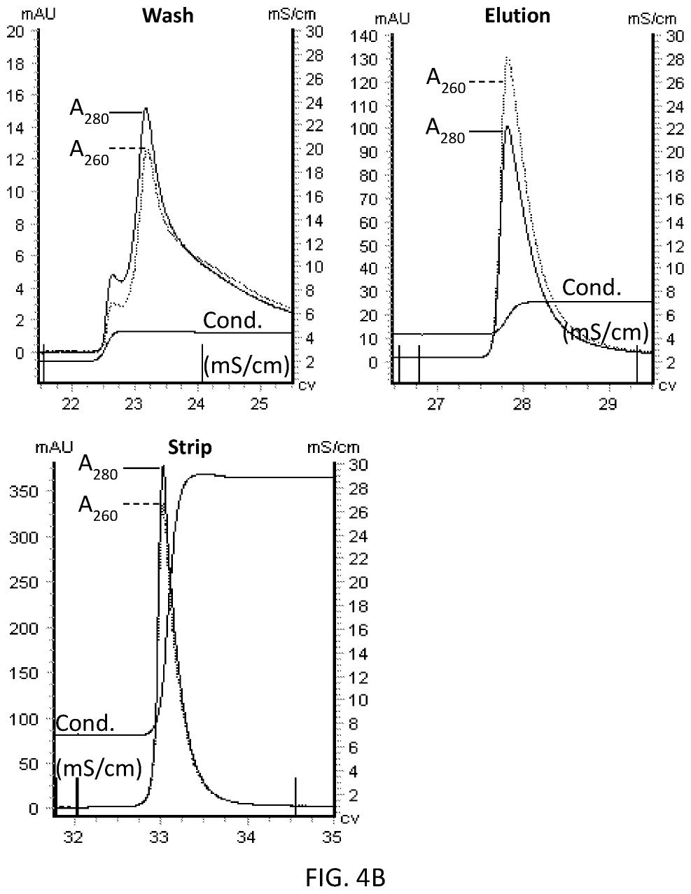

The chromatogram for the step wash and elution run with a 600 cm/hr elution, 5.1×10¹³ VG/mL resin challenge, and 57 mM NaAcetate wash is given as in FIG. 4A and FIG. 4B. Table 8 reports results and reveals that the developed step NaAcetate wash and elution AEX methods enriched for full AAV9 vector and reduced host cell protein (HCP) levels. The developed step methods increased the % full (as judged by AUC) from 18% to 40-53% and increased the SEC A₂₆₀/A₂₈₀ from 0.95 to 1.25-1.27. In addition to enriching for AAV9 full capsids, the developed step method cleared high amounts of HCP and moderate amounts of host cell DNA (HC-DNA) at low column challenges. The step NaAcetate wash and elution method did not provide % VG yields or % full of AAV9 as high as ultracentrifugation or AEX chromatography via NaAcetate gradient elution (Examples 6, 7 and 8, below). However, the step elution approach avoids complex manipulations associated with ultracentrifugation.

TABLE 7
AEX methods employing Step NaAcetate elution. Runs utilized different
load challenges, wash conditions, and elution residence times.

| Column Bed Height: 15 cm | | | Column Cross Sectional Area: 0.34 cm² | | Load Challenge: |
|---|---|---|---|---|---|
| Column Diameter: 0.66 cm | | | Column Volume: 5.1 mL | | 5 × 10¹³-1.2 × 10¹⁵ VG/mL |
| | | Linear | Residence | | resin |
| | | Velocity | Time | | % of B |
| Step | CV | (cm/hr) | (min/CV) | Solution | Buffer* |
| Ethanol Washout | 10 | 600 | 1.5 | 20 mM Tris, pH 9 | 0 |
| Equilibration | 20 | 600 | 1.5 | 20 mM Tris, 500 mM NaAcetate, pH 9 | 0 |
| Equilibration | 5 | 600 | 1.5 | 20 mM Tris, 7 mM NaAcetate, pH 9 | 5 |
| Equilibration | 10 | 1800 | 0.5 | 20 mM Tris, pH 9 | 0 |
| Sample Loading | Vary | 1800 | 0.5 | Affinity Pool diluted 7.1-fold with | N/A |
| | | | | 20 mM Tris, pH 9 and 1M Tris Base, | |
| Load Chase | 10 | 1800 | 0.5 | 20 mM Tris, pH 9 | 0 |
| pH Stabilization | 2 | 600 | 1.5 | 20 mM Tris, 7 mM NaAcetate, pH 9 | 5 |
| pH Stabilization | 2 | 600 | 1.5 | 20 mM Tris, 14 mM NaAcetate, pH 9 | 10 |
| pH Stabilization | 2 | 600 | 1.5 | 20 mM Tris, 21 mM NaAcetate, pH 9 | 15 |
| Wash | 5 | 600 | 1.5 | 20 mM Tris, 57 mM NaAcetate, pH 9 or | 41 or 48 |
| | | | | 20 mM Tris, 67 mM NaAcetate, pH 9 | |
| Elution | 5 | 600, 150, | 1.5, 6, | 20 mM Tris, 100 mM NaAcetate, pH 9 | 72 |
| | | or 75 | or 12 | | |
| Strip | 5 | 600 | 1.5 | 20 mM Tris, 500 mM NaAcetate, pH 9 | 100 |
| Regeneration | 10 | 1800 | 0.5 | 25 mM Tris, 2M NaCl, pH 9 | 0 |
| Sanitization | 10 | 150 | 6 | 0.5M NaOH | 0 |
| Equilibration | 10 | 1800 | 0.5 | 20 mM Tris, pH 9 | 0 |
| Storage | 10 | 1800 | 0.5 | 20% Ethanol | 0 |

*Wash, pH stabilization and elution buffers were made in the FPLC system by mixing A & B Buffers; A buffer: 20 mM Tris, pH 9; B buffer: 20 mM Tris, 140 mM NaAcetate, pH 9

TABLE 8
Results from AEX runs carried out with NaAcetate step elution methods
carried out on a 5.1 mL POROS ™ 50 HQ column.

| Process Inputs | | | | Process Performance: Analysis of Step | | | | | |
|---|---|---|---|---|---|---|---|---|---|
| Conc. of | | Elution | % VG | Elution Fraction | | | | | |
| NaAcetate | Challenge | Res. | Column | % Capsid Species | | | A₂₆₀/ | ng HCP/ | ng DNA/ |
| (mM) in | (VG/mL | Time | Yield | (AUC) | | | A₂₈₀ | 1 × 10¹⁴ | 1 × 10¹⁴ |
| Wash | resin) | (min) | (qPCR) | Full | Inter. | Empty | (SEC) | VG | VG |
| AEX Starting Material | | | | 18 | 9 | 73 | 0.95 | 19157 | 1070 |
| 57 | 5.1 × 10¹³ | 1.5 | 14 | 50 | 28 | 22 | 1.27 | LLOQ | 387 |
| 67 | 5.1 × 10¹³ | 1.5 | 7 | 38 | 39 | 23 | 1.27 | LLOQ | 457 |
| 57 | 2.5 × 10¹⁴ | 1.5 | 28 | 53 | 18 | 29 | 1.26 | LLOQ | 153 |
| 57 | 5.7 × 10¹⁴ | 1.5 | 31 | 49 | 20 | 31 | 1.26 | 94 | 783 |
| 57 | 1.1 × 10¹⁵ | 1.5 | 26 | 50 | 16 | 34 | 1.27 | 95 | 328 |
| 57 | 1.6 × 10¹⁴ | 6.0 | 33 | 49 | 25 | 26 | 1.27 | LLOQ | 1038 |
| 57 | 1.6 × 10¹⁴ | 12.0 | 43 | 40 | 36 | 24 | 1.25 | 170 | 972 |

% VG Column Yield was determined as (VG in AEX pooled eluate)/(VG in AEX load); and thus, did not account for losses incurred upon load preparation.

### Example 3: Screening of Methods to Prepare AAV9 Affinity Eluates for AEX Chromatography

One embodiment of large-scale downstream processing of AAV9 involves cell lysis, filtration, and affinity chromatography, with product elution at low pH and moderate conductivity. Studies on viral proteins of various AAV serotypes report calculated isoelectric points of ˜6.2 and ˜5.8 for empty and full AAV9 capsids, respectively (Venkatakrishnan et al., J. Virology (2013) 87.9:4974-4984). Screening of various conditions revealed that AAV9 only binds to AEX resins at relatively alkaline, low conductivity environments (data not shown). Therefore, preparation of acidic AAV9 affinity eluates for AEX chromatography requires raising the pH and lowering the conductivity of the vector containing buffer. This process traverses through the AAV9 isoelectric point, which is an unstable transition that can lead to vector loss.

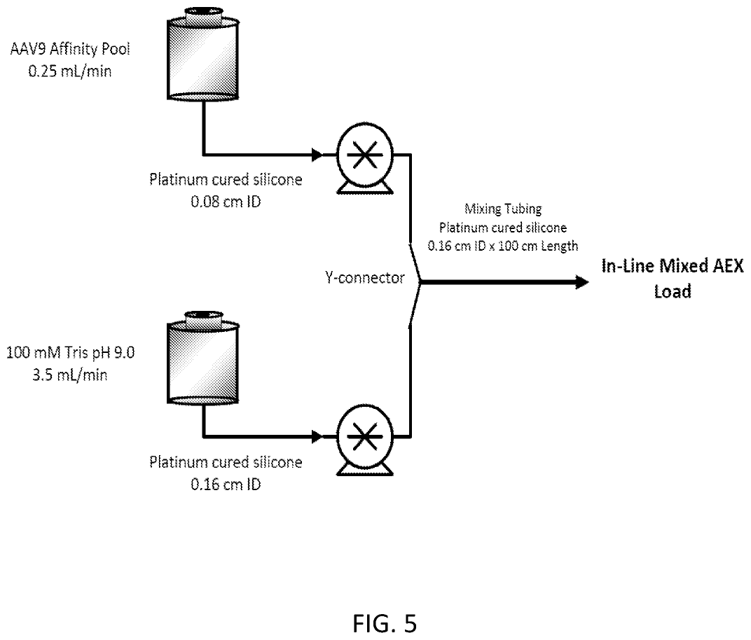

This Example details various approaches to process acidic affinity pools into AEX chromatography loads. Load preparation methods of dilution, in-line mixing (FIG. 5), and tangential flow filtration (TFF) were studied. The results show that processing AAV9 affinity eluates into AEX chromatography loads with high product yield and low aggregation required specialized development of novel and inventive processes and procedures.

Dilution Methods to Prepare AAV9 Affinity Pools for AEX Chromatography

Affinity elution pools have a pH of about 3.8-4.4, a conductivity of about 5.5-6.5 mS/cm, and comprise 7×10¹³-1.4×10¹⁴ AAV9 VG/mL. Affinity pools may be prepared for AEX chromatography as described in Example 1, that is, by dilution of the pool with alkaline buffers. Consistent with Table 9, AAV9 affinity pools were diluted with combinations of alkaline buffers in PETG vessels to raise pH and decrease conductivity. Resulting solutions were passed through 0.2 μm filters pre-wetted with diluent buffers. The diluted samples were 0.2 μm filtered to mimic large scale downstream processing, in which filters are placed at the inlet of chromatography columns. The resulting filtrates were pH 8.7-9.0, with conductivity in the range of 1.8-2.1 mS/cm, conditions that would enable high binding of AAV9 to AEX resins. Samples were taken after dilution, and after filtration, and assayed for VG titer by qPCR of the ITRs.

Table 9 reports results and reveals that high amounts of AAV9 were lost during dilution and filtration. Sequential dilution with 20 mM Tris, pH 9 and pH adjustment with 1 M Tris Base, pH 11, followed by 0.2 μm filtration resulted in 39% VG loss. Reversing the order of these diluents led to a similar result and a post filtration vector loss of 37%. Larger dilutions led to higher amounts of VG loss. A 25-fold dilution with 100 mM Tris, pH 9, followed by pH adjustment with 1 M Tris, pH 9 yielded a post filtration VG yield of only 36%. 15-fold dilution with the same buffers gave post filtration yields of 65%.

In order to reduce non-specific binding of AAV9 to the surfaces of dilution vessels and filters, 0.01% (v/v) poloxamer 188 (P188) was added to dilution buffer 100 mM Tris, pH 9. This approach only marginally increased the post filtration VG yield from 65% to 74%, without and with 0.01% P188, respectively. These dilution techniques afforded % VG yields that were lower than desired, so additional buffer exchange techniques were investigated.

TABLE 9
Screening of methods to prepare AAV9 Affinity Eluates for AEX chromatography.

| | | | | | | % VG Yield (qPCR) | |
|---|---|---|---|---|---|---|---|
| | | | | | | Post | Post |
| | | | | | | Buffer | Buffer |
| Buffer | | | Final Metrics | | | Exchange | Exchange |
| Exchange | | | Dilution | | Cond. | (Dilution | and 0.2 μm |
| Method | Buffer 1 | Buffer 2 | Factor | pH | (mS/cm) | or TFF) | Filtration |
| Dilution, pH | 20 mM | 1M Tris | 8.6 | 8.9 | 1.8 | 70 | 61 |
| Adjustment | Tris, pH 9 | Base, | | | | | |
| | | pH 11 | | | | | |
| pH | 1M Tris | 20 mM | 7.6 | 9.0 | 1.9 | 63 | 63 |
| Adjustment, | Base, pH 11 | Tris, pH 9 | | | | | |
| Dilution | | | | | | | |
| 25- Fold | 100 mM | 1M Tris, | 25 | 9.0 | 2.0 | 44 | 36 |
| Dilution | Tris, pH 9 | pH 9 | | | | | |
| 15- Fold | 100 mM | 1M Tris, | 15 | 8.9 | 2.1 | 70 | 65 |
| Dilution | Tris, pH 9 | pH 9 | | | | | |
| 15- Fold | 100 mM | | 15 | 8.8 | 2.0 | 76 | 74 |
| Dilution, | Tris, 0.01% | — | | | | | |
| 0.01% | P188, pH 9 | | | | | | |
| P188 | | | | | | | |
| In-Line | 100 mM | N/A | 15 | 8.7 | 2.1 | 79 | N/A |
| Mixing | Tris, pH 9 | | | | | | |
| TFF #1 | 100 mM | N/A | 1.7 | 9.0 | 2.0 | 66 | 65 |
| | Tris, 2 mM | | | | | | |
| | MgCl2, | | | | | | |
| | 0.01% | | | | | | |
| | P188 pH 9.0 | | | | | | |
| TFF #2 with | 100 mM | 100 mM | 1.7 | 9.0 | 2.0 | 78 | 78 |
| Arginine | Tris, 500 mM | Tris, 2 mM | | | | | |
| Buffer | Arginine, | MgCl2, | | | | | |
| | 2 mM | 0.01% | | | | | |
| | MgCl2, | P188 | | | | | |
| | 0.01% | pH 9.0 | | | | | |
| | P188, pH 9.0 | | | | | | |

% VG Yield was determined as (VG in AEX load)/(VG in Affinity Pool); 1M Tris Base, pH 11, and 1M Tris, pH 9 were used for pH adjustment, and employed at dilution factors of less than 1-fold relative to the affinity pool.

In-Line Dilution to Prepare AAV9 Affinity Pools for AEX Chromatography

In-line dilution of AAV9 affinity pools was investigated to generate AEX loads while reducing surface area the vector was exposed to and reducing the amount of time the vector was exposed to said surfaces. The in-line dilution apparatus is shown in FIG. 5.

Three pieces of platinum cured silicone tubing were joined together by a Y-connector. A peristaltic pump delivered 100 mM Tris, pH 9 to the Y-connector at flow rate of 3.5 mL/min. A second peristaltic pump delivered AAV9 affinity pool to the Y-connector at a flow rate of 0.25 mL/min. The ratio of these flow rates (14 parts diluent to 1-part affinity pool) was selected to achieve an 15-fold dilution and enable direct comparison to similar dilution factors (Table 9 and Table 12). The joined fluids passed through platinum cured silicone tubing with an inner diameter of 0.16 cm and a length of 100 cm. The mixing tube dimensions were designed based on buffer mixing studies that showed these conditions achieved a stable, well blended solution.

The in-line mixed solution was collected, neutralized with 250 mM sodium citrate, pH 3.5, and analyzed by qPCR of the ITRs. 79% of the of the VG pumped into the apparatus was recovered at the outlet of the mixing tubing. While this result represented a slight improvement in % VG yield compared to batch dilution experiments, the yield was still lower than desired. Therefore, further AEX load preparation experiments were carried out via tangential flow filtration.

Tangential Flow Filtration (TFF) to Prepare AAV9 Affinity Pools for AEX Chromatography

In order to avoid VG losses incurred during AEX load preparation, TFF was used to keep the VG concentration high and temporarily incorporate arginine into the vector containing solution. Consistent with Table 10, two TFF runs were carried out with fresh 20 cm² mPES hollow fiber membranes. The membrane was equilibrated, loaded with AAV9 affinity pool, and diafiltered against 150 mM acetate, 100 mM glycine, 25 mM MgCl₂, pH 4.2, the same buffer of the AAV9 affinity pool. At the end of each step, the TFF system was paused and the retentate vessel was sampled. Samples were analyzed for VG titer by qPCR of the ITRs and results are shown in Table 9 and Table 10.

In run #1, diafiltration directly from the affinity elution pool analog buffer into 100 mM Tris, 2 mM MgCl₂, 0.01% P188, pH 9 resulted in a 66% VG yield. In run #2, addition of 500 mM arginine (a co-solvent known to reduce protein aggregation (Arakawa et al. Biophysical Chemistry (2007) 127(1): 1-8)) into diafiltration buffer 2 improved the VG yield to 93% compared to the 66% yield obtained in the absence of arginine. However, removal of arginine from the system in run #2 incurred significant losses and a VG yield of 78%. 500 mM arginine was not included in the final diafiltration buffer because it would generate AEX loads with high conductivity (˜20 mS/cm), which would interfere with binding to AEX resins. However, this finding encouraged the study of other amino acid cosolvents that can stabilize AAV9, but with lower solvent conductivities at alkaline pH. These studies are described in Example 6.

TABLE 10
Tangential flow filtration to prepare AAV9 affinity eluates for AEX chromatography.

| | | Membrane Challenge: | | | |
|---|---|---|---|---|---|
| Membrane: 300 kDa | | 1.2 × 10¹⁴ VG/cm² | Transmembrane Pressure: | | |
| mPES Hollowfiber, 20 cm² | | Permeate Flux: | 5 PSI | | |
| Inlet Pressure: 5-10 PSI | | 75 liters/m²/hr | Diafiltration | Precip- | % VG |
| Run | Step | Buffer | Volumes | itation? | Yield |
| TFF | Membrane | 150 mM Acetate, 100 mM | 2.0 | N/A | N/A |
| #1 | Equilibration | Glycine, 25 mM MgCl₂, pH 4.2 | | | |
| | Sample Load | Affinity Eluate | N/A | no | 100% |
| | Diafiltration 1 | 150 mM Acetate, 100 mM | 2.0 | no | 80% |
| | | Glycine, 25 mM MgCl₂, pH 4.2 | | | |
| | Diafiltration 2 | 100 mM Tris, 2 mM MgCl₂, | 2.5 | yes | 66% |
| | | 0.01% P188 pH 9.0 | | | |
| | Retentate 0.2 | N/A | N/A | N/A | 65% |
| | um Filtration | | | | |
| #2) | Membrane | 150 mM Acetate, 100 mM | 2.0 | N/A | N/A |
| TFF | Equilibration | Glycine, 25 mM MgCl₂, pH 4.2 | | | |
| with | Sample Load | Affinity Eluate | N/A | no | 100% |
| Arginine | Diafiltration 1 | 150 mM Acetate, 100 mM | 2.0 | no | 95% |
| Buffer | | Glycine, 25 mM MgCl₂, pH 4.2 | | | |
| | Diafiltration 2 | 100 mM Tris, 500 mM Arginine, | 1.5 | no | 93% |
| | | 2 mM MgCl₂, 0.01% P188, pH 9.0 | | | |
| | Diafiltration 3 | 100 mM Tris, 2 mM MgCl₂, | 2.5 | yes | 78% |
| | | 0.01% P188 pH 9.0 | | | |
| | Retentate 0.2 | N/A | N/A | N/A | 78% |
| | um Filtration | | | | |

Dynamic Light Scattering Analysis of AAV9 Affinity Pools Upon Dilution with 100 mM Tris, pH 9

Dynamic light scattering (DLS) was used to measure the Z-average (Z-AVG) as an estimate of capsid aggregation in AAV9 affinity pools diluted with 100 mM Tris, pH 9. The Z-average is a reliable measure of the average size of particles in solution. As shown in Table 11, AAV9 affinity pools were diluted (0 to 30-fold) with 100 mM Tris, pH 9 in polypropylene tubes and immediately analyzed by DLS. For each dilution factor, a separate experiment was carried out with fresh AAV9 affinity pool in a new polypropylene tube. Once DLS analysis was complete the solution was measured for pH and conductivity.

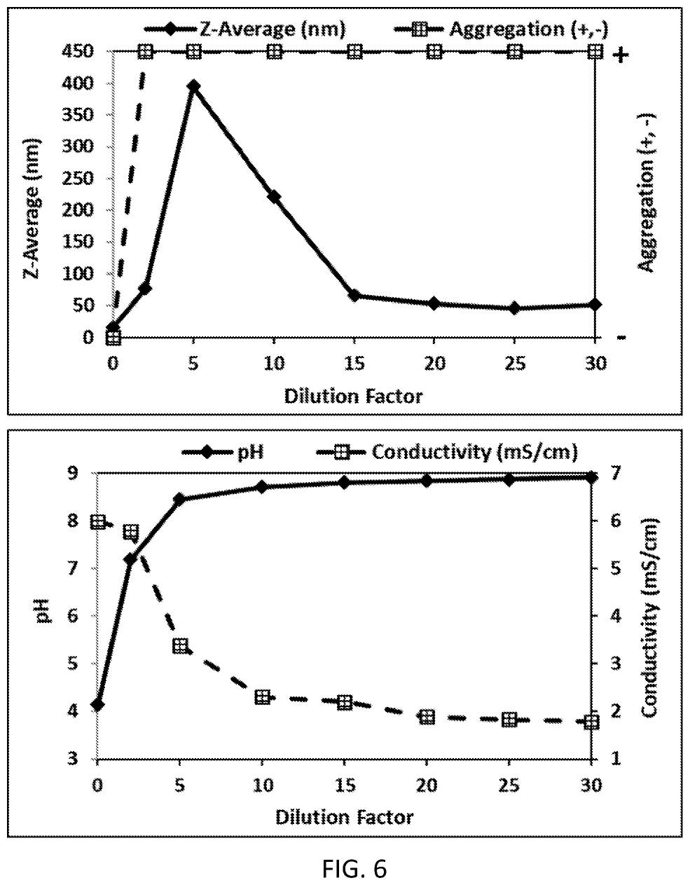

The results are summarized in Table 11, graphed in FIG. 6, and show that dilution of AAV9 affinity pools led to aggregation and an increased Z-average. The undiluted AAV9 affinity pool had a pH of 4.1, conductivity of 6.0 mS/cm, a Z-average of 15 nm, and no aggregation. Two-fold dilution with 100 mM Tris, pH 9 increased solution pH to 7.2, maintained conductivity at 5.8 mS/cm, led to a 5-fold increase in aggregation and a 77 nm Z-average, compared to the AAV9 affinity pool. This result implied that raising solution pH through the estimated AAV9 isoelectric point ((calculated to be ˜5.8 and ˜6.2 for full and empty AAV9 capsids, respectively (Venkatakrishnan et al. (2013) J. Virology 87(9):4974-4984)) destabilized the vector, leading to product loss. Dilution factors of 5- and 10-fold led to aggregation and very high Z-averages of 395 nm, and 221 nm, respectively. Larger dilution factors in the range of 15- to 30-fold displayed Z-averages of 46-66 nm and aggregation.

Collectively, the results given in Table 9 and Table 10 revealed that high amounts of AAV9 vector were lost during AEX load preparation. Load preparation methods of TFF at high VG concentration, in-line dilution, and dilution in the presence of 0.01% P188 were unable to prevent VG losses. Data in Table 11 demonstrated that the mechanism behind the VG losses was aggregation. Based on this observation, a series of dilution experiments were carried out with diluents that could prevent the aggregation and are described in Example 6.

TABLE 11
pH, conductivity, Z-Average, and Aggregation of AAV9
affinity pool diluted with 100 mM Tris, pH 9.

| Dilution | | Conductivity | Z-Average | |
|---|---|---|---|---|
| Factor | pH | (mS/cm) | (nm) | Aggregation # |
| 0 | 4.1 | 6.0 | 15 | − |
| 2 | 7.2 | 5.8 | 77 | + |
| 5 | 8.5 | 3.4 | 395 | + |
| 10 | 8.7 | 2.3 | 221 | + |
| 15 | 8.8 | 2.2 | 66 | + |
| 20 | 8.9 | 1.9 | 53 | + |
| 25 | 8.9 | 1.8 | 46 | + |
| 30 | 8.9 | 1.8 | 52 | + |

# aggregation present (+) or aggregation not present (−)

### Example 4: Screening of Diluent Co-Solvents to Prepare AAV9 Affinity Eluates for AEX

AAV9 affinity eluates were diluted 15-fold with various diluent co-solvents to identify conditions that maximized % VG yield during AEX load preparation. The screened cosolvents included detergents, iodixanol, glycerol, magnesium chloride, and amino acids. In order to study the effect of dilution alone, without altering pH or conductivity, AAV9 affinity eluate was diluted with affinity eluate pool buffer, namely 150 mM acetate, 100 mM glycine, 25 mM MgCl₂, pH 4.2. 14 mL of diluent was added to polypropylene tubes, followed by 1 mL of AAV9 affinity eluate. The resulting solution was gently mixed via end-to-end agitation, measured for pH and conductivity, and a pre-filtration sample was taken. The diluted sample was then filtered through a 0.2 μm filter pre-wetted with diluent. Post dilution and post filtration samples were neutralized with 250 mM sodium citrate, pH 3.5. Neutralized samples were analyzed by dynamic light scattering to estimate particle Z-AVG and relative amounts of aggregation and analyzed by qPCR of the ITRs to determine VG titer.

Results of diluent co-solvent screening revealed some cosolvents reduced aggregation, maintained Z-AVG near 30 nm, and increased % VG yield, compared to baseline diluent 100 mM Tris, pH 9 (Table 12). The undiluted AAV9 affinity elution pool had a Z-AVG of 29 nm, with apparently no aggregation. Dilution of the AAV9 affinity pool with affinity eluate pool buffer resulted in no increase in Z-AVG, no aggregation, but only 69% VG yield. This data suggested that aggregation did not occur at the conditions of the affinity pool (pH 4.2, 7 mS/cm), but VG loss occurred via non-specific binding to increased surface area (by dilution).

In agreement with results from Example 3 (above), dilution of the AAV9 affinity pool with 100 mM Tris, pH 9 led to increased Z-AVG, high aggregation, and VG yields of only 59%. Addition of 0.01-1% P188 to 100 mM Tris, pH 9 did not significantly improve performance, with similar Z-AVG and aggregation, compared to the baseline buffer. Dilution of AAV9 affinity eluate with 50 mM arginine, 2 mM MgCl₂, 0.1% P188, 100 mM Tris, pH 9 led to a 33 nm Z-AVG, no aggregation, and ˜80% VG yield, but led to a conductivity of 4.5 mS/cm, which would interfere with binding of AAV9 to AEX resins. Multiple histidine-containing diluents provided desirable results. For example, dilution of AAV9 with 200 mM Histidine, 200 mM Tris, 10 mM MgCl₂, 25% lodixanol, pH 8.8 led to a 99% VG yield. lodixanol is strongly UV active and would interfere with UV readings in chromatography systems, so this and similar buffers were not employed in AEX runs.

Importantly, 15-fold dilution of the AAV9 affinity eluate with 0.5% P188, 200 mM histidine, 200 mM Tris, pH 8.8, followed by filtration through a pre-wetted filter resulted in a 35 nm Z-AVG, and 101% VG yield. The resulting diluted, filtered solution was pH 8.8, with a conductivity of 2.5 mS/cm, conditions that were likely amenable to binding to AEX resins. Therefore, this dilution scheme was optimized in the example that follows.

Studies of AAV2 aggregation involved screening of various cosolvents via a dilution stress test in combination with DLS and found that AAV2 aggregation was prevented by dilution into buffers that contained various salts at ionic strength 200 mM (Fraser et al. Molecular Therapy (2005) 12(1):171-178). Similar approaches to prepare AAV9 for AEX chromatography were not applicable to the present Example because solutions with ionic strengths 200 mM reduced vector binding to AEX resins (data not shown). Interestingly, addition of the amino acids histidine, arginine, or glycine to the diluent did not inhibit AAV2 aggregation (Fraser et al. Molecular Therapy (2005) 12(1):171-178).

In this Example, mixtures of histidine, arginine, and glycine, in combination with MgCl₂, P188, and/or glycerol reduced AAV9 aggregation, but only afforded % VG yields of 69-80% (see diluent results R2, H2, H3 in Table 12). High % VG yields were only achieved when histidine was employed in synergy with detergents P188 and Triton X-100 (see diluent results H7 and H8 in Table 12). These results indicate that high VG yielding diluent cosolvents for AAV capsid (e.g., AAV9) AEX load preparation should contain detergent to reduce non-specific binding to surfaces (e.g. dilution vessel and filter) and employ histidine or similar moieties to modulate charge interactions and/or hydrogen bonding between AAV capsid particles (e.g., AAV9 vector particles).

TABLE 12
Screening of diluent cosolvents for AEX load preparation of AAV9 vector capsids.
AAV9 affinity eluates were diluted 15-fold with diluents, filtered, analyzed
by dynamic light scattering to determine Z-average (Z-AVG) as an estimate
of capsid aggregation, and analyzed by qPCR to determine % VG Yield.

| | | | | | | % VG |
|---|---|---|---|---|---|---|
| | Diluent Buffer Matrix/ | | Cond. | Z-aver. | Aggregation | Yield |
| Code | Cosolvent Subset | pH | (mS/cm) | (nm) | (−, +) | (qPCR) |
| AE | Affinity Eluate | | | | | |
| AE1 | Affinity Elution Pool- No Dilution | 4.2 | 6.0 | 29.1 | − | 100 |
| AE2 | 150 mM Acetate, 100 mM Glycine, 25 | 4.2 | 7.3 | 29.4 | − | 69 |
| | mM MgCl₂, pH 4.2 | | | | | |
| BL | Baseline, 100 mM Tris, pH 9 | | | | | |
| BL1 | 100 mM Tris, pH 9.0 | 8.8 | 2.0 | 74.2 | + | 59 |
| PO | Baseline + P188 | | | | | |
| PO1 | 100 mM Tris, 0.01% P188, pH 9.0 | 8.8 | 2.3 | 54.8 | + | NT |
| PO2 | 100 mM Tris, 0.1% P188, pH 9.0 | 8.8 | 2.2 | 60.8 | + | NT |
| PO3 | 100 mM Tris, 1% P188, pH 9.0 | 8.8 | 2.1 | 87.2 | + | NT |
| R, G | Arginine and Glycine | | | | | |
| R1 | 5 mM Arginine, 2 mM MgCl₂, 0.1% | 8.9 | 2.8 | 68.3 | + | 81 |
| | P188, 100 mM Tris, pH 8.9 | | | | | |
| R2 | 50 mM Arginine, 2 mM MgCl₂, 0.1% | 9.0 | 4.5 | 33.0 | − | 80 |
| | P188, 100 mM Tris, pH 9.0 | | | | | |
| R2 | 500 mM Arginine, 2 mM MgCl₂, 0.1% | 9.1 | 20.6 | 16.2 | + | 88 |
| | P188, 400 mM Tris, pH 9.1 | | | | | |
| G1 | 200 mM Glycine, 5 mM MgCl₂, 200 | 8.9 | 2.0 | N/A | NR | 70 |
| | mM Tris, pH 8.9 | | | | | |
| H | Histidine | | | | | |
| H1 | 200 mM Histidine, 200 mM Tris, pH | 8.9 | 2.1 | 31.3 | + | NT |
| | 8.9 | | | | | |
| H2 | 200 mM Histidine, 200 mM Tris, 5 mM | | | | | |
| H2 | 200 mM Histidine, 200 mM Tris, 5 mM | 8.9 | 2.6 | 30.4 | − | 69 |
| | MgCl₂, pH 8.9 | | | | | |
| H3 | 200 mM Histidine, 200 mM Tris, 5 mM | 8.9 | 2.3 | 31.8 | − | 74 |
| | MgCl₂, 5% Glycerol, pH 8.9 | | | | | |
| H4 | 200 mM Histidine, 250 mM Tris, 10 | 8.9 | 1.8 | 19.6 | + | 76 |
| | mM MgCl₂, 25% Glycerol, pH 8.9 | | | | | |
| H5 | 200 mM Histidine, 200 mM Tris, 5 mM | 8.8 | 2.4 | 12.9 | + | 74 |
| | MgCl₂, 5% Iodixanol, pH 8.8 | | | | | |
| H6 | 200 mM Histidine, 200 mM Tris, 10 | 8.8 | 2.5 | 3.5 | NR | 99 |
| | mM MgCl₂, 25% Iodixanol, pH 8.8 | | | | | |
| H7 | 200 mM Histidine, 200 mM Tris, 0.5% | 8.8 | 2.5 | 6.5 | NR | 105 |
| | Triton X-100, pH 8.9 | | | | | |
| H8 | 200 mM Histidine, 200 mM Tris, 0.5% | 8.8 | 2.5 | 35.0 | NR | 101 |
| | P188, pH 8.8 | | | | | |

NR—value not reported due to interference in DLS readout.
NT—experiment not conducted.

### Example 5: Optimization of Diluent Co-Solvents to Prepare Recombinant AAV9 Vector Affinity Eluates for AEX Chromatography

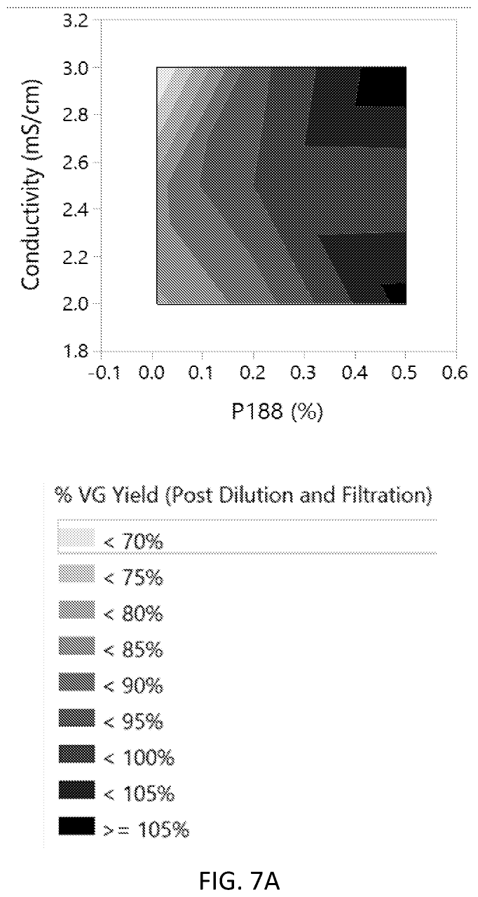

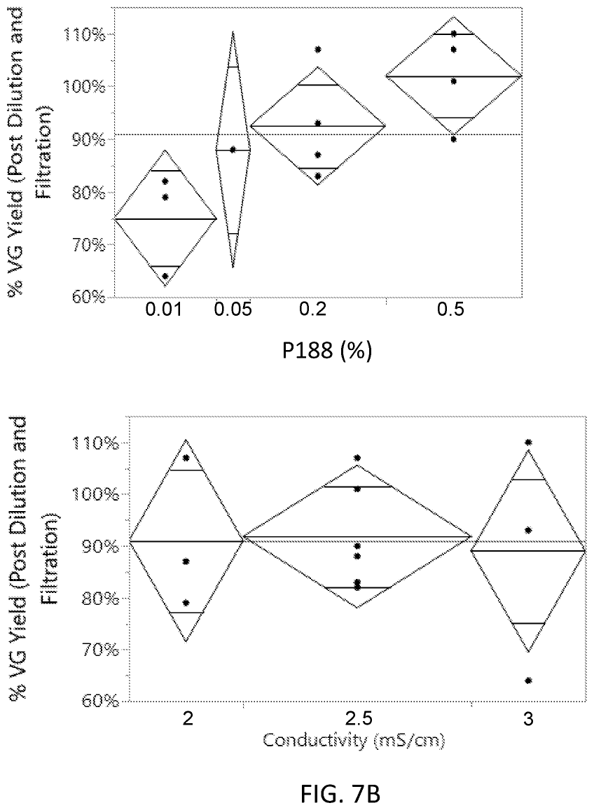

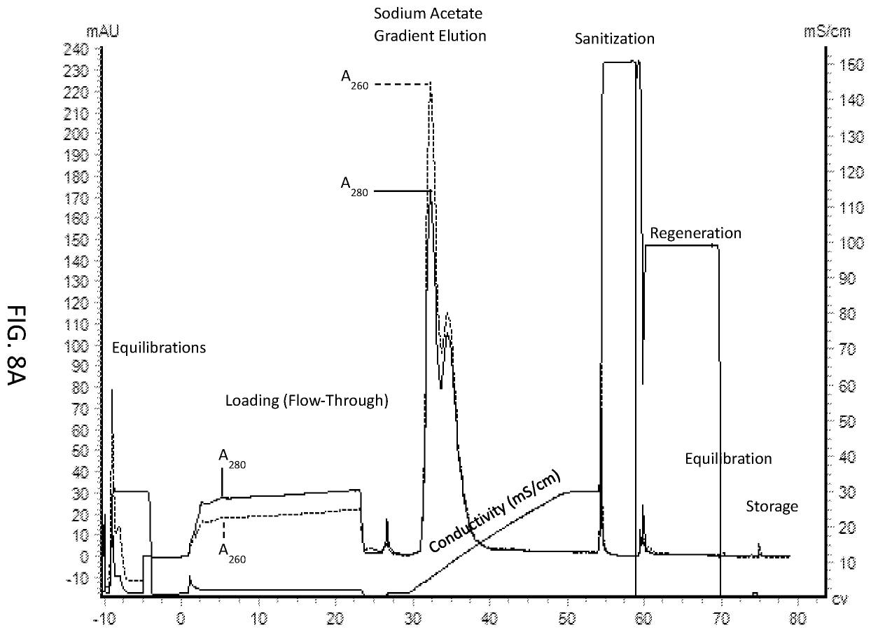

The concentration of P188 in the diluent, and the resulting conductivity of the diluted sample were optimized to achieve maximum recovery of AAV9 vector. Diluent P188 concentrations of 0.01%, 0.05%, 0.2%, and 0.5% were tested in concert with diluted sample conductivities of 2, 2.5, and 3 mS/cm. In order to achieve the varying conductivities, varying dilution factors were employed. Diluted AAV9 vector affinity eluates were passed through 0.2 μm filters that were pre-wetted with each corresponding diluent. The resulting filtrates were assayed by qPCR of the transgene to determine VG titer, and results are shown in Table 13 and FIG. 7A and FIG. 7B. The data shown in FIG. 7A and FIG. 7B demonstrates that 0.5% P188 is required to achieve maximal VG yields and that use of lower concentrations (i.e., less than 0.5%) of P188 lead to lower VG yields. Neither final conductivity (or by association, dilution factor) significantly influenced the % VG yield within the ranges studied.

TABLE 13
Optimization of P188 concentration, dilution
factor, and final conductivity.

| | | | % VG Yield |
|---|---|---|---|
| | Conductivity | Dilution | Post Dilution |
| P188 (%) | (mS/cm) | Factor | and Filtration |
| 0.2 | 3.0 | 5x | 93% |
| 0.2 | 2.5 | 9x | 107% |
| 0.01 | 3.0 | 5x | 64% |
| 0.05 | 2.5 | 9x | 88% |
| 0.5 | 2.0 | 25x | 107% |
| 0.01 | 2.0 | 25x | 79% |
| 0.2 | 2.5 | 9x | 83% |
| 0.2 | 2.0 | 25x | 87% |
| 0.5 | 3.0 | 5x | 110% |
| 0.5 | 2.5 | 9x | 90% |
| 0.01 | 2.5 | 9x | 82% |
| 0.5 | 2.5 | 9x | 101% |

### Example 6: Enrichment of Full AAV9 Via Optimized Dilution and AEX Chromatography Techniques

The top performing diluent from Examples 4 and 5 was combined with the top performing elution salt from Example 1 to form an optimized AEX chromatography method capable of enriching for full AAV9 vector capsids with high % VG yields.

AAV9 affinity eluate was diluted 15-fold with a novel buffer comprising an amino acid and detergent cosolvent (200 mM Histidine, 200 mM Tris, 0.5% P188, pH 8.9) and filtered with a 0.2 μm filter. The 15-fold dilution is lower than the dilution used in other methods (see, US 2019-0002841; US 2019-0002842; US 2018-0002844), is easier to implement in large scale manufacturing and results in high VG yields.

The resulting filtrate was pH 8.8, with a conductivity of 2.3 mS/cm. Consistent with Table 14, the filtrate was loaded onto a POROS™ 50 HQ column, eluted via a sodium acetate gradient, and fractions equivalent to 0.39 of a CV were collected during the gradient elution.

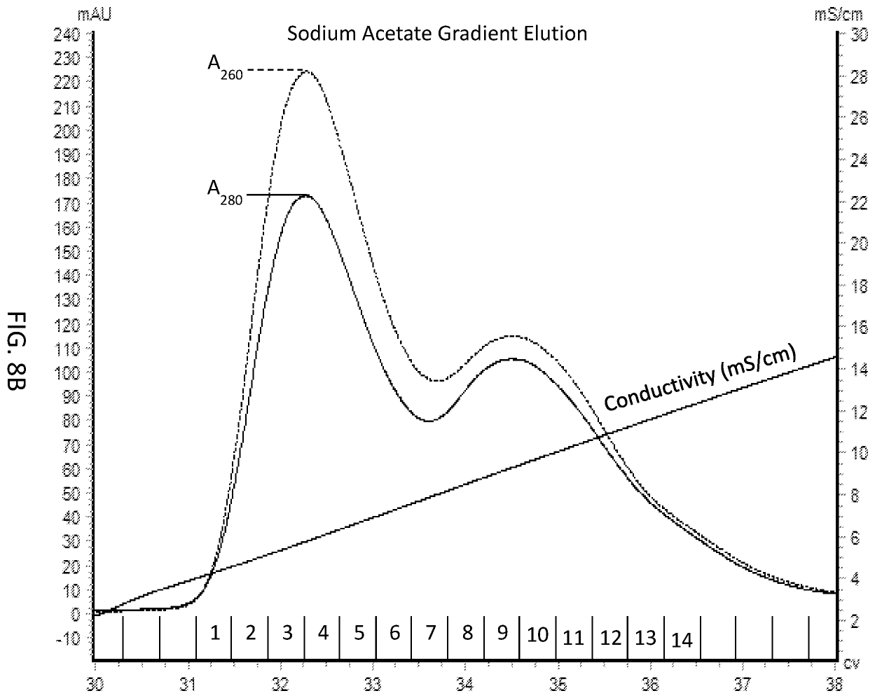

Load and chromatographic fractions were neutralized with 250 mM sodium citrate, pH 3.5, and assayed by SEC A₂₆₀/A₂₈₀, AUC, and qPCR of the ITRs. To test the reproducibility of the AEX process, a second run was carried out using the same materials and methods of the first run. The AEX chromatogram of the first run is depicted in FIG. 8A and FIG. 8B. SEC A₂₆₀/A₂₈₀ of the gradient elution fractions is provided in Table 15 and shows that the optimized AEX method enriched for full AAV9 vector as compared to the load. The percentage of full, intermediate and empty capsid in the affinity pool (which was loaded on the column), the flow-through fraction and elution pools was determined using analytical ultracentrifugation. The data is provided in Table 16 and shows that the optimized AEX method enriched for the percentage of full AAV9 vector capsid while affording high % VG yield.

TABLE 14
Optimized AEX chromatography method performed on a 5.1 ml POROS ™ 50 HQ column.

| Column Bed | | Load Challenge: | | |
|---|---|---|---|---|
| Height: 15 cm | | 1.7 × 10¹⁴ VG/mL of Resin (run 1); | | |
| Column | Column Cross Sectional | or 7.5 × 10¹³ VG/mL of Resin (run 2) | | |
| Diameter: 0.66 cm | Area: 0.34 cm² Column | | Residence | |
| Step | Volume = 5.1 mL | | Time | Flow Rate |
| Description | Solution Description | CV | (min/CV) | (mL/min) |
| Equilibration 1 | 500 mM sodium acetate, 100 mM | 5 | 4 | 1.28 |
| | Tris, 0.01% P188, pH 8.9 | | | |
| Equilibration 2 | 0.5% P188, Tris, pH 9.0 (run 1) or | 5 | 4 | 1.28 |
| | 200 mM Histidine, 200 mM Tris, 0.5% | | | |
| | P188, pH 8.8 (run 2) | | | |
| Sample Loading | Affinity Eluate, diluted 15X with 0.5% | N/A | 4 | 1.28 |
| | P188, 200 mM Histidine, 200 mM Tris | | | |
| | pH 8.8; filtered through 0.2 μm filter | | | |
| Equilibration 3 | 100 mM Tris, 0.01% P188, pH 8.9 | 5 | 4 | 1.28 |
| Sodium Acetate | A - 100 mM Tris, 0.01% P188, pH 8.9 | 20 | 11 | 0.47 |
| Gradient Elution | B - 500 mM NaAcetate, 100 mM Tris, | | | |
| | 0.01% P188, pH 8.9 | | | |
| | Gradient is run from 0-100% B | | | |
| | over 20 CV | | | |
| | Collect 2.0 mL fractions (0.39 of a | | | |
| | CV) throughout elution | | | |
| Gradient Hold | 500 mM sodium acetate, 100 mM | 5 | 11 | 0.47 |
| | Tris, 0.01% P188, pH 8.9 | | | |
| Sanitization | 0.5M NaOH | 5 | 11 | 0.47 |
| Regeneration | 2M NaCl, 100 mM Tris pH 9 | 5 | 11 | 0.47 |
| Equilibration 4 | 100 mM Tris, pH 9 | 5 | 11 | 0.47 |
| Storage | 17% Ethanol | 5 | 11 | 0.47 |

TABLE 15
SEC A₂₆₀/A₂₈₀ analysis of fractions from two
replicates of the optimized AEX process.

| Run | Load | F/T | 1 | 2 | 3 | 4 | 5 | 6 | 7 | 8 | 9 | 10 |
|---|---|---|---|---|---|---|---|---|---|---|---|---|
| 1 | 0.98 | 0.70 | 1.23 | 1.28 | 1.30 | 1.31 | 1.31 | 1.28 | 1.24 | 1.17 | 1.12 | 1.06 |
| 2 | 0.97 | 0.64 | 1.23 | 1.28 | 1.29 | 1.30 | 1.28 | 1.25 | 1.20 | 1.14 | 1.09 | 1.03 |

| Run | Load | F/T | 11 | 12 | 13 | 14 |
|---|---|---|---|---|---|---|
| 1 | 0.98 | 0.70 | 1.04 | 1.01 | 0.96 | 0.92 |
| 2 | 0.97 | 0.64 | 0.96 | 0.93 | 0.85 | 0.86 |

F/T: flow-through (unbound fraction); Gradient elution fractions are numbered 1-14.

TABLE 16
Performance of the optimized AEX method as judged by analysis
of process intermediates and chromatographic fractions.

| | | | | Analytical Ultracentrifugation | | |
|---|---|---|---|---|---|---|
| | | % VG | A₂₆₀/A₂₈₀ | (AUC) | | |
| | | Yield | (HPLC- | | % | |
| | Sample Name | (qPCR) | SEC) | % Full | Intermediate | % Empty |
| | Affinity Pool | 100% | 0.99 | 18% | 15% | 67% |
| Run 1 | Diluted Affinity Pool | 87% | 0.98 | — | — | — |
| | Filtered AEX Load | 93% | 0.98 | — | — | — |
| | Flow-Through | 10% | 0.70 | 1% | 14% | 85% |
| | Broad Pool, Fractions | 53% | 1.27 | 47% | 24% | 29% |
| | 1-8 | | | | | |
| | Narrow Pool, Fractions | 36% | 1.29 | 55% | 24% | 21% |
| | 2-6 | | | | | |
| | Second Peak, | 20% | 1.07 | 28% | 10% | 63% |
| | Fractions 8-13 | | | | | |
| Run 2 | Broad Pool, Fractions | 52% | 1.25 | — | — | — |
| | 1-8 | | | | | |
| | Narrow Pool, Fractions | 39% | 1.28 | 50% | 22% | 28% |
| | 2-6 | | | | | |
| | Second Peak, | 11% | 1.03 | — | — | — |
| | Fractions 8-13 | | | | | |

% VG yields were calculated as (VG amount at each step or fraction)/(VG amount in the Affinity Pool), and thus, accounts for losses upon load preparation. A dash (—) indicates assay not performed.

Dilution and filtration of the AAV9 affinity eluate resulted in a 93% VG yield, providing confirmation of results in Examples 3 and 4. The flow-through fraction contained 10% of the VG from the affinity pool starting material, had an SEC A₂₆₀/A₂₈₀ of 0.70, and had a capsid population of 1% full, 14% intermediate, and 85% empty. This result indicates the developed AEX method partitions some empty capsids through the column, facilitating further enrichment by sodium acetate gradient elution.

For both AEX runs, three virtual elution pools were formed, namely a broad pool that consisted of fractions 1-8, a narrow pool comprised of fractions 2-6, and a second peak pool made from fractions 8-13. The second peak pool had a 20% VG yield and was comprised of mostly empty capsids. The broad pool contained 53% VG yield, had an average SEC A₂₆₀/A₂₈₀ of 1.26, and 47% full AAV9 vector capsids, thus representing a 2.6-fold enrichment in % full. The narrow elution pools contained an average of 38% VG yield, a 1.28-1.29 SEC A₂₆₀/A₂₈₀ ratio, and an average AAV9 vector capsid population of 53% full, 23% intermediate and 24% empty. Thus, the optimized AEX method enriched for full AAV9 vector 2.9-fold, and depleted empty capsids 2.8-fold.

Results obtained from the narrow pool represented a desirable balance between % full and % VG yield. Therefore, to obtain similar results in the majority of examples that follow, we adopted a fraction pooling threshold based on SEC A₂₆₀/A₂₈₀≥1.25 (similar to the 1.25 minimum value obtained in narrow pool fractions 2-6, Table 16). Thus, in the 8 out of 10 large scale AEX runs in the examples that follow, fractions with SEC A₂₆₀/A₂₈₀≥1.25 were included in pools and all fractions with SEC A₂₆₀/A₂₈₀≤1.24 were excluded.

The data and strategy above illustrate a powerful characteristic of the optimized AEX method: flexibility in pooling strategy. Utilizing high resolution gradient elution chromatography in coordination with SEC A₂₆₀/A₂₈₀ analysis of collected fractions ensures high % full of the recovered product, regardless of slight changes in feed stream or process operation.

In contrast to other chromatographic methods developed for the purification of recombinant AAV vectors and in particular, the separation of empty capsids from full AAV vectors (US 2019-0002841; US 2019-0002842; US 2018-0002844), the present disclosed method utilizes a lower dilution factor, a dilution buffer that contains histidine, a steeper elution gradient, NaAcetate as the elution salt and less alkaline conditions (pH 9). The method disclosed herein, and exemplified in Examples 1-9, also differs from other reported methods, such as those of Tomono et al. (Molec. Ther. Meth. Clin. Dev. (2018) 11:180-190), in that the disclosed methods do not utilize an AEX load preparation with a conductivity of about 7 mS/cm, do not utilize ammonium sulfate precipitation and do not utilize size exclusion chromatography. The novel and inventive methods disclosed herein can be implemented at a large scale and produce a high yield of VG from an affinity chromatography eluate.

To further illustrate the flexibility and robustness of the optimized AEX method, Example 7 tested feed streams with varying % full vector capsid.

### Example 7: Effect of the Percentage of Full Capsids in an Affinity Eluate on Performance of the Optimized AEX Process

The optimized AEX method was tested for its ability to enrich for full AAV9 vectors from feed materials with varying percentages of AAV9 empty capsids. Briefly, HEK293 cells were grown in suspension culture and transfected with an adenoviral helper plasmid and a Rep2Cap9 plasmid (a plasmid comprising the transgene cassette was not included). Cells were cultured for three days post transfection, harvested, lysed, flocculated, depth filtered and absolute filtered. Affinity chromatography was performed on the resulting filtrate to generate an affinity pool containing AAV9 particles that did not contain a vector genome (null transfection AAV9 affinity eluate, referred to herein as null capsids). In order to generate AEX starting materials with varying percentages of full capsids, null transfection AAV9 affinity eluate was mixed with a standard AAV9 affinity eluate at volumetric ratios of 0%, 20%, 40%, 60%, 80%, and 100% null capsids.

The mixtures were diluted 15-fold with 200 mM histidine, 200 mM Tris, 0.5% P188, pH 8.8, and 0.2 μm filtered to generate AEX loads that were pH 8.8, with a conductivity of 2.6 mS/cm. Consistent with Table 17, the optimized AEX method was performed on the 6 loads that contained varying percentages of null-transfection generated capsids. For each of the 6 AEX runs, a 6.67 mL POROS™ 50 HQ column was uniformly challenged with 1.5×10¹⁵ total viral particles (VP), or 2.2×10¹⁴ VP/mL resin. Chromatographic load, flow-through, and elution fractions were neutralized with 250 mM sodium citrate, pH 3.5, and analyzed by SEC A₂₆₀/A₂₈₀.

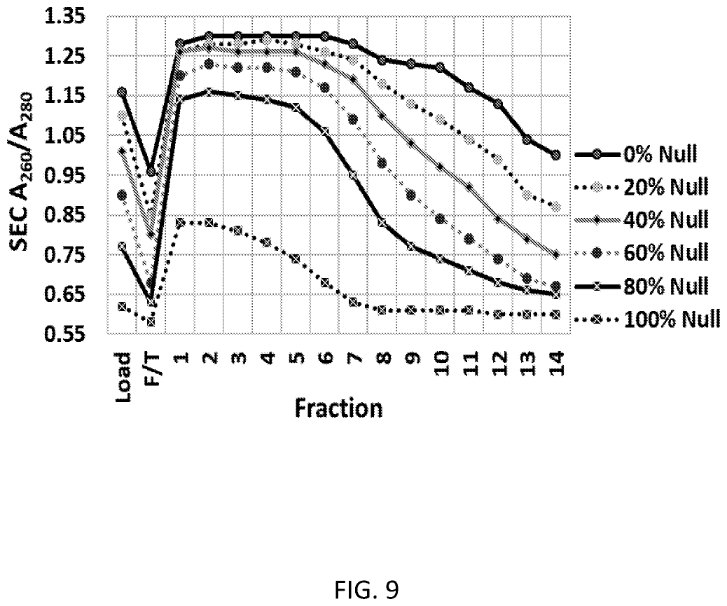

SEC A₂₆₀/A₂₈₀ data is reported in Table 18 and FIG. 9, and show that the optimized AEX method generated individual elution fractions with SEC A₂₆₀/A₂₈₀≥1.25 when 0%, 20%, and 40% null capsid transfection starting materials were employed. Column loads that contained 0%, 20%, and 40% null capsid had respective SEC A₂₆₀/A₂₈₀ values of 1.16, 1.10, and 1.01. The optimized AEX method used these materials to generate 6 or 7 contiguous elution fractions each with SEC A₂₆₀/A₂₈₀≥1.25.

Loads containing 60%, 80%, and 100% null capsid had respective SEC A₂₆₀/A₂₈₀ values of 0.90, 0.77, and 0.62. The optimized AEX method enriched starting materials with 60%, 80%, and 100% null capsid to generate elution fractions with maximum SEC A₂₆₀/A₂₈₀ values of 1.23, 1.16, and 0.83, respectively.

The AAV9 upstream process, implemented at the 250 L and 2000 L single use bioreactor (SUB) scale, combined with downstream operations of harvest and affinity chromatography produced AAV9 affinity eluate with SEC A₂₆₀/A₂₈₀ of 1.10±0.1, (n=7). Therefore, upstream and downstream processing (including the optimized AEX process described here) generated AAV9 capsids with a % full that is useful for gene therapy applications. Based on these results, the optimized AEX process was scaled up to enable manufacturing at the 250 L and 2000 L SUB scale, described in the example that follows.

TABLE 17
Optimized AEX chromatography method performed with a POROS ™ 50
HQ column on starting materials with a varying % of full capsids. All runs
involved uniformly challenging columns at 2.2 × 10¹⁴ VP/mL resin
with a varying % of null capsid transfection starting material.

Column Bed

| Height: 19.5 cm | | Load Challenge: | | |
|---|---|---|---|---|
| Column | Column Cross Sectional | 2.2 × 10¹⁴ VP/mL of Resin | | |
| Diameter: 0.66 cm | Area: 0.34 cm² | | Residence | |
| Step | Column Volume = 6.67 mL | | Time | Flow Rate |
| Description | Solution Description | CV | (min/CV) | (mL/min) |
| Sanitization | 0.5M NaOH | 7.5 | 4 | 1.67 |
| Regeneration | 2M NaCl, 100 mM Tris pH 9 | 5 | 4 | 1.67 |
| Equilibration 1 | 100 mM Tris, pH 9 | 5 | 4 | 1.67 |
| Equilibration 2 | 500 mM sodium acetate, 100 mM Tris, | 5 | 4 | 1.67 |
| | 0.01% P188, pH 8.9 | | | |
| Equilibration 3 | 200 mM Histidine, 200 mM Tris, 0.5% | 5 | 4 | 1.67 |
| | P188, pH 8.8 | | | |
| Sample Loading | Affinity Eluate^{N}, diluted 15X with 0.5% | 10 | 4 | 1.67 |
| | P188, 200 mM Histidine, 200 mM Tris | | | |
| | pH 8.8; 0.2 μm filtered | | | |
| Equilibration 4 | 100 mM Tris, 0.01% P188, pH 8.9 | 5 | 4 | 1.67 |
| Sodium Acetate | A - 100 mM Tris, 0.01% P188, pH 8.9 | 20 | 4 | 1.67 |
| Gradient Elution | B - 500 mM NaAcetate, 100 mM Tris, | | | |
| | 0.01% P188, pH 8.9 | | | |
| | Gradient is run from 0-100% B over | | | |
| | 20 CV | | | |
| | Collect fractions of ⅓ CV starting at | | | |
| | A₂₈₀ ≥10 mAU, and ending at A₂₈₀ <10 | | | |
| | mAU* | | | |
| Gradient Hold | 500 mM NaAcetate, 100 mM Tris, 0.01% | 5 | 4 | 1.67 |
| | P188, pH 8.9 | | | |
| Sanitization | 0.5M NaOH | 7.5 | 4 | 1.67 |
| Regeneration | 2M NaCl, 100 mM Tris pH 9 | 5 | 4 | 1.67 |
| Equilibration 5 | 100 mM Tris, pH 9 | 5 | 4 | 1.67 |
| Storage | 17% Ethanol | 5 | 4 | 1.67 |

*A 2 mm path length UV monitor was employed.
NMixture of standard AAV9 affinity eluate and null capsid transfection AAV9 affinity eluate at volumetric ratios of 0%, 20%, 40%, 60%, 80%, and 100% Null capsids.

TABLE 18
SEC A₂₆₀/A₂₈₀ of chromatographic fractions generated using
the optimized AEX method performed on feed materials with
varying mixtures of % Null and a standard affinity pool.

| Mixture | | Load | F/T | 1 | 2 | 3 | 4 | 5 | 6 | 7 | 8 | 9 | 10 |
|---|---|---|---|---|---|---|---|---|---|---|---|---|---|
| 100% | Null | 0.62 | 0.58 | 0.83 | 0.83 | 0.81 | 0.78 | 0.74 | 0.68 | 0.63 | 0.61 | 0.61 | 0.61 |
| 80% | Null | 0.77 | 0.63 | 1.14 | 1.16 | 1.15 | 1.14 | 1.12 | 1.06 | 0.95 | 0.83 | 0.77 | 0.74 |
| 60% | Null | 0.90 | 0.68 | 1.20 | 1.23 | 1.22 | 1.22 | 1.21 | 1.17 | 1.09 | 0.98 | 0.90 | 0.84 |
| 40% | Null | 1.01 | 0.80 | 1.26 | 1.27 | 1.26 | 1.26 | 1.26 | 1.23 | 1.19 | 1.10 | 1.03 | 0.97 |
| 20% | Null | 1.10 | 0.85 | 1.26 | 1.28 | 1.28 | 1.29 | 1.28 | 1.26 | 1.24 | 1.18 | 1.13 | 1.09 |
| 0% | Null | 1.16 | 0.96 | 1.28 | 1.30 | 1.30 | 1.30 | 1.30 | 1.30 | 1.28 | 1.24 | 1.23 | 1.22 |

| | | | | | | |
|---|---|---|---|---|---|---|
| Mixture | Load | F/T | 11 | 12 | 13 | 14 |

| | | | | | | | |
|---|---|---|---|---|---|---|---|
| 100% | Null | 0.62 | 0.58 | 0.61 | 0.60 | 0.60 | 0.60 |
| 80% | Null | 0.77 | 0.63 | 0.71 | 0.68 | 0.66 | 0.65 |
| 60% | Null | 0.90 | 0.68 | 0.79 | 0.74 | 0.69 | 0.67 |
| 40% | Null | 1.01 | 0.80 | 0.92 | 0.84 | 0.79 | 0.75 |
| 20% | Null | 1.10 | 0.85 | 1.04 | 0.99 | 0.90 | 0.87 |
| 0% | Null | 1.16 | 0.96 | 1.17 | 1.13 | 1.04 | 1.00 |

F/T: flow through (unbound) fraction. Elution fractions are given as numbers.
Ratios ≥ 1.25 are bolded.

### Example 8: Scale-Up of Optimized AEX Process for Enrichment of Full AAV9 Vectors

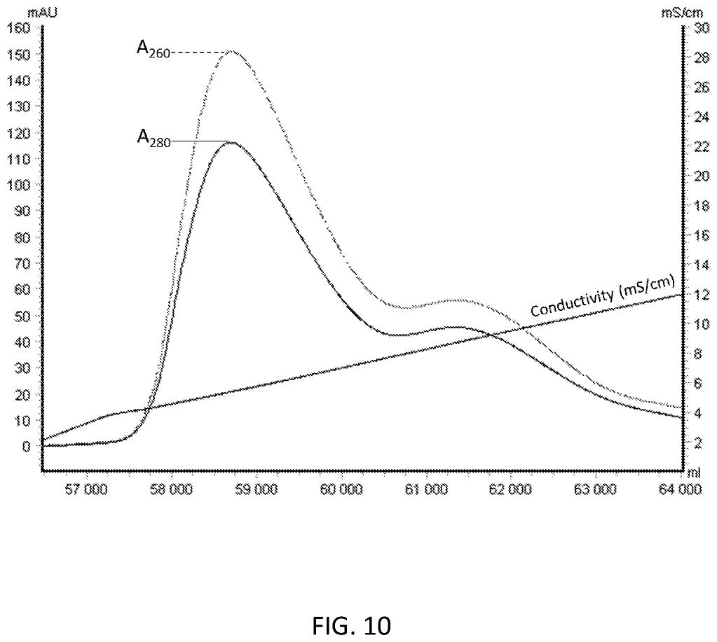

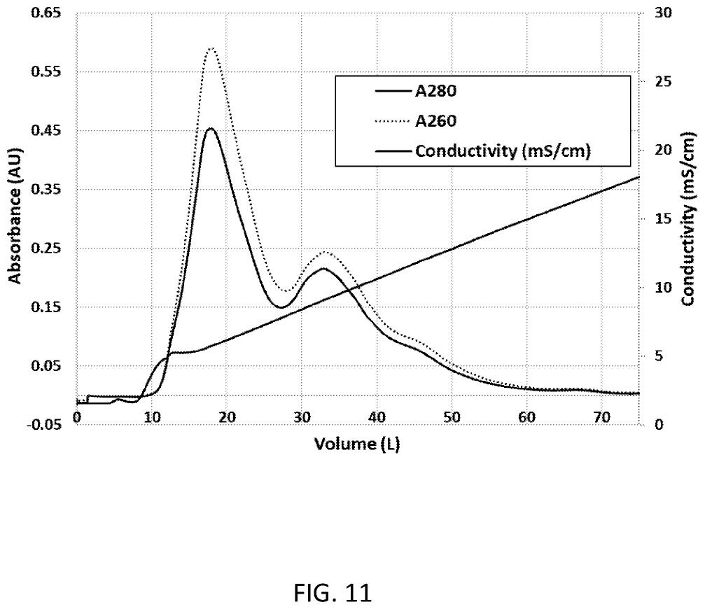

An optimized AEX method was scaled-up to enrich for full AAV9 vectors for downstream processing of AAV vectors produced in 250 L and 2000 L single use bioreactors (SUBs). POROS™ 50 HQ columns were sized based on a scale-independent maximum challenge of 3×10¹⁴ VG/mL resin. Table 19 and Table 20 provide optimized AEX methods implemented for the 250 L and 2000 L SUB processes, respectively. At the 250 L SUB scale, a 1.3 L AEX column, with a 10 cm inner diameter (ID) and a 16 cm bed height was used. The AEX process was implemented at the 2000 L SUB scale using a 6.4 L AEX column with a 20 cm ID and a 20.5 cm bed height. A residence time of 4±0.5 minutes/CV was fixed across both scales, leading to volumetric flow rates of 314 mL/min and 1.8 L/min for all steps within the AEX processes for the 250 L and 2000 L SUBs, respectively. These flow rates were in acceptable ranges to enable pumps and mixers on chromatography skids to form smoothly shaped linear sodium acetate gradients during product elution. Across both scales, each chromatography step used the same set of buffers for the same CV length with the exception that the 2000 L SUB AEX process included Water For Injection Flushes and pre-use sanitization and regeneration steps. FIG. 10 and FIG. 11 provide chromatograms of representative AEX runs carried out at the 250 L SUB scale and the 2000 L SUB scale. Across all scales tested (various small scale runs, 250 L and 2000 L SUB scales), the optimized AEX process produced similar A260/A280 chromatographic profiles.

At both the 250 L and 2000 L scales, elution fractions sized ⅓^{rd} column volume (CV) were collected. Fractions were neutralized with 250 mM sodium citrate, pH 3.5 and assayed by various analytical techniques. SEC was carried out to determine the A260/A280 ratio and the percentage of high molar mass species (% HMMS). Residual amounts of host cell protein (HCP) and host cell DNA (HC-DNA) were determined via ELISA and qPCR, respectively. At the 250 L scale, qPCR was used to measure ITR copies to quantify the VG. At the 2000 L scale, qPCR was used to measure transgene copies to quantify the VG.

TABLE 19
Optimized AEX chromatography method implemented at 250 L
SUB scale on a 1.3 L POROS ™ 50 HQ column.

Column Bed

| Height: 16 cm | | Load Challenge: | | | |
|---|---|---|---|---|---|
| Column | Column Cross Sectional | 1.7 × 10¹³-5.3 × 10¹³ VG/mL of Resin | | | |
| Diameter: 10 cm | Area: 78.5 cm² | | Residence | Linear | Flow |
| Step | Column Volume = 1256 mL | | Time | Velocity | Rate |
| Description | Solution Description | CV | (min/CV) | (cm/hr) | (mL/min) |
| Equilibration 1 | 500 mM sodium acetate, 100 mM | 5 | 4 | 240 | 314 |
| | Tris, 0.01% P188, pH 8.9 | | | | |
| Equilibration 2 | 200 mM Histidine, 200 mM Tris, 0.5% | 5 | 4 | 240 | 314 |
| | P188, pH 8.8 | | | | |
| Sample | Affinity Eluate, diluted 15X with 0.5% | N/A | 4 | 240 | 314 |
| Loading | P188, 200 mM Histidine, 200 mM Tris | | | | |
| | pH 8.8; filtered through 0.2 μm filter | | | | |
| Equilibration 3 | 100 mM Tris, 0.01% P188, pH 8.9 | 5 | 4 | 240 | 314 |
| Sodium Acetate | A - 100 mM Tris, 0.01% P188, pH 8.9 | 20 | 4 | 240 | 314 |
| Gradient Elution | B - 500 mM sodium acetate, 100 mM | | | | |
| | Tris, 0.01% P188, pH 8.9 | | | | |
| | Run gradient from 0-100% B over | | | | |
| | 20 CV | | | | |
| | Collect ≥10 fractions of ⅓ CV | | | | |
| | starting at A₂₈₀ ≥10 mAU (2 mm path | | | | |
| | length) | | | | |
| Sanitization | 0.5M NaOH | 5 | 4 | 240 | 314 |
| Regeneration | 2M NaCl, 100 mM Tris pH 9 | 5 | 4 | 240 | 314 |
| Equilibration 4 | 100 mM Tris, pH 9 | 5 | 4 | 240 | 314 |
| Storage | 17.5% Ethanol | 3 | 4 | 240 | 314 |

TABLE 20
Optimized AEX chromatography method performed at 2000 L
SUB scale on a 6.4 L POROS ™ 50 HQ column.

Column Bed

| Height: 20.5 cm | | Load Challenge*: | | | |
|---|---|---|---|---|---|
| Column | Column Cross Sectional | 2.7 × 10¹²-6.8 × 10¹³ VG/mL of Resin | | | |
| Diameter: 20 cm | Area: 314 cm² | | Residence | Linear | Flow |
| Step | Column Volume = 6.4 L | | Time | Velocity | Rate |
| Description | Solution Description | CV | (min/CV) | (cm/hr) | (L/min) |
| Pre-Use Flush | Water for Injection (upward flow) | 5 | 3.6 | 300 | 1.8 |
| Sanitization 1 | 0.5M NaOH (upward flow) | 16 | 3.6 | 300 | 1.8 |
| Regeneration 1 | 2M NaCl, 100 mM Tris, pH 9 | 5 | 3.6 | 300 | 1.8 |
| Equilibration 1 | 100 mM Tris, pH 9 | 5 | 3.6 | 300 | 1.8 |
| Equilibration 2 | 500 mM sodium acetate, 100 mM | 5 | 3.6 | 300 | 1.8 |
| | Tris, 0.01% P188, pH 8.9 | | | | |
| Equilibration 3 | 200 mM Histidine, 200 mM Tris, | 5 | 3.6 | 300 | 1.8 |
| | 0.5% P188, pH 8.8 | | | | |
| Product | Affinity Eluate, diluted 15X with 0.5% | N/A | 3.6 | 300 | 1.8 |
| Loading | P188, 200 mM Histidine, 200 mM | | | | |
| | Tris, pH 8.8; filtered through 0.2 μm | | | | |
| | filter (in-line) | | | | |
| Equilibration 4 | 100 mM Tris, 0.01% P188, pH 8.9 | 5 | 3.6 | 300 | 1.8 |
| Sodium Acetate | A - 100 mM Tris, 0.01% P188, pH 8.9 | 20 | 3.6 | 300 | 1.8 |
| Gradient Elution | B - 500 mM sodium acetate, 100 mM | | | | |
| | Tris, 0.01% P188, pH 8.9 | | | | |
| | Gradient is run from 0-100% B | | | | |
| | over 20 CV | | | | |
| | Collect 10 fractions of 1/3 CV | | | | |
| | starting at A₂₈₀ ≥0.5-5 mAU/mm | | | | |
| | path length* | | | | |
| Sanitization 2 | 0.5M NaOH (upward flow) | 16 | 3.6 | 300 | 1.8 |
| Regeneration 2 | 2M NaCl, 100 mM Tris pH 9 | 5 | 3.6 | 300 | 1.8 |
| Equilibration 4 | 100 mM Tris, pH 9 | 5 | 3.6 | 300 | 1.8 |
| Post-use | Water for injection | 5 | 3.6 | 300 | 1.8 |
| flush | | | | | |
| Storage | 17.5% Ethanol | 3 | 3.6 | 300 | 1.8 |

*Resin challenges were determined using a qPCR method that measured transgene copies.

Table 21 provides SEC A260/A280 ratios of elution fractions generated with the 250 L and 2000 L SUB AEX processes, respectively. The process demonstrated robustness to a broad range of column challenge: 2.7×10¹²-6.8×10¹³ VG/mL resin. Nine out of ten AEX runs produced at least 6 fractions with SEC A₂₆₀/A₂₈₀ ratios≥1.25. Batch 250 L-1 used the affinity pool with the lowest SEC ratio in the study (0.94) and was the only AEX run that did not generate fractions with a ratio≥1.25. For each AEX run, fraction 5 yielded the highest SEC A₂₆₀/A₂₈₀ ratio in 7 out of the 10 runs, thus showing high consistency in chromatography and fraction collection operations across two different scales and various VG/mL resin challenges.

Table 22 and Table 23 provide impurity profiles, % HMMS, and SEC A₂₆₀/A₂₈₀ of individual AEX fractions from the 250 L-4 and the 2000 L-4 batches, respectively. The optimized AEX process cleared high amounts of HCP from affinity pools. For instance, the 250 L-4 affinity pool contained 51 pg HCP/1×10⁹ VG, and the optimized AEX process cleared the HCP to LLOQ in elution fractions 2-8, used to form the AEX pool. The 2000 L-4 affinity pool contained 331 pg HCP/1×10⁹ VG that was cleared to LLOQ in elution fractions 2-9, used to form the AEX pool.

The AEX process does not significantly reduce HC-DNA levels. The AEX process, using the sodium acetate elution gradient, resolved HMMS. Early elution fractions were relatively depleted in HMMS (e.g., fractions 1-5 in both 250 L-4 and 2000 L-4 runs contained <3% HMMS). Conversely, later elution fractions contained higher relative levels of HMMS (e.g., fractions 8-10 from the 2000 L-4 run contained >7% HMMS).

TABLE 21
SEC A₂₆₀/A₂₈₀ of affinity eluate pool and AEX chromatography
fractions generated at 250 L and 2000 L SUB scale.

| | | | VG/mL | | | | | | | | | | | |
|---|---|---|---|---|---|---|---|---|---|---|---|---|---|---|
| | Batch | | resin | Affinity | Fraction number | | | | | | | | | |
| Scale | ID | | challenge | Pool | 1 | 2 | 3 | 4 | 5 | 6 | 7 | 8 | 9 | 10 |
| 250 L | 250 | L-1 | 5.3 × 10¹³ | 0.94 | 1.11 | 1.20 | 1.23 | 1.23 | 1.23 | 1.22 | 1.19 | 1.13 | 1.03 | 1.05 |
| SUB; | 250 | L-2 | 4.7 × 10¹³ | 0.99 | 1.20 | 1.26 | 1.27 | 1.27 | 1.27 | 1.26 | 1.27 | 1.18 | 1.14 | 1.18 |
| 1.3 L | 250 | L-3 | 3.0 × 10¹³ | 1.25 | 1.29 | 1.30 | 1.31 | 1.32 | 1.33 | 1.32 | 1.32 | 1.31 | 1.33 | 1.34 |
| AEX | 250 | L-4 | 1.7 × 10¹³ | 1.15 | 1.21 | 1.27 | 1.29 | 1.30 | 1.30 | 1.30 | 1.28 | 1.24 | 1.19 | 1.17 |
| CV | 250 | L-5 | 1.9 × 10¹³ | 1.19 | 1.27 | 1.30 | 1.31 | 1.32 | 1.32 | 1.32 | 1.30 | 1.25 | 1.22 | 1.23 |
| 2000 L | 2000 | L-1 | 2.7 × 10¹² | N/A | 1.20 | 1.25 | 1.27 | 1.27 | 1.28 | 1.27 | 1.31 | 1.22 | 1.18 | 1.14 |
| SUB; | 2000 | L-2 | 1.1 × 10¹³ | N/A | 1.10 | 1.22 | 1.26 | 1.28 | 1.28 | 1.29 | 1.28 | 1.25 | 1.20 | 1.17 |
| 6.4 L | 2000 | L-3 | 2.2 × 10¹³ | 1.06 | 1.06 | 1.23 | 1.28 | 1.30 | 1.31 | 1.31 | 1.31 | 1.28 | 1.24 | 1.19 |
| AEX | 2000 | L-4 | 4.1 × 10¹³ | 1.10 | 1.18 | 1.28 | 1.31 | 1.32 | 1.33 | 1.33 | 1.33 | 1.29 | 1.25 | 1.21 |
| CV | 2000 | L-5 | 6.8 × 10¹³ | 1.04 | 1.11 | 1.26 | 1.31 | 1.32 | 1.33 | 1.33 | 1.32 | 1.29 | 1.22 | 1.15 |

Elution fractions are given as numbers.
Ratios ≥ 1.25 are bolded.
250 L SUB VG/mL resin challenges were determined using a qPCR method that measured ITR copies.
2000 L SUB VG/mL resin challenges were determined using a qPCR method that measured transgene copies.

TABLE 22
SEC A₂₆₀/A₂₈₀, % HMMS, and impurity profile of chromatography fractions
from an AEX run at the 250 L SUB scale (batch 250 L-4).

| | Affinity | | | | | | | | | | |
|---|---|---|---|---|---|---|---|---|---|---|---|
| Metric | Pool | 1 | 2 | 3 | 4 | 5 | 6 | 7 | 8 | 9 | 10 |
| SEC A₂₆₀/A₂₈₀ | 1.15 | 1.21 | 1.27 | 1.29 | 1.30 | 1.30 | 1.30 | 1.28 | 1.24 | 1.19 | 1.17 |
| % HMMS | 0.7 | 0.0 | 0.2 | 0.3 | 0.5 | 0.8 | 1.2 | 2.0 | 2.9 | 3.2 | 2.6 |
| (SEC) | | | | | | | | | | | |
| % Purity | — | 99.2 | 99.4 | 99.3 | 99.3 | 99.3 | 99.2 | 99.1 | 99.1 | 99.3 | 99.2 |
| (RP-HPLC) | | | | | | | | | | | |
| HC-DNA (pg)/ | 4.0 | 1.9 | 5.0 | 5.7 | 3.4 | 1.8 | 1.3 | 1.2 | 1.3 | 1.2 | 0.6 |
| 1 × 10⁹ VG | | | | | | | | | | | |
| HCP (pg)/ | 51.1 | LLoQ | LLoQ | LLoQ | LLoQ | LLoQ | LLoQ | LLoQ | LLoQ | LLoQ | LLoQ |
| 1 × 10⁹ VG | | | | | | | | | | | |

Elution fractions are given as numbers.
Elution fractions 2-8 had SEC A₂₆₀/A₂₈₀ ≥ 1.24 (bolded), and thus were pooled and processed forward.
LLoQ—detected amount was lower than the limit of quantitation of the assay.

TABLE 23
SEC A₂₆₀/A₂₈₀, % HMMS, and impurity profile of chromatography fractions
from an AEX run at the 2000 L SUB scale (batch 2000 L-4).

| | Affinity | | | | | | | | | | |
|---|---|---|---|---|---|---|---|---|---|---|---|
| Metric | Pool | 1 | 2 | 3 | 4 | 5 | 6 | 7 | 8 | 9 | 10 |
| SEC A₂₆₀/A₂₈₀ | 1.10 | 1.18 | 1.28 | 1.31 | 1.32 | 1.33 | 1.33 | 1.33 | 1.29 | 1.25 | 1.21 |
| % HMMS | 0.8 | 0 | 1.2 | 1.5 | 2.4 | 2.8 | 4.2 | 5.5 | 7.7 | 8.3 | 7.7 |
| (SEC) | | | | | | | | | | | |
| % Purity | — | 79.6 | 97.6 | 98.5 | 98.7 | 98.8 | 98.4 | 97.7 | 97.2 | 97.0 | 98.0 |
| (RP-HPLC) | | | | | | | | | | | |
| HC-DNA (pg)/ | 9.1 | 26.5 | 20.2 | 15.0 | 12.0 | 9.3 | 6.5 | 4.7 | 2.7 | 4.0 | 5.8 |
| 1 × 10⁹ VG | | | | | | | | | | | |
| HCP (pg)/ | 331 | LLoQ | LLoQ | LLoQ | LLoQ | LLoQ | LLoQ | LLoQ | LLoQ | LLoQ | LLoQ |
| 1 × 10⁹ VG | | | | | | | | | | | |

Elution fractions are given as numbers.
Elution fractions 2-9 had SEC A₂₆₀/A₂₈₀ ≥ 1.25 (bolded), and thus were pooled and processed forward.
LLoQ—detected amount was lower that the limit of quantitation of the assay.

The 250 L AEX runs used various fraction pooling thresholds based on SEC A₂₆₀/A₂₈₀ ratios. Fractions from 250 L AEX runs 250 L-1 and 250 L-4 were pooled based on where SEC A₂₆₀/A₂₈₀≥1.22 and≥1.24, respectively. Fractions from 250 L AEX runs 250 L-2, 250 L-3, and 250 L-5, and all five 2000 L AEX runs were pooled based on where SEC A₂₆₀/A₂₈₀≥1.25.

Resulting AEX product pools from 2000 L batches 2000 L-2, 2000 L-3, 2000 L-4 and 2000 L-5 were processed through a step of viral filtration while 250 L batches and batch 2000 L-1 were not viral filtered. Resulting AEX pools and/or viral filtration pools were processed through ultrafiltration/diafiltration (UF/DF) and 0.2 μm filtration to generate drug substance (DS). None of the steps after AEX chromatography significantly impacted AAV9 empty/full ratios. qPCR was performed on affinity and AEX pools to determine % VG yield of the AEX step. Drug substance material was analyzed by AUC and SEC A260/A280 to determine % full of the purified AAV9 vector.

Table 24 reports results and shows the scaled-up AEX processes increased % full of the recovered AAV9 vector with high yields. The scaled-up AEX process enriched the % full of vectors to 45-65% of total capsids, and reduced the amount of empty capsids to ≤28% of total capsids in 9 out of 10 drug substance batches, as measured by AUC. In all 10 runs, the AEX process increased SEC A260/A280 ratios in affinity pools from 0.94-1.25 to 1.24-1.32 in drug substance. The average % VG step yield of the AEX process implemented at 250 L and 2000 L scales was 47+/−11%.

TABLE 24
AEX performance at 250 L and 2000 L SUB scale.

| | AEX Process Inputs | | | | | | | | | | |
|---|---|---|---|---|---|---|---|---|---|---|---|
| | | | | Affinity | AEX Process Outputs | | | | | | |
| | | | | Pool | DS¹ Analytical | | | DS¹ | | | |
| | | | VG/mL | SEC | Ultracentrifugation | | | SEC | % VG | % VG | % VG |
| | Batch | | resin | A260/ | % | % | % | A260/ | Dilution | Column | Step |
| | ID | | challenge | A280 | full | inter | empty | A280 | Yield² | Yield³ | Yield⁴ |
| 250 L | 250 | L-1 | 5.3 × 10¹³ | 0.94 | 45 | 19 | 37 | 1.24 | 92 | 40 | 37 |
| SUB; | 250 | L-2 | 4.7 × 10¹³ | 0.99 | 50 | 22 | 28 | 1.27 | 54 | 84 | 45 |
| 1.3 L | 250 | L-3 | 3.0 × 10¹³ | 1.25 | 65 | 25 | 10 | 1.32 | 73 | 82 | 60 |
| AEX | 250 | L-4 | 1.7 × 10¹³ | 1.15 | 55 | 28 | 17 | 1.30 | 41 | 98 | 40 |
| CV | 250 | L-5 | 1.9 × 10¹³ | 1.19 | 62 | 26 | 13 | 1.31 | 41 | 100 | 41 |
| 2000 L | 2000 | L-1 | 2.7 × 10¹²* | N/A | 47 | 35 | 18 | 1.28 | 94 | 43 | 40 |
| SUB; | 2000 | L-2 | 1.1 × 10¹³* | N/A | 45 | 37 | 18 | 1.29 | 154 | 20 | 31 |
| 6.4 L | 2000 | L-3 | 2.2 × 10¹³* | 1.06 | 49 | 31 | 20 | 1.30 | 88 | 61 | 54 |
| AEX | 2000 | L-4 | 4.1 × 10¹³* | 1.10 | 52 | 27 | 21 | 1.31 | 105 | 52 | 55 |
| CV | 2000 | L-5 | 6.8 × 10¹³* | 1.04 | 50 | 28 | 22 | 1.30 | 137 | 48 | 66 |

| | | | | | | | | |
|---|---|---|---|---|---|---|---|---|
| average ± standard | 1.09 ± 0.10 | 52 ± 7 | 28 ± 5 | 20 ± 7 | 1.29 ± 0.02 | 88 ± 36 | 63 ± 26 | 47 ± 11 |
| deviation | | | | | | | | |

¹‘DS’ indicates that AUC and SEC A₂₆₀/A₂₈₀ was performed on Drug substance (DS). None of the steps following AEX used to make drug substance impact AAV9 vector empty/full ratio.
²% VG Dilution Yield was calculated as ((VG in diluted affinity pool)/(VG in affinity pool)) * 100%. “Affinity pool” refers to material collected from an affinity column; and is also referred to herein as an “affinity eluate.”
³% VG Column Yield was calculated as ((VG in AEX pool)/(VG in diluted affinity pool)) * 100%. “Diluted affinity pool,” also referred to herein as a “diluted affinity eluate.” In this Example the diluted affinity eluate has not been filtered.
⁴% VG Step Yield was calculated as ((VG in AEX pool)/(VG in affinity pool)) * 100%.
250 L SUB VG/mL resin challenges and % VG Yields were determined using a qPCR method that measured ITR copies.
^{*}2000 L SUB VG/mL resin challenges and % VG Yields were determined using a qPCR method that measured transgene copies.

### Example 9: Ultracentrifugation and Cation Exchange Chromatography for Separation of Empty Capsids from Full AAV Vector Capsids

In another embodiment of the AAV9 vector 250 L SUB downstream process, density gradient ultracentrifugation (UC) was employed to separate empty capsids from full vectors. Similar to a previously described method (Grieger et al. Molecular Therapy (2016) 24(2):287-297), bands that contained 40% and 60% iodixanol were formed in UC tubes. Affinity eluates containing 25% iodixanol were added to the UC tubes and ultra-centrifuged. A fraction enriched in AAV capsids containing a full length vector genome was collected from the interface of the 40% and 60% iodixanol bands. To remove iodixanol, the fraction was diluted and loaded onto a cation exchange (CEX) chromatography column. AAV9 vector capsids bound to the CEX column, and the majority of iodixanol passed through the column in the unbound fraction. AAV9 vector capsids were eluted from the CEX column in a fraction that was substantially free of iodixanol. The CEX pool was processed forward through a UF/DF and 0.2 μm filtration to generate drug substance (DS) comprising AAV9 vector capsids.

Table 25 provides process performance of the UC+CEX and the optimized AEX methods and a comparison of resulting analytics on drug substances produced by these methods. The optimized AEX process, implemented at both the 250 L and 2000 L scales, provided average VG yields of 45±8% and 50±13%, respectively. These values were higher than the average 33±9% VG yield provided by the UC+CEX process. All three methods produced highly similar average DS readouts of SEC A₂₆₀/A280 (1.26-1.30), % full (49-55%), and % empty (20-25%). The average percentage of intermediate capsids was slightly higher in DS produced by the 2000 L AEX process (32±4%) as compared to DS produced by the 250 L AEX (24±3) and 250 L UC+CEX (23±4) processes.

The three methods produced DS with high similarity in % HMMS, % Purity, and levels of HCP and HC-DNA. Collectively, this data shows that the AEX process, implemented at the 250 L and 2000 L SUB scales, provided process performance and product quality highly similar to the 250 L UC+CEX process.

TABLE 25
Drug Substance characterization of AAV9 purified via downstream processes
that enriched for full vectors either via ultracentrifugation and cation
exchange chromatography (UC + CEX), or the optimized AEX method.

| | Capsid Separation Method | UC + CEX | AEX | AEX |
|---|---|---|---|---|
| Inputs | SUB Scale | 250 L | 250 L | 2000 L |
| | No. of DS Batches | 4 | 5 | 5 |
| | No. of Capsid Separation Runs (DS | 26 | 5 | 5 |
| | Sublots) | | | |
| | Capsid Separation % VG Step Yield | 33 ± 9 ^{α} | 45 ± 8 ^{β} | 50 ± 13 ^{β} |
| | (qPCR ^{γ}) | | | |
| Full | A₂₆₀/A₂₈₀ (SEC) | 1.26 ± 0.07 | 1.29 ± 0.03 | 1.30 ± 0.01 |
| Capsid | % Full Capsids (AUC) | 52 ± 11 | 55 ± 7 | 49 ± 2 |
| Characterization | % Intermediate Capsids (AUC) | 23 ± 4 | 24 ± 3 | 32 ± 4 |
| | % Empty Capsids (AUC) | 25 ± 14 | 21 ± 10 | 20 ± 2 |
| Impurity | % HMMS (SEC) | 3.4 ± 0.9 | 2.6 ± 0.8 | 2.9 ± 0.4 |
| Characte | % Purity (RP-HPLC, Non-Reduced) | 98.4 ± 1.3 | 98.6 ± 0.6 | 99.3 ± 0.3 |
| | Residual HCP (pg/1 × 10⁹ VG, | LLOQ, < 2.6 ^{δ} | LLOQ | LLOQ |
| | ELISA) | | | |
| | Residual HC-DNA (pg/1 × 10⁹ VG, | 7.1 ± 3.4 | 17.4 ± 6.7 | 9.3 ± 1.2 |
| | qPCR) | | | |

^{α} UC + CEX % VG step yield accounts for UC and CEX operations.
^{β} AEX % VG step yields account for both Dilution and AEX Chromatography operations.
^{γ} 250 L SUB % VG Step Yield was determined using a qPCR method that measured ITR copies.
2000 L SUB % VG Step Yield was determined using a qPCR method that measured transgene copies.
^{δ} Three UC + CEX drug substance lots had HCP levels that were LLOQ, while one drug substance lot had 2.6 pg HCP/1 × 10⁹ VG.
LLOQ- detected amount was lower that the limit of quantitation of the assay.

### Example 10: Optimized AEX Process for Enrichment of Full AAV3B Vectors

HEK 293 cells were grown in suspension culture and transfected with 2 plasmids to produce AAV3B vector per standard methods known in the art. HEK 293 cells were harvested, lysed, flocculated, and the resulting lysate was filtered. AAV3B vector was purified from the clarified lysate by affinity chromatography. An affinity column was equilibrated, loaded with clarified lysate, washed, and the purified AAV3B vector was eluted. The AAV3B vector affinity pool (also referred to as affinity eluate) was spiked with 25 mM MgCl₂ to achieve a final MgCl₂ concentration of about 1.7 mM in the diluted affinity pool. The pH of the affinity pool was pH 7.6. The affinity pool was diluted about 15-fold (14-fold to 17.8-fold depending on the run) with a buffer comprising 200 mM Histidine, 200 mM Tris, 0.5% P188, pH 8.9. The resulting diluted affinity eluate had a pH of ≥8.6, and a conductivity of ≤2.5 mS/cm (target 2.3 mS/cm), and was loaded on to an AEX column.

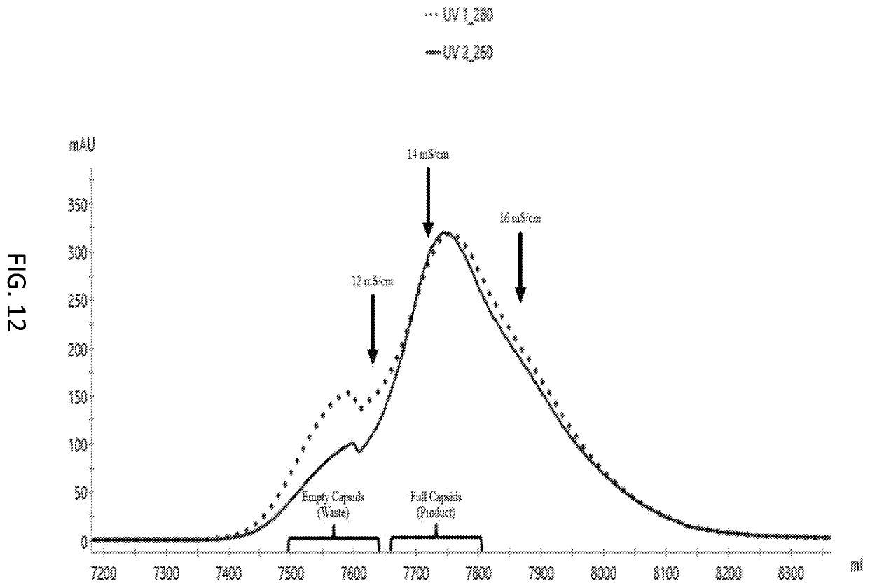

A POROS™ 50 HQ column with a 49 mL column volume, a bed height of cm and a diameter of 2.5 cm was used. The target column load challenge was 1×10¹⁴ to 3×10¹⁴ vg/mL resin (e.g., about 2.4×10¹⁴ vg/mL). Table 26 provides the optimized AEX method conditions. A residence time of 2 minutes/CV was fixed for all steps within the AEX process to accommodate the shallower elution gradient (as compared to previous Examples) and the relatively smaller column. The elution gradient was 2.5 fold more shallow than previous Examples in order to maximize empty/full resolution. Empty capsids eluted first, followed by full AAV3B capsids (FIG. 12). In this Example, the full AAV3B capsids contained a vector genome with a transgene encoding the amino acid of SEQ ID NO:15 (copper transporting ATPase 2 with deletion of metal binding sites 1-4). Consistent with the shallower gradient and broader elution peak, the fraction volume was increased to 0.5 CV (as opposed to 0.33 CV of previous Examples).

TABLE 26
Optimized AEX chromatography method for purification of full rAAV3B vectors.

| | | Load Challenge: | | |
|---|---|---|---|---|
| Bed Height: 10 cm | | 1-3E14 vg/mL resin | | |
| Diameter: 2.5 cm | Cross Sectional Area: 4.9 cm² | | Residence | Linear |
| Step | Column Volume = 49 mL | | Time | Velocity |
| Description | Solution Description | CVs | (min/cv) | (cm/hr) |
| Sanitization 1 | 0.5M NaOH (downward flow) | 8 | 2 | 298 |
| Regeneration 1 | 2M NaCl, 100 mM Tris, pH 9.0 | 5 | 2 | 298 |
| Equilibration 1 | 0.01% P188, 500 mM sodium acetate | 5 | 2 | 298 |
| | 100 mM Tris pH 8.9 | | | |
| Equilibration 2 | 0.5% P188, 200 mM histidine, 200 mM | 5 | 2 | 298 |
| | Tris pH 8.8 | | | |
| Load | Affinity Pool, diluted with 0.5% P188, | N/A | 2 | 298 |
| | 200 mM histidine, 200mM Tris, pH 8.8 | | | |
| Equilibration 3 | 100 mM Tris, 0.01% P188, pH 8.9 | 5 | 2 | 298 |
| Sodium acetate | A - 100 mM Tris, 0.01% P188, pH 8.9 | 37.5 | 2 | 298 |
| gradient elution | B - 500 mM sodium acetate, 100 mM | | | |
| | Tris, 0.01% P188, pH 8.9 | | | |
| | Gradient run from 0-75% over 37.5 CV | | | |
| | Collected 20 fractions of 0.5 CV | | | |
| | from 32%-52% B into vessels | | | |
| Sanitization 2 | 0.5N NaOH (downward flow) | 8 | 2 | 298 |
| Regeneration 2 | 2M NaCl, 100 mM Tris, pH 9.0 | 5 | 2 | 298 |
| Equilibration 4 | 100 mM Tris pH 9.0 | 5 | 2 | 298 |
| Storage | 17% ethanol | 5 | 2 | 298 |

The gradient elution was run from 100% Buffer A to 25% Buffer A/75% Buffer B over 37.5 CV for a slope of 2% Buffer B/CV. When the percentage of Buffer B was 32% to 52% across the gradient, a total of 20 elution fractions were collected. Fractions were collected into vessels pre-charged with 13.2% v/v (0.066 CV) of 250 mM sodium citrate, pH 3.5 to neutralize the fraction and reduce exposure of the capsids to alkaline pH. The pH of the neutralized fractions ranged from pH 7.5 to 7.7. The first elution fraction with an A260/A280≥0.98 (determined by SEC) was pooled with consecutive elution fractions, but no more than a total of 11 factions (Table 27).

TABLE 27
Elution fractions

| | 001 | | 002 | | 004 | | 006 | | 009 | |
|---|---|---|---|---|---|---|---|---|---|---|
| | % vg | A₂₆₀/ | % vg | A₂₆₀/ | % vg | A₂₆₀/ | % vg | A₂₆₀/ | % vg | A₂₆₀/ |
| Batch | per | A₂₈₀ | per | A₂₈₀ | per | A₂₈₀ | per | A₂₈₀ | per | A₂₈₀ |
| No. | load vg | Ratio | load vg | Ratio | load vg | Ratio | load vg | Ratio | load vg | Ratio |
| Fxn1 | 0.35% | 0.62 | 0.34% | 0.62 | 0.44% | 0.62 | 1.02% | 0.66 | 1.01% | 0.69 |
| Fxn2 | 0.71% | 0.65 | 0.78% | 0.66 | 1.03% | 0.67 | 1.99% | 0.74 | 1.76% | 0.77 |
| Fxn3 | 0.15% | 0.71 | 1.84% | 0.75 | 2.09% | 0.75 | 3.74% | 0.86 | 3.05% | 0.89 |
| Fxn4 | 3.25% | 0.81 | 3.89% | 0.88 | 3.89% | 0.88 | 6.52% | 0.97 | 4.65% | 0.99 |
| Fxn5 | 4.29% | 0.91 | 6.08% | 1.01 | 6.28% | 0.99 | 8.63% | 1.05 | 6.44% | 1.02 |
| Fxn6 | 6.59% | 0.99 | 8.81% | 1.08 | 8.50% | 1.05 | 10.61% | 1.08 | 7.34% | 1.02 |
| Fxn7 | 8.21% | 1.00 | 9.88% | 1.06 | 9.17% | 1.03 | 7.70% | 1.06 | 7.07% | 0.98 |
| Fxn8 | 7.84% | 0.97 | 9.71% | 1.04 | 8.75% | 0.98 | 8.88% | 1.01 | 6.64% | 0.94 |
| Fxn9 | 7.11% | 0.92 | 8.02% | 0.99 | 7.53% | 0.91 | 6.83% | 0.94 | 5.47% | 0.91 |
| Fxn10 | 4.79% | 0.89 | 6.82% | 0.95 | 5.29% | 0.85 | 6.03% | 0.87 | 4.42% | 0.91 |
| Fxn11 | 4.56% | 0.87 | 5.51% | 0.93 | 4.61% | 0.81 | 4.55% | 0.81 | 4.05% | 0.91 |
| Fxn12 | 4.42% | 0.86 | 4.89% | 0.91 | 3.73% | 0.78 | 3.94% | 0.78 | 3.45% | 0.91 |
| Fxn13 | 3.78% | 0.86 | 3.99% | 0.90 | 2.93% | 0.77 | 2.58% | 0.78 | 2.70% | 0.90 |
| Fxn14 | 2.96% | 0.86 | 2.94% | 0.91 | 2.33% | 0.76 | 2.02% | 0.77 | 2.14% | 0.90 |
| Fxn15 | 2.23% | 0.85 | 2.21% | 0.90 | 1.73% | 0.75 | 1.66% | 0.79 | 1.93% | 0.91 |
| Fxn16 | 1.70% | 0.85 | 1.76% | 0.89 | 1.31% | 0.81 | 1.08% | 0.81 | 1.43% | 0.91 |
| Fxn17 | 1.15% | 0.85 | 1.21% | 0.90 | 0.95% | 0.82 | 0.50% | 0.80 | 0.99% | 0.93 |
| Fxn18 | 0.78% | 0.86 | 0.98% | 0.90 | 0.71% | 0.84 | 0.51% | 0.83 | 0.77% | 0.92 |
| Fxn19 | 0.57% | 0.85 | 0.63% | 0.88 | 0.46% | 0.80 | 0.33% | 0.85 | 0.55% | 0.93 |
| Fxn20 | 0.42% | 0.82 | 0.48% | 0.84 | 0.32% | 0.65 | 0.18% | 0.78 | 0.34% | 0.90 |

Fractions in bold and underlined were pooled.

The pooled fractions were assayed by various analytical techniques. The actual vg/mL resin challenge ranged from 6.3E13 to 9.4E13 with an average of 7.4E13±1.2E13. SEC was carried out to determine the A260/A280 ratio. The A260/A280 of the AEX pool was increased in all runs as compared to the A260/A280 of the affinity pool (Table 28). The percent full, intermediate and empty capsids of the affinity pool and AEX elute pool were determined by analytical ultracentrifugation. Affinity pools, with an average percent full vectors of 11.2±2.1%, were enriched to 22.9±2.9% full in the AEX pool. The same affinity pools were depleted of empty capsids from 79.7±2.5% to 67.5±3.8% in the AEX pool.

The vg titer was determined by transgene QPCR (Table 28). The average % vg dilution yield was 120%±12%. The average % vg column yield was 47%±11%.

TABLE 28
AEX performance characteristics for
the purification of AAV3B vectors.

| | SEC A260/A280 | | |
|---|---|---|---|
| | Affinity | | |
| Batch ID | Pool | AEX Pool SEC | Δ (AEX-Affinity) |
| 001 | 0.81 | 1.03 | 0.22 |
| 002 | 0.88 | 1.01 | 0.13 |
| 004 | 0.84 | 0.98 | 0.14 |
| 006 | 0.87 | 0.97 | 0.10 |
| 009 | 0.86 | 1.01 | 0.15 |
| AVG +/− StdDev | 0.85 ± 0.025 | 1.00 ± 0.022 | 0.15 ± 0.040 |

| | | | | | | | Δ % Full |
|---|---|---|---|---|---|---|---|
| | Affinity Pool | | | AEX Pool | | | (AEX- |
| Batch ID | % full | % inter. | % empty | % full | % inter. | % empty | Affinity) |
| 001 | 7.2 | 12.9 | 79.9 | 18.7 | 9.9 | 71.4 | 11.50 |
| 002 | 12.5 | 12.7 | 74.8 | 27.7 | 12.1 | 60.3 | 15.20 |
| 004 | 10.7 | 8.5 | 80.7 | 22.8 | 7.7 | 69.5 | 12.10 |
| 006 | 13.1 | 5.0 | 81.9 | 22.3 | 9.1 | 68.7 | 9.20 |
| 009 | 12.1 | 6.8 | 81.0 | 23.1 | 9.3 | 67.6 | 11.00 |
| AVG +/− | 11.1 ± 2.1 | 9.2 ± 3.2 | 79.7 ± 2.5 | 22.9 ± 2.9 | 9.6 ± 1.4 | 67.5 ± 3.8 | 11.8 ± 2.0 |
| StdDev | | | | | | | |

| | VG Titer via qPCR | | |
|---|---|---|---|
| | VG/mL resin | % VG Dilution | % VG Column |
| Batch ID | Challenge | Yield | Yield |
| 001 | 6.4E+13 | 113% | 35% |
| 002 | 7.1E+13 | 119% | 67% |
| 004 | 6.3E+13 | 121% | 45% |
| 006 | 9.4E+13 | 141% | 47% |
| 009 | 8.0E+13 | 105% | 42% |
| AVG +/− StdDev | 7.4E13 ± 1.2E13 | 120% ± 12% | 47 ± 11% |

The method described in this Example was used to produce purified rAAV3B vector pools that were enriched for full capsids and depleted of empty capsids as compared to the starting material, that is an affinity eluate.

We claim:

1. A method of purifying an rAAV vector by AEX, the method comprising a step of:
i) loading a solution comprising the rAAV vector to be purified onto an AEX stationary phase in a column;
ii) performing gradient elution of a material from the stationary phase in the column wherein a percentage of a first gradient elution buffer is varied in a manner inversely proportional to variation in a percentage of a second gradient elution buffer; and
iii) collecting at least one fraction of eluate from the column during the gradient elution beginning when the absorbance of a column flow-through reaches an absorbance threshold, and wherein the at least one fraction of eluate comprises the rAAV vector to be purified.

2. The method of purifying a rAAV vector by AEX of claim 1, wherein the method further comprises measuring an absorbance of the at least one fraction of eluate collected from the column and determining an A260/A280 ratio.

3. The method of purifying a rAAV vector by AEX of claim 1 or 2, wherein the solution comprising the rAAV vector to be purified is diluted about 2-fold to 25-fold (e.g., 15-fold) with a dilution solution comprising histidine, Tris and P188, and optionally filtered prior to application to the stationary phase.

4. The method of purifying a rAAV vector by AEX of any one of claims 1-3, wherein the solution is an affinity eluate.

5. The method of purifying a rAAV vector by AEX of claim 5, wherein a pH of the diluted, and optionally filtered affinity eluate is increased as compared to a pH of the solution; and wherein a conductivity of the diluted, and optionally filtered affinity eluate is decreased as compared to a conductivity of the solution.

6. The method of purifying a rAAV vector by AEX of any one of claims 1-5, wherein the first gradient elution buffer comprises about 50 mM to about 150 mM Tris, about 0.005% to about 0.015% P188 and has a pH of about pH 8.5 to 9.5; wherein the second gradient elution buffer comprises about 400 mM to about 600 mM sodium acetate, about 50 mM to about 150 mM Tris, about 0.005% to about 0.015% P188 and has a pH of about pH 8.5 to 9.5; and wherein 10 to 60 column volumes (CV) (e.g., about 20 CV, about 37.5 CV) of the first gradient elution buffer, the second gradient elution buffer or a mixture of both are applied to the stationary phase during the gradient elution.

7. The method of purifying a rAAV vector by AEX of any one of claims 1-6, wherein at a start of the gradient elution the percentage of the first gradient elution buffer is 50%-100% and at an end of the gradient elution the percentage of the second gradient elution buffer is 50%-100% and wherein the percentage of the second elution buffer increases at a rate of about 2% to 5% per CV over the gradient elution.

8. The method of purifying a rAAV vector by AEX of any one of claims 1-7, wherein a concentration of sodium acetate of the first gradient elution buffer, the second gradient elution buffer or the mixture of both increases continuously during the gradient elution; and wherein the concentration of the sodium acetate increases at a rate of about 10 mM/CV to 50 mM/CV (e.g., about 10 mM/CV, about 25 mM/CV) over the gradient elution.

9. The method of purifying a rAAV vector by AEX of any one of claims 1-8, wherein full capsids are eluted from the stationary phase in a first elution peak and/or in a first portion of a second elution peak during the gradient elution.

10. The method of purifying a rAAV vector by AEX of any one of claims 1-9, wherein empty capsids are recovered in the column flow-through, in a first elution peak and/or in a last portion of a second elution peak during the gradient elution.

11. The method of purifying a rAAV vector by AEX of any one of claims 1-10, wherein an absorbance of the at least one fraction of eluate is measured at 280 nm, and wherein optionally, an absorbance threshold is ≥0.5 mAU/mm path length measured at 280 nm.

12. The method of purifying a rAAV vector by AEX of any one of claims 1-11, wherein a volume of the at least one fraction of eluate is equivalent to ⅛ of a CV to 10 CV, e.g., ⅛ of a CV, ¼ of a CV, ⅓ of a CV, ½ of a CV, ¾ of a CV, 1 CV, 2 CV, 3 CV, 4 CV, 5 CV, 6 CV, 7 CV, 8 CV, 9 CV, 10 CV or more of a CV, and optionally, wherein an A260/A280 ratio of the at least one fraction of eluate is ≥ to 1.25.

13. The method of purifying a rAAV vector by AEX of any one of claims 1-12, wherein at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more, fractions of eluate are collected.

14. The method of purifying a rAAV vector by AEX of any one of claims 1-13, wherein the method further comprises combining at least two fractions of eluate collected from the column, each having an A260/A280 ratio of ≥0.98 or ≥1.0 to form a pooled eluate comprising the rAAV vector.

15. The method of purifying a rAAV vector by AEX of claim 14, wherein the pooled eluate has a % VG column yield of 20% to 100% (e.g., 63+/−26%), a % VG step yield of 31% to 66% (e.g., 47+/−11%) and/or an A260/A280 ratio of ≥1.0.

16. The method of purifying a rAAV vector by AEX of claim 14 or 15, wherein the pooled eluate is enriched for full capsids, and/or depleted of empty capsids, as compared to the solution loaded onto the column.

17. The method of purifying a rAAV vector by AEX of any one of claims 1-16, wherein a purified rAAV vector is produced.

18. The method of purifying a rAAV vector by AEX of any one of claims 14-17, further comprising filtering the pooled eluate by a method selected from the group consisting of viral filtration, ultrafiltration/diafiltration (UF/DF), filtration through a 0.2 μm filter and a combination thereof, to produce a drug substance.

19. The method of purifying a rAAV vector by AEX of claim 18, wherein the drug substance comprises: i) 45% to 65% (e.g., 52+/−7%) full capsids of total capsids; ii) 19% to 37% (e.g., 28+/−5%) intermediate capsids of total capsids; and/or iii) 10% to 37% (e.g., 20+/−7%) empty capsids of total capsids.

20. The method of purifying a rAAV vector by AEX of claim 18 or 19, wherein the drug substance is enriched for full capsids, and/or depleted of empty capsids, as compared to the solution loaded onto the column.

21. The method of purifying a rAAV vector by AEX of any one of claims 1-20, wherein the rAAV vector comprises an AAV capsid protein from an AAV serotype selected from the group consisting of AAV1, AAV2, AAV3, AAV3A, AAV3B, AAV4, AAV5, AAV6, AAV7, AAV8, AAV9, AAV10, AAVrh10, AAVrh74, AAV12, AAV2i8, NP4, NP22, NP66, AAVDJ, AAVDJ/8, AAVDJ/9, AAVLK03, AAV1.1, AAV2.5, AAV6.1, AAV6.3.1, AAV9.45, RHM4-1 (SEQ ID NO:5 of WO 2015/013313), RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15.

22. The method of purifying a rAAV vector by AEX of any one of claims 1-21, wherein the rAAV vector comprises an AAV9 capsid protein and a transgene comprising the nucleic acid of SEQ ID NO:1.

23. The method of purifying a rAAV vector by AEX of any one of claims 1-21, wherein the rAAV vector comprises an AAV3B capsid protein and a transgene comprising a nucleic acid encoding the amino acid sequence of SEQ ID NO:15.

24. A method of preparing a solution comprising a rAAV vector for purification by AEX, the method comprising a step of:
i) diluting a first solution 2 to 25-fold (e.g., 15-fold) with a dilution solution comprising histidine, Tris and P188; and optionally
ii) filtering the first solution from step i) through a filter to produce a diluted, and optionally filtered solution;
wherein the pH of the diluted, and optionally filtered solution is increased as compared to the pH of the first solution; and wherein the conductivity of the diluted, and optionally filtered solution is decreased as compared to the conductivity of the first solution.

25. The method of preparing a solution comprising a rAAV vector for purification by AEX of claim 24, wherein the first solution comprising the rAAV vector is selected from the group consisting of an affinity eluate, a supernatant from a cell lysate and a post-harvest solution.

26. The method of preparing a solution comprising a rAAV vector for purification by AEX of claim 24 or 25, wherein the dilution solution comprises about 100 mM to 300 mM (e.g., about 200 mM) histidine, 100 mM to 300 mM (e.g., about 200 mM) Tris, 0.1% to 1.0% (e.g., about 0.5%) P188 and has a pH of pH 8.5 to 9.5.

27. The method of preparing a solution comprising a rAAV vector for purification by AEX of any one of claims 24-26, wherein i) a pH of the diluted, and optionally filtered solution is 8.5 to 9.5; ii) a conductivity of the diluted, and optionally filtered solution is 1.7 to 3.3 mS/cm; and/or iii) a % VG dilution yield of the diluted solution is 35% to 100%.

28. The method of preparing a solution comprising a rAAV vector for purification by AEX of any one of claims 24-27, wherein the rAAV vector comprises an AAV9 capsid protein or an AAV3B capsid protein.

29. A purified rAAV vector prepared by a method comprising a step of:
i) loading a solution comprising the rAAV vector to be purified onto an AEX stationary phase in a column;
ii) performing gradient elution of a material from the stationary phase in the column wherein a first gradient elution buffer, a second gradient elution buffer or a mixture of both are applied to the stationary phase and a concentration of a salt is varied from 0 mM to 500 mM such that the rate of increase in concentration of the salt over the course of the gradient elution is about 10 mM/CV to 50 mM/CV (e.g., about 25 mM/CV);
iii) collecting at least one fraction of eluate from the column during gradient elution beginning when absorbance of a column flow-through reaches an absorbance threshold; and/or
vi) measuring an absorbance of the at least one fraction of eluate collected from the column and determining an A260/A280 ratio.

30. The purified rAAV vector prepared by the method of claim 29, wherein the method further comprises combining at least two fractions of eluate collected from the column when the A260/A280 ratio is ≥1.0 to form a pooled eluate comprising the purified rAAV vector.

31. The purified rAAV vector prepared by the method of claim 29 or 30, wherein the salt is sodium acetate.

32. The purified rAAV vector prepared by the method of any one of claims 29-31, wherein the rAAV vector comprises an AAV9 capsid protein or an AAV3B capsid protein.

33. The purified rAAV vector prepared by the method of any one of claims 29-32, wherein the solution comprising the rAAV vector is an affinity eluate that has been diluted and optionally filtered prior to loading onto the stationary phase.

34. The purified rAAV vector prepared by the method of any one of claims 29-33, wherein the material eluted from the stationary phase comprises the rAAV vector.

35. The purified rAAV vector prepared by the method of any one of claims 30-34, wherein the method further comprises filtering the pooled eluate by a method selected from the group consisting of viral filtration, ultrafiltration/diafiltration (UF/DF), filtration through a 0.2 μm filter and a combination thereof, to produce a drug substance.

36. The purified rAAV vector prepared by the method of claim 35, wherein the drug substance is used to make a drug product suitable for administration to a human subject to treat a disease, disorder or condition.

37. The purified rAAV vector prepared by the method of claim 36, wherein the disease, disorder or condition is DMD or Wilson's disease, and optionally wherein the rAAV vector comprises a nucleic acid encoding the amino acid sequence of SEQ ID NO:2 or SEQ ID NO:15.

38. An solution comprising a rAAV vector for purification by AEX prepared by a method comprising a step of:
i) diluting an affinity eluate 2 to 25-fold (e.g., 15-fold) with a dilution solution comprising histidine, Tris, and P188; and optionally
ii) filtering the affinity eluate from step i) through a 0.2 μm filter to produce a diluted, and optionally filtered affinity eluate;
wherein a pH of the diluted, and optionally filtered affinity eluate is increased as compared to a pH of the affinity eluate; and wherein a conductivity of the diluted, and optionally filtered affinity eluate is decreased as compared to a conductivity of the affinity eluate.

39. The solution comprising a rAAV vector for purification by AEX prepared by a method of claim 38, wherein the dilution solution comprises about 100 mM to 300 mM (e.g., about 200 mM) histidine, 100 mM to 300 mM (about 200 mM) Tris, 0.1% to 1.0% (about 0.5%) P188, pH 8.5 to 9.5.

40. The solution comprising a rAAV vector for purification by AEX prepared by a method of claim 38 or 39, wherein the pH the affinity eluate is 3.0 to 4.4 prior to a step of diluting, and optionally filtering, and the pH of the affinity eluate after the step of diluting, and optionally filtering, is 8.5 to 9.5 or 8.7 to 9.0 (e.g., 8.8, 9.0).

41. The solution comprising a rAAV vector for purification by AEX prepared by a method of any one of claims 38-40, wherein the conductivity of the affinity eluate is 5.0 to 7.0 mS/cm (e.g., 5.5 to 6.5 mS/cm) prior to the step of diluting, and optionally filtering, and the conductivity of the affinity eluate after the step of diluting, and optionally filtering, is 1.7 to 3.3 mS/cm, 1.8 to 2.8 mS/cm, and/or 2.2 to 2.6 mS/cm.

42. The solution comprising a rAAV vector for purification by AEX prepared by a method of any one of claims 38-41, wherein the % VG dilution yield of the diluted and optionally filtered affinity eluate is 35% to 100%.

43. The solution comprising a rAAV vector for purification by AEX prepared by a method of any one of claims 38-42, wherein the rAAV vector comprises an AAV9 or an AAV3B capsid protein.

44. The solution comprising a rAAV vector for purification by AEX prepared by a method of any one of claims 38-43, wherein the diluted, and optionally filtered affinity eluate is loaded on an AEX stationary phase.

45. A method of preparing a stationary phase for use in a method of purifying a rAAV vector by AEX of any one of claims 1-23, the method comprising at least one step of:
i) pre-use flushing comprising application of ≥4.5 CV (e.g., about 5 CV) of water for injection to an AEX stationary phase in a column;
ii) sanitizing comprising application of about 14.4 to 17.6 CV (e.g., about 16 CV) of a solution comprising about 0.1 M to 1.0 M NaOH to the AEX stationary phase in the column, optionally by upward flow; and/or
iii) regenerating comprising application of about 4.5 to 5.5 CV (e.g., about 5 CV) of a solution comprising about 1 M to 3 M NaCl, 50 mM to 150 mM Tris, pH 8.5 to 9.5 to the AEX stationary phase in the column.

46. A method of regenerating an AEX stationary phase, the method comprising a step of:
i) post-use sanitizing the stationary phase comprising application of 14.4 to 17.6 CV (e.g., about 16 CV) of a solution comprising about 0.1 M to 1.0 M NaOH to the stationary phase, optionally by upward flow;
ii) regenerating the stationary phase comprising application of 4.5 to 5.5 CV (e.g., about 5 CV) of a solution comprising about 1 M to 3 M NaCl, 50 mM to 150 mM Tris, pH 8.5 to 9.5 to the stationary phase;
iii) equilibrating the stationary phase comprising application of 4.5 to 5.5 CV (e.g., about 5 CV) of a solution comprising about 50 mM to 150 mM Tris, pH 8.5 to 9.5 to the stationary phase;
iv) post-use flushing the stationary phase comprising application of ≥4.5 (e.g., about 5 CV) of water for injection to the stationary phase; and/or
v) applying a storage solution to the stationary phase comprising application 2.7 to 3.3 CV (e.g., about 3 CV) of a solution comprising about 17% to 17.5% ethanol to the stationary phase.

47. The method of regenerating an AEX stationary phase of claim 46, wherein any one of steps i)-v) step follows a chromatography elution step of a method of purifying a rAAV vector by AEX.

48. A regenerated AEX stationary phase prepared by a method comprising a step of:
i) post-use sanitizing of the stationary phase comprising application of 14.4 to 17.6 CV (e.g., about 16 CV) of a solution comprising about 0.1 M to 1.0 M NaOH to the stationary phase, optionally by upward flow;
ii) regenerating the stationary phase comprising application of 4.5 to 5.5 CV (e.g., about 5 CV) of a solution comprising about 1 M to 3 M NaCl, 50 mM to 150 mM Tris, pH 8.5 to 9.5 to the stationary phase;
iii) equilibrating the stationary phase comprising application of 4.5 to 5.5 CV (e.g., about 5 CV) of a solution comprising about 50 mM to 150 mM, Tris, pH 8.5 to 9.5 to the stationary phase;
iv) post-use flushing of the stationary phase comprising application of ≥4.5 (e.g., about 5 CV) of water for injection to the stationary phase; and/or
v) applying a storage solution to the stationary phase comprising application 2.7 to 3.3 CV (e.g., about 3 CV) of a solution comprising about 17% to 17.5% ethanol to the stationary phase.

49. A regenerated AEX stationary phase of claim 48, wherein the regenerated AEX stationary phase is used for purification of a rAAV vector.

50. A method of purifying an rAAV vector by AEX, the method comprising a step of:
i) loading a solution comprising the rAAV vector to be purified onto an AEX stationary phase in a column;
ii) performing gradient elution of a material from the stationary phase in the column wherein a percentage of a first gradient elution buffer is varied in a manner inversely proportional to variation in a percentage of a second gradient elution buffer; wherein at the start of the gradient elution the percentage of the first gradient elution buffer is about 75% to about 100% and at the end of the gradient elution the percentage of the second gradient elution buffer is about 60% to about 100%; and wherein the percentage of the second elution buffer increases at a rate of about 2% to 5% per CV over the gradient elution;
iii) collecting at least one fraction of eluate from the column when performing the gradient elution beginning when the percentage of the second gradient elution buffer is about 30% to about 35%,
and wherein the at least one fraction of eluate comprises the rAAV vector to be purified.

51. The method of purifying an rAAV vector by AEX of claim 50, wherein the solution comprising the rAAV vector is an affinity elutate that has been diluted about fold with a buffer comprising histidine, Tris and P188.

52. The method of purifying an rAAV vector by AEX of claim 50 or 51, wherein the first gradient elution buffer comprises 50 mM to 150 mM (e.g., about 100 mM) Tris, 0.005% to 0.015% (e.g., about 0.01%) P188, pH 8.5 to 9.5 (e.g., 8.9) and/or the second gradient elution buffer comprises 400 mM to 600 mM (e.g., about 500 mM) sodium acetate, 50 mM to 150 mM (e.g., about 100 mM) Tris, 0.005% to 0.015% (e.g., about 0.01%) P188, pH 8.5 to 9.5 (e.g., pH 8.9).

53. The method of purifying an rAAV vector by AEX of any one of claims 50-52, wherein collecting at least one fraction of eluate from the column comprises collecting the at least one fraction of eluate into a vessel comprising about 0.01 CV to 0.1 CV (e.g., about 0.066 CV) of a solution comprising 200 mM to 300 mM (e.g., about 250 mM) sodium citrate, pH 3.0 to 4.0 (e.g., about 3.5).

54. The method of purifying a rAAV vector by AEX of any one of claims 51-53, wherein the at least one fraction of eluate is enriched for full capsids, and/or depleted of empty capsids, as compared to the affinity eluate; optionally wherein the rAAV vector is a rAAV3B vector; and optionally wherein the AEX stationary phase is POROS™ 50 HQ.

55. The method of purifying a rAAV vector by AEX of any one of claims 50-54, wherein the collecting at least one fraction of eluate from the column when performing the gradient elution ends when the percentage of the second gradient elution buffer is about 50% to about 55%.
